SEQUENCE LISTING (309 sequences)

SEQ ID NO: 1 - protein, 907 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIIIYLIYTRQRRLCMQPLQNLFPYLVSADGTTVTSGNTKDTSLQAPPSYEESVYNSGRKGPGPPSSDASTAAPPYTNEQAYQMLLALVRLDAEQRAQQNGTDSLDGQTGTQDKGQKPNLLDRLRHRKNGYRHLKDSDEEENV

SEQ ID NO: 2 - protein, 907 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGRTGTQDKGQKPNLLDRLRHRKNGYRHLKDSDEEENV

SEQ ID NO: 3 - protein, 906 aa, synthetic construct
MESRIWCLVVCVNLCIVCLGAAVSSSSTSHATSSTHNGSHTSRTTSAQTRSVYSQHVTSSEAVSHRANETIYNTTLKYGDVVGVNTTKYPYRVCSMAQGTDLIRFERNIICTSMKPINEDLDEGIMVVYKRNIVAHTFKVRVYQKVLTFRRSYAYIYTTYLLGSNTEYVAPPMWEIHHINKFAQCYSSYSRVIGGTVFVAYHRDSYENKTMQLIPDDYSNTHSTRYVTVKDQWHSRGSTWLYRETCNLNCMLTITTARSKYPYHFFATSTGDVVYISPFYNGTNRNASYFGENADKFFIFPNYTIVSDFGRPNAAPETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETSGGLVVFWQGIKQKSLVELERLANRSSLNITHRTRRSTSDNNTTHLSSMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGQTGTQDKGQKPNLLDRLRHRKNGYRHLKDSDEEENV

SEQ ID NO: 4 - protein, 682 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKV

SEQ ID NO: 5 - protein, 659 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVF

SEQ ID NO: 6 - protein, 681 aa, synthetic construct
GAAVSSSSTSHATSSTHNGSHTSRTTSAQTRSVYSQHVTSSEAVSHRANETIYNTTLKYGDVVGVNTTKYPYRVCSMAQGTDLIRFERNIICTSMKPINEDLDEGIMVVYKRNIVAHTFKVRVYQKVLTFRRSYAYIYTTYLLGSNTEYVAPPMWEIHHINKFAQCYSSYSRVIGGTVFVAYHRDSYENKTMQLIPDDYSNTHSTRYVTVKDQWHSRGSTWLYRETCNLNCMLTITTARSKYPYHFFATSTGDVVYISPFYNGTNRNASYFGENADKFFIFPNYTIVSDFGRPNAAPETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETSGGLVVFWQGIKQKSLVELERLANRSSLNITHRTRRSTSDNNTTHLSSMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKV

SEQ ID NO: 7 - protein, 681 aa, synthetic construct; identical to SEQ ID NO: 6

SEQ ID NO: 8 - protein, 205 aa, synthetic construct
MKMEELFKKHKIVAVLRANSVEEAKEKALAVFEGGVHLIEITFTVPDADTVIKELSFLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 9 - protein, 205 aa, synthetic construct
  REGION 1..2: note = optional residues
MKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 10 - protein, 205 aa, synthetic construct
  VARIANT 33: note = E
  VARIANT 61: note = K
  VARIANT 187: note = D
  VARIANT 190: note = R
  REGION 1..2: note = optional residues
MKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 11 - protein, 205 aa, synthetic construct
  VARIANT 33: note = E
  VARIANT 61: note = K
  VARIANT 187: note = D
  VARIANT 190: note = R
  REGION 1..2: note = optional residues
MKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 12 - protein, 205 aa, synthetic construct
  VARIANT 33: note = E
  VARIANT 61: note = K
  VARIANT 187: note = D
  VARIANT 190: note = R
  REGION 1..2: note = optional residues
MKMEELFKKHKIVAVLRANSVEEAKMKALAVFVGGVHLIEITFTVPDADTVIKELSFLKELGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTIAEVAAKAAAFVEKIRGCTE

SEQ ID NO: 13 - protein, 205 aa, synthetic construct
  VARIANT 33: note = E
  VARIANT 61: note = K
  VARIANT 187: note = D
  VARIANT 190: note = R
  REGION 1..2: note = optional residues
MKMEELFKKHKIVAVLRANSVEEAKKKALAVFLGGVDLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVQAVGVGEALNKGTPVEVAEKAKAFVEKIRGCTE

SEQ ID NO: 14 - protein, 205 aa, synthetic construct
  VARIANT 33: note = E
  VARIANT 61: note = K
  VARIANT 187: note = D
  VARIANT 190: note = R
  REGION 1..2: note = optional residues
MKMEELFKKHKIVAVLRANSVEEAKKKALAVFMGGVDLIEITFTVPDADTVIKELSFLKELGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVQAVGVGEALNKGTPAEVAEKAKAFVEKIRGCTE

SEQ ID NO: 15 - protein, 155 aa, synthetic construct
  REGION 1..2: note = N-terminal optional residues
MGKYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 16 - protein, 155 aa, synthetic construct
  REGION 1..2: note = N-terminal optional residues
MGKYDGSKLRIGILHARGNAEIILALVLGALKRLQEFGVKRENIIIETVPGSFELPYGSKLFVEKQKRLGKPLDAIIPIGVLIRGSTPHFDYIADSTTHQLMKLNFELGIPVIFGVITADTDEQAEARAGLIEGKMHNHGEDWGAAAVEMATKFN

SEQ ID NO: 17 - protein, 152 aa, synthetic construct
  REGION 1..2: note = N-terminal optional residues
MGKYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESDEQAEERAGTKAGNHGEDWGAAAVEMATKFN

SEQ ID NO: 18 - protein, 120 aa, synthetic construct
  REGION 1: note = N-terminal optional residue
MEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 19 - protein, 205 aa, synthetic construct
  REGION 1: note = N-terminal optional residue
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 20 - protein, 157 aa, synthetic construct
  REGION 1: note = N-terminal optional residue
MNQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAA

SEQ ID NO: 21 - protein, 205 aa, synthetic construct
  REGION 1: note = N-terminal optional residue
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCEWFKAGVLAVGVGDALVKGDPDEVREKAKKFVEKIRGCTE

SEQ ID NO: 22 - protein, 205 aa, synthetic construct
  REGION 1: note = N-terminal optional residue
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVEAMKGPFPNVKFVPTGGVDLDDVCEWFDAGVLAVGVGDALVEGDPDEVREDAKEFVEEIRGCTE

SEQ ID NO: 23 - protein, 205 aa, synthetic construct
  REGION 1: note = N-terminal optional residue
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIRGCTE

SEQ ID NO: 24 - protein, 157 aa, synthetic construct
  REGION 1: note = N-terminal optional residue
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVIDGMMNVQLDTGVPVLSAVLTPHRYRDSDAHTLLFLALFAVKGMEAARACVEILAAREKIAA

SEQ ID NO: 25 - protein, 157 aa, synthetic construct
  REGION 1: note = N-terminal optional residue
MNQHSHKDHETVRIAVVRARWHAEIVDACVSAFEAAMRDIGGDRFAVDVFDVPGAYEIPLHARTLAETGRYGAVLGTAFVVDGGIYDHEFVASAVIDGMMNVQLDTGVPVLSAVLTPHEYEDSDADTLLFLALFAVKGMEAARACVEILAAREKIAA

SEQ ID NO: 26 - protein, 157 aa, synthetic construct
  REGION 1: note = N-terminal optional residue
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 27 - protein, 167 aa, synthetic construct
MLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKVELIGNENHGLYLADQYVKGIAKSRKS

SEQ ID NO: 28 - protein, 165 aa, synthetic construct
MLKPEMIEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDAQN

SEQ ID NO: 29 - protein, 166 aa, synthetic construct
NFHQDCEAGLNRTVNLKFHSSYVYLSMASYFNRDDVALSNFAKFFRERSEEEKEHAEKLIEYQNQRGGRVFLQSVEKPERDDWANGLEALQTALKLQKSVNQALLDLHAVAADKSDPHMTDFLESPYLSESVETIKKLGDHITSLKKLWSSHPGMAEYLFNKHTLG

SEQ ID NO: 30 - protein, 169 aa, synthetic construct
QFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 31 - protein, 172 aa, synthetic construct
SGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 32 - protein, 25 aa, synthetic construct
MELLILKANAITTILTAVTFCFASG

SEQ ID NO: 33 - protein, 19 aa, synthetic construct
MSWKVMIIISLLITPQHGL

SEQ ID NO: 34 - protein, 17 aa, synthetic construct
MKAILVVLLYTFTTANA

SEQ ID NO: 35 - protein, 18 aa, synthetic construct
MPISILLIITTMIMASHC

SEQ ID NO: 36 - protein, 15 aa, synthetic construct
MFVFLVLLPLVSSQC

SEQ ID NO: 37 - protein, 19 aa, synthetic construct
MVPQVLLFVPLLGFSLCFG

SEQ ID NO: 38 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYV

SEQ ID NO: 39 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 40 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 41 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFNENEIMQLFNSFKQVLDYV

SEQ ID NO: 42 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFNENEIMQLFNSFKQVLDYVEDALQ

SEQ ID NO: 43 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNNENSIMQLFESFKQVLDYV

SEQ ID NO: 44 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNNENSIMQLFESFKQVLDYVEKALQ

SEQ ID NO: 45 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNHENSISQLAESFKSVLDRV

SEQ ID NO: 46 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNHENSISQLAESFKSVLDRVEKALE

SEQ ID NO: 47 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 48 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 49 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFNENEIMQLFNSFKQVLDYV

SEQ ID NO: 50 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFNENEIMQLFNSFKQVLDYVEDALQ

SEQ ID NO: 51 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNNENSIMQLFESFKQVLDYV

SEQ ID NO: 52 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNNENSIMQLFESFKQVLDYVEKALQ

SEQ ID NO: 53 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNHENSISQLAESFKSVLDRV

SEQ ID NO: 54 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNHENSISQLAESFKSVLDRVEKALE

SEQ ID NO: 55 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 56 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 57 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 58 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 59 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 60 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 61 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 62 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 63 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 64 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 65 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTTYVAPPMWEIHHINSHSQCYSSYSRVIAGTVFVAYHRDSYENKTMQLMPDDFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 66 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTTYVAPPMWEIHHINSHSQCYSSYSRVIAGTVFVAYHRDSYENKTMQLMPDDFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 67 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 68 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 69 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 70 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 71 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 72 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 73 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 74 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 75 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 76 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 77 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTTYVAPPMWEIHHINSHSQCYSSYSRVIAGTVFVAYHRDSYENKTMQLMPDDFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 78 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTTYVAPPMWEIHHINSHSQCYSSYSRVIAGTVFVAYHRDSYENKTMQLMPDDFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 79 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 80 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 81 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 82 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 83 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 84 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 85 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 86 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 87 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 88 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 89 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 90 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 91 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 92 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 93 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 94 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 95 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 96 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 97 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 98 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 99 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 100 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 101 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 102 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 103 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYV

SEQ ID NO: 104 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 105 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 106 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFNENEIMQLFNSFKQVLDYV

SEQ ID NO: 107 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFNENEIMQLFNSFKQVLDYVEDALQ

SEQ ID NO: 108 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNNENSIMQLFESFKQVLDYV

SEQ ID NO: 109 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNNENSIMQLFESFKQVLDYVEKALQ

SEQ ID NO: 110 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNHENSISQLAESFKSVLDRV

SEQ ID NO: 111 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNHENSISQLAESFKSVLDRVEKALE

SEQ ID NO: 112 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 113 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 114 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFNENEIMQLFNSFKQVLDYV

SEQ ID NO: 115 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFNENEIMQLFNSFKQVLDYVEDALQ

SEQ ID NO: 116 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNNENSIMQLFESFKQVLDYV

SEQ ID NO: 117 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNNENSIMQLFESFKQVLDYVEKALQ

SEQ ID NO: 118 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNHENSISQLAESFKSVLDRV

SEQ ID NO: 119 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNHENSISQLAESFKSVLDRVEKALE

SEQ ID NO: 120 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 121 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 122 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 123 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 124 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 125 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 126 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 127 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 128 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 129 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 130 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTTYVAPPMWEIHHINSHSQCYSSYSRVIAGTVFVAYHRDSYENKTMQLMPDDFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 131 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTTYVAPPMWEIHHINSHSQCYSSYSRVIAGTVFVAYHRDSYENKTMQLMPDDFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 132 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 133 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 134 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 135 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 136 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 137 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 138 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 139 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 140 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 141 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 142 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTTYVAPPMWEIHHINSHSQCYSSYSRVIAGTVFVAYHRDSYENKTMQLMPDDFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 143 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTTYVAPPMWEIHHINSHSQCYSSYSRVIAGTVFVAYHRDSYENKTMQLMPDDFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 144 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 145 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 146 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 147 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 148 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 149 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 150 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 151 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 152 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 153 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 154 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 155 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 156 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 157 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 158 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 159 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 160 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 161 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 162 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 163 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 164 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 165 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 166 - protein, 678 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 167 - protein, 683 aa, synthetic construct
GAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 168 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYV

SEQ ID NO: 169 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 170 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 171 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFNENEIMQLFNSFKQVLDYV

SEQ ID NO: 172 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFNENEIMQLFNSFKQVLDYVEDALQ

SEQ ID NO: 173 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNNENSIMQLFESFKQVLDYV

SEQ ID NO: 174 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNNENSIMQLFESFKQVLDYVEKALQ

SEQ ID NO: 175 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNHENSISQLAESFKSVLDRV

SEQ ID NO: 176 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNHENSISQLAESFKSVLDRVEKALE

SEQ ID NO: 177 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 178 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 179 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFNENEIMQLFNSFKQVLDYV

SEQ ID NO: 180 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFNENEIMQLFNSFKQVLDYVEDALQ

SEQ ID NO: 181 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNNENSIMQLFESFKQVLDYV

SEQ ID NO: 182 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNNENSIMQLFESFKQVLDYVEKALQ

SEQ ID NO: 183 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNHENSISQLAESFKSVLDRV

SEQ ID NO: 184 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNHENSISQLAESFKSVLDRVEKALE

SEQ ID NO: 185 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 186 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 187 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 188 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 189 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 190 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 191 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 192 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 193 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 194 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 195 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTTYVAPPMWEIHHINSHSQCYSSYSRVIAGTVFVAYHRDSYENKTMQLMPDDFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 196 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTTYVAPPMWEIHHINSHSQCYSSYSRVIAGTVFVAYHRDSYENKTMQLMPDDFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 197 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 198 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 199 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 200 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 201 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 202 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 203 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 204 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 205 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 206 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 207 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTTYVAPPMWEIHHINSHSQCYSSYSRVIAGTVFVAYHRDSYENKTMQLMPDDFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 208 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTTYVAPPMWEIHHINSHSQCYSSYSRVIAGTVFVAYHRDSYENKTMQLMPDDFSNTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 209 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 210 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 211 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 212 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 213 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 214 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 215 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 216 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDDFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 217 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 218 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 219 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 220 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 221 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 222 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 223 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 224 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 225 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 226 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 227 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 228 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCFSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 229 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 230 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 231 - protein, 607 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 232 - protein, 612 aa, synthetic construct
YRVCSMAQGTDLIRFERNIVCTSMKPINEDLDEGIMVVYKRNIVAHTFKVRVYQKVLTFRRSYAYHRTTYLLGSNTEYVAPPMWEIHHINSHSQCYSSYSRVIAGTVFVAYHRDSYENKTMQLMPDCYSSTHSTRYVTVKDQWHSRGSTNLTRETSNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNSTKSSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 233 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYV

SEQ ID NO: 234 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 235 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 236 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFNENEIMQLFNSFKQVLDYV

SEQ ID NO: 237 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFNENEIMQLFNSFKQVLDYVEDALQ

SEQ ID NO: 238 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNNENSIMQLFESFKQVLDYV

SEQ ID NO: 239 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNNENSIMQLFESFKQVLDYVEKALQ

SEQ ID NO: 240 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNHENSISQLAESFKSVLDRV

SEQ ID NO: 241 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNHENSISQLAESFKSVLDRVEKALE

SEQ ID NO: 242 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 243 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 244 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFNENEIMQLFNSFKQVLDYV

SEQ ID NO: 245 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFNENEIMQLFNSFKQVLDYVEDALQ

SEQ ID NO: 246 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNNENSIMQLFESFKQVLDYV

SEQ ID NO: 247 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNNENSIMQLFESFKQVLDYVEKALQ

SEQ ID NO: 248 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNHENSISQLAESFKSVLDRV

SEQ ID NO: 249 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNTNHENSISQLAESFKSVLDRVEKALE

SEQ ID NO: 250 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 251 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 252 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 253 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 254 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 255 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 256 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 257 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 258 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 259 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 260 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTTYVAPPMWEIHHINSHSQCYSSYSRVIAGTVFVAYHRDSYENKTMQLMPDDFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 261 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTTYVAPPMWEIHHINSHSQCYSSYSRVIAGTVFVAYHRDSYENKTMQLMPDDFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 262 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 263 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 264 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 265 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 266 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 267 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 268 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 269 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 270 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 271 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSCVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 272 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTTYVAPPMWEIHHINSHSQCYSSYSRVIAGTVFVAYHRDSYENKTMQLMPDDFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 273 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTTYVAPPMWEIHHINSHSQCYSSYSRVIAGTVFVAYHRDSYENKTMQLMPDDFSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 274 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 275 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 276 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 277 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 278 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 279 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 280 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 281 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANSSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 282 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 283 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 284 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 285 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 286 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 287 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 288 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 289 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 290 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 291 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFISAKMTAFFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 292 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYV

SEQ ID NO: 293 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCFSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYAQKELRSSNVFDLEEIMRLFNSFKQVLKYVEDAVQ

SEQ ID NO: 294 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 295 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVYYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 296 - protein, 607 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYV

SEQ ID NO: 297 - protein, 612 aa, synthetic construct
YRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDCYSSTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCLTINQTSVKVLRDMNVKESPGRCYSRPVVIFNFANCSYVFYGQLGEDNEILLGNHRTEECQLPSLKIFIAGNSAYEYVDYLFKRMIDLSSISTVDSMIALDIDPLENTDFRVLELYSQKELRSSNVFDLEEIMRLFNSFKQVLKYVEDAVQ

SEQ ID NO: 298-299 - skipped (2 entries)

SEQ ID NO: 300 - protein, 4 aa, synthetic construct
GSGS

SEQ ID NO: 301 - protein, 12 aa, synthetic construct
GSGGSGSGSGGS

SEQ ID NO: 302 - protein, 12 aa, synthetic construct
GSGGSGSGSGGS

SEQ ID NO: 303 - protein, 14 aa, synthetic construct
GSEKAAKAEEAARK

SEQ ID NO: 304 - protein, 32 aa, synthetic construct
MGILPSPGMPALLSLVSLLSVLLMGCVAETGT

SEQ ID NO: 305 - protein, 11 aa, synthetic construct
GGSHHHHHHHH

SEQ ID NO: 306 - protein, 39 aa, synthetic construct
GSGYIPEAPRDGQAYVRKDGEWVLLSTFLGGHHHHHHHH

SEQ ID NO: 307 - protein, 45 aa, synthetic construct
GSRMKQIEDKIEEILSKIYHIENEIARIKKLIGERGGHHHHHHHH

SEQ ID NO: 308 - protein, 26 aa, synthetic construct
GYIPEAPRDGQAYVRKDGEWVLLSTF

SEQ ID NO: 309 - protein, 26 aa, synthetic construct
IEDKIEEILSKIYHIENEIARIKKLI

(19) United States
(12) Patent Application Publication
(10) Pub. No.: US 2026/0132171 A1
(43) Pub. Date: May 14, 2026

(54) PRE-FUSION-STABILIZED CMV GB PROTEIN NANOSTRUCTURES

(71) Applicant: University of Washington, Seattle, WA (US)

(72) Inventors: Neil P. KING, Seattle, WA (US); Quinton M. DOWLING, Seattle, WA (US); Daniel ELLIS, Seattle, WA (US); David VEESLER, Seattle, WA (US); Alexandra C. WALLS, Seattle, WA (US)

(21) Appl. No.: 19/478,009

(22) PCT Filed: Apr. 30, 2024
(86) PCT No.: PCT/US2024/026962

§ 371 Date: Oct. 23, 2025

Related U.S. Application Data
(60) Provisional application No. 63/499,381, filed on May 1, 2023.

(51) Int. Cl. C07K 14/03; A61K 39/00; A61K 39/245; C12N 15/63

(52) U.S. Cl. CPC C07K 14/03; A61K 39/245; C12N 15/63; A61K 2039/6031; A61K 2039/62; A61K 2039/645; C07K 2319/00; C12N 2710/16122; C12N 2710/16134

(57) ABSTRACT

Provided herein are compositions and methods related to CMV gB proteins in which amino acid substitutions have been made to disrupt the postfusion state and/or stabilize the prefusion state.

### SEQUENCE LISTING STATEMENT

This application contains a sequence listing; which is submitted electronically and is hereby incorporated by reference in its entirety. The sequence listing submitted herewith is contained in the XML filed created Apr. 25, 2024 entitled “21-1661-WO_Sequence-Listing.xml” and is 449,423 bytes in size.

### BACKGROUND

Cytomegalovirus (CMV) infections maintain a high prevalence, with viruses capable of infecting both fibroblasts and epithelial cells, and commonly causing persistent latent infections through immune system evasion. CMV is a leading cause of congenital diseases worldwide, and also poses higher threats of severe disease to immunocompromised individuals. No vaccine is currently commercially available for either prevention of congenital disease or general infection. Two viral surface glycoprotein complexes are of primary interest for the elicitation of protective antibody responses, including glycoprotein B (gB) and the pentamer complex, gB is a class Ill viral fusion protein that is used for entry to all cell types, while the pentamer complex mediates attachment to epithelial cells, gB is metastable and can adopt multiple distinct conformations, with recombinant forms often characterized in a postfusion state that does not structurally represent the functional prefusion state observed on virions. Consequently, there is a need for altering the structural conformation of gB antigens to stabilize their prefusion state and for use in vaccines to more effectively prevent viral entry into cells.

Accordingly, there is an unmet need for CMV gB proteins in which amino acid substitutions have been made to disrupt the postfusion state and/or stabilize the prefusion state.

### SUMMARY

The present disclosure relates generally to CMV gB proteins in which mutations have been added to disrupt the postfusion state and/or stabilize the prefusion state. Further, the disclosure provides nanostructures that incorporate such CMV gB proteins.

In one aspect, the disclosure provides a polypeptide comprising an ectodomain of CMV gB in the prefusion conformation, wherein the ectodomain comprises, 1, 2, 3, 4 or more amino acid substitutions selected from the group consisting of E686L, R693V, V694L, Y690F, K700A, V702E, D679H, E681N, K695D, L680E, R685Q, V701L, E682S, F678N, N688E, V677T, D699K, F687A, M684S, Q692S, and Y696R, or substitutions at the same amino acid positions; the amino acid substitution S367I, the amino acid substitution T374F, or the amino acid substitutions S367I and T374F, or substitutions at positions 367 and/or 374; 1, 2, 3, 4 or more amino acid substitutions selected from the group consisting of Q591F, Q591Y, S668A, Y218F, N220S, V552L, or substitutions at the same amino acid positions; the amino acid substitution E167T; and/or 1, 2, 3, 4 or more amino acid substitutions selected from the group consisting; of C246S, H157R, I156H, R457S, R460S, W240N, and Y242T at positions relative to SEQ ID NO: 1.

In some embodiments, the ectodomain comprises the amino acid subsitutions D217C and S587C. In some embodiments, the ectodomain comprises the amino acid subsitutions D217C and Y589C.

In some embodiments, the ectodomain comprises 1, 2, 3, 4 or more amino acid substitutions selected from the group consisting of E686L, R693V, V694L, Y690F, K700A, V702E, D679H, E681N, K695D, L680E, R685Q, V701L, E682S, E678N, N688E, V677T, D699K, F687A, M684S, Q692S, and Y696R.

In some embodiments, the ectodomain comprises the amino acid substitutions E686L, R693V, V694L, and Y690F. In some embodiments, the ectodomain comprises the amino acid substitutions E686L, R693V, V694L, Y690F, K700A, and V702Q, In some embodiments, the ectodomain comprises the amino acid substitutions E686L, R693V, V694L, Y690F, D679N. E681N, K695D, L680E, and R685Q. In some embodiments, the ectodomain comprises the amino acid substitutions E686L, R693V, V694L, Y690F, K700A, V702Q, D679N. E681N, K695D, L680E, R685Q, and V70 IL. In some embodiments, the ectodomain comprises the amino acid substitutions E686L, R693V, V694L, Y690F. D679N, E681N, K695D, L680E, R685Q, E682S, F678N, N688E, and V677T. In some embodiments, the ectodomain comprises the amino acid substitutions E686L, R693V, V6941L, Y690F, K700A, V702Q, D679N, E68 IN, K695D, L680E, R685Q, V701L, E682S, F678N, N688E, V677T, and D699K. In some embodiments, the ectodomain comprises the amino acid substitutions E686L, R693V, V694L, Y690F, D679H, E681N, K695D, L680E, R685Q, E682S, F678N, N688E, V677T, F687A, M684S, Q692S, and Y696R. In some embodiments, the ectodomain comprises the amino acid substitutions E686L, R693V, V694L, Y690F, K700A, V702E. D679H, E681N, K695D, L680E, R685Q, V701L, E682S, F678N, N688E, V677T, D699K, F687A, M684S, Q692S, and Y696R.

In sone embodiments, the ectodomain comprises the amino acid substitution S367I or the amino acid substitution T374F. In some embodiments, the ectodomain comprises the amino acid substitutions S367I and T374F.

In some embodiments, the ectodomain comprises 1, 2, 3, 4 or more amino acid substitutions selected from the group consisting of Q591F, Q591 Y, S668A, Y218F, N220S. and V552L. In some embodiments, the ectodomain comprises the amino acid substitutions Q591F, S668A, and Y218F. In some embodiments, the ectodomain comprises the amino acid substitutions N220S and V552L. In some embodiments, the ectodomain comprises the amino acid substitutions Q591F, S668A, Y218F, N220S, and V552L. In some embodiments, the ectodomain comprises the amino acid substitutions Q591Y, S668A, Y218F, N220S, and V552L. In some embodiments, the ectodomain comprises the amino acid substitutions Q591Y, N220S, and V552L. In some embodiments, the ectodomain comprises the amino acid substitutions Q591F, N220S, and V552L.

In some embodiments, the ectodomain comprises amino acid substitution E167T or E167S. In some embodiments, the ectodomain comprises 1, 2, 3, 4 or more amino acid substitutions selected from the group consisting of C246S 1-1157R, 115611, R457S, R460S, W240N, and Y242T.

In some embodiments, the polypetide comprises any one combination of amino acid substitutions listed in Table 10.

In some embodiments, the polypetide comprises a polypeptide sequence at least 75%, 80%, 85%, 90%, 95%, 96%, 97%, 98%, 99%, or 100% identical to a polypeptide sequence in Table 10 without the signal peptide, or an antigenic fragment thereof.

In some embodiments, the CMV gB protein adopts a prefusion conformation in the absence of a fusion inhibitor. In some embodiments, the fusion inhibitor is N-{4-({(1S)-1-3,5-bis(trifluoromethyl)phenyl]ethyl}carbonothioyl) amino]phenyl}-1,3-thiazole-4-carboxamide.

In some embodiments, the polypeptide comprises, as a C-terminal fusion to the ectodomain, a trimerization domain. In some embodiments, the polypeptide comprises, as a C-terminal fusion to the ectodomain, a nanostructure assembly domain.

In some embodiments, the assembly domain comprises a polypeptide sequence at least 75%, 80%, 85%, 90%, 95%, 96%, 97%, 98%, 99%, or 100% identical to 153-50A (SEQ ID NO: 19), 153-50A.1 (SEQ ID NO: 21) 153-50A.1NegT2 (SEQ ID NO: 22), or 153-50A.1PosT1 (SEQ ID NO: 23). In some embodiments, the assembly domain comprises a polypeptide sequence at least 75%, 80%, 85%, 90%, 95%, 96%, 97%, 98%, 99%, or 100% identical to 13-01 (SEQ ID NO: 8). In some embodiments, the assembly domain comprises a polypeptide sequence at least 75%, 80%, 85%, 90%, 95%, 96%, 97%, 98%, 99%, or 100% identical to SEQ ID NO: 27, SEQ ID NO: 28, SEQ ID NO: 29, SEQ ID NO: 30, or SEQ ID NO: 31. In some embodiments, the assembly domain comprises a polypeptide sequence at least 75%, 80%, 85%, 90%, 95%, 96%, 97%, 98%, 99%, or 100% identical to 153 dn5B (SEQ ID NO: 18).

In another aspect, the disclosure provides a nanostructure comprising any polypeptide of the disclosure.

In some embodiments, the nanostructure comprises a second polypeptide. In some embodiments, the second polypetpide comprises a polypeptide sequence at least 75%, 80%, 85%, 90%, 95%, 96%, 97%, 98%, 99%, or 100% identical to 153-50B (SEQ ID NO: 20), 153-50B.1 (SEQ ID NO: 24), 153-50B.1NegT2 (SEQ ID NO: 25), or 153-50B.4PosT1 (SEQ ID NO: 26). In some embodiments, the second polypeptide comprises a polypeptide sequence at least 75%, 80%, 85%, 90%, 95%, 96%, 97%, 98%, 99%, or 100% identical to T53_dn5A* (SEQ ID NO: 15), 53_dn5A.1 (SEQ TD NO: 16), or I53_dn5A.2 (SEQ ID NO: 17).

In another aspect, the disclosure provides a polynucleotide encoding any polypeptide of the disclosure or any nanostructure of the disclosure.

In some embodiments, the polynucleotide is a messenger RNA (mnRNA).

In another aspect, the disclosure provides a lipid nanoparticle (LNP) comprising an mRNA encoding any polypeptide of the disclosure or any nanostructure of the disclosure.

In another aspect, the disclosure provides a pharmaceutical composition comprising any polypetide of the disclosure, any nanostructure of the disclosure, any polynucleotide of the disclosure or the LNP of the disclosure.

In another aspect, the disclosure provides a method of generating an immune response to CMV gB protein in a subject in need thereof, comprising administering to the subject, an effective amount of the pharmaceutical composition of the disclosure.

In another aspect, the disclosure provides a method of treating or preventing CMV infection in a subject in need thereof, comprising administering to the subject, an effective amount of the pharmaceutical composition of the disclosure.

In another aspect, the disclosure provides an expression vector comprising any polynucleotide of the disclosure operatively linked to a suitable control sequence.

In another aspect, the disclosure provides a host cell comprising any polynucleotide of the disclosure, any expression vector of the disclosure, and/or any polypeptide of the disclosure.

In another aspect, the disclosure provides a polypeptide comprising an amino acid sequence at least 90%, 91%, 92%, 93%, 94%, 95%, 96%, 97%, 98%, or 99% identical to the amino acid sequence of SEQ TD NO:1 or SEQ ID NO:3 residues 91-702, or an antigenic fragment thereof, wherein residues 698-702 are optional if they do not include a mutation, SEQ ID NO:3 residue 456 is absent, and the polypeptide or antigenic fragment thereof comprises 1 or more combination of mutations relative to SEQ ID NO:1 or SEQ ID NO:3 residues 91-702 listed in Table 2. Table 3, or Table 4. In aspects, any polypeptide or antigenic fragment of the disclosure thereof further comprises 1, 2, 3, 4, 5, 6, 7, 8, 9, 10, I1, 12, 13, 14, 15, 16, 17, 18, 19, 20, or more mutations relative to relative to SEQ ID NO:1 or SEQ ID NO:3 residues 91-702 listed in Table 5, Table 6, Table 7, or Table 8. In aspects, the polypeptide or antigenic fragment thereof includes an amino acid sequence at least 90%, 91%, 92%, 93%, 94%, 95%, 96%, 97%, 98%, or 99% identical to an amino acid sequence listed in Table 9. In aspects, the polypeptide or antigenic fragment thereof includes an amino acid sequence at least 90%, 91%, 92%, 93%, 94%, 95%, 96%, 97%, 98%, or 99% identical to an amino acid sequence sequences listed in Table 10. In another aspect, the disclosure provides a fusion protein comprising the polypeptide or antigenic fragment thereof of the disclosure and a multimerization domain.

In some embodiments, the multimerization domain of the fustion protein of the disclosure comprises a polypeptide with sequence identity of at least 50%, 55%, 60%, 65%, 70%, 75%, 80%, 85%, 90% 95%, 96%, 97%, 98%, 99%, or 100% to the sequence of a protein listed in Table 12, wherein residues in parentheses are optional.

In another aspect, the disclosure provides a composition comprising one or more of the polypeptides or fusion proteins of the disclosure linked to a scaffold. In aspects, the scaffold is a protein scaffold.

In another aspect, the disclosure provides a nucleic acid encoding the polypeptide or fusion protein of any of the disclosure.

In another aspect, the disclosure provides an expression vector comprising the nucleic acid of the disclosure operatively linked to a suitable control sequence.

In another aspect, the disclosure provides a host cell comprising any nucleic acid of the disclosure, any expression vector of the disclosure, and/or any polypeptide or fusion protein of the disclosure.

In another aspect, the disclosure provides a pharmaceutical composition, comprising one or more of the polypeptides, fusion protein, composition, nucleic acid, expression vector, and/or the host cell of the disclosure and a pharmaceutically acceptable carrier.

In another aspect, the disclosure provides a vaccine comprising one or more of the polypeptides, fusion protein, composition, nucleic acid, expression vector, and/or the host cell of the disclosure and a pharmaceutically acceptable carrier.

In some embodiments, the vaccine of the disclosure further comprising any other component as appropriate for an intended use, including but not limited to any other CMV antigens, including but not limited to one or more of CMV proteins gH, gL, UL128, UL130 and UL131, or antigenic portions thereof; or a pentamer complex of CMV proteins gH, gL, UL128, UL130 and UL131 or antigenic portions thereof.

In another aspect, the disclosure provides a method for treating or limiting development of an CMV infection, comprising administering to a subject in need thereof an amount effective to treat or limit development of the CMV infection of a polypeptide, fusion protein, composition, vaccine, nucleic acid, expression vector, host cell, pharmaceutical composition, and/or vaccine of the disclosure.

In another aspect, the disclosure provides a composition, method, or use as described herein.

### BRIEF DESCRIPTION OF THE DRAWINGS

FIG. 1 shows structural representation of regions targeted in prefusion CMV gB structure for addition of engineered mutations. A) Prefusion structure of CMV gB (PDB 7KDP), with only the ectodomain shown. Highlighted regions contain sets of stabilizing mutations according to the names displayed. Without being bound by theory, mutations listed as “Bg-KOs” are used to reduce aggregation, improve expression and/or prevent cleavage by furin. B) Postfusion structure of CMV gB (PDB 7KDD).

FIG. 2 shows addition of glycan at N165. A) N165 (shown with spheres) is solvent-exposed in the prefusion structure (PDB 7KDP, shown with transparent surface). Two views are shown, one of which is further zoomed-in (right). B) N165 (shown with spheres) is buried in the postfusion structure. Two views are shown, one from the side of gB (left) and one from the bottom of gB looking down the 3-fold axis (right).

FIG. 3 shows recombinant gB designs including at least the soluble ectodomain of gB from the Towne strain from residues 20-698, with some designs containing additional residues up to residue 704. All designs tested featured an exogenous N-terminal signal peptide and a C-terminal octa-histidine tag, with some designs also featuring an exogenous trimerization domain between the C-terminus of the gB ectodomain and the octa-histidine tag.

FIG. 4 shows chromatographic elution profiles of multiple gB antigens featuring designed mutations, showing homogenous profiles consistent with expected elution profiles for trimeric gB antigens. A comparable gB antigen lacking designed mutations is shown as a reference.

FIG. 5 shows DLS of multiple gB antigens featuring designed mutations, showing distinctly different hydrodynamic diameters and polydispersities, which is indicative of conformational changes. A comparable gB antigen lacking designed mutations is shown as a reference.

FIG. 6 shows nanoDSF of multiple gB antigens featuring designed mutations, with nanoDSF measured using intrinsic tryptophan fluorescence. A comparable gB antigen lacking designed mutations is shown as a reference.

FIG. 7 shows NS-EM micrographs of multiple gB antigens featuring designed mutations, showing disruption of the postfusion state. A comparable gB antigen lacking designed mutations is shown as a reference, which more monodispersely features an elongated profile consistent with the postfusion state.

FIG. 8 shows NS-EM 2D class averages of one gB antigen featuring designed mutations and a comparable gB antigen lacking designed mutations.

### DETAILED DESCRIPTION

### Definitions

Throughout the disclosure, reference is made to particular features (including method steps). It is to be understood that the disclosure includes all possible combinations of such particular features. For example, where a particular feature is disclosed in the context of a particular aspect or embodiment, or a particular claim, that feature can also be used, to the extent possible, in combination with and/or in the context of other particular aspects and embodiments.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The practice of the technology will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. It is to be understood that this disclosure is not limited to the particular methodology, protocols, and reagents described, as these may vary, depending upon the context in which they are used by those of skill in the art.

All publications and patents mentioned herein are hereby incorporated by reference in their entirety as if each individual publication or patent was specifically and individually indicated to be incorporated by reference. In case of conflict, the present application, including any definitions herein, controls. However, mention of any reference, article, publication, patent, patent publication, and patent application cited herein is not, and should not be taken as an acknowledgment, or any form of suggestion, that they constitute valid prior art or form part of the common general knowledge in any country in the world.

The section headings used herein are for organizational purposes only and are not to be construed as limiting the subject matter described. Specifically, features described in one section may be combined with features in any other section of the description.

While illustrative embodiments are described and depicted, it will be appreciated that various changes can be made to these illustrative embodiments without departing from the spirit and scope of the invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole.

The singular forms “a”, “an”, and “the” are intended to include the plural forms as well, unless the context clearly indicates otherwise.

As used herein, the term “about” means a range of values including the specified value, which a person of ordinary skill in the art would consider reasonably similar to the specified value. In some embodiment, about means within a standard deviation using measurements generally acceptable in the art. In some embodiment, about means a range extending to +/−10%, +/−5%, +/−3%, or +/−1% of the specified value.

The term “at least” followed by a number is used herein to denote the start of a range beginning with that number (which may be a range having an upper limit or no upper limit, depending on the variable being defined). For example, “at least 1” means 1 or more than 1.

The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)−(a second number)” this means a range whose lower limit is the first number and whose upper limit is the second number. For example, 25 to 100 mm means a range whose lower limit is 25 mm, and whose upper limit is 100 mm.

Throughout this specification, unless the context requires otherwise, the words “comprise”, “comprises” and “comprising” will be understood to imply the inclusion of a stated step or element or group of steps or elements but not the exclusion of any other step or element or group of steps or elements. For example, a composition “comprising” (or “which comprises”) components A, B, and C can consist of (i.e., contain only) components A, B, and C, or can contain not only components A, B, and C but also one or more other components.

As used herein, the term “consisting of” refers to including, and limited to, whatever follows the phrase “consisting of,” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The term “protein nanostructure,” as used herein, refers to symmetric protein assemblies in which the subunits self-assemble in aqueous solution, without requiring lipids or macromolecules other than the protein nanostructure for assembly. Illustrative protein nanostructures are described in Hsia et al. Nature 35:136-9 (2016) and Bale et al. Science 353:389-394 (2016). In some embodiments, the protein nanostructure is a one-component protein nanostructure, in which a single polypeptide type provides the building blocks to self-assemble to form the protein nanostructure. In some embodiments, the protein nanostructure is a two-component protein nanostructure, in which two polypeptide types provide the building blocks to self-assemble to form the protein nanostructure. In some embodiments, the polypeptide types include an assembly domain, which causes the polypeptides to form symmetric dimeric, trimeric, tetrameric, hetaxameric components, or another multimeric component. In a two-component nanostructure, the two components differ in the selection of an assembly domain. In some embodiments, the assembly domain of the first polypeptide type causes the polypetide to form a trimer; and the assembly domain of the second polypeptide type causes the polypetide to form a pentamer.

In sone embodiments of, or relating to, one-component nanostructures, two or more copies of the component further symmetrically self-assemble to form the nanostructure. In some embodiments of, or relating to, two-component nanostructures, two or more of each of the two, different components symmetrically self-assemble to form the nanostructure.

The protein nanostructures disclosed herein can display potentially antigenic polypeptides intended to elicit immune responses to viruses and can be administered as vaccines. In some embodiments, the components include atrimer of antigenic polypeptides.

The vaccines of the present disclosure are useful for preventing and/or decreasing the severity of viral infection.

The term “ferritin protein nanostructure,” as vised herein, refers to using ferritin to generate a symmetric, protein-based protein nanostructure using naturally occurring ferritin sequences, or engineered variants thereof. Ferritin-based protein nanostructures are prepared by fusing an antigen to the ferritin molecule, Illustrative ferritin protein nanostructures are described (vising the term nanoparticle rather than protein nanostructure) in U.S. Pat. Appl. Pub. No. US 2018/0021258 A1 and US 2019/033027 A1, the contents of which are incorporated by reference herein. Further, the molecular architecture of ferritin, which consists of 24 subunits assembling into an octahedral cage with 432 symmetry has the potential to display multimeric antigens on its surface.

As used herein, the term “assembly domain” refers to the portion of a subunit of a component involved in forming the protein nanostructure through intra-component interations and interactions with either other copies of the same component (in a one-component nanostructure) or with other components (e.g., in a two-component nanostructure).

The term “icosahedral particle” refers to protein nanostructures having a core with icosahedral symmetry. 153 refers to an icosahedral particle constructed from pentamers and trimers. 152 refers to an icosahedral particle constructed from pentamers and dimers. T33 refers to a tetrahedral particle constructed from two sets of trimers. T32 refers to a tetrahedral particle constructed from trimers and dimers.

The potentially antigenic polypeptides may be attached to the core of the protein nanostructure either non-covalently or covalently, including as a fusion protein or by other means disclosed herein. Multimeric polypeptides may optionally be displayed along a symmetry axis of the protein nanostructure. Also provided are proteins and nucleic acid molecules encoding such proteins, formulations, and methods of use.

The term “antigen” or “immunogen” refers to its plain and ordinary meaning of a compound or composition that induces an immune response, cellular or humoral, e.g., cytotoxic T lymphocyte (CTL) response, a B cell response (for example, production of antibodies that specifically bind the epitope), an NK cell response or any combinations thereof, when administered to or expressed in an immunocompetent subject. Antigens can include polypeptides (including glycoproteins). In some embodiment, an antigen is a polypeptide or polypeptide complex that elicits an immune response. For example, an antigen can include one or more immunogenic epitopes associated with a viral pathogen. The tern antigen, as used herein, is not limited to the portion of the polypeptide or polypeptide complex that contains antigenic epitopes. An “epitope” or “antigenic determinant” refers to its plain an ordinary meaning as the part of an antigen that is recognized by the immune system, specifically by antibodies, B cells, or T cells.

The terms “polypeptide”, “peptide” and “protein” are used interchangeably herein to refer to a polymer of amino acid residues and optionally one or more post-translational modifications (e.g., glycosylation) and/or other modifications.

The term “isolated” when applied to a polynucleotide or polypeptide, denotes that the polynucleotide or polypeptide is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high-performance liquid chromatography. A polynucleotide or polypeptide that is the predominant species present in a preparation is substantially purified.

The terms “virus” or “virus particle” are used according to its plain ordinary meaning within Virology and refers to a virion including the viral genome (e.g., DNA, RNA, single strand, double strand), viral capsid and associated proteins, and in the case of enveloped viruses (e.g., herpesvirus), an envelope including lipids and optionally components of host cell membranes, and/or viral proteins.

The term “viral infection” or “viral disease” refers to a disease or condition that is caused by a virus, such as by Cytomegalovirus (CMV).

The ten-n “ectodomain” refers to the portion of a transmembrane protein or glycoprotein that, in the native state of the protein, is on the outside of the cellular or viral membrane.

The term “variant” refers to a polypeptide or polynucleotide having one or more insertions, deletions, or amino acid substitutions relative to a reference polypeptide or polynucleotide.

The term “antigenic variant” refers to a variant that has one or more epitopes in common with a reference polypeptide and/or generates the same or similar immune response when administered to a subject as a reference polypeptide.

The term “functional variant” refers to a variant that exhibits at least some of the activity as a reference polypeptide. For example, a functional variant of an assembly domain is able to promote multimerization and self-assembly to the same extent, or to similar extent, as a reference assembly domain and/or is able to multimerize and assembly with the same cognate assembly domains as a reference assembly domain.

As used herein, the term “bound” when referring to two moieties means the two moieties are bonded, wherein the bond or bonds connecting the two moieties may be covalent or non-covalent. In some embodiment, the two moieties are covalently bonded to each other (e.g., directly or through a covalently bonded intermediary (a “linker”)). In some embodiment, the two moieties are non-covalently bonded (e.g., through ionic bond(s), Van der Waal's bond(s)/interactions, hydrogen bond(s), polar bond(s), or combinations or mixtures thereof). For a fusion polypeptide that is N-terminally and C-terminally joined the linker can be a peptide bond or peptide of any length.

The term “domain” refers to refers to any portion of a polypeptide that adopts a tertiary structure.

The terms “multimerization domain” and “multimerize” refer to the ability of a polypeptide, or domain of a polypeptide, to form a tertiary structure with another polypeptide of domain of a polypeptide. In some embodiments, multimerization domains can form dimers, trimers, tetramers, pentamers, or hexamers and/or to form heteromers with other multimerization domains.

The ten-n “trimerization domain” refers to a multimerization domain that forms trimers.

The term “fragment” refers to a polypeptide having one or more N-terminal or C-terminal truncations compared to a reference polypeptide.

The term “functional fragment” refers to a fragment that retains at least one function of its reference polypeptide.

The terms “helix” or “helical” refer to an α-helical secondary structure in a polypeptide that is known to occur, or predicted to occur. For example, a sequence may be described as helical when computational modeling suggests the sequence is likely to adopt a helical conformation.

The term “component” refers to a protein, or protein complex, capable of assembly into a protein nanostructure under appropriate conditions (e.g., a fusion protein comprising an assembly domain).

The term “vaccine” refers to a composition that can provide active acquired immunity to and/or therapeutic effect (e.g., treatment) of a particular disease or a pathogen. A vaccine typically contains one or more agents that can induce an immune response in a subject against a pathogen or disease, i.e., a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g., preventing or ameliorating the effects of a future infection by any natural or pathogen, or of an anticipated occurrence of cancer in a predisposed subject) or therapeutic (e.g., treating; cancer in a subject who has been diagnosed with the cancer). The administration of vaccines is referred to vaccination.

The term “pharmaceutically acceptable excipients” and “pharmaceutically acceptable carrier” refer to a substance that aids the administration of an active agent to and absorption by a subject and can be included in the compositions of the present disclosure without causing a significant adverse toxicological effect on the patient and can mean excipients approved by a regulatory agency of the Federal or a state government or listed in the U.S. Pharmacopeia or other generally recognized pharmacopeia for use in animals, and more particularly in humans. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “adjuvant” refers to a pharmaceutically acceptable substance that enhances the immune response to an antigen when co-administered with the antigen or administered before, during, or after administration of the antigen to a subject. In some cases, the LNP polynucleotide (e.g., mRNA) can serve as the adjuvant.

The term “TLR4 immunostimulant” refers to an adjuvant that stimulates Toll-like Receptor 4 (TLR4) in the immune cells of a subject to modulate an immune response. Exemplary TLR4 immunostimulants include, but are not limited to, Monophosphoryl Lipid A (MPL), Glucopyranosyl Lipid A (GLA), and/or synthetic lipid A (SLA). In some embodiment, the antigen is a TLR4 immunostimulant.

The term “effective amount” refers to the amount of a composition that, when administered to a patient for treating a state, disorder or condition is sufficient to effect such treatment or when administered to a patient for generating an immune response is sufficient to generate such an immune response. The exact amounts will depend on the active ingredient, the state, disorder, or condition to be treated and its severity, and the age, weight, physical condition and responsiveness of the subject to be treated, and will be ascertainable by one skilled in the art using known techniques (see, e.g., Liebennan, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The terms “immunization” and “immunizing” refer to administering a composition to a subject in an amount sufficient to elicit, after one or more administering steps, a desired immune response, immunization may comprise between one and ten, or more administrations (e.g., injections) of the composition, such as 1, 2, 3, 4, 5, 6, 7, 8, 9, 10, or more administrations. The first administration may elicit no detectable immune response as generally each subsequent administration will boost the immune response generated by prior administrations. The term “immunizing” as used herein includes post-exposure prophylaxis.

The term “protective immune response” refers to an immune response that prevents and/or reduces the severity of infection with a pathogen when the subject is later challenged with the pathogen, or to an immune response that generates a level of immune response that correlates with protection. For example, vaccination may generate a protective immune response if it results in production, in the plasma or serum, of the subject (e.g., human, pet, or agricultural animal), of neutralizing antibodies that protect the subject against subsequent infection and/or are present in a quantity observed to confer protection upon test subjects (e.g., New Zealand White (NZW) rabbits).

The term “polyclonal antibody response” refers to an antibody response comprising antibodies having more than one specificities and/or variation in their antibody sequences.

The term “neutralizing” (e.g., “neutralizing antibody response”) refers to antibodies that prevent infection and/or reduce the level of infection by a pathogen. A neutralizing antibody response can be measured either in in vitro assays (e.g., infection of cells in culture by a pathogen in the presence of the antibody) or in an in vivo assay (e.g., by determining a protective dose of an antibody through administering the antibody to a subject prior to challenge with an infective dose of a pathogen).

An antibody “binds to” or is “specific to” or “specifically binds” (used interchangeably herein) to a target (e.g., viral protein) are terms well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances.

The term “post-exposure prophylaxis” refers to administering an antigen (e.g., a vaccine) to a subject previously exposed to and/or infected with a pathogen in order to elicit an immune response to protect against infection by the pathogen and/or decrease the severity of one or more symptoms of infection by the pathogen.

The term “administering” refers to providing a composition to a subject in a manner that permits the composition to have its intended effect. Administration for vaccination or post-exposure prophylaxis may be performed by intramuscular injection, intravenous injection, intraperitoneal injection, or any other suitable route.

“Co-administer” means that a composition described herein is administered at the same time, just prior to, or just after the administration of one or more additional therapies. The composition provided herein can be administered alone or can be coadministered to the subject. Co-administration is meant to include simultaneous or sequential administration of the compounds individually or in combination (more than one compound). Thus, the preparations can also be combined, when desired, with other active substances (e.g., to reduce metabolic degradation).

The term “subject” refers to a human or non-human animal to which a composition may be administered for vaccination, treatment, or other purpose. In some embodiment, the non-human animal is a non-human primate, rabbit, hamster, gerbil, pig, cow, sheep, goat, guinea pig, rat, mouse, squirrel, wolf, fox, horse, zebra, giraffe, elephant, cat, dog, llama, or ferret.

The term “manufacturing” refers to production of a recombinant polynucleotide, polypeptide, delivery vehicle, or protein nanostructure at any scale, including at least 25-mL, 50-mL, 1-L, 1,000-L, 50,000-L, or greater scale.

The terms “culturing” and “culture medium” refers to standard cell culture and recombinant protein expression techniques.

The term “host cell” refers to any cell capable of use in expression of a recombinant polypeptide or polynucleotide.

The term “secretes” refers to the ability of host cells to release expressed polypeptides into the media in which they are cultured.

The term “signal sequence” refers to a polypeptide sequence, typically at the N terminus of a polypeptide, expressed in a host cell that directs the polypeptide to a particular cellular compartment. A signal sequence may be a secretion signal to cause the host cell to secrete the polypeptide into the media in which with host cell is cultured. The signal sequence can be the “native” signal sequence, a signal sequence that occurs in nature as part of the polypeptide. The signal sequence can be a sequence that does not naturally occur with the polypeptide as found in nature. Various signal sequences are known and it is within the skill of an ordinary artisan to select an appropriate signal sequence.

The term “purify” refers to separating a molecule from other substances present in a composition. Polypeptides may be purified by affinity (e.g., to an antibody or to a tag, e.g., using a His-tag capture resin), by charge (e.g., ion-exchange chromatography), by size (e.g., preparative ultracentrifugation, size exclusion chromatography), or otherwise.

The terms “polynucleotide” and “nucleic acid,” used interchangeably herein, refer to a polymeric form of nucleotides of more than about 100 nucleotides, either ribonucleotides or deoxyribonucleotides. Thus, this term includes, but is not limited to, single-, double-, or multi-stranded DNA or RNA, mRNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligoners” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

The terms “identity”, “identical”, and “sequence identity” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region). Such sequences are then said to be “substantially identical.” This definition also refers to, or may be applied to, the compliment of a test sequence. The definition also includes sequences that have deletions and/or additions, as well as those that have substitutions. The term “amino acid substitution” refers to replacing a single amino acid in a sequence with another amino acid residue. The standard form of abbreviations for amino acid substitution are used. For example, V94R refers to substitution of valine (V) in a reference sequence with arginine (R). The abbreviation Arg94 refers to any sequence in which the 94th residue, relative to a reference sequence, is arginine (Arg). As described below, the preferred algorithms can account for gaps and the like. Preferably, identity exists over a region that is at least about 25 amino acids or nucleotides in length, or more preferably over a region that is 50-100 amino acids or nucleotides in length. For sequence comparison, typically one sequence acts as a reference sequence, to which test sequences are compared. The term “reference sequence” refers to a molecule to which a test sequence is compared.

Methods of sequence alignment for comparison and determination of percent sequence identity is well known in the art. Optimal alignment of sequences for comparison can be conducted, e.g., by the homology alignment algorithm of Needleman and Wunsch, J. Mol. Biol. 48:443-53 (1970).

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “treating” means one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

### EMBODIMENTS

Provided are polynucleotides that encode a polypeptide monomer of a trimeric component for a one-component protein nanostructure. Such protein nanostructures, when assembled, display viral protein trimers as antigens.

### gB Protein

In some embodiment, the protein nanostructure comprises a Cytomegalovirus gB protein monomer or variant thereof. Illustrative gB proteins are provided in Table 1. Signal peptides are in parentheses and N-terminal residues, membrane-proximal region, transmembrane, and cytoplasmic residues are underlined. In each case, the underlined portions may be truncated to leave an ectodomain; and the same or different signal peptide may be used. In some embodiment, the Cytomegalovirus gB viral protein monomer comprises a polypeptide sequence at least 70%, at least 80%, at least 90%, at least 95%, at least 98% identical to any one of the polypeptide sequences in Table 1, or to the ectodomain thereof. In some embodiments, the Cytomegalovirus gB viral protein monomer comprises a polypeptide sequence at least 70%, at least 80%, at least 90%, at least 95%, at least 98%, or at least 99% identical to any one of the polypeptide sequences disclosed therein.

TABLE 1
HCMV GB PROTEIN

| SEQ | | |
|---|---|---|
| ID | | |
| NO: | Sequence | Virus |
| 7 | (MESRIWCLVVCVNLCIVCL)GAAVSSSSTRGTSATHSHHSS | hCMV gB, |
| | HTTSAAHSRSGSVSQRVISSQTVSHGVNETIYNTTLKYGDVV | Towne strain, full |
| | GVNTTKYPYRVCSMAQGTDLIRFERNIVCTSMKPINEDLDEG | length |
| | IMVVYKRNIVAHTFKVRVYQKVLTFRRSYAYIHTTYLLGSNT | |
| | EYVAPPMWEIHHINSHSQCYSSYSRVIAGTVEVAYHRDSYEN | |
| | KTMQLMPDDYSNTHSTRYVTVKDQWHSRGSTWLYRETCNINC | |
| | MVTITTARSKYPYHFFATSTGDVVDISPFYNGTNRNASYEGE | |
| | NADKFFIFPNYTIVSDEGRPNSALETHRLVAFLERADSVISW | |
| | DIQDEKNVTCQLTFWEASERTIRSEAEDSYHESSAKMTATEL | |
| | SKKQEVNMSDSALDCVRDEAINKLQQIFNTSYNQTYEKYGNV | |
| | SVFETTGGLVVFWQGIKQKSLVELERLANRSSLNLTHNRTKR | |
| | STDGNNATHLSNMESVHNLVYAQLQFTYDTLRGYINRALAQI | |
| | AEAWCVDQRRTLEVEKELSKINPSAILSAIYNKPIAAREMGD | |
| | VLGLASCVTINQTSVKVLRDMNVKESPGRCYSRPVVIENEAN | |
| | SSYVQYGQLGEDNEILLGNHRTEECQLPSLKIFIAGNSAYEY | |
| | VDYLFKRMIDLSSISTVDSMIALDIDPLENTDERVLELYSQK | |
| | ELRSSNVEDLEEIMREFNSYKQRVKYVEDKVVDPLPPYLKGL | |
| | DDLMSGLGAAGKAVGVAIGAVGGAVASVVEGVATFLKNPFGA | |
| | FTIILVAIAVVIIIYLIYTRQRRLCMQPLQNLFPYLVSADGT | |
| | TVTSGNTKDTSLQAPPSYEESVYNSGRKGPGPPSSDASTAAP | |
| | PYTNEQAYQMLLALVRLDAEQRAQQNGTDSLDGQTGTQDKGQ | |
| | KPNLLDRLRHRKNGYRHLKDSDEEENV | |
| 2 | (MESRIWCLVVCVNICIVCL)GAAVSSSSTRGTSATHSHHSS | hCMV gB, |
| | HTTSAAHSRSGSVSQRVTSSQTVSHGVNETIYNTTLKYGDVV | Merlin strain, full |
| | GVNTTKYPYRVCSMAQGTDLIRFERNIVCTSMKPINEDLDEG | length |
| | IMVVYKRNIVAHTFKVRVYQKVLTFRRSYAYIHTTYLLGSNT | |
| | EYVAPPMWEIHHINSHSQCYSSYSRVIAGTVEVAYHRDSYEN | |
| | KTMQLMPDDYSNTHSTRYVTVKDQWHSRGSTWLYRETCNINC | |
| | MVTITTARSKYPYHFFATSTGDVVDISPFYNGTNRNASYFGE | |
| | NADKFFIFPNYTIVSDEGRPNSALETHRIVAFLERADSVISW | |
| | DIQDEKNVTCQLTFWEASERTIRSEAEDSYHFSSAKMTATFL | |
| | SKKQEVNMSDSALDCVRDEAINKLQQIFNTSYNQTYEKYGNV | |
| | SVFETTGGLVVFWQGIKQKSLVELERLANRSSLNLTHNRTKR | |
| | STDGNNATHISNMESVHNLVYAQLQFTYDTLRGYINRALAQI | |
| | AEAWCVDQRRTLEVEKELSKINPSAILSAIYNKPIAAREMGD | |
| | VLGLASCVTINQTSVKVLRDMNVKESPGRCYSRPVVIENFAN | |
| | SSYVQYGQLGEDNEILLGNHRTEECQLPSLKIFIAGNSAYEY | |
| | VDYLFKRMIDLSSISTVDSMIALDIDPLENTDERVLELYSQK | |
| | ELRSSNVEDLEEIMREENSYKQRVKYVEDKVVDPLPPYLKGL | |
| | DDLMSGLGAAGKAVGVAIGAVGGAVASVVEGVATFLKNPFGA | |
| | FTIILVAIAVVIITYLIYTRQRRLCTQPLQNLFPYLVSADGT | |
| | TVTSGSTKDTSLQAPPSYEESVYNSGRKGPGPPSSDASTAAP | |
| | PYTNEQAYQMLLALARLDAEQRAQQNGTDSLDGRTGTQDKGQ | |
| | KPNLLDRLRHRKNGYRHLKDSDEEENV | |
| 3 | (MESRIWCLVVCVNICIVCL)GAAVSSSSTSHATSSTHNGSH | hCMV gB, strain |
| | TSRTTSAQTRSVYSQHVTSSEAVSHRANETIYNTTLKYGDVV | AD169, full |
| | GVNTTKYPYRVCSMAQGTDLIRFERNIICTSMKPINEDLDEG | length |
| | IMVVYKRNIVAHTFKVRVYQKVLTFRRSYAYIYTTYLLGSNT | |
| | EYVAPPMWEIHHINKFAQCYSSYSRVIGGTVEVAYHRDSYEN | |
| | KIMQLIPDDYSNTHSTRYVTVKDQWHSRGSTWLYRETCNINC | |
| | MLTITTARSKYPYHFFATSTGDVVYISPFYNGTNRNASYFGE | |
| | NADKFFIFPNYTIVSDFGRPNAAPETHRLVAFLERADSVISW | |
| | DIQDEKNVICQLTFWEASERTIRSEAEDSYHESSAKMTATEL | |
| | SKKQEVNMSDSALDCVRDEAINKLQQIFNTSYNQTYEKYGNV | |
| | SVFETSGGLVVFWQGIKQKSLVELERLANRSSINITHRTRRS | |
| | TSDNNTTHLSSMESVHNLVYAQLQFTYDTLRGYINRALAQIA | |
| | EAWCVDQRRTLEVEKELSKINPSAILSAIYNKPIAAREMGDV | |
| | LGLASCVTINQTSVKVLRDMNVKESPGRCYSRPVVIENFANS | |
| | SYVQYGQLGEDNEILLGNHRTEECQLPSLKIFIAGNSAYEYV | |
| | DYLFKRMIDLSSISTVDSMIALDIDPLENTDERVLELYSQKE | |
| | LRSSNVEDLEEIMREFNSYKQRVKYVEDKVVDPLPPYLKGLD | |
| | DLMSGLGAAGKAVGVAIGAVGGAVASVVEGVATELKNPFGAF | |
| | TIILVAIAVVIITYLIYTRQRRLCTQPLQNLFPYLVSADGTT | |
| | VTSGSTKDTSLQAPPSYEESVYNSGRKGPGPPSSDASTAAPP | |
| | YTNEQAYQMLLALARLDAEQRAQQNGTDSLDGQTGTQDKGQK | |
| | PNLLDRLRHRKNGYRHLKDSDEEENV | |
| 4 | GAAVSSSSTRGTSATHSHHSSHTTSAAHSRSGSVSQRVTSSQ | hCMV gB |
| | TVSHGVNETIYNTTLKYGDVVGVNTTKYPYRVCSMAQGTDLI | extracellular |
| | RFERNIVCTSMKPINEDLDEGIMVVYKRNIVAHTFKVRVYQK | domain, Towne or |
| | VLTFRRSYAYIHTTYLLGSNTEYVAPPMWEIHHINSHSQCYS | Merlin strain |
| | SYSRVIAGTVFVAYHRDSYENKTMQLMPDDYSNTHSTRYVTV | |
| | KDQWHSRGSTWLYRETCNLNCMVTITTARSKYPYHFFATSTG | |
| | DVVDISPFYNGTNRNASYFGENADKFFIFPNYTIVSDFGRPN | |
| | SALETHRLVAFLERADSVISWDIQDEKNVTCQLTFWEASERT | |
| | IRSEAEDSYHESSAKMTATFLSKKQEVNMSDSALDCVRDEAI | |
| | NKLQQIFNTSYNQTYEKYGNVSVFETTGGLVVFWQGIKQKSL | |
| | VELERLANRSSLNLTHNRTKRSTDGNNATHISNMESVHNLVY | |
| | AQLQFTYDTLRGYINRALAQIAEAWCVDQRRTLEVEKELSKI | |
| | NPSAILSAIYNKPIAARFMGDVLGLASCVTINQTSVKVLRDM | |
| | NVKESPGRCYSRPVVIENFANSSYVQYGQLGEDNEILLGNHR | |
| | TEECQLPSLKIFIAGNSAYEYVDYLFKRMIDLSSISTVDSMI | |
| | ALDIDPLENTDERVLELYSQKELRSSNVEDLEEIMREFNSYK | |
| | QRVKYVEDKV | |
| 5 | GAAVSSSSTRGTSATHSHHSSHTTSAAHSRSGSVSQRVTSSQ | hCMV gB |
| | TVSHGVNETIYNTTLKYGDVVGVNTTKYPYRVCSMAQGTDLI | extracellular |
| | RFERNIVCTSMKPINEDLDEGIMVVYKRNIVAHTFKVRVYQK | domain, Towne or |
| | VLTFRRSYAYIHTTYLLGSNTEYVAPPMWEIHHINSHSQCYS | Merlin strain-C- |
| | SYSRVIAGTVFVAYHRDSYENKTMQLMPDDYSNTHSTRYVTV | terminus shorten |
| | KDQWHSRGSTWLYRETCNLNCMVTITTARSKYPYHFFATSTG | |
| | DVVDISPEYNGTNRNASYFGENADKFFIEPNYTIVSDEGRPN | |
| | SALETHRLVAFLERADSVISWDIQDEKNVTCQLTFWEASERT | |
| | IRSEAEDSYHFSSAKMTATFLSKKQEVNMSDSALDCVRDEAI | |
| | NKLQQIFNTSYNQTYEKYGNVSVFETTGGLVVFWQGIKQKSL | |
| | VELERLANRSSLNLTHNRTKRSTDGNNATHLSNMESVHNLVY | |
| | AQLQFTYDTLRGYINRALAQIAEAWCVDQRRTLEVFKELSKI | |
| | NPSAILSAIYNKPIAAREMGDVLGLASCVTINQTSVKVLRDM | |
| | NVKESPGRCYSRPVVIENFANSSYVQYGQLGEDNEILLGNHR | |
| | TEECQLPSLKIFIAGNSAYEYVDYLFKRMIDLSSISTVDSMI | |
| | ALDIDPLENTDERVLELYSQKELRSSNVE | |
| 6 | GAAVSSSSTSHATSSTHNGSHTSRTTSAQTRSVYSQHVTSSE | hCMV gB, strain |
| | AVSHRANETIYNTTLKYGDVVGVNTTKYPYRVCSMAQGTDLI | AD169, |
| | RFERNIICTSMKPINEDLDEGIMVVYKRNIVAHTFKVRVYQK | extracellular |
| | VLTFRRSYAYIYTTYLLGSNTEYVAPPMWEIHHINKFAQCYS | domain |
| | SYSRVIGGTVFVAYHRDSYENKTMQLIPDDYSNTHSTRYVTV | |
| | KDQWHSRGSTWLYRETCNUNCMLTITTARSKYPYHFFATSTG | |
| | DVVYISPFYNGTNRNASYFGENADKFFIFPNYTIVSDEGRPN | |
| | AAPETHRLVAFLERADSVISWDIQDEKNVTCQLTFWEASERT | |
| | IRSEAEDSYHESSAKMTATFLSKKQEVNMSDSALDCVRDEAI | |
| | NKLQQIFNTSYNQTYEKYGNVSVFETSGGLVVEWQGIKQKSL | |
| | VELERLANRSSLNITHRTRRSTSDNNTTHLSSMESVHNLVYA | |
| | QLQFTYDTLRGYINRALAQIAEAWCVDQRRTLEVEKELSKIN | |
| | PSAILSAIYNKPIAAREMGDVLGLASCVTINQTSVKVLRDMN | |
| | VKESPGRCYSRPVVIFNFANSSYVQYGQLGEDNEILLGNHRT | |
| | EECQLPSLKIFIAGNSAYEYVDYLEKRMIDLSSISTVDSMIA | |
| | LDIDPLENTDERVLELYSQKELRSSNVEDLEEIMREFNSYKQ | |
| | RVKYVEDKV | |
| 7 | GAAVSSSSTSHATSSTHNGSHTSRTTSAQTRSVYSQHVTSSE | hCMV gB, strain |
| | AVSHRANETIYNTTLKYGDVVGVNTTKYPYRVCSMAQGTDLI | AD169, |
| | RFERNIICTSMKPINEDLDEGIMVVYKRNIVAHTFKVRVYQK | extracellular |
| | VLTERRSYAYIYTTYLLGSNTEYVAPPMWEIHHINKFAQCYS | domain-C- |
| | SYSRVIGGTVFVAYHRDSYENKTMQLIPDDYSNTHSTRYVTV | terminus shorten |
| | KDQWHSRGSTWLYRETCNLNCMLTITTARSKYPYHFFATSTG | |
| | DVVYISPFYNGINRNASYFGENADKFFIEPNYTIVSDEGRPN | |
| | AAPETHRLVAFLERADSVISWDIQDEKNVTCQLTFWEASERT | |
| | IRSEAEDSYHFSSAKMTATFLSKKQEVNMSDSALDCVRDEAI | |
| | NKLQQIFNTSYNQTYEKYGNVSVFETSGGLVVFWQGIKQKSL | |
| | VELERLANRSSLNITHRTRRSTSDNNTTHLSSMESVHNLVYA | |
| | QLQFTYDTLRGYINRALAQIAEAWCVDQRRTLEVEKELSKIN | |
| | PSAILSAIYNKPIAARFMGDVLGLASCVTINQTSVKVLRDMN | |
| | VKESPGRCYSRPVVIENFANSSYVQYGQLGEDNEILLGNHRT | |
| | EECQLPSLKIFIAGNSAYEYVDYLFKRMIDLSSISTVDSMIA | |
| | LDIDPLENTDERVLELYSQKELRSSNVEDLEEIMREENSYKQ | |
| | RVKYVEDKV | |

SEQ ID NO: 1 is the amino acid sequence for the cytomegalovirus (CMV) Towne strain glycoprotein B (gB). SEQ ID NO: 2 is the amino acid sequence for the CMV Merlin strain gB. SEQ ID NO 3 is the amino acid sequence for CMV strain AD169 gB, which shares about 95% identity to SEQ ID NO: 1 and has a deletion relative to SEQ ID NO: 1 and 2 at residue 456.

### Amino Acid Substitutions

Provided are sequences of recombinant CMV gB proteins in which, while not wishing to be held by theory, amino acid substitutions have been added to disrupt the postfusion state and/or stabilize the prefusion state.

In some embodiment, the Cytomegalovirus gB protein includes one of the following combinations of mutations:

TABLE 2
Category 1

367I and 374F
686L and 690F
686L and 693V
690F and 693V
686L, 690F, and 693V
686L, 690F, 693V, and 694L
686L, 690F, 693V, and 694L, 700A
686L, 690F, 693V, 694L, 700A, and 702Q
218F, 591F, and 668A
218F, 591Y, and 668A
220S and 591F
220S and 591Y
552L and 591F
552L and 591Y
220S and 552L
220S and 552L, 591F
220S, 552L, 591Y
218F, 220S, 552L, 591F, and 668A
218F, 220S, 552L, 591Y, and 668A
217C and 587C
217C and 589C

TABLE 3
Category 2

217C, 587C, 686L, 690F, 693V
217C, 587C, 686L, 690F, 693V, 694L
217C, 587C, 686L, 690F, 693V, 694L, 700A
217C, 589C, 686L, 690F, 693V
217C, 589C, 686L, 690F, 693V, 694L
217C, 589C, 686L, 690F, 693V, 694L, 700A
217C, 587C, 591Y, 686L, 690F, 693V
217C, 587C, 591Y, 686L, 690F, 693V, 694L
217C, 587C, 591Y, 686L, 690F, 693V, 694L, 700A
217C, 589C, 591Y, 686L, 690F, 693V
217C, 589C, 591Y, 686L, 690F, 693V, 694L
217C, 589C, 591Y, 686L, 690F, 693V, 694L, 700A
217C, 587C, 591F, 686L, 690F, 693V
217C, 587C, 591F, 686L, 690F, 693V, 694L
217C, 587C, 591F, 686L, 690F, 693V, 694L, 700A
217C, 589C, 591F, 686L, 690F, 693V
217C, 589C, 591F, 686L, 690F, 693V, 694L
217C, 589C, 591F, 686L, 690F, 693V, 694L, 700A
220S, 552L, 686L, 690F, 693V
220S, 552L, 686L, 690F, 693V, 694L
220S, 552L, 686L, 690F, 693V, 694L, 700A
218F, 220S, 552L, 591F, 668A, 686L, 690F, 693V
218F, 220S, 552L, 591F, 668A, 686L, 690F, 693V, 694L
218F, 220S, 552L, 591F, 668A, 686L, 690F, 693V, 694L, 700A
218F, 220S, 552L, 591Y, 668A, 686L, 690F, 693V
218F, 220S, 552L, 591Y, 668A, 686L, 690F, 693V, 694L
218F, 220S, 552L, 591Y, 668A, 686L, 690F, 693V, 694L, 700A
218F, 591F, 668A, 686L, 690F, 693V
218F, 591F, 668A, 686L, 690F, 693V, 694L
218F, 591F, 668A, 686L, 690F, 693V, 694L, 700A
218F, 591Y, 668A, 686L, 690F, 693V
218F, 591Y, 668A, 686L, 690F, 693V, 694L
218F, 591Y, 668A, 686L, 690F, 693V, 694L, 700A

TABLE 4
Category 3

217C, 587C, 686L, 690F
217C, 589C, 686L, 690F
217C, 587C, 591Y, 686L, 690F
217C, 589C, 591Y, 686L, 690F
217C, 587C, 591F, 686L, 690F
217C, 589C, 591F, 686L, 690F
217C, 220S, 587C, 591F, 686L, 690F
217C, 220S, 587C, 591F, 686L, 690F, 693V
217C, 220S, 587C, 591F, 686L, 690F, 693V, 694L
217C, 220S, 587C, 591F, 686L, 690F, 693V, 694L, 700A
217C, 220S, 589C, 591F, 686L, 690F
217C, 220S, 589C, 591F, 686L, 690F, 693V
217C, 220S, 589C, 591F, 686L, 690F, 693V, 694L
217C, 220S, 589C, 591F, 686L, 690F, 693V, 694L, 700A
217C, 220S, 587C, 591Y, 686L, 690F
217C, 220S, 587C, 591Y, 686L, 690F, 693V
217C, 220S, 587C, 591Y, 686L, 690F, 693V, 694L
217C, 220S, 587C, 591Y, 686L, 690F, 693V, 694L, 700A
217C, 220S, 589C, 591Y, 686L, 690F
217C, 220S, 589C, 591Y, 686L, 690F, 693V
217C, 220S, 589C, 591Y, 686L, 690F, 693V, 694L
217C, 220S, 589C, 591Y, 686L, 690F, 693V, 694L, 700A
217C, 220S, 552L, 587C, 686L, 690F
217C, 220S, 552L, 587C, 686L, 690F, 693V
217C, 220S, 552L, 587C, 686L, 690F, 693V, 694L
217C, 220S, 552L, 587C, 686L, 690F, 693V, 694L, 700A
217C, 220S, 552L, 589C, 686L, 690F
217C, 220S, 552L, 589C, 686L, 690F, 693V
217C, 220S, 552L, 589C, 686L, 690F, 693V, 694L
217C, 220S, 552L, 589C, 686L, 690F, 693V, 694L, 700A
217C, 220S, 552L, 587C, 591F, 686L, 690F
217C, 220S, 552L, 587C, 591F, 686L, 690F, 693V
217C, 220S, 552L, 587C, 591F, 686L, 690F, 693V, 694L
217C, 220S, 552L, 587C, 591F, 686L, 690F, 693V, 694L, 700A
217C, 220S, 552L, 589C, 591F, 686L, 690F
217C, 220S, 552L, 589C, 591F, 686L, 690F, 693V
217C, 220S, 552L, 589C, 591F, 686L, 690F, 693V, 694L
217C, 220S, 552L, 589C, 591F, 686L, 690F, 693V, 694L, 700A
217C, 220S, 552L, 587C, 591Y, 686L, 690F
217C, 220S, 552L, 587C, 591Y, 686L, 690F, 693V
217C, 220S, 552L, 587C, 591Y, 686L, 690F, 693V, 694L
217C, 220S, 552L, 587C, 591Y, 686L, 690F, 693V, 694L, 700A
217C, 220S, 552L, 589C, 591Y, 686L, 690F
217C, 220S, 552L, 589C, 591Y, 686L, 690F, 693V
217C, 220S, 552L, 589C, 591Y, 686L, 690F, 693V, 694L
217C, 220S, 552L, 589C, 591Y, 686L, 690F, 693V, 694L, 700A
217C, 218F, 220S, 552L, 587C, 591F, 668A, 686L, 690F
217C, 218F, 220S, 552L, 587C, 591F, 668A, 686L, 690F, 693V
217C, 218F, 220S, 552L, 587C, 591F, 668A, 686L, 690F, 693V, 694L
217C, 218F, 220S, 552L, 587C, 591F, 668A, 686L, 690F, 693V, 694L, 700A
217C, 218F, 220S, 552L, 589C, 591F, 668A, 686L, 690F
217C, 218F, 220S, 552L, 589C, 591F, 668A, 686L, 690F, 693V
217C, 218F, 220S, 552L, 589C, 591F, 668A, 686L, 690F, 693V, 694L
217C, 218F, 220S, 552L, 589C, 591F, 668A, 686L, 690F, 693V, 694L, 700A
217C, 218F, 220S, 552L, 587C, 591Y, 668A, 686L, 690F
217C, 218F, 220S, 552L, 587C, 591Y, 668A, 686L, 690F, 693V
217C, 218F, 220S, 552L, 587C, 591Y, 668A, 686L, 690F, 693V, 694L
217C, 218F, 220S, 552L, 587C, 591Y, 668A, 686L, 690F, 693V, 694L, 700A
217C, 218F, 220S, 552L, 589C, 591Y, 668A, 686L, 690F
217C, 218F, 220S, 552L, 589C, 591Y, 668A, 686L, 690F, 693V
217C, 218F, 220S, 552L, 589C, 591Y, 668A, 686L, 690F, 693V, 694L
217C, 218F, 220S, 552L, 589C, 591Y, 668A, 686L, 690F, 693V, 694L, 700A
217C, 218F, 587C, 591F, 668A, 686L, 690F
217C, 218F, 587C, 591F, 668A, 686L, 690F, 693V
217C, 218F, 587C, 591F, 668A, 686L, 690F, 693V, 694L
217C, 218F, 587C, 591F, 668A, 686L, 690F, 693V, 694L, 700A
217C, 218F, 589C, 591F, 668A, 686L, 690F
217C, 218F, 589C, 591F, 668A, 686L, 690F, 693V
217C, 218F, 589C, 591F, 668A, 686L, 690F, 693V, 694L
217C, 218F, 589C, 591F, 668A, 686L, 690F, 693V, 694L, 700A
217C, 218F, 587C, 591Y, 668A, 686L, 690F
217C, 218F, 587C, 591Y, 668A, 686L, 690F, 693V
217C, 218F, 587C, 591Y, 668A, 686L, 690F, 693V, 694L
217C, 218F, 587C, 591Y, 668A, 686L, 690F, 693V, 694L, 700A
217C, 218F, 589C, 591Y, 668A, 686L, 690F
217C, 218F, 589C, 591Y, 668A, 686L, 690F, 693V
217C, 218F, 589C, 591Y, 668A, 686L, 690F, 693V, 694L
217C, 218F, 589C, 591Y, 668A, 686L, 690F, 693V, 694L, 700A
217C, 587C, 591Y
217C, 589C, 591Y
217C, 587C, 591F
217C, 589C, 591F
217C, 220S, 587C, 591F
217C, 220S, 589C, 591F
217C, 220S, 587C, 591Y
217C, 220S, 589C, 591Y
217C, 220S, 552L, 587C
217C, 220S, 552L, 589C
217C, 220S, 552L, 587C, 591F
217C, 220S, 552L, 589C, 591F
217C, 220S, 552L, 587C, 591Y
217C, 220S, 552L, 589C, 591Y
217C, 218F, 220S, 552L, 587C, 591F, 668A
217C, 218F, 220S, 552L, 589C, 591F, 668A
217C, 218F, 220S, 552L, 587C, 591Y, 668A
217C, 218F, 220S, 552L, 589C, 591Y, 668A
217C, 218F, 587C, 591F, 668A
217C, 218F, 589C, 591F, 668A
217C, 218F, 587C, 591Y, 668A
217C, 218F, 589C, 591Y, 668A
591Y, 686L, 690F
591Y, 686L, 690F, 693V
591Y, 686L, 690F, 693V, 694L
591Y, 686L, 690F, 693V, 694L, 700A
591F, 686L, 690F
591F, 686L, 690F, 693V
591F, 686L, 690F, 693V, 694L
591F, 686L, 690F, 693V, 694L, 700A
220S, 591F, 686L, 690F
220S, 591F, 686L, 690F, 693V
220S, 591F, 686L, 690F, 693V, 694L
220S, 591F, 686L, 690F, 693V, 694L, 700A
220S, 591Y, 686L, 690F
220S, 591Y, 686L, 690F, 693V
220S, 591Y, 686L, 690F, 693V, 694L
220S, 591Y, 686L, 690F, 693V, 694L, 700A
220S, 552L. 686L. 690F
220S, 552L, 591F, 686L, 690F
220S, 552L, 591F, 686L, 690F, 693V
220S, 552L, 591F, 686L, 690F, 693V, 694L
220S, 552L, 591F, 686L, 690F, 693V, 694L, 700A
220S, 552L, 591Y, 686L, 690F
220S, 552L, 591Y, 686L, 690F, 693V
220S, 552L, 591Y, 686L, 690F, 693V, 694L
220S, 552L, 591Y, 686L, 690F, 693V, 694L, 700A
218F, 220S, 552L, 591F, 668A, 686L, 690F
218F, 220S, 552L, 591Y, 668A, 686L, 690F
218F, 591F, 668A, 686L, 690F
218F, 591Y, 668A, 686L, 690F

Without adherence to theory, the above mutation combinations serve to disrupt the postfusion state and/or stabilize the prefusion state, thus making the polypeptides particularly useful as immunogens to induce an immune response against CMV, and/or for vaccinating against CMV. In some embodiments, residues 698-702 are optional if none of the mutations in the combination of mutations includes a mutation at residues 698-702.

Tables 2-4 list useful stabilizing gB mutations.

Table 5 lists additional mutations that are useful in combination with the mutations listed in Tables 2-4.

TABLE 5
Category 4

93I
93L
93M
96T
96V
96I
96L
221P
552M
552F
552Y
582P
584L
584M
584Y
603P
664F
664Y
664W
664S
664A
677T
678N
679N
679H
680E
681N
682S
684S
685Q
687A
688E
692S
695D
696R
699K

Table 6 lists additional mutations useful in combination with the mutations listed in Tables 2-5.

TABLE 6
Category 5

155G
156H
157R
206H
240A
240N
241T
242T
242H
246S
457S
460S

Table 7 lists additional mutations that are useful in combination with the mutations listed in Tables 2-6.

TABLE 7
Category 6

155H
155D
155E
155S
155N
156T
156D
156E
156Q
156K
156R
157D
157E
157S
157K
157N
157T
206G
206A
206S
206N
206Q
206K
206R
240S
240Q
240D
240E
240K
240R
240H
241S
241A
241G
241N
242S
242A
242G
242N
242Q
246G
246A
246N
246T
246Q
246K
246R
149T, 246T
149T, 246V
149T, 246N
149T, 246Q
149T, 246K
149T, 246R
149V, 246T
149V, 246V
149V, 246N
149V, 246Q
149V, 246K
149V, 246R
149I, 246T
149I, 246V
149I, 246N
149I, 246Q
149I, 246K
149I, 246R
457N
457Q
457A
457T
457G
457D
457E
459N
459Q
459A
459T
459G
459D
459E
460N
460Q
460A
460T
460G
460D
460E

Mutations listed in Table 8 are known to known volatile elements in the gB protein which make the protein difficult to express/produce/isolate.

TABLE 8
Category 7

156S, 157K, 240D, 242S, 246T
156D, 157K, 240S, 242S, 246T
156S, 157E, 240D, 242S, 246T
156D, 157E, 240S, 242S, 246T
156S, 157K, 240D, 242N, 246T
156D, 157K, 240S, 242N, 246T
156S, 157E, 240D, 242N, 246T
156D, 157E, 240S, 242N, 246T
156S, 157K, 240D, 242S, 246A
156D, 157K, 240S, 242S, 246A
156S, 157E, 240D, 242S, 246A
156D, 157E, 240S, 242S, 246A
156S, 157K, 240D, 242N, 246A
156D, 157K, 240S, 242N, 246A
156S, 157E, 240D, 242N, 246A
156D, 157E, 240S, 242N, 246A
149T, 156S, 157K, 240D, 242S, 246T
149T, 156D, 157K, 240S, 242S, 246T
149T, 156S, 157E, 240D, 242S, 246T
149T, 156D, 157E, 240S, 242S, 246T
149T, 156S, 157K, 240D, 242N, 246T
149T, 156D, 157K, 240S, 242N, 246T
149T, 156S, 157E, 240D, 242N, 246T
149T, 156D, 157E, 240S, 242N, 246T
149T, 156S, 157K, 240D, 242S, 246N
149T, 156D, 157K, 240S, 242S, 246N
149T, 156S, 157E, 240D, 242S, 246N
149T, 156D, 157E, 240S, 242S, 246N
149T, 156S, 157K, 240D, 242N, 246N
149T, 156D, 157K, 240S, 242N, 246N
149T, 156S, 157E, 240D, 242N, 246N
149T, 156D, 157E, 240S, 242N, 246N

In one aspect, the disclosure provides a polypeptide comprising an ectodomain of CMV gB in the prefusion conformation, wherein the ectodomain comprises, 1, 2, 4 or more amino acid substitutions selected from the group consisting of E686L, R693V, V694L, Y690F, K700A, V702E, D6791H, E681N, K695D, L680E, R685Q, V701L, E682S, F678N, N688E, V677T, D699K, F687A, M684S, Q692S, and Y696R. or substitutions at the same amino acid positions; the amino acid substitution S3671L the amino acid substitution T374F, or tie amino acid substitutions S367I and T374F, or substitutions at positions 367 and/or 374; 1, 2, 3, 4 or more amino acid substitutions selected from the group consisting of Q591F, Q591Y, S668A, Y218F, N220S, V552L, or substitutions at the same amino acid positions; the amino acid substitution E167T; and/or 1, 2, 3, 4 or more amino acid substitutions selected from the group consisting of C246S, 11157R, 1561-1, R457S, R460S, W240N, and Y242T at positions relative to SEQ ID NO: 1.

In some embodiments, the ectodomain comprises the amino acid subsitutions D217C and S587C. In some embodiments, the ectodomain comprises the amino acid subsitutions D217C and Y589C. 1012211n some embodiments, the ectodomain comprises 1, 2, 3, 4 or more amino acid substitutions selected from the group consisting of E686L, R693V, V694L, Y690F, K700A, V702E, D679H, E681N, K695D, L680E, R685Q. V701K, E682S, F678N, N688E. V677T. D699K, F687A, M684S, Q692S, and Y696R.

In some embodiments, the ectodomain comprises the amino acid substitutions E686L, R693V, V694L, and Y690F. In some embodiments, the ectodomain comprises the amino acid substitutions E686L, R693, V694L, Y690P, K700A, and V702Q. In some embodiments, the ectodomain comprises the amino acid substitutions E686L, R693V, V694L, Y690F, D679N, E681N, K695D, L680E, and R685Q. In some embodiments, the ectodomain comprises the amino acid substitutions E686L, R693V, V694L, Y690F, K700A, V702Q, D679N. E681N, K695D, L680E, R685Q, and V170L. In some embodiments, the ectodomain comprises the amino acid substitutions E686L, R693V, V694L, Y690F, D679N, E681N K695D, L680E, R685Q, E682S, F678N, N688E, and V677T. In some embodiments, the ectodomain comprises tie amino acid substitutions E686L, R693V, V694L, Y690F, K700A, V702Q, D679N, E681N, K695D, L680E, R685Q, V701L, E682S, F678N, N688E, V677T, and D699K. In some embodiments, the ectodomain comprises the amino acid substitutions E686L, R693V, V694L, Y690F, D679H, E681N, K695D, L680E, R685Q, E682S, F678N, N688E, V677T, F687A, M684S, Q692S, and Y696R. In some embodiments, the ectodomain comprises the amino acid substitutions E686L, R693V, V694L, Y690F, K700A, V702E, D679H, E681N, K695D, L680E, R685Q, V701L, E682S, F678N, N688E, V677T, D699K, F687A, M684S, Q692S, and Y696R.

In some embodiments, the ectodomain comprises the amino acid substitution S367I or the amino acid substitution T374F. In some embodiments, the ectodomain comprises the amino acid substitutions S367I and T374F.

In some embodiments, the ectodomain comprises e, 2, 3, 4 or more amino acid substitutions selected from the group consisting of Q591F, Q591Y, S668A, Y218F, N220S, and V552L. In some embodiments, the ectodomain comprises the amino acid substitutions Q591F, S668A, and Y218F. In some embodiments, the ectodomain comprises the amino acid substitutions N220S and V552L. In some embodiments, the ectodomain comprises the amino acid substitutions Q591F, S668A, Y218F, N220S, and V552L. In some embodiments, the ectodomain comprises the amino acid substitutions Q591Y, 5668A, Y218F, N220S, and V552L. In some embodiments, the ectodomain comprises the amino acid substitutions Q591Y, N220S, and V552L. In some embodiments, the ectodomain comprises the amino acid substitutions Q591F, N220S, and V552L.

In some embodiments, the ectodomain comprises amino acid substitution E167T or E167S. In sonic embodiments, the ectodomain comprises 1, 2, 3, 4 or more amino acid substitutions selected from the group consisting of C246S, H157R, 1156H, R457S, R460S, W240N, and Y242T.

In some embodiments, the polypetide comprises any one combination of amino acid substitutions listed in Table 10.

In sonic embodiments, the polypetide comprises a polypeptide sequence at least 75%, 80%85%, 90%, 95%, 96%, 97%, 98%, 99%, or 100% identical to a polypeptide sequence in Table 10 without the signal peptide, or an antigenic fragment thereof.

In some embodiments, the CMV gB protein adopts in a prefusion conformation in the absence of a fusion inhibitor, optionally N-{4-[({(]S)-1-[3,5-bis(trifluoromethyl)phenyl]ethyl}carbamothioyl) amino]phenyl}-1,3-thiazole-4-carboxamide.

In another aspect, the disclosure provides a polypeptide comprising an amino acid sequence at least 90%, 91%, 92%, 93%, 94%,95%, 96%, 97% 98%, or 99% identical to the amino acid sequence of SEQ ID NO:1 or SEQ ID NO:3 residues 91-702, or an antigenic fragment thereof, wherein residues 698-702 are optional if they do not include a mutation, SEQ ID NO:3 residue 456 is absent, and the polypeptide or antigenic fragment thereof comprises 1 or more combination of mutations relative to SEQ ID NO:1 or SEQ ID NO:3 residues 91-702 listed in Table 2, Table 3, or Table 4.

In some embodiments, the polypeptide or antigenic fragment thereof comprises 1 or more combination of mutations relative to SEQ ID NO:1 or SEQ ID NO:3 residues 91-702 listed in Table 2 or Table 3. In some embodiments, the polypeptide or antigenic fragment thereof comprises 1 or more combination of mutations relative to SEQ ID NO: 1 or SEQ ID NO:3 residues 91-702 listed in Table 2. In some embodiments, any polypeptide or antigenic fragment of the disclosure thereof further comprises 1, 2, 3, 4, 5, 6, 7, 8, 9, 10, I1, 12, 13, 14, 15, 16, 17, 18, 19, 20, or more mutations relative to relative to SEQ ID NO: 1 or SEQ ID NO:3 residues 91-702 listed in Table 5. In some embodiments, any polypeptide or antigenic fragment of the disclosure thereof further comprises 1, 2, 3, 4, 5, 6, 7, 8, 9, or all 10 mutations relative to relative to SEQ ID NO:1 or SEQ ID NO:3 residues 91-702 listed in Table 6. In some embodiments, any polypeptide or antigenic fragment of the disclosure thereof further comprises 1, 2, 3, 4, 5, 6, 7, 8, 9, 10, 11, or all 12, relative to relative to SEQ ID NO.: or SEQ ID NO:3 residues 91-702 listed in Table 7. In some embodiments, any polypeptide or antigenic fragment of the disclosure thereof further comprises a combination of mutations relative to relative to SEQ ID NO: 1 or SEQ ID NO:3 residues 91-702 listed in Table 8.

In some embodiments, any polypeptide of the disclosure, comprising an amino acid sequence at least 90%, 91%, 92%, 93%, 94% 95%, 96%, 97% 98%, or 99% identical to the amino acid sequence selected from the sequences listed in Table 9, or an antigenic fragment thereof. In some embodiments, any polypeptide of the disclosure, comprising an amino acid sequence at least 90%, 91%, 92%, 93%, 94%, 95%, 96%, 97%, 98%, or 99% identical to the amino acid sequence selected from the sequences listed in Table 10, or an antigenic fragment thereof. In some embodiments, any polypeptide of the disclosure further comprising any other functional domain as appropriate for an intended use, including but not limited to a secretion signal located at the N-terminus of the polypeptide, wherein the signal sequence may be any suitable signal sequence as appropriate for an intended use.

### Signal Peptide

In some embodiments, the encoded polypeptide includes a peptide region that is a signal peptide. Signal peptides are well known in the art. The signal peptide may be the native signal peptide or can be replaced with another signal peptide. Signal peptides function to prompt a cell to translocate the protein, usually to the cellular membrane. The core of the signal peptide often contains a long stretch of hydrophobic amino acids (about 5-16 residues long) that has a tendency to form a single alpha-helix and is also referred to as the “h-region”. In addition, many signal peptides begin with a short positively charged stretch of amino acids, which may help to enforce proper topology of the polypeptide during translocation by what is known as the positive-inside rule. Because of its close location to the N-terminus it is called the “n-region”. At the end of the signal peptide there is typically a stretch of amino acids that is recognized and cleaved by signal peptidase.

In some embodiments, the polypeptide does not include a signal peptide.

Nonlimiting examples of signal peptides are provided below:

(SEQ ID NO: 32)
MELLILKANAITTILTAVTFCFASG
(SEQ ID NO: 33)
MSWKVMIIISLLITPQHGL
(SEQ ID NO: 34)
MKAILVVLLYTFTTANA
(SEQ ID NO: 35)
MPISILLIITTMIMASHC
(SEQ ID NO: 36)
MFVFLVLLPLVSSQC
(SEQ ID NO: 37)
MVPQVLLFVPLLGFSLCFG

Illustrative sequences of fusions that include a signal peptide are provided in Table 9. In each case, the fusion protein is shown with a signal peptide that would be cleaved during secretion of the fusion protein. The signal peptide is shown in parentheses to identicate that sequence identity to each sequence should be calculated without the signal peptide. Alternative signal peptides may be substituted using routine methods known in the art.

TABLE 9

| | | |
|---|---|---|
| SEQ ID | gB-Towne-ext-N-term_Bg- | GAAVSSSSTRGTSATHSHHSSHTTSAAHSRSGSVSQRVTSSQTV |
| NO: 38 | KOs_dII-dIII-pack1 | SHGVNETIYNTTLKYGDVVGVNTTKYPYRVCSMAQGTDLIRFER |
| | Bg-KOs: I156H, H157R, | NIVCTSMKPINEDLDEGIMVVYKRNIVAHTFKVRVYQKVLTERR |
| | W240N, Y242T, C246S, | SYAYHRTTYLLGSNTEYVAPPMWEIHHINSHSQCYSSYSRVIAG |
| | R457S, R460S | TVEVAYHRDSYENKTMQLMPDDYSNTHSTRYVTVKDQWHSRGST |
| | dII-dIII-pack1: S367I, | NLTRETSNLNCMVTITTARSKYPYHFFATSTGDVVDISPFYNGT |
| | T374F | NRNASYFGENADKFFIFPNYTIVSDFGRPNSALETHRLVAFLER |
| | | ADSVISWDIQDEKNVTCQLTFWEASERTIRSEAEDSYHFISAKM |
| | | TAFFLSKKQEVNMSDSALDCVRDEAINKLQQIFNTSYNQTYEKY |
| | | GNVSVFETTGGLVVFWQGIKQKSLVELERLANRSSLNLTANSTK |
| | | SSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVED |
| | | LEEIMREFNSYKQRVKYV |
| SEQ ID | gB-Towne-ext-N-term_Bg- | GAAVSSSSTRGTSATHSHHSSHTTSAAHSRSGSVSQRVTSSQTV |
| NO: 39 | KOs_Cpack1-short | SHGVNETIYNTTLKYGDVVGVNTTKYPYRVCSMAQGTDLIRFER |
| | Bg-KOs: I156H, H157R, | NIVCTSMKPINEDLDEGIMVVYKRNIVAHTFKVRVYQKVLTERR |
| | W240N, Y242T, C246S, | SYAYHRTTYLLGSNTEYVAPPMWEIHHINSHSQCYSSYSRVIAG |
| | R457S, R460S | TVFVAYHRDSYENKTMQLMPDDYSNTHSTRYVTVKDQWHSRGST |
| | Cpack1-short: E686L, | NLTRETSNLNCMVTITTARSKYPYHFFATSTGDVVDISPFYNGT |
| | Y690F, R693V, V694L | NRNASYFGENADKFFIFPNYTIVSDEGRPNSALETHRLVAFLER |
| | | ADSVISWDIQDEKNVTCQLIFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSLVELERLANRSSINLTHNSTK |
| | | SSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVFD |
| | | LEEIMRLFNSFKQVLKYV |
| SEQ ID | gB-Towne-ext-N-term_Bg- | GAAVSSSSTRGTSATHSHHSSHTTSAAHSRSGSVSQRVTSSQTV |
| NO: 40 | KOs_Cpack1-long | SHGVNETIYNTTLKYGDVVGVNTTKYPYRVCSMAQGTDLIRFER |
| | Bg-KOs: I156H, H157R, | NIVCTSMKPINEDLDEGIMVVYKRNIVAHTFKVRVYQKVLTERR |
| | W240N, Y242T, C246S, | SYAYHRTTYLLGSNTEYVAPPMWEIHHINSHSQCYSSYSRVIAG |
| | R457S, R460S | TVFVAYHRDSYENKTMQLMPDDYSNTHSTRYVTVKDQWHSRGST |
| | Cpack1-long: E686L, Y690F, | NLTRETSNLNCMVTITTARSKYPYHFFATSTGDVVDISPFYNGT |
| | R693V, V694D, K700A, V702Q | NRNASYFGENADKFFIFPNYTIVSDFGRPNSALETHRLVAFLER |
| | | ADSVISWDIQDEKNVTCQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNSTK |
| | | SSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIFNFANSSYVQ |
| | | YGQLGEDNEILLGNHRTEECQLPSLKIFIAGNSAYEYVDYLFKR |
| | | MIDLSSISTVDSMIALDIDPLENTDERVLELYSQKELRSSNVED |
| | | LEEIMRLENSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 41 | KOs_Cpack2-short | SHGVNETIYNTTLKYGDVVGVNTTKYPYRVCSMAQGTDLIRFER |
| | Bg-KOs: I156H, H157R, | NIVCTSMKPINEDLDEGIMVVYKRNIVAHTFKVRVYQKVLTERR |
| | W240N, Y242T, C246S, | SYAYHRTTYLLGSNTEYVAPPMWEIHHINSHSQCYSSYSRVIAG |
| | R457S, R460S | TVFVAYHRDSYENKTMQLMPDDYSNTHSTRYVTVKDQWHSRGST |
| | Cpack2-short: D679N, | NLTRETSNLNCMVTITTARSKYPYHFFATSTGDVVDISPFYNGT |
| | L680E, E681N, R685Q, | NRNASYFGENADKFFIFPNYTIVSDFGRPNSALETHRLVAFLER |
| | E686L, Y690F, R693V, | ADSVISWDIQDEKNVICQLTFWEASERTIRSEAEDSYHFSSAKM |
| | V694L, K695D | TATFLSKKQEVNMSDSALDCVRDEAINKLQQIFNTSYNQTYEKY |
| | | GNVSVFETTGGLVVFWQGIKQKSLVELERLANRSSINLTHNSTK |
| | | SSTDGNNATHLSNMESVHNLVYAQLQFTYDTLRGYINRALAQIA |
| | | EAWCVDQRRTLEVFKELSKINPSAILSAIYNKPIAARFMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDFRVLELYSQKELRSSNVEN |
| | | ENEIMQLENSFKQVLDYV |
| SEQ ID | gB-Towne-ext-N-term_Bg- | GAAVSSSSTRGTSATHSHHSSHTTSAAHSRSGSVSQRVTSSQTV |
| NO: 42 | KOs_Cpack2-long | SHGVNETIYNTTLKYGDVVGVNITKYPYRVCSMAQGTDLIRFER |
| | Bg-KOs: I156H, H157R, | NIVCTSMKPINEDLDEGIMVVYKRNIVAHTEKVRVYQKVLTERR |
| | W240N, Y242T, C246S, | SYAYHRTTYLLGSNTEYVAPPMWEIHHINSHSQCYSSYSRVIAG |
| | R457S, R460S | TVFVAYHRDSYENKTMQLMPDDYSNTHSTRYVTVKDQWHSRGST |
| | Cpack2-long: D679N, L680E, | NLTRETSNLNCMVTITTARSKYPYHFFATSTGDVVDISPFYNGT |
| | E681N, R685Q, E686L, | NRNASYFGENADKFFIFPNYTIVSDFGRPNSALETHRLVAFLER |
| | Y690F, R693V, V694L, | ADSVISWDIQDEKNVTCQLTFWEASERTIRSEAEDSYHFSSAKM |
| | K695D, K700A, V701L, V702Q | TATFLSKKQEVNMSDSALDCVRDEAINKLQQIFNTSYNQTYEKY |
| | | GNVSVFETTGGLVVFWQGIKQKSIVELERLANRSSLNLTHNSTK |
| | | SSTDGNNATHLSNMESVHNLVYAQLQFTYDTLRGYINRALAQIA |
| | | EAWCVDQRRTLEVFKELSKINPSAILSAIYNKPIAARFMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVEN |
| | | ENEIMQLFNSFKQVLDYVEDALQ |
| SEQ ID | gB-Towne-ext-N-term_Bg- | GAAVSSSSTRGTSATHSHHSSHTTSAAHSRSGSVSQRVTSSQTV |
| NO: 43 | KOs_Cpack3-short | SHGVNETIYNTTLKYGDVVGVNTTKYPYRVCSMAQGTDLIRFER |
| | Bg-KOs: I156H, H157R, | NIVCTSMKPINEDLDEGIMVVYKRNIVAHTFKVRVYQKVLTERR |
| | W240N, Y242T, C246S, | SYAYHRTTYLLGSNTEYVAPPMWEIHHINSHSQCYSSYSRVIAG |
| | R457S, R460S | TVEVAYHRDSYENKTMQLMPDDYSNTHSTRYVTVKDQWHSRGST |
| | Cpack3-short: V677T, | NLTRETSNLNCMVTITTARSKYPYHFFATSIGDVVDISPFYNGT |
| | F678N, D679N, L680E, | NRNASYFGENADKFFIFPNYTIVSDFGRPNSALETHRLVAFLER |
| | E681N, E682S, R685Q, | ADSVISWDIQDEKNVICQLIFWEASERTIRSEAEDSYHESSAKM |
| | E686L, N688E, Y690F, | TATFLSKKQEVNMSDSALDCVRDEAINKLQQIFNTSYNQTYEKY |
| | R693V, V694L, K695D | GNVSVFETTGGLVVFWQGIKQKSLVELERLANRSSLNLTANSTK |
| | | SSTDGNNATHLSNMESVHNLVYAQLQFTYDTLRGYINRALAQIA |
| | | EAWCVDQRRTLEVFKELSKINPSAILSAIYNKPIAARFMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLFKR |
| | | MIDLSSISTVDSMIALDIDPLENTDERVLELYSQKELRSSNTNN |
| | | ENSIMQLFESFKQVLDYV |
| SEQ ID | gB-Towne-ext-N-term_Bg- | GAAVSSSSTRGTSATHSHHSSHTTSAAHSRSGSVSQRVTSSQTV |
| NO: 44 | KOs_Cpack3-long | SHGVNETIYNTTLKYGDVVGVNTTKYPYRVCSMAQGTDLIRFER |
| | Bg-KOs: I156H, H157R, | NIVCTSMKPINEDLDEGIMVVYKRNIVAHTFKVRVYQKVLTERR |
| | W240N, Y242T, C246S, | SYAYHRTTYLLGSNTEYVAPPMWEIHHINSHSQCYSSYSRVIAG |
| | R457S, R460S | TVFVAYHRDSYENKTMQLMPDDYSNTHSTRYVTVKDQWHSRGST |
| | Cpack3-long: V677T, F678N, | NLTRETSNLNCMVTITTARSKYPYHFFATSTGDVVDISPFYNGT |
| | D679N, L680E, E681N, | NRNASYFGENADKFFIFPNYTIVSDFGRPNSALETHRLVAFLER |
| | E682S, R685Q, E686L, | ADSVISWDIQDEKNVTCQLTFWEASERTIRSEAEDSYHESSAKM |
| | N688E, Y690F, R693V, | TATFLSKKQEVNMSDSALDCVRDEAINKLQQIFNTSYNQTYEKY |
| | V694L, K695D, D699K, | GNVSVFETTGGLVVFWQGIKQKSLVELERLANRSSLNLTHNSTK |
| | K700A, V701L, V702Q | SSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLFKR |
| | | MIDLSSISTVDSMIALDIDPLENTDFRVLELYSQKELRSSNTNN |
| | | ENSIMQLFESFKQVLDYVEKALQ |
| SEQ ID | gB-Towne-ext-N-term_Bg- | GAAVSSSSTRGTSATHSHHSSHTTSAAHSRSGSVSQRVTSSQTV |
| NO: 45 | KOs_Cpack4-short | SHGVNETIYNTTLKYGDVVGVNITKYPYRVCSMAQGTDLIRFER |
| | Bg-KOs: I156H, H157R, | NIVCTSMKPINEDLDEGIMVVYKRNIVAHTFKVRVYQKVLTERR |
| | W240N, Y242T, C246S, | SYAYHRTTYLLGSNTEYVAPPMWEIHHINSHSQCYSSYSRVIAG |
| | R457S, R460S | TVFVAYHRDSYENKTMQLMPDDYSNTHSTRYVTVKDQWHSRGST |
| | Cpack4-short: V677T, | NLTRETSNLNCMVTITTARSKYPYHFFATSTGDVVDISPFYNGT |
| | F678N, D679H, L680E, | NRNASYFGENADKFFIFPNYTIVSDFGRPNSALETHREVAFLER |
| | E681N, E682S, M684S, | ADSVISWDIQDEKNVTCQLIFWEASERTIRSEAEDSYHESSAKM |
| | R685Q, E686L, F687A, | TATFLSKKQEVNMSDSALDCVRDEAINKLQQIFNTSYNQTYEKY |
| | N688E, Y690F, Q692S, | GNVSVFETTGGLVVFWQGIKQKSLVELERLANRSSLNLTHNSTK |
| | R693V, V694L, K695D, Y696R | SSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNINH |
| | | ENSISQLAESFKSVLDRV |
| SEQ ID | gB-Towne-ext-N-term_Bg- | GAAVSSSSTRGTSATHSHHSSHTTSAAHSRSGSVSQRVTSSQTV |
| NO: 46 | KOs_Cpack4-long | SHGVNETIYNTTLKYGDVVGVNTTKYPYRVCSMAQGTDLIRFER |
| | Bg-KOs: I156H, H157R, | NIVCTSMKPINEDLDEGIMVVYKRNIVAHTEKVRVYQKVLTERR |
| | W240N, Y242T, C246S, | SYAYHRTTYLLGSNTEYVAPPMWEIHHINSHSQCYSSYSRVIAG |
| | R457S, R460S | TVFVAYHRDSYENKTMQLMPDDYSNTHSTRYVTVKDQWHSRGST |
| | Cpack4-long: V677T, F678N, | NLTRETSNLNCMVTITTARSKYPYHFFATSTGDVVDISPFYNGT |
| | D679H, L680E, E681N, | NRNASYFGENADKFFIFPNYTIVSDFGRPNSALETHRLVAFLER |
| | E682S, M684S, R685Q, | ADSVISWDIQDEKNVTCQLTFWEASERTIRSEAEDSYHFSSAKM |
| | E686L, F687A, N688E, | TATFLSKKQEVNMSDSALDCVRDEAINKLQQIFNTSYNQTYEKY |
| | Y690F, Q6925, R693V, | GNVSVFETTGGLVVFWQGIKQKSLVELERLANRSSLNLTHNSTK |
| | V694L, K695D, Y696R, | SSTDGNNATHLSNMESVHNLVYAQLQFTYDTLRGYINRALAQIA |
| | D699K, K700A, V701L, V702E | EAWCVDQRRTLEVEKELSKINPSAILSAIYNKPIAAREMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDFRVLELYSQKELRSSNTNH |
| | | ENSISQLAESFKSVLDRVEKALE |
| SEQ ID | gB-Towne-ext-N-term_Bg- | GAAVSSSSTRGTSATHSHHSSHTTSAAHSRSGSVSQRVTSSQTV |
| NO: 47 | KOs_Cpack1-short_ | SHGVNETIYNTTLKYGDVVGVNTTKYPYRVCSMAQGTDLIRFER |
| | dII-dIII-pack1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDDYSNTHSTRYVTVKDQWHSRGST |
| | R457S, R460S | NLTRETSNLNCMVTITTARSKYPYHFFATSIGDVVDISPFYNGT |
| | dII-dIII-pack1: | NRNASYFGENADKFFIFPNYTIVSDFGRPNSALETHRIVAFLER |
| | S367I, T374F | ADSVISWDIQDEKNVTCQLIFWEASERTIRSEAEDSYHFISAKM |
| | Cpack1-short: E686L, | TAFFLSKKQEVNMSDSALDCVRDEAINKLQQIFNTSYNQTYEKY |
| | Y690F, R693V, V694L | GNVSVFETTGGLVVFWQGIKQKSIVELERLANRSSLNLTHNSTK |
| | | SSTDGNNATHLSNMESVHNLVYAQLQFTYDTLRGYINRALAQIA |
| | | EAWCVDQRRTLEVFKELSKINPSAILSAIYNKPIAARFMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVED |
| | | LEEIMRLENSFKQVLKYV |
| SEQ ID | gB-Towne-ext-N-term_Bg- | GAAVSSSSTRGTSATHSHHSSHITSAAHSRSGSVSQRVTSSQTV |
| NO: 48 | KOs_Cpack1-long_ | SHGVNETIYNTTLKYGDVVGVNTTKYPYRVCSMAQGTDLIRFER |
| | dII-dIII-pack1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDDYSNTHSTRYVTVKDQWHSRGST |
| | R457S, R460S | NLTRETSNLNCMVTITTARSKYPYHFFATSTGDVVDISPFYNGT |
| | dII-dIII-pack1: | NRNASYFGENADKFFIFPNYTIVSDFGRPNSALETHRLVAFLER |
| | S367I, T374F | ADSVISWDIQDEKNVICQLTFWEASERTIRSEAEDSYHFISAKM |
| | Cpack1-long: | TAFFLSKKQEVNMSDSALDCVRDEAINKLQQIFNTSYNQTYEKY |
| | E686L, Y690F, | GNVSVFETTGGLVVFWQGIKQKSLVELERLANRSSLNLTANSTK |
| | R693V, V694L, K700A, V702Q | SSTDGNNATHLSNMESVHNLVYAQLQFTYDTLRGYINRALAQIA |
| | | EAWCVDQRRTLEVFKELSKINPSAILSAIYNKPIAARFMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVED |
| | | LEEIMRLFNSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 49 | KOs_Cpack2-short_ | SHGVNETIYNTTLKYGDVVGVNTTKYPYRVCSMAQGTDLIRFER |
| | dII-dIII-pack1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVEVAYHRDSYENKTMQLMPDDYSNTHSTRYVTVKDQWHSRGST |
| | R457S, R460S | NLTRETSNLNCMVTITTARSKYPYHFFATSTGDVVDISPFYNGT |
| | dII-dIII-pack1: S367I, | NRNASYFGENADKFFIFPNYTIVSDEGRPNSALETHRLVAFLER |
| | T374F | ADSVISWDIQDEKNVTCQLIFWEASERTIRSEAEDSYHFISAKM |
| | Cpack2-short: D679N, | TAFFLSKKQEVNMSDSALDCVRDEAINKLQQIFNTSYNQTYEKY |
| | L680E, E681N, R685Q, | GNVSVFETTGGLVVFWQGIKQKSLVELERLANRSSLNLTHNSTK |
| | E686L, Y690F, R693V, | SSTDGNNATHLSNMESVHNLVYAQLQFTYDTLRGYINRALAQIA |
| | V694L, K695D | EAWCVDQRRTLEVEKELSKINPSAILSAIYNKPIAAREMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVEN |
| | | ENEIMQLFNSFKQVLDYV |
| SEQ ID | gB-Towne-ext-N-term_Bg- | GAAVSSSSTRGTSATHSHHSSHTTSAAHSRSGSVSQRVTSSQTV |
| NO: 50 | KOs_Cpack2-long_ | SHGVNETIYNTTLKYGDVVGVNTTKYPYRVCSMAQGTDLIRFER |
| | dII-dIII-pack1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDDYSNTHSTRYVTVKDQWHSRGST |
| | R457S, R460S | NLTRETSNLNCMVTITTARSKYPYHFFATSTGDVVDISPFYNGT |
| | dII-dIII-pack1: S367I, | NRNASYFGENADKFFIFPNYTIVSDFGRPNSALETHRLVAFLER |
| | T374F | ADSVISWDIQDEKNVTCQLTFWEASERTIRSEAEDSYHFISAKM |
| | Cpack2-long: D679N, L680E, | TAFFLSKKQEVNMSDSALDCVRDEAINKLQQIFNTSYNQTYEKY |
| | E681N, R685Q, E686L, | GNVSVFETTGGLVVFWQGIKQKSLVELERLANRSSLNLTHNSTK |
| | Y690F, R693V, V694L, | SSTDGNNATHLSNMESVHNLVYAQLQFTYDTLRGYINRALAQIA |
| | K695D, K700A, V701L, V702Q | EAWCVDQRRTLEVEKELSKINPSAILSAIYNKPIAARFMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLFKR |
| | | MIDLSSISTVDSMIALDIDPLENTDFRVLELYSQKELRSSNVEN |
| | | ENEIMQLENSFKQVLDYVEDALQ |
| SEQ ID | gB-Towne-ext-N-term_Bg- | GAAVSSSSTRGTSATHSHHSSHTTSAAHSRSGSVSQRVTSSQTV |
| NO: 51 | KOs_Cpack3-short_ | SHGVNETIYNTTLKYGDVVGVNITKYPYRVCSMAQGTDLIRFER |
| | pdII-dIII-ack1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDDYSNTHSTRYVTVKDQWHSRGST |
| | R457S, R460S | NLTRETSNLNCMVTITTARSKYPYHFFATSTGDVVDISPFYNGT |
| | dII-dIII-pack1: S367I, | NRNASYFGENADKFFIFPNYTIVSDFGRPNSALETHRLVAFLER |
| | T374F | ADSVISWDIQDEKNVICQLTFWEASERTIRSEAEDSYHFISAKM |
| | Cpack3-short: V677T, | TAFFLSKKQEVNMSDSALDCVRDEAINKLQQIFNTSYNQTYEKY |
| | F678N, D679N, L680E, | GNVSVFETTGGLVVFWQGIKQKSLVELERLANRSSLNITANSTK |
| | E681N, E682S, R685Q, | SSTDGNNATHLSNMESVHNLVYAQLQFTYDTLRGYINRALAQIA |
| | E686L, N688E, Y690F, | EAWCVDQRRILEVEKELSKINPSAILSAIYNKPIAARFMGDVLG |
| | R693V, V694L, K695D | LASCVTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNINN |
| | | ENSIMQLFESFKQVLDYV |
| SEQ ID | gB-Towne-ext-N-term_Bg- | GAAVSSSSTRGTSATHSHHSSHTTSAAHSRSGSVSQRVTSSQTV |
| NO: 52 | KOs_Cpack3-long_ | SHGVNETIYNTTLKYGDVVGVNTTKYPYRVCSMAQGTDLIRFER |
| | dII-dIII-pack1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDDYSNTHSTRYVTVKDQWHSRGST |
| | R457S, R460S | NLTRETSNLNCMVTITTARSKYPYHFFATSTGDVVDISPFYNGT |
| | dII-dIII-pack1: S367I, | NRNASYFGENADKFFIFPNYTIVSDEGRPNSALETHRLVAFLER |
| | T374F | ADSVISWDIQDEKNVTCQLIFWEASERTIRSEAEDSYHFISAKM |
| | Cpack3-long: V677T, F678N, | TAFFLSKKQEVNMSDSALDCVRDEAINKLQQIFNTSYNQTYEKY |
| | D679N, L680E, E681N, | GNVSVFETTGGLVVFWQGIKQKSLVELERLANRSSLNLTHNSTK |
| | E682S, R685Q, E686L, | SSTDGNNATHLSNMESVHNLVYAQLQFTYDTLRGYINRALAQIA |
| | N688E, Y690F, R693V, | EAWCVDQRRTLEVEKELSKINPSAILSAIYNKPIAAREMGDVLG |
| | V694L, K695D, D699K, | LASCVTINQTSVKVLRDMNVKESPGRCYSRPVVIENFANSSYVQ |
| | K700A, V701L, V702Q | YGQLGEDNEILLGNHRTEECQLPSLKIFIAGNSAYEYVDYLEKR |
| | | MIDLSSISTVDSMIALDIDPLENTDERVLELYSQKELRSSNTNN |
| | | ENSIMQLFESEKQVLDYVEKALQ |
| SEQ ID | gB-Towne-ext-N-term_Bg- | GAAVSSSSTRGTSATHSHHSSHTTSAAHSRSGSVSQRVTSSQTV |
| NO: 53 | KOs_Cpack4-short_ | SHGVNETIYNTTLKYGDVVGVNTTKYPYRVCSMAQGTDLIRFER |
| | dII-dIII-pack1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVEVAYARDSYENKTMQLMPDDYSNTHSTRYVTVKDQWHSRGST |
| | R457S, R460S | NLTRETSNLNCMVTITTARSKYPYHFFATSTGDVVDISPFYNGT |
| | dII-dIII-pack1: S367I, | NRNASYFGENADKFFIFPNYTIVSDFGRPNSALETHRLVAFLER |
| | T374F | ADSVISWDIQDEKNVTCQLTFWEASERTIRSEAEDSYHFISAKM |
| | Cpack4-short: V677T, | TAFFLSKKQEVNMSDSALDCVRDEAINKLQQIFNTSYNQTYEKY |
| | F678N, D679H, L680E, | GNVSVFETTGGLVVFWQGIKQKSLVELERLANRSSLNLTHNSTK |
| | E681N, E682S, M684S, | SSTDGNNATHLSNMESVHNLVYAQLQFTYDTLRGYINRALAQIA |
| | R685Q, E686L, F687A, | EAWCVDQRRTLEVEKELSKINPSAILSAIYNKPIAARFMGDVLG |
| | N688E, Y690F, Q692S, | LASCVTINQTSVKVLRDMNVKESPGRCYSRPVVIENFANSSYVQ |
| | R693V, V694L, K695D, Y696R | YGQLGEDNEILLGNHRTEECQLPSLKIFIAGNSAYEYVDYLEKR |
| | | MIDLSSISTVDSMIALDIDPLENTDERVLELYSQKELRSSNINH |
| | | ENSISQLAESFKSVLDRV |
| SEQ ID | gB-Towne-ext-N-term_Bg- | GAAVSSSSTRGTSATHSHHSSHTTSAAHSRSGSVSQRVTSSQTV |
| NO: 54 | KOs_Cpack4-long_ | SHGVNETIYNTTLKYGDVVGVNTTKYPYRVCSMAQGTDLIRFER |
| | dII-dIII-pack1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVEVAYHRDSYENKTMQLMPDDYSNTHSTRYVTVKDQWHSRGST |
| | R457S, R460S | NLTRETSNLNCMVTITTARSKYPYHFFATSIGDVVDISPFYNGT |
| | dII-dIII-pack1: S367I, | NRNASYFGENADKFFIFPNYTIVSDFGRPNSALETHRIVAFLER |
| | T374F | ADSVISWDIQDEKNVTCQLIFWEASERTIRSEAEDSYHFISAKM |
| | Cpack4-long: V677T, F678N, | TAFFLSKKQEVNMSDSALDCVRDEAINKLQQIFNTSYNQTYEKY |
| | D679H, L680E, E681N, | GNVSVFETTGGLVVFWQGIKQKSIVELERLANRSSLNLTHNSTK |
| | E682S, M684S, R685Q, | SSTDGNNATHLSNMESVHNLVYAQLQFTYDTLRGYINRALAQIA |
| | E686L, F687A, N688E, | EAWCVDQRRTLEVFKELSKINPSAILSAIYNKPIAAREMGDVLG |
| | Y690F, Q692S, R693V, | LASCVTINQTSVKVLRDMNVKESPGRCYSRPVVIENFANSSYVQ |
| | V6941, K695D, Y696R, | YGQLGEDNEILLGNHRTEECQLPSLKIFIAGNSAYEYVDYLFKR |
| | D699K, K700A, V701L, V702E | MIDLSSISTVDSMIALDIDPLENTDERVLELYSQKELRSSNINH |
| | | ENSISQLAESFKSVLDRVEKALE |
| SEQ ID | gB-Towne-ext-N-term_Bg- | GAAVSSSSTRGTSATHSHHSSHTTSAAHSRSGSVSQRVTSSQTV |
| NO: 55 | KOs_Cpack1-short_dI-dIV- | SHGVNETIYNTTLKYGDVVGVNTTKYPYRVCSMAQGTDLIRFER |
| | dV-pack1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDDFSNTHSTRYVTVKDQWHSRGST |
| | R457S, R460S | NLTRETSNLNCMVTITTARSKYPYHFFATSTGDVVDISPFYNGT |
| | Cpack1-short: E686L, | NRNASYFGENADKFFIFPNYTIVSDFGRPNSALETHRLVAFLER |
| | Y690F, R693V, V694L | ADSVISWDIQDEKNVICQLTFWEASERTIRSEAEDSYHFSSAKM |
| | dI-dIV-dV-pack1: Y218F, | TATFLSKKQEVNMSDSALDCVRDEAINKLQQIFNTSYNQTYEKY |
| | Q591F, S668A | GNVSVFETTGGLVVFWQGIKQKSLVELERLANRSSLNLTHNSTK |
| | | SSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCVTINQTSVKVLRDMNVKESPGRCYSRPVVIENFANSSYVE |
| | | YGQLGEDNEILLGNHRTEECQLPSLKIFIAGNSAYEYVDYLFKR |
| | | MIDLSSISTVDSMIALDIDPLENTDFRVLELYAQKELRSSNVFD |
| | | LEEIMRLENSFKQVLKYV |
| SEQ ID | gB-Towne-ext-N-term_Bg- | GAAVSSSSTRGTSATHSHHSSHTTSAAHSRSGSVSQRVTSSQTV |
| NO: 56 | KOs_Cpack1-long_ | SHGVNETIYNTTLKYGDVVGVNTTKYPYRVCSMAQGTDLIRFER |
| | dI-dIV-dV-pack1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDDESNTHSTRYVTVKDQWHSRGST |
| | R457S, R460S | NLTRETSNLNCMVTITTARSKYPYHFFATSTGDVVDISPFYNGT |
| | Cpack1-long: E686L, Y690F, | NRNASYFGENADKFFIFPNYTIVSDFGRPNSALETHRLVAFLER |
| | R693V, V694L, K700A, V702Q | ADSVISWDIQDEKNVTCQLTFWEASERTIRSEAEDSYHFSSAKM |
| | dI-dIV-dV-pack1: Y218F, | TATFLSKKQEVNMSDSALDCVRDEAINKLQQIFNTSYNQTYEKY |
| | Q591F, S668A | GNVSVFETTGGLVVFWQGIKQKSIVELERLANRSSLNLTANSTK |
| | | SSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCVTINQTSVKVLRDMNVKESPGRCYSRPVVIENFANSSYVE |
| | | YGQLGEDNEILLGNHRTEECQLPSLKIFIAGNSAYEYVDYLEKR |
| | | MIDLSSISTVDSMIALDIDPLENTDERVLELYAQKELRSSNVED |
| | | LEEIMRLFNSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 57 | KOs_Cpack1-short_Disulf1 | SHGVNETIYNTTLKYGDVVGVNTTKYPYRVCSMAQGTDLIRFER |
| | Bg-KOs: I156H, H157R, | NIVCTSMKPINEDLDEGIMVVYKRNIVAHTFKVRVYQKVLTERR |
| | W240N, Y242T, C246S, | SYAYHRTTYLLGSNTEYVAPPMWEIHHINSHSQCYSSYSRVIAG |
| | R457S, R460S | TVEVAYHRDSYENKTMQLMPDCYSNTHSTRYVTVKDQWHSRGST |
| | Cpack1-short: E686L, | NLTRETSNLNCMVTITTARSKYPYHFFATSTGDVVDISPFYNGT |
| | Y690F, R693V, V694L | NRNASYFGENADKFFIFPNYTIVSDFGRPNSALETHRLVAFLER |
| | Disulf1: D217C, S587C | ADSVISWDIQDEKNVICQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNSTK |
| | | SSTDGNNATHLSNMESVHNLVYAQLQFTYDTLRGYINRALAQIA |
| | | EAWCVDQRRTLEVFKELSKINPSAILSAIYNKPIAARFMGDVLG |
| | | LASCVTINQTSVKVLRDMNVKESPGRCYSRPVVIENFANCSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVED |
| | | LEEIMRLENSFKQVLKYV |
| SEQ ID | gB-Towne-ext-N-term_Bg- | GAAVSSSSTRGTSATHSHHSSHTTSAAHSRSGSVSQRVTSSQTV |
| NO: 58 | KOs_Cpack1-long_Disulf1 | SHGVNETIYNTTLKYGDVVGVNTTKYPYRVCSMAQGTDLIRFER |
| | Bg-KOs: I156H, H157R, | NIVCTSMKPINEDLDEGIMVVYKRNIVAHTFKVRVYQKVLTERR |
| | W240N, Y242T, C246S, | SYAYHRTTYLLGSNTEYVAPPMWEIHHINSHSQCYSSYSRVIAG |
| | R457S, R460S | TVFVAYHRDSYENKTMQLMPDCYSNTHSTRYVTVKDQWHSRGST |
| | Cpack1-long: E686L, Y690F, | NLTRETSNINCMVTITTARSKYPYHFFATSTGDVVDISPFYNGT |
| | R693V, V694L, K700A, V702Q | NRNASYFGENADKFFIFPNYTIVSDFGRPNSALETHRLVAFLER |
| | Disulf1: D217C, S587C | ADSVISWDIQDEKNVICQLTFWEASERTIRSEAEDSYHESSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNSTK |
| | | SSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCVTINQTSVKVLRDMNVKESPGRCYSRPVVIENFANCSYVQ |
| | | YGQLGEDNEILLGNHRTEECQLPSLKIFIAGNSAYEYVDYLEKR |
| | | MIDLSSISTVDSMIALDIDPLENTDFRVLELYSQKELRSSNVFD |
| | | LEEIMRLFNSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 59 | KOs_Cpack1-short_Disulf2 | SHGVNETIYNTTLKYGDVVGVNITKYPYRVCSMAQGTDLIRFER |
| | Bg-KOs: I156H, H157R, | NIVCTSMKPINEDLDEGIMVVYKRNIVAHTFKVRVYQKVLTERR |
| | W240N, Y242T, C246S, | SYAYHRTTYLLGSNTEYVAPPMWEIHHINSHSQCYSSYSRVIAG |
| | R457S, R460S | TVEVAYHRDSYENKTMQLMPDCYSNTHSTRYVTVKDQWHSRGST |
| | Cpack1-short: E686L, | NLTRETSNLNCMVTITTARSKYPYHFFATSTGDVVDISPFYNGT |
| | Y690F, R693V, V694L | NRNASYFGENADKFFIFPNYTIVSDFGRPNSALETHREVAFLER |
| | Disulf2: D217C, Y589C | ADSVISWDIQDEKNVICQLTFWEASERTIRSEAEDSYHESSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTANSTK |
| | | SSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCVTINQTSVKVLRDMNVKESPGRCYSRPVVIENFANSSCVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVED |
| | | LEEIMRLENSFKQVLKYV |
| SEQ ID | gB-Towne-ext-N-term_Bg- | GAAVSSSSTRGTSATHSHHSSHTTSAAHSRSGSVSQRVTSSQTV |
| NO: 60 | KOs_Cpack1-long_Disulf2 | SHGVNETIYNTTLKYGDVVGVNTTKYPYRVCSMAQGTDLIRFER |
| | Bg-KOs: I156H, H157R, | NIVCTSMKPINEDLDEGIMVVYKRNIVAHTEKVRVYQKVLTERR |
| | W240N, Y242T, C246S, | SYAYHRTTYLLGSNTEYVAPPMWEIHHINSHSQCYSSYSRVIAG |
| | R457S, R460S | TVFVAYHRDSYENKTMQLMPDCYSNTHSTRYVTVKDQWHSRGST |
| | Cpack1-long: E6861, Y690F, | NLTRETSNLNCMVTITTARSKYPYHFFATSTGDVVDISPFYNGT |
| | R693V, V694L, K700A, V702Q | NRNASYFGENADKFFIFPNYTIVSDFGRPNSALETHRLVAFLER |
| | Disulf2: D217C, Y589C | ADSVISWDIQDEKNVTCQLTFWEASERTIRSEAEDSYHESSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNSTK |
| | | SSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIENFANSSCVQ |
| | | YGQLGEDNEILLGNHRTEECQLPSLKIFIAGNSAYEYVDYLFKR |
| | | MIDLSSISTVDSMIALDIDPLENTDFRVLELYSQKELRSSNVED |
| | | LEEIMRLFNSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 61 | KOs_Cpack1-short_ | SHGVNETIYNTTLKYGDVVGVNTTKYPYRVCSMAQGTDLIRFER |
| | dI-dIV-dV-pack1_Disulf1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVEVAYHRDSYENKTMQLMPDCFSNTHSTRYVTVKDQWHSRGST |
| | R457S, R460S | NLTRETSNLNCMVTITTARSKYPYHFFATSIGDVVDISPFYNGT |
| | Cpack1-short: E686L, | NRNASYFGENADKFFIFPNYTIVSDFGRPNSALETHRIVAFLER |
| | Y690F, R693V, V694L | ADSVISWDIQDEKNVICQLTFWEASERTIRSEAEDSYHFSSAKM |
| | dI-dIV-dV-pack1: Y218F, | TATFLSKKQEVNMSDSALDCVRDEAINKLQQIFNTSYNQTYEKY |
| | Q591F, S668A | GNVSVFETTGGLVVFWQGIKQKSIVELERLANRSSLNLTHNSTK |
| | Disulf1: D217C, S587C | SSTDGNNATHLSNMESVHNLVYAQLQFTYDTLRGYINRALAQIA |
| | | EAWCVDQRRTLEVFKELSKINPSAILSAIYNKPIAARFMGDVLG |
| | | LASCVTINQTSVKVLRDMNVKESPGRCYSRPVVIENFANCSYVE |
| | | YGQLGEDNEILLGNHRTEECQLPSLKIFIAGNSAYEYVDYLFKR |
| | | MIDLSSISTVDSMIALDIDPLENTDERVLELYAQKELRSSNVED |
| | | LEEIMPLENSFKQVLKYV |
| SEQ ID | gB-Towne-ext-N-term_Bg- | GAAVSSSSTRGTSATHSHHSSHTTSAAHSRSGSVSQRVTSSQTV |
| NO: 62 | KOs_Cpack1-long_dI-dIV-dV- | SHGVNETIYNTTLKYGDVVGVNITKYPYRVCSMAQGTDLIRFER |
| | pack1_Disulf1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDCFSNTHSTRYVTVKDQWHSRGST |
| | R457S, R460S | NLTRETSNLNCMVTITTARSKYPYHFFATSTGDVVDISPFYNGT |
| | Cpack1-long: E686L, Y690F, | NRNASYFGENADKFFIFPNYTIVSDFGRPNSALETHRLVAFLER |
| | R693V, V694L, K700A, V702Q | ADSVISWDIQDEKNVTCQLTFWEASERTIRSEAEDSYHESSAKM |
| | dI-dIV-dV-pack1: Y218F, | TATFLSKKQEVNMSDSALDCVRDEAINKLQQIFNTSYNQTYEKY |
| | Q591F, S668A | GNVSVFETTGGLVVFWQGIKQKSIVELERLANRSSLNITHNSTK |
| | Disulf1: D217C, S587C | SSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCVTINQTSVKVLRDMNVKESPGRCYSRPVVIENFANCSYVE |
| | | YGQLGEDNEILLGNHRTEECQLPSLKIFIAGNSAYEYVDYLEKR |
| | | MIDLSSISTVDSMIALDIDPLENTDERVLELYAQKELRSSNVED |
| | | LEEIMRLFNSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 63 | KOs_Cpack1-short_dI-dIV- | SHGVNETIYNTTLKYGDVVGVNTTKYPYRVCSMAQGTDLIRFER |
| | dV-pack1_Disulf2 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDCFSNTHSTRYVTVKDQWHSRGST |
| | R457S, R460S | NLTRETSNLNCMVTITTARSKYPYHFFATSTGDVVDISPFYNGT |
| | Cpack1-short: E686L, | NRNASYFGENADKFFIFPNYTIVSDEGRPNSALETHRLVAFLER |
| | Y690F, R693V, V694L | ADSVISWDIQDEKNVTCQLIFWEASERTIRSEAEDSYHFSSAKM |
| | dI-dIV-dV-pack1: Y218F, | TATFLSKKQEVNMSDSALDCVRDEAINKLQQIFNTSYNQTYEKY |
| | Q591F, S668A | GNVSVFETTGGLVVFWQGIKQKSLVELERLANRSSLNLTHNSTK |
| | Disulf2: D217C, Y589C | SSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCVTINQTSVKVLRDMNVKESPGRCYSRPVVIENFANSSCVE |
| | | YGQLGEDNEILLGNHRTEECQLPSLKIFIAGNSAYEYVDYLEKR |
| | | MIDLSSISTVDSMIALDIDPLENTDFRVLELYAQKELRSSNVED |
| | | LEEIMRLFNSFKQVLKYV |
| SEQ ID | gB-Towne-ext-N-term_Bg- | GAAVSSSSTRGTSATHSHHSSHTTSAAHSRSGSVSQRVTSSQTV |
| NO: 64 | KOs_Cpack1-long_dI-dIV-dV- | SHGVNETIYNTTLKYGDVVGVNTTKYPYRVCSMAQGTDLIRFER |
| | pack1_Disulf2 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDCFSNTHSTRYVTVKDQWHSRGST |
| | R457S, R460S | NLTRETSNLNCMVTITTARSKYPYHFFATSTGDVVDISPFYNGT |
| | Cpack1-long: E686L, Y690F, | NRNASYFGENADKFFIFPNYTIVSDFGRPNSALETHRLVAFLER |
| | R693V, V694L, K700A, V702Q | ADSVISWDIQDEKNVICQLTFWEASERTIRSEAEDSYHFSSAKM |
| | dI-dIV-dV-pack1: Y218F, | TATFLSKKQEVNMSDSALDCVRDEAINKLQQIFNTSYNQTYEKY |
| | Q591F, S668A | GNVSVFETTGGLVVFWQGIKQKSLVELERLANRSSLNLTHNSTK |
| | Disulf2: D217C, Y589C | SSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCVTINQTSVKVLRDMNVKESPGRCYSRPVVIENFANSSCVE |
| | | YGQLGEDNEILLGNHRTEECQLPSLKIFIAGNSAYEYVDYLFKR |
| | | MIDLSSISTVDSMIALDIDPLENTDFRVLELYAQKELRSSNVFD |
| | | LEEIMRLENSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 65 | KOs_Cpack1-short_dI-dIV- | SHGVNETIYNTTLKYGDVVGVNITKYPYRVCSMAQGTDLIRFER |
| | dV-pack1 N165glyc | NIVCTSMKPINEDLDEGIMVVYKRNIVAHTFKVRVYQKVLTERR |
| | Bg-KOs: I156H, H157R, | SYAYHRTTYLLGSNTTYVAPPMWEIHHINSHSQCYSSYSRVIAG |
| | W240N, Y242T, C246S, | TVEVAYHRDSYENKTMQLMPDDESNTHSTRYVTVKDQWHSRGST |
| | R457S, R460S | NLTRETSNLNCMVTITTARSKYPYHFFATSTGDVVDISPFYNGT |
| | Cpack1-short: E686L, | NRNASYFGENADKFFIFPNYTIVSDFGRPNSALETHRLVAFLER |
| | Y690F, R693V, V694L | ADSVISWDIQDEKNVTCQLTFWEASERTIRSEAEDSYHFSSAKM |
| | dI-dIV-dV-pack1: Y218F, | TATFLSKKQEVNMSDSALDCVRDEAINKLQQIFNTSYNQTYEKY |
| | Q591F, S668A | GNVSVFETTGGLVVFWQGIKQKSLVELERLANRSSLNLTANSTK |
| | N165glyc: E167T | SSTDGNNATHLSNMESVHNLVYAQLQFTYDTLRGYINRALAQIA |
| | | EAWCVDQRRILEVFKELSKINPSAILSAIYNKPIAAREMGDVLG |
| | | LASCVTINQTSVKVLRDMNVKESPGRCYSRPVVIENFANSSYVE |
| | | YGQLGEDNEILLGNHRTEECQLPSLKIFIAGNSAYEYVDYLEKR |
| | | MIDLSSISTVDSMIALDIDPLENTDERVLELYAQKELRSSNVED |
| | | LEEIMRLFNSFKQVLKYV |
| SEQ ID | gB-Towne-ext-N-term_Bg- | GAAVSSSSTRGTSATHSHHSSHTTSAAHSRSGSVSQRVTSSQTV |
| NO: 66 | KOs_Cpack1-long_dI-dIV-dV- | SHGVNETIYNTTLKYGDVVGVNTTKYPYRVCSMAQGTDLIRFER |
| | pack1_N165glyc | NIVCTSMKPINEDLDEGIMVVYKRNIVAHTFKVRVYQKVLTERR |
| | Bg-KOs: I156H, H157R, | SYAYHRTTYLLGSNTTYVAPPMWEIHHINSHSQCYSSYSRVIAG |
| | W240N, Y242T, C246S, | TVFVAYHRDSYENKTMQLMPDDESNTHSTRYVTVKDQWHSRGST |
| | R457S, R460S | NLTRETSNLNCMVTITTARSKYPYHFFATSTGDVVDISPFYNGT |
| | Cpack1-long: E686L, Y690F, | NRNASYFGENADKFFIFPNYTIVSDEGRPNSALETHRLVAFLER |
| | R693V, V694L, K700A, V702Q | ADSVISWDIQDEKNVTCQLIFWEASERTIRSEAEDSYHESSAKM |
| | dI-dIV-dV-pack1: Y218F, | TATFLSKKQEVNMSDSALDCVRDEAINKLQQIFNTSYNQTYEKY |
| | Q591F, S668A | GNVSVFETTGGLVVFWQGIKQKSIVELERLANRSSLNLTHNSTK |
| | N165glyc: E167T | SSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCVTINQTSVKVLRDMNVKESPGRCYSRPVVIENFANSSYVE |
| | | YGQLGEDNEILLGNHRTEECQLPSLKIFIAGNSAYEYVDYLEKR |
| | | MIDLSSISTVDSMIALDIDPLENTDFRVLELYAQKELRSSNVED |
| | | LEEIMRLFNSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 67 | KOs_Cpack1-short_dII-dIII- | SHGVNETIYNTTLKYGDVVGVNTTKYPYRVCSMAQGTDLIRFER |
| | pack1_dI-dIV-dV-pack1 | NIVCTSMKPINEDLDEGIMVVYKRNIVAHTEKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVEVAYHRDSYENKTMQLMPDDESNTHSTRYVTVKDQWHSRGST |
| | R457S, R460S | NLTRETSNLNCMVTITTARSKYPYHFFATSTGDVVDISPFYNGT |
| | dII-dIII-pack1: S367I, | NRNASYFGENADKFFIFPNYTIVSDFGRPNSALETHRIVAFLER |
| | T374F | ADSVISWDIQDEKNVTCQLIFWEASERTIRSEAEDSYHFISAKM |
| | Cpack1-short: E686L, | TAFFLSKKQEVNMSDSALDCVRDEAINKLQQIFNTSYNQTYEKY |
| | Y690F, R693V, V694L | GNVSVFETTGGLVVFWQGIKQKSLVELERLANRSSINLTHNSTK |
| | dI-dIV-dV-pack1: Y218F, | SSTDGNNATHLSNMESVHNLVYAQLQFTYDTLRGYINRALAQIA |
| | Q591F, S668A | EAWCVDQRRTLEVFKELSKINPSAILSAIYNKPIAAREMGDVLG |
| | | LASCVTINQTSVKVLRDMNVKESPGRCYSRPVVIFNFANSSYVF |
| | | YGQLGEDNEILLGNHRTEECQLPSLKIFIAGNSAYEYVDYLEKR |
| | | MIDLSSISTVDSMIALDIDPLENTDERVLELYAQKELRSSNVED |
| | | LEEIMRLENSFKQVLKYV |
| SEQ ID | gB-Towne-ext-N-term_Bg- | GAAVSSSSTRGTSATHSHHSSHTTSAAHSRSGSVSQRVTSSQTV |
| NO: 68 | KOs_Cpack1-long_dII-dIII- | SHGVNETIYNTTLKYGDVVGVNTTKYPYRVCSMAQGTDLIRFER |
| | pack1_dI-dIV-dV-pack1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDDFSNTHSTRYVTVKDQWHSRGST |
| | R457S, R460S | NLTRETSNLNCMVTITTARSKYPYHFFATSTGDVVDISPFYNGT |
| | dII-dIII-pack1: S367I, | NRNASYFGENADKFFIFPNYTIVSDFGRPNSALETHRLVAFLER |
| | T374F | ADSVISWDIQDEKNVICQLTFWEASERTIRSEAEDSYHFISAKM |
| | Cpack1-long: E686L, Y690F, | TAFFLSKKQEVNMSDSALDCVRDEAINKLQQIFNTSYNQTYEKY |
| | R693V, V694L, K700A, V702Q | GNVSVFETTGGLVVFWQGIKQKSLVELERLANRSSLNLTHNSTK |
| | dI-dIV-dV-pack1: Y218F, | SSTDGNNATHLSNMESVHNLVYAQLQFTYDTLRGYINRALAQIA |
| | Q591F, S668A | EAWCVDQRRTLEVEKELSKINPSAILSAIYNKPIAARFMGDVLG |
| | | LASCVTINQTSVKVLRDMNVKESPGRCYSRPVVIENFANSSYVE |
| | | YGQLGEDNEILLGNHRTEECQLPSLKIFIAGNSAYEYVDYLFKR |
| | | MIDLSSISTVDSMIALDIDPLENTDFRVLELYAQKELRSSNVFD |
| | | LEEIMRLFNSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 69 | KOs_Cpack1-short_dII-dIII- | SHGVNETIYNTTLKYGDVVGVNTTKYPYRVCSMAQGTDLIRFER |
| | pack1_Disulf1 | NIVCTSMKPINEDLDEGIMVVYKRNIVAHTEKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDCYSNTHSTRYVTVKDQWHSRGST |
| | R457S, R460S | NLTRETSNLNCMVTITTARSKYPYHFFATSTGDVVDISPFYNGT |
| | dII-dIII-pack1: S367I, | NRNASYFGENADKFFIFPNYTIVSDFGRPNSALETHRLVAFLER |
| | T374F | ADSVISWDIQDEKNVTCQLTFWEASERTIRSEAEDSYHFISAKM |
| | Cpack1-short: E686L, | TAFFLSKKQEVNMSDSALDCVRDEAINKLQQIFNTSYNQTYEKY |
| | Y690F, R693V, V694L | GNVSVFETTGGLVVFWQGIKQKSLVELERLANRSSLNLTHNSTK |
| | Disulf1: D217C, S587C | SSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIENFANCSYVQ |
| | | YGQLGEDNEILLGNHRTEECQLPSLKIFIAGNSAYEYVDYLEKR |
| | | MIDLSSISTVDSMIALDIDPLENTDFRVLELYSQKELRSSNVED |
| | | LEEIMRLFNSFKQVLKYV |
| SEQ ID | gB-Towne-ext-N-term_Bg- | GAAVSSSSTRGTSATHSHHSSHTTSAAHSRSGSVSQRVTSSQTV |
| NO: 70 | KOs_Cpack1-long_dII-dIII- | SHGVNETIYNTTLKYGDVVGVNTTKYPYRVCSMAQGTDLIRFER |
| | pack1_Disulf1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVEVAYHRDSYENKIMQLMPDCYSNTHSTRYVTVKDQWHSRGST |
| | R457S, R460S | NLTRETSNLNCMVTITTARSKYPYHFFATSIGDVVDISPFYNGT |
| | dII-dIII-pack1: S367I, | NRNASYFGENADKFFIFPNYTIVSDFGRPNSALETHRIVAFLER |
| | T374F | ADSVISWDIQDEKNVICQLIFWEASERTIRSEAEDSYHFISAKM |
| | Cpack1-long: E686L, Y690F, | TAFFLSKKQEVNMSDSALDCVRDEAINKLQQIFNTSYNQTYEKY |
| | R693V, V694L, K700A, V702Q | GNVSVFETTGGLVVFWQGIKQKSIVELERLANRSSLNLTHNSTK |
| | Disulf1: D217C, S587C | SSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIENFANCSYVQ |
| | | YGQLGEDNEILLGNHRTEECQLPSLKIFIAGNSAYEYVDYLFKR |
| | | MIDLSSISTVDSMIALDIDPLENTDFRVLELYSQKELRSSNVED |
| | | LEEIMRLENSFKQVLKYVEDAVQ |
| SEQ ID | gB-Towne-ext-N-term_Bg- | GAAVSSSSTRGTSATHSHHSSHITSAAHSRSGSVSQRVTSSQTV |
| NO: 71 | KOs_Cpack1-short_dII-dIII- | SHGVNETIYNTTLKYGDVVGVNTTKYPYRVCSMAQGIDLIRFER |
| | pack1_Disulf2 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDCYSNTHSTRYVTVKDQWHSRGST |
| | R457S, R460S | NLTRETSNLNCMVTITTARSKYPYHFFATSTGDVVDISPFYNGT |
| | dII-dIII-pack1: S367I, | NRNASYFGENADKFFIFPNYTIVSDFGRPNSALETHRLVAFLER |
| | T374F | ADSVISWDIQDEKNVTCQLTFWEASERTIRSEAEDSYHFISAKM |
| | Cpack1-short: E686L, | TAFFLSKKQEVNMSDSALDCVRDEAINKLQQIFNTSYNQTYEKY |
| | Y690F, R693V, V694L | GNVSVFETTGGLVVFWQGIKQKSIVELERLANRSSLNLTANSTK |
| | Disulf2: D217C, Y589C | SSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCVTINQTSVKVLRDMNVKESPGRCYSRPVVIENFANSSCVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVED |
| | | LEEIMRLFNSFKQVLKYV |
| SEQ ID | gB-Towne-ext-N-term_Bg- | GAAVSSSSTRGTSATHSHHSSHTTSAAHSRSGSVSQRVTSSQTV |
| NO: | KOs_Cpack1-long_dII-dIII- | SHGVNETIYNTTLKYGDVVGVNTTKYPYRVCSMAQGTDLIRFER |
| | pack1_Disulf2 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDCYSNTHSTRYVTVKDQWHSRGST |
| | R457S, R460S | NLTRETSNLNCMVTITTARSKYPYHFFATSTGDVVDISPFYNGT |
| | dII-dIII-pack1: S367I, | NRNASYFGENADKFFIFPNYTIVSDEGRPNSALETHRLVAFLER |
| | T374F | ADSVISWDIQDEKNVTCQLIFWEASERTIRSEAEDSYHFISAKM |
| | Cpack1-long: E686L, Y690F, | TAFFLSKKQEVNMSDSALDCVRDEAINKLQQIFNTSYNQTYEKY |
| | R693V, V694L, K700A, V702Q | GNVSVFETTGGLVVFWQGIKQKSLVELERLANRSSLNLTHNSTK |
| | Disulf2: D217C, Y589C | SSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCVTINQTSVKVLRDMNVKESPGRCYSRPVVIENFANSSCVQ |
| | | YGQLGEDNEILLGNHRTEECQLPSLKIFIAGNSAYEYVDYLEKR |
| | | MIDLSSISTVDSMIALDIDPLENTDFRVLELYSQKELRSSNVED |
| | | LEEIMRLFNSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 73 | KOs_Cpack1-short_dII-dIII- | SHGVNETIYNTTLKYGDVVGVNTTKYPYRVCSMAQGTDLIRFER |
| | pack1_dI-dIV-dV- | NIVCTSMKPINEDLDEGIMVVYKRNIVAHTFKVRVYQKVLTERR |
| | pack1_Disulf1 | SYAYHRTTYLLGSNTEYVAPPMWEIHHINSHSQCYSSYSRVIAG |
| | Bg-KOs: I156H, H157R, | TVFVAYHRDSYENKTMQLMPDCFSNTHSTRYVTVKDQWHSRGST |
| | W240N, Y242T, C246S, | NLTRETSNLNCMVTITTARSKYPYHFFATSTGDVVDISPFYNGT |
| | R457S, R460S | NRNASYFGENADKFFIFPNYTIVSDFGRPNSALETHRLVAFLER |
| | dII-dIII-pack1: S367I, | ADSVISWDIQDEKNVTCQLTFWEASERTIRSEAEDSYHFISAKM |
| | T374F | TAFFLSKKQEVNMSDSALDCVRDEAINKLQQIFNTSYNQTYEKY |
| | Cpack1-short: E686L, | GNVSVFETTGGLVVFWQGIKQKSLVELERLANRSSLNLTHNSTK |
| | Y690F, R693V, V694L | SSTDGNNATHLSNMESVHNLVYAQLQFTYDTLRGYINRALAQIA |
| | dI-dIV-dV-pack1: Y218F, | EAWCVDQRRTLEVFKELSKINPSAILSAIYNKPIAAREMGDVLG |
| | Q591F, S668A | LASCVTINQTSVKVLRDMNVKESPGRCYSRPVVIENFANCSYVE |
| | Disulf1: D217C, S587C | YGQLGEDNEILLGNHRTEECQLPSLKIFIAGNSAYEYVDYLEKR |
| | | MIDLSSISTVDSMIALDIDPLENTDERVLELYAQKELRSSNVED |
| | | LEEIMRLENSFKQVLKYV |
| SEQ ID | gB-Towne-ext-N-term_Bg- | GAAVSSSSTRGTSATHSHHSSHTTSAAHSRSGSVSQRVTSSQTV |
| NO: 74 | KOs_Cpack1-long_dII-dIII- | SHGVNETIYNTTLKYGDVVGVNITKYPYRVCSMAQGTDLIRFER |
| | pack1_dI-dIV-dV- | NIVCTSMKPINEDLDEGIMVVYKRNIVAHTFKVRVYQKVLTERR |
| | pack1_Disulf1 | SYAYHRTTYLLGSNTEYVAPPMWEIHHINSHSQCYSSYSRVIAG |
| | Bg-KOs: I156H, H157R, | TVEVAYHRDSYENKTMQLMPDCFSNTHSTRYVTVKDQWHSRGST |
| | W240N, Y242T, C246S, | NLTRETSNLNCMVTITTARSKYPYHFFATSTGDVVDISPFYNGT |
| | R457S, R460S | NRNASYFGENADKFFIFPNYTIVSDFGRPNSALETHRLVAFLER |
| | dII-dIII-pack1: S367I, | ADSVISWDIQDEKNVTCQLTFWEASERTIRSEAEDSYHFISAKM |
| | T374F | TAFFLSKKQEVNMSDSALDCVRDEAINKLQQIFNTSYNQTYEKY |
| | Cpack1-long: E686L, Y690F, | GNVSVFETTGGLVVFWQGIKQKSLVELERLANRSSLNLTHNSTK |
| | R693V, V694L, K700A, V702Q | SSTDGNNATHLSNMESVHNLVYAQLQFTYDTLRGYINRALAQIA |
| | dI-dIV-dV-pack1: Y218F, | EAWCVDQRRTLEVFKELSKINPSAILSAIYNKPIAAREMGDVLG |
| | Q591F, S668A | LASCVTINQTSVKVLRDMNVKESPGRCYSRPVVIENFANCSYVE |
| | Disulf1: D217C, S587C | YGQLGEDNEILLGNHRTEECQLPSLKIFIAGNSAYEYVDYLEKR |
| | | MIDLSSISTVDSMIALDIDPLENTDERVLELYAQKELRSSNVED |
| | | LEEIMRLFNSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 75 | KOs_Cpack1-short_dII-dIII- | SHGVNETIYNTTLKYGDVVGVNTTKYPYRVCSMAQGTDLIRFER |
| | pack1_dI-dIV-dV- | NIVCTSMKPINEDLDEGIMVVYKRNIVAHTFKVRVYQKVLTERR |
| | pack1_Disulf2 | SYAYHRTTYLLGSNTEYVAPPMWEIHHINSHSQCYSSYSRVIAG |
| | Bg-KOs: I156H, H157R, | TVFVAYHRDSYENKTMQLMPDCFSNTHSTRYVTVKDQWHSRGST |
| | W240N, Y242T, C246S, | NLTRETSNLNCMVTITTARSKYPYHFFATSTGDVVDISPFYNGT |
| | R457S, R460S | NRNASYFGENADKFFIFPNYTIVSDFGRPNSALETHRLVAFLER |
| | dII-dIII-pack1: S367I, | ADSVISWDIQDEKNVTCQLTFWEASERTIRSEAEDSYHFISAKM |
| | T374F | TAFFLSKKQEVNMSDSALDCVRDEAINKLQQIFNTSYNQTYEKY |
| | Cpack1-short: E686L, | GNVSVFETTGGLVVFWQGIKQKSLVELERLANRSSLNLTHNSTK |
| | Y690F, R693V, V694L | SSTDGNNATHLSNMESVHNLVYAQLQFTYDTLRGYINRALAQIA |
| | dI-dIV-dV-pack1: Y218F, | EAWCVDQRRTLEVFKELSKINPSAILSAIYNKPIAAREMGDVLG |
| | Q591F, S668A | LASCVTINQTSVKVLRDMNVKESPGRCYSRPVVIENFANSSCVE |
| | Disulf2: D217C, Y589C | YGQLGEDNEILLGNHRTEECQLPSLKIFIAGNSAYEYVDYLFKR |
| | | MIDLSSISTVDSMIALDIDPLENTDERVLELYAQKELRSSNVED |
| | | LEEIMRLENSFKQVLKYV |
| SEQ ID | gB-Towne-ext-N-term_Bg- | GAAVSSSSTRGTSATHSHHSSHTTSAAHSRSGSVSQRVTSSQTV |
| NO: 76 | KOs_Cpack1-long_dII-dIII- | SHGVNETIYNTTLKYGDVVGVNTTKYPYRVCSMAQGTDLIRFER |
| | pack1_dI-dIV-dV- | NIVCTSMKPINEDLDEGIMVVYKRNIVAHTFKVRVYQKVLTERR |
| | pack1_Disulf2 | SYAYHRTTYLLGSNTEYVAPPMWEIHHINSHSQCYSSYSRVIAG |
| | Bg-KOs: I156H, H157R, | TVEVAYHRDSYENKTMQLMPDCFSNTHSTRYVTVKDQWHSRGST |
| | W240N, Y242T, C246S, | NLTRETSNLNCMVTITTARSKYPYHFFATSTGDVVDISPFYNGT |
| | R457S, R460S | NRNASYFGENADKFFIFPNYTIVSDFGRPNSALETHRLVAFLER |
| | dII-dIII-pack1: S367I, | ADSVISWDIQDEKNVTCQLTFWEASERTIRSEAEDSYHFISAKM |
| | T374F | TAFFLSKKQEVNMSDSALDCVRDEAINKLQQIFNTSYNQTYEKY |
| | Cpack1-long: E686L, Y690F, | GNVSVFETTGGLVVFWQGIKQKSLVELERLANRSSINLTHNSTK |
| | R693V, V694L, K700A, V702Q | SSTDGNNATHLSNMESVHNLVYAQLQFTYDTLRGYINRALAQIA |
| | dI-dIV-dV-pack1: Y218F, | EAWCVDQRRTLEVEKELSKINPSAILSAIYNKPIAAREMGDVLG |
| | Q591F, S668A | LASCVTINQTSVKVLRDMNVKESPGRCYSRPVVIENFANSSCVF |
| | Disulf2: D217C, Y589C | YGQLGEDNEILLGNHRTEECQLPSLKIFIAGNSAYEYVDYLFKR |
| | | MIDLSSISTVDSMIALDIDPLENTDERVLELYAQKELRSSNVED |
| | | LEEIMRLFNSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 77 | KOs_Cpack1-short_dII-dIII- | SHGVNETIYNTTLKYGDVVGVNTTKYPYRVCSMAQGTDLIRFER |
| | pack1_dI-dIV-dV- | NIVCTSMKPINEDLDEGIMVVYKRNIVAHTEKVRVYQKVLTERR |
| | pack1_N165glyc | SYAYHRTTYLLGSNTTYVAPPMWEIHHINSHSQCYSSYSRVIAG |
| | Bg-KOs: I156H, H157R, | TVFVAYHRDSYENKTMQLMPDDFSNTHSTRYVTVKDQWHSRGST |
| | W240N, Y242T, C246S, | NLTRETSNLNCMVTITTARSKYPYHFFATSTGDVVDISPFYNGT |
| | R457S, R460S | NRNASYFGENADKFFIFPNYTIVSDFGRPNSALETHRLVAFLER |
| | dII-dIII-pack1: S367I, | ADSVISWDIQDEKNVTCQLTFWEASERTIRSEAEDSYHFISAKM |
| | T374F | TAFFLSKKQEVNMSDSALDCVRDEAINKLQQIFNTSYNQTYEKY |
| | Cpack1-short: E686L, | GNVSVFETTGGLVVFWQGIKQKSLVELERLANRSSLNLTHNSTK |
| | Y690F, R693V, V694L | SSTDGNNATHLSNMESVHNLVYAQLQFTYDTLRGYINRALAQIA |
| | dI-dIV-dV-pack1: Y218F, | EAWCVDQRRTLEVFKELSKINPSAILSAIYNKPIAARFMGDVLG |
| | Q591F, S668A | LASCVTINQTSVKVLRDMNVKESPGRCYSRPVVIENFANSSYVE |
| | N165glyc: E167T | YGQLGEDNEILLGNHRTEECQLPSLKIFIAGNSAYEYVDYLFKR |
| | | MIDLSSISTVDSMIALDIDPLENTDERVLELYAQKELRSSNVED |
| | | LEEIMRLENSFKQVLKYV |
| SEQ ID | gB-Towne-ext-N-term_Bg- | GAAVSSSSTRGTSATHSHHSSHTTSAAHSRSGSVSQRVTSSQTV |
| NO: 78 | KOs_Cpack1-long_dII-dIII- | SHGVNETIYNTTLKYGDVVGVNTTKYPYRVCSMAQGTDLIRFER |
| | pack1_dI-dIV-dV- | NIVCTSMKPINEDLDEGIMVVYKRNIVAHTFKVRVYQKVLTERR |
| | pack1_N165glyc | SYAYHRTTYLLGSNTTYVAPPMWEIHHINSHSQCYSSYSRVIAG |
| | Bg-KOs: I156H, H157R, | TVFVAYHRDSYENKTMQLMPDDESNTHSTRYVTVKDQWHSRGST |
| | W240N, Y242T, C246S, | NLTRETSNLNCMVTITTARSKYPYHFFATSTGDVVDISPFYNGT |
| | R457S, R460S | NRNASYFGENADKFFIFPNYTIVSDFGRPNSALETHRLVAFLER |
| | dII-dIII-pack1: 3367I, | ADSVISWDIQDEKNVTCQLTFWEASERTIRSEAEDSYHFISAKM |
| | T374F | TAFFLSKKQEVNMSDSALDCVRDEAINKLQQIFNTSYNQTYEKY |
| | Cpack1-long: E686L, Y690F, | GNVSVFETTGGLVVFWQGIKQKSLVELERLANRSSLNLTHNSTK |
| | R693V, V694L, K700A, V702Q | SSTDGNNATHLSNMESVHNLVYAQLQFTYDTLRGYINRALAQIA |
| | dI-dIV-dV-pack1: Y218F, | EAWCVDQRRTLEVFKELSKINPSAILSAIYNKPIAAREMGDVLG |
| | Q591F, S668A | LASCVTINQTSVKVLRDMNVKESPGRCYSRPVVIENFANSSYVE |
| | N165glyc: E167T | YGQLGEDNEILLGNHRTEECQLPSLKIFIAGNSAYEYVDYLFKR |
| | | MIDLSSISTVDSMIALDIDPLENTDERVLELYAQKELRSSNVED |
| | | LEEIMRLENSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 79 | KOs_Cpack1-short_dI-dIV- | SHGVNETIYNTTLKYGDVVGVNTTKYPYRVCSMAQGTDLIRFER |
| | dV-pack2 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDDYSSTHSTRYVTVKDQWHSRGST |
| | R457S, R460S | NLTRETSNLNCMVTITTARSKYPYHFFATSTGDVVDISPFYNGT |
| | Cpack1-short: E686L, | NRNASYFGENADKFFIFPNYTIVSDFGRPNSALETHRIVAFLER |
| | Y690F, R693V, V694L | ADSVISWDIQDEKNVTCQLTFWEASERTIRSEAEDSYHESSAKM |
| | dI-dIV-dV-pack2: N220S, | TATFLSKKQEVNMSDSALDCVRDEAINKLQQIFNTSYNQTYEKY |
| | V5521 | GNVSVFETTGGLVVFWQGIKQKSLVELERLANRSSLNLTHNSTK |
| | | SSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCLTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVED |
| | | LEEIMRLFNSFKQVLKYV |
| SEQ ID | gB-Towne-ext-N-term_Bg- | GAAVSSSSTRGTSATHSHHSSHTTSAAHSRSGSVSQRVTSSQTV |
| NO: 80 | KOs_Cpack1-long_dI-dIV-dV- | SHGVNETIYNTTLKYGDVVGVNTTKYPYRVCSMAQGTDLIRFER |
| | pack2 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVEVAYHRDSYENKTMQLMPDDYSSTHSTRYVTVKDQWHSRGST |
| | R457S, R460S | NLTRETSNLNCMVTITTARSKYPYHFFATSIGDVVDISPFYNGT |
| | Cpack1-long: E686L, Y690F, | NRNASYFGENADKFFIFPNYTIVSDFGRPNSALETHRLVAFLER |
| | R693V, V694L, K700A, V7020 | ADSVISWDIQDEKNVTCQLTFWEASERTIRSEAEDSYHFSSAKM |
| | dI-dIV-dV-pack2: N220S, | TATFLSKKQEVNMSDSALDCVRDEAINKLQQIFNTSYNQTYEKY |
| | V552L | GNVSVFETTGGLVVFWQGIKQKSIVELERLANRSSLNLTHNSTK |
| | | SSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCLTINQTSVKVLRDMNVKESPGRCYSRPVVIFNFANSSYVQ |
| | | YGQLGEDNEILLGNHRTEECQLPSLKIFIAGNSAYEYVDYLFKR |
| | | MIDLSSISTVDSMIALDIDPLENTDERVLELYSQKELRSSNVED |
| | | LEEIMRLENSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 81 | KOs_Cpackl-short_dI-dIV- | SHGVNETIYNTTLKYGDVVGVNTTKYPYRVCSMAQGTDLIRFER |
| | dV-pack3 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDDESSTHSTRYVTVKDQWHSRGST |
| | R457S, R460S | NLTRETSNLNCMVTITTARSKYPYHFFATSTGDVVDISPFYNGT |
| | Cpack1-short: E686L, | NRNASYFGENADKFFIFPNYTIVSDFGRPNSALETHRLVAFLER |
| | Y690F, R693V, V694L | ADSVISWDIQDEKNVTCQLTFWEASERTIRSEAEDSYHFSSAKM |
| | dI-dIV-dV-pack3: Y218F, | TATFLSKKQEVNMSDSALDCVRDEAINKLQQIFNTSYNQTYEKY |
| | N220S, V552L, Q591F, S668A | GNVSVFETTGGLVVFWQGIKQKSIVELERLANRSSLNLTHNSTK |
| | | SSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCLTINQTSVKVLRDMNVKESPGRCYSRPVVIENFANSSYVE |
| | | YGQLGEDNEILLGNHRTEECQLPSLKIFIAGNSAYEYVDYLEKR |
| | | MIDLSSISTVDSMIALDIDPLENTDFRVLELYAQKELRSSNVFD |
| | | LEEIMRLFNSFKQVLKYV |
| SEQ ID | gB-Towne-ext-N-term_Bg- | GAAVSSSSTRGTSATHSHHSSHTTSAAHSRSGSVSQRVTSSQTV |
| NO: 82 | KOs_Cpack1-long_dI-dIV-dV- | SHGVNETIYNTTLKYGDVVGVNTTKYPYRVCSMAQGTDLIRFER |
| | pack3 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVEVAYHRDSYENKTMQLMPDDESSTHSTRYVTVKDQWHSRGST |
| | R457S, R460S | NLTRETSNLNCMVTITTARSKYPYHFFATSTGDVVDISPFYNGT |
| | Cpack1-long: E686L, Y690F, | NRNASYFGENADKFFIFPNYTIVSDFGRPNSALETHRLVAFLER |
| | R693V, V694L, K700A, V702Q | ADSVISWDIQDEKNVTCQLTFWEASERTIRSEAEDSYHESSAKM |
| | dI-dIV-dV-pack3: Y218F, | TATFLSKKQEVNMSDSALDCVRDEAINKLQQIFNTSYNQTYEKY |
| | N220S, V552L, Q591F, S668A | GNVSVFETTGGLVVFWQGIKQKSLVELERLANRSSLNLTHNSTK |
| | | SSTDGNNATHLSNMESVHNLVYAQLQFTYDTLRGYINRALAQIA |
| | | EAWCVDQRRTLEVFKELSKINPSAILSAIYNKPIAARFMGDVLG |
| | | LASCLTINQTSVKVLRDMNVKESPGRCYSRPVVIENFANSSYVE |
| | | YGQLGEDNEILLGNHRTEECQLPSLKIFIAGNSAYEYVDYLFKR |
| | | MIDLSSISTVDSMIALDIDPLENTDERVLELYAQKELRSSNVED |
| | | LEEIMRLFNSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 83 | KOs_Cpack1-short_dII-dIII- | SHGVNETIYNTTLKYGDVVGVNTTKYPYRVCSMAQGTDLIRFER |
| | pack1_dI-dIV-dV-pack2 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVEVAYHRDSYENKTMQLMPDDYSSTHSTRYVTVKDQWHSRGST |
| | R457S, R460S | NLTRETSNLNCMVTITTARSKYPYHFFATSTGDVVDISPFYNGT |
| | dII-dIII-pack1: S367I, | NRNASYFGENADKFFIFPNYTIVSDFGRPNSALETHRLVAFLER |
| | T374F | ADSVISWDIQDEKNVTCQLTFWEASERTIRSEAEDSYHFISAKM |
| | Cpack1-short: E686L, | TAFFLSKKQEVNMSDSALDCVRDEAINKLQQIFNTSYNQTYEKY |
| | Y690F, R693V, V694L | GNVSVFETTGGLVVFWQGIKQKSLVELERLANRSSLNLTHNSTK |
| | dI-dIV-dV-pack2: N220S, | SSTDGNNATHLSNMESVHNLVYAQLQFTYDTLRGYINRALAQIA |
| | V552L | EAWCVDQRRTLEVEKELSKINPSAILSAIYNKPIAARFMGDVLG |
| | | LASCLTINQTSVKVLRDMNVKESPGRCYSRPVVIENFANSSYVQ |
| | | YGQLGEDNEILLGNHRTEECQLPSLKIFIAGNSAYEYVDYLFKR |
| | | MIDLSSISTVDSMIALDIDPLENTDFRVLELYSQKELRSSNVFD |
| | | LEEIMRLENSFKQVLKYV |
| SEQ ID | gB-Towne-ext-N-term_Bg- | GAAVSSSSTRGTSATHSHHSSHTTSAAHSRSGSVSQRVTSSQTV |
| NO: 84 | KOs_Cpack1-long_dII-dIII- | SHGVNETIYNTTLKYGDVVGVNTTKYPYRVCSMAQGTDLIRFER |
| | pack1_dI-dIV-dV-pack2 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDDYSSTHSTRYVTVKDQWHSRGST |
| | R457S, R460S | NLTRETSNLNCMVTITTARSKYPYHFFATSTGDVVDISPFYNGT |
| | dII-dIII-pack1: S367I, | NRNASYFGENADKFFIFPNYTIVSDFGRPNSALETHREVAFLER |
| | T374F | ADSVISWDIQDEKNVICQLTFWEASERTIRSEAEDSYHFISAKM |
| | Cpack1-long: E686L, Y690F, | TAFFLSKKQEVNMSDSALDCVRDEAINKLQQIFNTSYNQTYEKY |
| | R693V, V694L, K700A, V702Q | GNVSVFETTGGLVVFWQGIKQKSIVELERLANRSSLNLTANSTK |
| | dI-dIV-dV-pack2: N220S, | SSTDGNNATHLSNMESVHNLVYAQLQFTYDTLRGYINRALAQIA |
| | V552L | EAWCVDQRRILEVFKELSKINPSAILSAIYNKPIAAREMGDVLG |
| | | LASCLTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVED |
| | | LEEIMRLENSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 85 | KOs_Cpack1-short_dII-dIII- | SHGVNETIYNTTLKYGDVVGVNTTKYPYRVCSMAQGTDLIRFER |
| | pack1_dI-dIV-dV-pack3 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDDESSTHSTRYVTVKDQWHSRGST |
| | R457S, R460S | NLTRETSNLNCMVTITTARSKYPYHFFATSTGDVVDISPFYNGT |
| | dII-dIII-pack1: S367I, | NRNASYFGENADKFFIFPNYTIVSDEGRPNSALETHRLVAFLER |
| | T374F | ADSVISWDIQDEKNVTCQLTFWEASERTIRSEAEDSYHFISAKM |
| | Cpack1-short: E686L, | TAFFLSKKQEVNMSDSALDCVRDEAINKLQQIFNTSYNQTYEKY |
| | Y690F, R693V, V694L | GNVSVFETTGGLVVFWQGIKQKSIVELERLANRSSLNLTHNSTK |
| | dI-dIV-dV-pack3: Y218F, | SSTDGNNATHLSNMESVHNLVYAQLQFTYDTLRGYINRALAQIA |
| | N220S, V552L, Q591F, S668A | EAWCVDQRRTLEVFKELSKINPSAILSAIYNKPIAAREMGDVLG |
| | | LASCLTINQTSVKVLRDMNVKESPGRCYSRPVVIENFANSSYVE |
| | | YGQLGEDNEILLGNHRTEECQLPSLKIFIAGNSAYEYVDYLFKR |
| | | MIDLSSISTVDSMIALDIDPLENTDERVLELYAQKELRSSNVED |
| | | LEEIMRLFNSFKQVLKYV |
| SEQ ID | gB-Towne-ext-N-term_Bg- | GAAVSSSSTRGTSATHSHHSSHTTSAAHSRSGSVSQRVTSSQTV |
| NO: 86 | KOs_Cpack1-long_dII-dIII- | SHGVNETIYNTTLKYGDVVGVNTTKYPYRVCSMAQGTDLIRFER |
| | pack1_dI-dIV-dV-pack3 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVEVAYHRDSYENKTMQLMPDDESSTHSTRYVTVKDQWHSRGST |
| | R457S, R460S | NLTRETSNLNCMVTITTARSKYPYHFFATSTGDVVDISPFYNGT |
| | dII-dIII-pack1: S367I, | NRNASYFGENADKFFIFPNYTIVSDFGRPNSALETHRLVAFLER |
| | T374F | ADSVISWDIQDEKNVTCQLIFWEASERTIRSEAEDSYHFISAKM |
| | Cpack1-long: E686L, Y690F, | TAFFLSKKQEVNMSDSALDCVRDEAINKLQQIFNTSYNQTYEKY |
| | R693V, V694L, K700A, V702Q | GNVSVFETTGGLVVFWQGIKQKSLVELERLANRSSLNLTHNSTK |
| | dI-dIV-dV-pack3: Y218F, | SSTDGNNATHLSNMESVHNLVYAQLQFTYDTLRGYINRALAQIA |
| | N220S, V552L, Q591F, S668A | EAWCVDQRRTLEVFKELSKINPSAILSAIYNKPIAAREMGDVLG |
| | | LASCLTINQTSVKVLRDMNVKESPGRCYSRPVVIFNFANSSYVE |
| | | YGQLGEDNEILLGNHRTEECQLPSLKIFIAGNSAYEYVDYLEKR |
| | | MIDLSSISTVDSMIALDIDPLENTDERVLELYAQKELRSSNVED |
| | | LEEIMRLENSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 87 | KOs_Cpack1-short_dI-dIV- | SHGVNETIYNTTLKYGDVVGVNTTKYPYRVCSMAQGTDLIRFER |
| | dV-pack3_Disulf1 | NIVCTSMKPINEDLDEGIMVVYKRNIVAHTEKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDCFSSTHSTRYVTVKDQWHSRGST |
| | R457S, R460S | NLTRETSNLNCMVTITTARSKYPYHFFATSTGDVVDISPFYNGT |
| | Cpack1-short: E686L, | NRNASYFGENADKFFIFPNYTIVSDFGRPNSALETHRLVAFLER |
| | Y690F, R693V, V694L | ADSVISWDIQDEKNVICQLTFWEASERTIRSEAEDSYHESSAKM |
| | dI-dIV-dV-pack3: Y218F, | TATFLSKKQEVNMSDSALDCVRDEAINKLQQIFNTSYNQTYEKY |
| | N220S, V552D, Q591F, S668A | GNVSVFETTGGLVVFWQGIKQKSIVELERLANRSSLNLTHNSTK |
| | Disulf1: D217C, S587C | SSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCLTINQTSVKVLRDMNVKESPGRCYSRPVVIENFANCSYVE |
| | | YGQLGEDNEILLGNHRTEECQLPSLKIFIAGNSAYEYVDYLEKR |
| | | MIDLSSISTVDSMIALDIDPLENTDERVLELYAQKELRSSNVFD |
| | | LEEIMRLFNSFKQVLKYV |
| SEQ ID | gB-Towne-ext-N-term_Bg- | GAAVSSSSTRGTSATHSHHSSHTTSAAHSRSGSVSQRVTSSQTV |
| NO: 88 | KOs_Cpack1-long_dI-dIV-dV- | SHGVNETIYNTTLKYGDVVGVNITKYPYRVCSMAQGIDLIRFER |
| | pack3_Disulf1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDCESSTHSTRYVTVKDQWHSRGST |
| | R457S, R460S | NLTRETSNLNCMVTITTARSKYPYHFFATSTGDVVDISPFYNGT |
| | Cpack1-long: E686L, Y690F, | NRNASYFGENADKFFIFPNYTIVSDFGRPNSALETHRLVAFLER |
| | R693V, V694L, K700A, V702Q | ADSVISWDIQDEKNVICQLIFWEASERTIRSEAEDSYHFSSAKM |
| | dI-dIV-dV-pack3: Y218F, | TATFLSKKQEVNMSDSALDCVRDEAINKLQQIFNTSYNQTYEKY |
| | N220S, V552L, Q591F, S668A | GNVSVFETTGGLVVFWQGIKQKSLVELERLANRSSLNLTHNSTK |
| | Disulf1: D217C, S587C | SSIDGNNATHLSNMESVHNLVYAQLQFTYDTLRGYINRALAQIA |
| | | EAWCVDQRRILEVFKELSKINPSAILSAIYNKPIAAREMGDVLG |
| | | LASCLTINQTSVKVLRDMNVKESPGRCYSRPVVIENFANCSYVE |
| | | YGQLGEDNEILLGNHRTEECQLPSLKIFIAGNSAYEYVDYLEKR |
| | | MIDLSSISTVDSMIALDIDPLENTDERVLELYAQKELRSSNVED |
| | | LEEIMRLENSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 89 | KOs_Cpack1-short_dII-dIII- | SHGVNETIYNTTLKYGDVVGVNTTKYPYRVCSMAQGTDLIRFER |
| | pack1_dI-dIV-dV- | NIVCTSMKPINEDLDEGIMVVYKRNIVAHTFKVRVYQKVLTERR |
| | pack3_Disulf1 | SYAYHRTTYLLGSNTEYVAPPMWEIHHINSHSQCYSSYSRVIAG |
| | Bg-KOs: I156H, H157R, | TVEVAYHRDSYENKTMQLMPDCFSSTHSTRYVTVKDQWHSRGST |
| | W240N, Y242T, C246S, | NLTRETSNLNCMVTITTARSKYPYHFFATSTGDVVDISPFYNGT |
| | R457S, R460S | NRNASYFGENADKFFIFPNYTIVSDFGRPNSALETHRLVAFLER |
| | Cpack1-short: E686L, | ADSVISWDIQDEKNVTCQLTFWEASERTIRSEAEDSYHFISAKM |
| | Y690F, R693V, V694L | TAFFLSKKQEVNMSDSALDCVRDEAINKLQQIFNTSYNQTYEKY |
| | dII-dIII-pack1: S367I, | GNVSVFETTGGLVVFWQGIKQKSIVELERLANRSSLNLTHNSTK |
| | T374F | SSTDGNNATHLSNMESVHNLVYAQLQFTYDTLRGYINRALAQIA |
| | dI-dIV-dV-pack3: Y218F, | EAWCVDQRRTLEVEKELSKINPSAILSAIYNKPIAAREMGDVLG |
| | N220S, V552L, Q591F, S668A | LASCLTINQTSVKVLRDMNVKESPGRCYSRPVVIENFANCSYVF |
| | Disulf1: D217C, S587C | YGQLGEDNEILLGNHRTEECQLPSLKIFIAGNSAYEYVDYLFKR |
| | | MIDLSSISTVDSMIALDIDPLENTDERVLELYAQKELRSSNVED |
| | | LEEIMRLENSFKQVLKYV |
| SEQ ID | gB-Towne-ext-N-term_Bg- | GAAVSSSSTRGTSATHSHHSSHTTSAAHSRSGSVSQRVTSSQTV |
| NO: 90 | KOs_Cpack1-long_dII-dIII- | SHGVNETIYNTTLKYGDVVGVNTTKYPYRVCSMAQGTDLIRFER |
| | pack1_dI-dIV-dV- | NIVCTSMKPINEDLDEGIMVVYKRNIVAHTFKVRVYQKVLTERR |
| | pack3_Disulf1 | SYAYHRTTYLLGSNTEYVAPPMWEIHHINSHSQCYSSYSRVIAG |
| | Bg-KOs: I156H, H157R, | TVEVAYHRDSYENKTMQLMPDCFSSTHSTRYVTVKDQWHSRGST |
| | W240N, Y242T, C246S, | NLTRETSNLNCMVTITTARSKYPYHFFATSIGDVVDISPFYNGT |
| | R457S, R460S | NRNASYFGENADKFFIFPNYTIVSDFGRPNSALETHRLVAFLER |
| | Cpack1-long: E686L, Y690F, | ADSVISWDIQDEKNVTCQLIFWEASERTIRSEAEDSYHFISAKM |
| | R693V, V694D, K700A, V702Q | TAFFLSKKQEVNMSDSALDCVRDEAINKLQQIFNTSYNQTYEKY |
| | dII-dIII-pack1: S367I, | GNVSVFETTGGLVVFWQGIKQKSIVELERLANRSSLNLTHNSTK |
| | T374F | SSTDGNNATHLSNMESVHNLVYAQLQFTYDTLRGYINRALAQIA |
| | dI-dIV-dV-pack3: Y218F, | EAWCVDQRRTLEVEKELSKINPSAILSAIYNKPIAAREMGDVLG |
| | N220S, V552L, Q591F, S668A | LASCLTINQTSVKVLRDMNVKESPGRCYSRPVVIENFANCSYVE |
| | Disulf1: D217C, S587C | YGQLGEDNEILLGNHRTEECQLPSLKIFIAGNSAYEYVDYLFKR |
| | | MIDLSSISTVDSMIALDIDPLENTDERVLELYAQKELRSSNVED |
| | | LEEIMRLENSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 91 | KOs_Cpack1-short_dII-dIII- | SHGVNETIYNTTLKYGDVVGVNITKYPYRVCSMAQGTDLIRFER |
| | pack1_dI-dIV-dV- | NIVCTSMKPINEDLDEGIMVVYKRNIVAHTFKVRVYQKVLTFRR |
| | pack4_Disulf1 | SYAYHRTTYLLGSNTEYVAPPMWEIHHINSHSQCYSSYSRVIAG |
| | Bg-KOs: I156H, H157R, | TVEVAYHRDSYENKTMQLMPDCFSSTHSTRYVTVKDQWHSRGST |
| | W240N, Y242T, C246S, | NLTRETSNLNCMVTITTARSKYPYHFFATSTGDVVDISPFYNGT |
| | R457S, R460S | NRNASYFGENADKFFIFPNYTIVSDFGRPNSALETHRLVAFLER |
| | Cpack1-short: E686L, | ADSVISWDIQDEKNVICQLTFWEASERTIRSEAEDSYHFISAKM |
| | Y690F, R693V, V694L | TAFFLSKKQEVNMSDSALDCVRDEAINKLQQIFNTSYNQTYEKY |
| | dII-dIII-pack1: S367I, | GNVSVFETTGGLVVFWQGIKQKSIVELERLANRSSLNLTHNSTK |
| | T374F | SSTDGNNATHLSNMESVHNLVYAQLQFTYDTLRGYINRALAQIA |
| | dI-dIV-dV-pack4: Y218F, | EAWCVDQRRTLEVEKELSKINPSAILSAIYNKPIAARFMGDVLG |
| | N220S, V552L, Q591Y, S668A | LASCLTINQTSVKVLRDMNVKESPGRCYSRPVVIENFANCSYVY |
| | Disulf1: D217C, S587C | YGQLGEDNEILLGNHRTEECQLPSLKIFIAGNSAYEYVDYLFKR |
| | | MIDLSSISTVDSMIALDIDPLENTDERVLELYAQKELRSSNVED |
| | | LEEIMRLFNSFKQVLKYV |
| SEQ ID | gB-Towne-ext-N-term_Bg- | GAAVSSSSTRGTSATHSHHSSHTTSAAHSRSGSVSQRVTSSQTV |
| NO: 92 | KOs_Cpack1-long_dII-dIII- | SHGVNETIYNTTLKYGDVVGVNTTKYPYRVCSMAQGTDLIRFER |
| | pack1_dI-dIV-dV- | NIVCTSMKPINEDLDEGIMVVYKRNIVAHTFKVRVYQKVLTERR |
| | pack4_Disulf1 | SYAYHRTTYLLGSNTEYVAPPMWEIHHINSHSQCYSSYSRVIAG |
| | Bg-KOs: I156H, H157R, | TVEVAYHRDSYENKTMQLMPDCFSSTHSTRYVTVKDQWHSRGST |
| | W240N, Y242T, C246S, | NLTRETSNLNCMVTITTARSKYPYHFFATSIGDVVDISPFYNGT |
| | R457S, R460S | NRNASYFGENADKFFIFPNYTIVSDFGRPNSALETHRIVAFLER |
| | Cpack1-long: E686L, Y690F, | ADSVISWDIQDEKNVICQLTFWEASERTIRSEAEDSYHFISAKM |
| | R693V, V694L, K700A, V702Q | TAFFLSKKQEVNMSDSALDCVRDEAINKLQQIFNTSYNQTYEKY |
| | dII-dIII-pack1: S367I, | GNVSVFETTGGLVVFWQGIKQKSIVELERLANRSSLNLTHNSTK |
| | T374F | SSTDGNNATHLSNMESVHNLVYAQLQFTYDTLRGYINRALAQIA |
| | dI-dIV-dV-pack4: Y218F, | EAWCVDQRRTLEVFKELSKINPSAILSAIYNKPIAARFMGDVLG |
| | N220S, V552L, Q591Y, S668A | LASCLTINQTSVKVLRDMNVKESPGRCYSRPVVIFNFANCSYVY |
| | Disulf1: D217C, S587C | YGQLGEDNEILLGNHRTEECQLPSLKIFIAGNSAYEYVDYLFKR |
| | | MIDLSSISTVDSMIALDIDPLENTDFRVLELYAQKELRSSNVED |
| | | LEEIMRLENSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 93 | KOs_Cpack1-short_dII-dIII- | SHGVNETIYNTTLKYGDVVGVNTTKYPYRVCSMAQGTDLIRFER |
| | pack1_dI-dIV-dV- | NIVCTSMKPINEDLDEGIMVVYKRNIVAHTFKVRVYQKVLTERR |
| | pack5_Disulf1 | SYAYHRTTYLLGSNTEYVAPPMWEIHHINSHSQCYSSYSRVIAG |
| | Bg-KOs: I156H, H157R, | TVEVAYHRDSYENKTMQLMPDCYSSTHSTRYVTVKDQWHSRGST |
| | W240N, Y242T, C246S, | NLTRETSNLNCMVTITTARSKYPYHFFATSTGDVVDISPFYNGT |
| | R457S, R460S | NRNASYFGENADKFFIFPNYTIVSDFGRPNSALETHRLVAFLER |
| | Cpack1-short: E686L, | ADSVISWDIQDEKNVICQLTFWEASERTIRSEAEDSYHFISAKM |
| | Y690F, R693V, V694L | TAFFLSKKQEVNMSDSALDCVRDEAINKLQQIFNTSYNQTYEKY |
| | dII-dIII-pack1: S367I, | GNVSVFETTGGLVVFWQGIKQKSLVELERLANRSSLNLTHNSTK |
| | T374F | SSTDGNNATHLSNMESVHNLVYAQLQFTYDTLRGYINRALAQIA |
| | dI-dIV-dV-pack5: N220S, | EAWCVDQRRTLEVEKELSKINPSAILSAIYNKPIAAREMGDVLG |
| | V5521, Q591Y | LASCLTINQTSVKVLRDMNVKESPGRCYSRPVVIENFANCSYVY |
| | Disulf1: D217C, S587C | YGQLGEDNEILLGNHRTEECQLPSLKIFIAGNSAYEYVDYLFKR |
| | | MIDLSSISTVDSMIALDIDPLENTDERVLELYSQKELRSSNVED |
| | | LEEIMRLFNSFKQVLKYV |
| SEQ ID | gB-Towne-ext-N-term_Bg- | GAAVSSSSTRGTSATHSHHSSHTTSAAHSRSGSVSQRVTSSQTV |
| NO: 94 | KOs_Cpack1-long_dII-dIII- | SHGVNETIYNTTLKYGDVVGVNTTKYPYRVCSMAQGTDLIRFER |
| | pack1_dI-dIV-dV- | NIVCTSMKPINEDLDEGIMVVYKRNIVAHTFKVRVYQKVLTERR |
| | pack5_Disulf1 | SYAYHRTTYLLGSNTEYVAPPMWEIHHINSHSQCYSSYSRVIAG |
| | Bg-KOs: I156H, H157R, | TVEVAYHRDSYENKTMQLMPDCYSSTHSTRYVTVKDQWHSRGST |
| | W240N, Y242T, C246S, | NLTRETSNLNCMVTITTARSKYPYHFFATSIGDVVDISPFYNGT |
| | R457S, R460S | NRNASYFGENADKFFIFPNYTIVSDFGRPNSALETHRIVAFLER |
| | Cpack1-long: E686L, Y690F, | ADSVISWDIQDEKNVICQLTFWEASERTIRSEAEDSYHFISAKM |
| | R693V, V694D, K700A, V702Q | TAFFLSKKQEVNMSDSALDCVRDEAINKLQQIFNTSYNQTYEKY |
| | dII-dIII-pack1: S367I, | GNVSVFETTGGLVVFWQGIKQKSIVELERLANRSSLNLTHNSTK |
| | T374F | SSTDGNNATHLSNMESVHNLVYAQLQFTYDTLRGYINRALAQIA |
| | dI-dIV-dV-pack5: N220S, | EAWCVDQRRTLEVFKELSKINPSAILSAIYNKPIAARFMGDVLG |
| | V552L, Q591Y | LASCLTINQTSVKVLRDMNVKESPGRCYSRPVVIFNFANCSYVY |
| | Disulf1: D217C, S587C | YGQLGEDNEILLGNHRTEECQLPSLKIFIAGNSAYEYVDYLFKR |
| | | MIDLSSISTVDSMIALDIDPLENTDERVLELYSQKELRSSNVED |
| | | LEEIMRLENSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 95 | KOs_Cpack1-short_dII-dIII- | SHGVNETIYNTTLKYGDVVGVNTTKYPYRVCSMAQGTDLIRFER |
| | pack1_dI-dIV-dV- | NIVCTSMKPINEDLDEGIMVVYKRNIVAHTFKVRVYQKVLTERR |
| | pack6_Disulf1 | SYAYHRTTYLLGSNTEYVAPPMWEIHHINSHSQCYSSYSRVIAG |
| | Bg-KOs: I156H, H157R, | TVFVAYHRDSYENKTMQLMPDCYSSTHSTRYVTVKDQWHSRGST |
| | W240N, Y242T, C246S, | NLTRETSNLNCMVTITTARSKYPYHFFATSTGDVVDISPFYNGT |
| | R457S, R460S | NRNASYFGENADKFFIFPNYTIVSDFGRPNSALETHRLVAFLER |
| | Cpack1-short: E686L, | ADSVISWDIQDEKNVICQLTFWEASERTIRSEAEDSYHFISAKM |
| | Y690F, R693V, V694L | TAFFLSKKQEVNMSDSALDCVRDEAINKLQQIFNTSYNQTYEKY |
| | dII-dIII-pack1: S367I, | GNVSVFETTGGLVVFWQGIKQKSLVELERLANRSSLNLTHNSTK |
| | T374F | SSTDGNNATHLSNMESVHNLVYAQLQFTYDTLRGYINRALAQIA |
| | dI-dIV-dV-pack6: N220S, | EAWCVDQRRTLEVEKELSKINPSAILSAIYNKPIAAREMGDVLG |
| | V5521, Q591F | LASCLTINQTSVKVLRDMNVKESPGRCYSRPVVIENFANCSYVF |
| | Disulf1: D217C, S587C | YGQLGEDNEILLGNHRTEECQLPSLKIFIAGNSAYEYVDYLEKR |
| | | MIDLSSISTVDSMIALDIDPLENTDERVLELYSQKELRSSNVED |
| | | LEEIMRLENSFKQVLKYV |
| SEQ ID | gB-Towne-ext-N-term_Bg- | GAAVSSSSTRGTSATHSHASSHTTSAAHSRSGSVSQRVTSSQTV |
| NO: 96 | KOs_Cpack1-long_dII-dIII- | SHGVNETIYNTTLKYGDVVGVNTTKYPYRVCSMAQGTDLIRFER |
| | pack1_dI-dIV-dV- | NIVCTSMKPINEDLDEGIMVVYKRNIVAHTFKVRVYQKVLTERR |
| | pack6_Disulf1 | SYAYHRTTYLLGSNTEYVAPPMWEIHHINSHSQCYSSYSRVIAG |
| | Bg-KOs: I156H, H157R, | TVEVAYHRDSYENKTMQLMPDCYSSTHSTRYVTVKDQWHSRGST |
| | W240N, Y242T, C246S, | NLTRETSNLNCMVTITTARSKYPYHFFATSIGDVVDISPFYNGT |
| | R457S, R460S | NRNASYFGENADKFFIFPNYTIVSDFGRPNSALETHRIVAFLER |
| | Cpack1-long: E686L, Y690F, | ADSVISWDIQDEKNVTCQLTFWEASERTIRSEAEDSYHFISAKM |
| | R693V, V694D, K700A, V702Q | TAFFLSKKQEVNMSDSALDCVRDEAINKLQQIFNTSYNQTYEKY |
| | dII-dIII-pack1: S367I, | GNVSVFETTGGLVVFWQGIKQKSLVELERLANRSSLNLTHNSTK |
| | T374F | SSTDGNNATHLSNMESVHNLVYAQLQFTYDTLRGYINRALAQIA |
| | dI-dIV-dV-pack6: N220S, | EAWCVDQRRTLEVFKELSKINPSAILSAIYNKPIAARFMGDVLG |
| | V552L, Q591F | LASCLTINQTSVKVLRDMNVKESPGRCYSRPVVIFNFANCSYVE |
| | Disulf1: D217C, S587C | YGQLGEDNEILLGNHRTEECQLPSLKIFIAGNSAYEYVDYLFKR |
| | | MIDLSSISTVDSMIALDIDPLENTDERVLELYSQKELRSSNVFD |
| | | LEEIMRLENSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 97 | KOs_Cpack1-short_dI-dIV- | SHGVNETIYNTTLKYGDVVGVNTTKYPYRVCSMAQGTDLIRFER |
| | dV-pack4_Disulf1 | NIVCTSMKPINEDLDEGIMVVYKRNIVAHTEKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVEVAYHRDSYENKIMQLMPDCFSSTHSTRYVTVKDQWHSRGST |
| | R457S, R460S | NLTRETSNLNCMVTITTARSKYPYHFFATSTGDVVDISPFYNGT |
| | Cpack1-short: E686L, | NRNASYFGENADKFFIFPNYTIVSDFGRPNSALETHRIVAFLER |
| | Y690F, R693V, V694L | ADSVISWDIQDEKNVICQLTFWEASERTIRSEAEDSYHESSAKM |
| | dI-dIV-dV-pack4: Y218F, | TATFLSKKQEVNMSDSALDCVRDEAINKLQQIFNTSYNQTYEKY |
| | N220S, V552L, Q591Y, S668A | GNVSVFETTGGLVVFWQGIKQKSIVELERLANRSSLNLTHNSTK |
| | Disulf1: D217C, S587C | SSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCLTINQTSVKVLRDMNVKESPGRCYSRPVVIENFANCSYVY |
| | | YGQLGEDNEILLGNHRTEECQLPSLKIFIAGNSAYEYVDYLFKR |
| | | MIDLSSISTVDSMIALDIDPLENTDFRVLELYAQKELRSSNVED |
| | | LEEIMRLENSFKQVLKYV |
| SEQ ID | gB-Towne-ext-N-term_Bg- | GAAVSSSSTRGTSATHSHHSSHITSAAHSRSGSVSQRVTSSQTV |
| NO: 98 | KOs_Cpack1-long_dI-dIV-dV- | SHGVNETIYNTTLKYGDVVGVNTTKYPYRVCSMAQGTDLIRFER |
| | pack4_Disulf1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDCFSSTHSTRYVTVKDQWHSRGST |
| | R457S, R460S | NLTRETSNLNCMVTITTARSKYPYHFFATSTGDVVDISPFYNGT |
| | Cpack1-long: E686L, Y690F, | NRNASYFGENADKFFIFPNYTIVSDFGRPNSALETHRLVAFLER |
| | R693V, V694L, K700A, V702Q | ADSVISWDIQDEKNVICQLTFWEASERTIRSEAEDSYHFSSAKM |
| | dI-dIV-dV-pack4: Y218F, | TATFLSKKQEVNMSDSALDCVRDEAINKLQQIFNTSYNQTYEKY |
| | N220S, V552L, Q591Y, S668A | GNVSVFETTGGLVVFWQGIKQKSLVELERLANRSSLNLTHNSTK |
| | Disulf1: D217C, S587C | SSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCLTINQTSVKVLRDMNVKESPGRCYSRPVVIENFANCSYVY |
| | | YGQLGEDNEILLGNHRTEECQLPSLKIFIAGNSAYEYVDYLEKR |
| | | MIDLSSISTVDSMIALDIDPLENTDERVLELYAQKELRSSNVFD |
| | | LEEIMRLFNSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 99 | KOs_Cpack1-short_dI-dIV- | SHGVNETIYNTTLKYGDVVGVNITKYPYRVCSMAQGTDLIRFER |
| | dV-pack5_Disulf1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDCYSSTHSTRYVTVKDQWHSRGST |
| | R457S, R460S | NLTRETSNLNCMVTITTARSKYPYHFFATSTGDVVDISPFYNGT |
| | Cpack1-short: E686L, | NRNASYFGENADKFFIFPNYTIVSDFGRPNSALETHRLVAFLER |
| | Y690F, R693V, V694L | ADSVISWDIQDEKNVTCQLIFWEASERTIRSEAEDSYHFSSAKM |
| | dI-dIV-dV-pack5: N220S, | TATFLSKKQEVNMSDSALDCVRDEAINKLQQIFNTSYNQTYEKY |
| | V552L, Q591Y | GNVSVFETTGGLVVFWQGIKQKSLVELERLANRSSINLTHNSTK |
| | Disulf1: D217C, S587C | SSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCLTINQTSVKVLRDMNVKESPGRCYSRPVVIENFANCSYVY |
| | | YGQLGEDNEILLGNHRTEECQLPSLKIFIAGNSAYEYVDYLEKR |
| | | MIDLSSISTVDSMIALDIDPLENTDERVLELYSQKELRSSNVFD |
| | | LEEIMRLFNSFKQVLKYV |
| SEQ ID | gB-Towne-ext-N-term_Bg- | GAAVSSSSTRGTSATHSHHSSHTTSAAHSRSGSVSQRVTSSQTV |
| NO: 100 | KOs_Cpack1-long_dI-dIV-dV- | SHGVNETIYNTTLKYGDVVGVNTTKYPYRVCSMAQGTDLIRFER |
| | pack5_Disulf1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVEVAYHRDSYENKTMQLMPDCYSSTHSTRYVTVKDQWHSRGST |
| | R457S, R460S | NLTRETSNLNCMVTITTARSKYPYHFFATSTGDVVDISPFYNGT |
| | Cpack1-long: E686L, Y690F, | NRNASYFGENADKFFIFPNYTIVSDFGRPNSALETHRLVAFLER |
| | R693V, V694D, K700A, V702Q | ADSVISWDIQDEKNVTCQLTFWEASERTIRSEAEDSYHFSSAKM |
| | dI-dIV-dV-pack5: N220S, | TATFLSKKQEVNMSDSALDCVRDEAINKLQQIFNTSYNQTYEKY |
| | V552L, Q591Y | GNVSVFETTGGLVVFWQGIKQKSLVELERLANRSSINLTHNSTK |
| | Disulf1: D217C, S587C | SSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCLTINQTSVKVLRDMNVKESPGRCYSRPVVIENFANCSYVY |
| | | YGQLGEDNEILLGNHRTEECQLPSLKIFIAGNSAYEYVDYLFKR |
| | | MIDLSSISTVDSMIALDIDPLENTDERVLELYSQKELRSSNVED |
| | | LEEIMRLFNSFKQVLKYVEDAVQ |
| SEQ ID | gB-Towne-ext-N-term_Bg- | GAAVSSSSTRGTSATHSHHSSHTTSAAHSRSGSVSQRVTSSQTV |
| NO: 101 | KOs_Cpack1-short_dI-dIV- | SHGVNETIYNTTLKYGDVVGVNTTKYPYRVCSMAQGTDLIRFER |
| | dV-pack6_Disulf1 | NIVCTSMKPINEDLDEGIMVVYKRNIVAHTFKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVEVAYHRDSYENKTMQLMPDCYSSTHSTRYVTVKDQWHSRGST |
| | R457S, R460S | NLTRETSNLNCMVTITTARSKYPYHFFATSTGDVVDISPFYNGT |
| | Cpack1-short: E686L, | NRNASYFGENADKFFIFPNYTIVSDFGRPNSALETHRLVAFLER |
| | Y690F, R693V, V694L | ADSVISWDIQDEKNVICQLTFWEASERTIRSEAEDSYHFSSAKM |
| | dI-dIV-dV-pack6: N220S, | TATFLSKKQEVNMSDSALDCVRDEAINKLQQIFNTSYNQTYEKY |
| | V552L, Q591F | GNVSVFETTGGLVVFWQGIKQKSLVELERLANRSSLNLTHNSTK |
| | Disulf1: D217C, S587C | SSTDGNNATHLSNMESVHNLVYAQLQFTYDTLRGYINRALAQIA |
| | | EAWCVDQRRTLEVFKELSKINPSAILSAIYNKPIAARFMGDVLG |
| | | LASCLTINQTSVKVLRDMNVKESPGRCYSRPVVIENFANCSYVF |
| | | YGQLGEDNEILLGNHRTEECQLPSLKIFIAGNSAYEYVDYLFKR |
| | | MIDLSSISTVDSMIALDIDPLENTDFRVLELYSQKELRSSNVFD |
| | | LEEIMRLENSFKQVLKYV |
| SEQ ID | gB-Towne-ext-N-term_Bg- | GAAVSSSSTRGTSATHSHHSSHTTSAAHSRSGSVSQRVTSSQTV |
| NO: 102 | KOs_Cpack1-long_dI-dIV-dV- | SHGVNETIYNTTLKYGDVVGVNTTKYPYRVCSMAQGIDLIRFER |
| | pack6_Disulf1 | NIVCTSMKPINEDLDEGIMVVYKRNIVAHTEKVRVYQKVLTERR |
| | Bg-KOs: I156H, H157R, | SYAYHRTTYLLGSNTEYVAPPMWEIHHINSHSQCYSSYSRVIAG |
| | W240N, Y242T, C246S, | TVFVAYHRDSYENKTMQLMPDCYSSTHSTRYVTVKDQWHSRGST |
| | R457S, R460S | NLTRETSNLNCMVTITTARSKYPYHFFATSTGDVVDISPFYNGT |
| | Cpack1-long: E686L, Y690F, | NRNASYFGENADKFFIFPNYTIVSDFGRPNSALETHRLVAFLER |
| | R693V, V694L, K700A, V702Q | ADSVISWDIQDEKNVTCQLIFWEASERTIRSEAEDSYHFSSAKM |
| | dI-dIV-dV-pack6: N220S, | TATFLSKKQEVNMSDSALDCVRDEAINKLQQIFNTSYNQTYEKY |
| | V5521, Q591F | GNVSVFETTGGLVVFWQGIKQKSIVELERLANRSSLNLTHNSTK |
| | Disulf1: D217C, S587C | SSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCLTINQTSVKVLRDMNVKESPGRCYSRPVVIENFANCSYVE |
| | | YGQLGEDNEILLGNHRTEECQLPSLKIFIAGNSAYEYVDYLFKR |
| | | MIDLSSISTVDSMIALDIDPLENTDERVLELYSQKELRSSNVED |
| | | LEEIMRLFNSFKQVLKYVEDAVQ |
| SEQ ID | gB-Towne-ext-N-term_dII- | GAAVSSSSTRGTSATHSHHSSHTTSAAHSRSGSVSQRVTSSQTV |
| NO: 103 | dIII-pack1 | SHGVNETIYNTTLKYGDVVGVNTTKYPYRVCSMAQGTDLIRFER |
| | dII-dIII-pack1: S367I, | NIVCTSMKPINEDLDEGIMVVYKRNIVAHTEKVRVYQKVLTERR |
| | T374F | SYAYINTTYLLGSNTEYVAPPMWEIHHINSHSQCYSSYSRVIAG |
| | | TVEVAYHRDSYENKTMQLMPDDYSNTHSTRYVTVKDQWHSRGST |
| | | WLYRETCNLNCMVTITTARSKYPYHFFATSTGDVVDISPFYNGT |
| | | NRNASYFGENADKFFIFPNYTIVSDFGRPNSALETHRLVAFLER |
| | | ADSVISWDIQDEKNVTCQLIFWEASERTIRSEAEDSYHFISAKM |
| | | TAFFLSKKQEVNMSDSALDCVRDEAINKLQQIFNTSYNQTYEKY |
| | | GNVSVFETTGGLVVFWQGIKQKSIVELERLANRSSENLTHNRTK |
| | | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLFKR |
| | | MIDLSSISTVDSMIALDIDPLENTDERVLELYSQKELRSSNVED |
| | | LEEIMREFNSYKQRVKYV |
| SEQ ID | gB-Towne-ext-N- | GAAVSSSSTRGTSATHSHHSSHTTSAAHSRSGSVSQRVTSSQTV |
| NO: 104 | term_Cpack1-short | SHGVNETIYNTTLKYGDVVGVNTTKYPYRVCSMAQGIDLIRFER |
| | Cpack1-short: E686L, | NIVCTSMKPINEDLDEGIMVVYKRNIVAHTFKVRVYQKVLTERR |
| | Y690F, R693V, V694L | SYAYIHTTYLLGSNTEYVAPPMWEIHHINSHSQCYSSYSRVIAG |
| | | TVFVAYHRDSYENKTMQLMPDDYSNTHSTRYVTVKDQWHSRGST |
| | | WLYRETCNLNCMVTITTARSKYPYHFFATSTGDVVDISPFYNGT |
| | | NRNASYFGENADKFFIFPNYTIVSDFGRPNSALETHRLVAFLER |
| | | ADSVISWDIQDEKNVTCQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSIVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVFD |
| | | LEEIMRLFNSFKQVLKYV |
| SEQ ID | gB-Towne-ext-N- | GAAVSSSSTRGTSATHSHHSSHTTSAAHSRSGSVSQRVTSSQTV |
| NO: 105 | term_Cpack1-long | SHGVNETIYNTTLKYGDVVGVNTTKYPYRVCSMAQGIDLIRFER |
| | Cpack1-long: E686L, Y690F, | NIVCTSMKPINEDLDEGIMVVYKRNIVAHTFKVRVYQKVLTERR |
| | R693V, V694L, K700A, V702Q | SYAYIHTTYLLGSNTEYVAPPMWEIHHINSHSQCYSSYSRVIAG |
| | | TVFVAYHRDSYENKTMQLMPDDYSNTHSTRYVTVKDQWHSRGST |
| | | WLYRETCNLNCMVTITTARSKYPYHFFATSTGDVVDISPFYNGT |
| | | NRNASYFGENADKFFIFPNYTIVSDFGRPNSALETHRLVAFLER |
| | | ADSVISWDIQDEKNVTCQLIFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDILRGYINRALAQIA |
| | | EAWCVDQRRTLEVFKELSKINPSAILSAIYNKPIAARFMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVED |
| | | LEEIMRLENSFKQVLKYVEDAVQ |
| SEQ ID | gB-Towne-ext-N- | GAAVSSSSTRGTSATHSHHSSHTTSAAHSRSGSVSQRVTSSQTV |
| NO: 106 | term_Cpack2-short | SHGVNETIYNTTLKYGDVVGVNTTKYPYRVCSMAQGTDLIRFER |
| | Cpack2-short: D679N, | NIVCTSMKPINEDLDEGIMVVYKRNIVAHTFKVRVYQKVLTERR |
| | L680E, E681N, R685Q, | SYAYIHTTYLLGSNTEYVAPPMWEIHHINSHSQCYSSYSRVIAG |
| | E686L, Y690F, R693V, | TVFVAYHRDSYENKTMQLMPDDYSNTHSTRYVTVKDQWHSRGST |
| | V694L, K695D | WLYRETCNLNCMVTITTARSKYPYHFFATSTGDVVDISPFYNGT |
| | | NRNASYFGENADKFFIFPNYTIVSDFGRPNSALETHRLVAFLER |
| | | ADSVISWDIQDEKNVTCQLTFWEASERTIRSEAEDSYHESSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVEN |
| | | ENEIMQLFNSFKQVLDYV |
| SEQ ID | gB-Towne-ext-N- | GAAVSSSSTRGTSATHSHHSSHTTSAAHSRSGSVSQRVTSSQTV |
| NO: 107 | term_Cpack2-long | SHGVNETIYNTTLKYGDVVGVNTTKYPYRVCSMAQGTDLIRFER |
| | Cpack2-long: D679N, L680E, | NIVCTSMKPINEDLDEGIMVVYKRNIVAHTFKVRVYQKVLTERR |
| | E681N, R685Q, E686L, | SYAYIHTTYLLGSNTEYVAPPMWEIHHINSHSQCYSSYSRVIAG |
| | Y690F, R693V, V694L, | TVEVAYHRDSYENKTMQLMPDDYSNTHSTRYVTVKDQWHSRGST |
| | K695D, K700A, V701L, V702Q | WLYRETCNLNCMVTITTARSKYPYHFFATSIGDVVDISPFYNGT |
| | | NRNASYFGENADKFFIFPNYTIVSDFGRPNSALETHRIVAFLER |
| | | ADSVISWDIQDEKNVICQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLFKR |
| | | MIDLSSISTVDSMIALDIDPLENTDFRVLELYSQKELRSSNVEN |
| | | ENEIMQLENSFKQVLDYVEDALQ |
| SEQ ID | gB-Towne-ext-N- | GAAVSSSSTRGTSATHSHHSSHTTSAAHSRSGSVSQRVTSSQTV |
| NO: 108 | term_Cpack3-short | SHGVNETIYNTTLKYGDVVGVNITKYPYRVCSMAQGTDLIRFER |
| | Cpack3-short: V677T, | NIVCTSMKPINEDLDEGIMVVYKRNIVAHTFKVRVYQKVLTERR |
| | F678N, D679N, L680E, | SYAYIHTTYLLGSNTEYVAPPMWEIHHINSHSQCYSSYSRVIAG |
| | E681N, E682S, R685Q, | TVFVAYHRDSYENKTMQLMPDDYSNTHSTRYVTVKDQWHSRGST |
| | E686L, N688E, Y690F, | WLYRETCNLNCMVTITTARSKYPYHFFATSTGDVVDISPFYNGT |
| | R693V, V694L, K695D | NRNASYFGENADKFFIFPNYTIVSDFGRPNSALETHRLVAFLER |
| | | ADSVISWDIQDEKNVTCQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSIVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDFRVLELYSQKELRSSNINN |
| | | ENSIMQLFESFKQVLDYV |
| SEQ ID | gB-Towne-ext-N- | GAAVSSSSTRGTSATHSHHSSHTTSAAHSRSGSVSQRVTSSQTV |
| NO: 109 | term_Cpack3-long | SHGVNETIYNTTLKYGDVVGVNITKYPYRVCSMAQGTDLIRFER |
| | Cpack3-long: V677T, F678N, | NIVCTSMKPINEDLDEGIMVVYKRNIVAHTFKVRVYQKVLTERR |
| | D679N, L680E, E681N, | SYAYIHTTYLLGSNTEYVAPPMWEIHHINSHSQCYSSYSRVIAG |
| | E682S, R685Q, E686L, | TVEVAYHRDSYENKTMQLMPDDYSNTHSTRYVTVKDQWHSRGST |
| | N688E, Y690F, R693V, | WLYRETCNLNCMVTITTARSKYPYHFFATSIGDVVDISPFYNGT |
| | V694L, K695D, D699K, | NRNASYFGENADKFFIFPNYTIVSDFGRPNSALETHRLVAFLER |
| | K700A, V701L, V702Q | ADSVISWDIQDEKNVTCQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLFKR |
| | | MIDLSSISTVDSMIALDIDPLENTDERVLELYSQKELRSSNTNN |
| | | ENSIMQLFESFKQVLDYVEKALQ |
| SEQ ID | gB-Towne-ext-N- | GAAVSSSSTRGTSATHSHHSSHTTSAAHSRSGSVSQRVTSSQTV |
| NO: 110 | term_Cpack4-short | SHGVNETIYNTTLKYGDVVGVNTTKYPYRVCSMAQGTDLIRFER |
| | Cpack4-short: V677T, | NIVCTSMKPINEDLDEGIMVVYKRNIVAHTFKVRVYQKVLTERR |
| | F678N, D679H, L680E, | SYAYIHTTYLLGSNTEYVAPPMWEIHHINSHSQCYSSYSRVIAG |
| | E681N, E682S, M684S, | TVEVAYHRDSYENKTMQLMPDDYSNTHSTRYVTVKDQWHSRGST |
| | R685Q, E686L, F687A, | WLYRETCNINCMVTITTARSKYPYHFFATSTGDVVDISPFYNGT |
| | N688E, Y690F, Q692S, | NRNASYFGENADKFFIFPNYTIVSDFGRPNSALETHRLVAFLER |
| | R693V, V694L, K695D, Y696R | ADSVISWDIQDEKNVTCQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLFKR |
| | | MIDLSSISTVDSMIALDIDPLENTDFRVLELYSQKELRSSNINH |
| | | ENSISQLAESFKSVLDRV |
| SEQ ID | gB-Towne-ext-N- | GAAVSSSSTRGTSATHSHHSSHTTSAAHSRSGSVSQRVTSSQTV |
| NO: 111 | term_Cpack4-long | SHGVNETIYNTTLKYGDVVGVNITKYPYRVCSMAQGTDLIRFER |
| | Cpack4-long: V677T, F678N, | NIVCTSMKPINEDLDEGIMVVYKRNIVAHTFKVRVYQKVLTERR |
| | D679H, L680E, E681N, | SYAYIHTTYLLGSNTEYVAPPMWEIHHINSHSQCYSSYSRVIAG |
| | E682S, M684S, R685Q, | TVFVAYHRDSYENKTMQLMPDDYSNTHSTRYVTVKDQWHSRGST |
| | E686L, F687A, N688E, | WLYRETCNLNCMVTITTARSKYPYHFFATSTGDVVDISPFYNGT |
| | Y690F, Q692S, R693V, | NRNASYFGENADKFFIFPNYTIVSDFGRPNSALETHRLVAFLER |
| | V694L, K695D, Y696R, | ADSVISWDIQDEKNVTCQLTFWEASERTIRSEAEDSYHFSSAKM |
| | D699K, K700A, V701L, V702E | TATFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRILEVFKELSKINPSAILSAIYNKPIAARFMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNINH |
| | | ENSISQLAESFKSVLDRVEKALE |
| SEQ ID | gB-Towne-ext-N- | GAAVSSSSTRGTSATHSHHSSHTTSAAHSRSGSVSQRVTSSQTV |
| NO: 112 | term_Cpack1-short_dII- | SHGVNETIYNTTLKYGDVVGVNTTKYPYRVCSMAQGTDLIRFER |
| | dIII-pack1 | NIVCTSMKPINEDLDEGIMVVYKRNIVAHTFKVRVYQKVLTERR |
| | dII-dIII-pack1: S367I, | SYAYIHTTYLLGSNTEYVAPPMWEIHHINSHSQCYSSYSRVIAG |
| | T374F | TVFVAYHRDSYENKTMQLMPDDYSNTHSTRYVTVKDQWHSRGST |
| | Cpack1-short: E686L, | WLYRETCNLNCMVTITTARSKYPYHFFATSTGDVVDISPFYNGT |
| | Y690F, R693V, V694L | NRNASYFGENADKFFIFPNYTIVSDFGRPNSALETHRLVAFLER |
| | | ADSVISWDIQDEKNVICQLTFWEASERTIRSEAEDSYHFISAKM |
| | | TAFFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLFKR |
| | | MIDLSSISTVDSMIALDIDPLENTDERVLELYSQKELRSSNVFD |
| | | LEEIMRLFNSFKQVLKYV |
| SEQ ID | gB-Towne-ext-N- | GAAVSSSSTRGTSATHSHHSSHTTSAAHSRSGSVSQRVTSSQTV |
| NO: 113 | term_Cpack1-long_dII-dIII- | SHGVNETIYNTTLKYGDVVGVNTTKYPYRVCSMAQGTDLIRFER |
| | pack1 | NIVCTSMKPINEDLDEGIMVVYKRNIVAHTEKVRVYQKVLTERR |
| | dII-dIII-pack1: S367I, | SYAYINTTYLLGSNTEYVAPPMWEIHHINSHSQCYSSYSRVIAG |
| | T374F | TVEVAYHRDSYENKTMQLMPDDYSNTHSTRYVTVKDQWHSRGST |
| | Cpack1-long: E686L, Y690F, | WLYRETCNLNCMVTITTARSKYPYHFFATSTGDVVDISPFYNGT |
| | R693V, V694L, K700A, V702Q | NRNASYFGENADKFFIFPNYTIVSDFGRPNSALETHRLVAFLER |
| | | ADSVISWDIQDEKNVTCQLIFWEASERTIRSEAEDSYHFISAKM |
| | | TAFFLSKKQEVNMSDSALDCVRDEAINKLQQIFNTSYNQTYEKY |
| | | GNVSVFETTGGLVVFWQGIKQKSIVELERLANRSSLNLTHNRTK |
| | | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLFKR |
| | | MIDLSSISTVDSMIALDIDPLENTDFRVLELYSQKELRSSNVFD |
| | | LEEIMRLENSFKQVLKYVEDAVQ |
| SEQ ID | gB-Towne-ext-N- | GAAVSSSSTRGTSATHSHHSSHITSAAHSRSGSVSQRVTSSQTV |
| NO: 114 | term_Cpack2-short_dII- | SHGVNETIYNTTLKYGDVVGVNTTKYPYRVCSMAQGTDLIRFER |
| | dIII-pack1 | NIVCTSMKPINEDLDEGIMVVYKRNIVAHTFKVRVYQKVLTERR |
| | dII-dIII-pack1: S367I, | SYAYIHTTYLLGSNTEYVAPPMWEIHHINSHSQCYSSYSRVIAG |
| | T374F | TVFVAYHRDSYENKTMQLMPDDYSNTHSTRYVTVKDQWHSRGST |
| | Cpack2-short: D679N, | WLYRETCNLNCMVTITTARSKYPYHFFATSTGDVVDISPFYNGT |
| | L680E, E681N, R685Q, | NRNASYFGENADKFFIFPNYTIVSDFGRPNSALETHRLVAFLER |
| | E686L, Y690F, R693V, | ADSVISWDIQDEKNVICQLTFWEASERTIRSEAEDSYHFISAKM |
| | V694L, K695D | TAFFLSKKQEVNMSDSALDCVRDEAINKLQQIFNTSYNQTYEKY |
| | | GNVSVFETTGGLVVFWQGIKQKSIVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVFN |
| | | ENEIMQLFNSFKQVLDYV |
| SEQ ID | gB-Towne-ext-N- | GAAVSSSSTRGTSATHSHHSSHTTSAAHSRSGSVSQRVTSSQTV |
| NO: 115 | term_Cpack2-long_dII-dIII- | SHGVNETIYNTTLKYGDVVGVNTTKYPYRVCSMAQGTDLIRFER |
| | pack1 | NIVCTSMKPINEDLDEGIMVVYKRNIVAHTFKVRVYQKVLTERR |
| | dII-dIII-pack1: S367I, | SYAYIHTTYLLGSNTEYVAPPMWEIHHINSHSQCYSSYSRVIAG |
| | T374F | TVEVAYHRDSYENKTMQLMPDDYSNTHSTRYVTVKDQWHSRGST |
| | Cpack2-long: D679N, L680E, | WLYRETCNLNCMVTITTARSKYPYHFFATSTGDVVDISPFYNGT |
| | E681N, R685Q, E686L, | NRNASYFGENADKFFIFPNYTIVSDFGRPNSALETHRLVAFLER |
| | Y690F, R693V, V694L, | ADSVISWDIQDEKNVICQLIFWEASERTIRSEAEDSYHFISAKM |
| | K695D, K700A, V701L, V702Q | TAFFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTERGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVEN |
| | | ENEIMQLENSFKQVLDYVEDALQ |
| SEQ ID | gB-Towne-ext-N- | GAAVSSSSTRGTSATHSHHSSHTTSAAHSRSGSVSQRVTSSQTV |
| NO: 116 | term_Cpack3-short_dII- | SHGVNETIYNTTLKYGDVVGVNTTKYPYRVCSMAQGTDLIRFER |
| | dIII-pack1 | NIVCTSMKPINEDLDEGIMVVYKRNIVAHTEKVRVYQKVLTERR |
| | dII-dIII-pack1: S367I, | SYAYIHTTYLLGSNTEYVAPPMWEIHHINSHSQCYSSYSRVIAG |
| | T374F | TVEVAYHRDSYENKTMQLMPDDYSNTHSTRYVTVKDQWHSRGST |
| | Cpack3-short: V677T, | WLYRETCNINCMVTITTARSKYPYHFFATSTGDVVDISPFYNGT |
| | F678N, D679N, L680E, | NRNASYFGENADKFFIFPNYTIVSDFGRPNSALETHRLVAFLER |
| | E681N, E682S, R685Q, | ADSVISWDIQDEKNVICQLTFWEASERTIRSEAEDSYHFISAKM |
| | E686L, N688E, Y690F, | TAFFLSKKQEVNMSDSALDCVRDEAINKLQQIFNTSYNQTYEKY |
| | R693V, V694L, K695D | GNVSVFETTGGLVVFWQGIKQKSIVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLFKR |
| | | MIDLSSISTVDSMIALDIDPLENTDERVLELYSQKELRSSNTNN |
| | | ENSIMQLFESEKQVLDYV |
| SEQ ID | gB-Towne-ext-N- | GAAVSSSSTRGTSATHSHHSSHTTSAAHSRSGSVSQRVTSSQTV |
| NO: 117 | term Cpack3-long dII-dIII- | SHGVNETIYNTTLKYGDVVGVNITKYPYRVCSMAQGTDLIRFER |
| | pack1 | NIVCTSMKPINEDLDEGIMVVYKRNIVAHTEKVRVYQKVLTERR |
| | dII-dIII-pack1: S367I, | SYAYIHTTYLLGSNTEYVAPPMWEIHHINSHSQCYSSYSRVIAG |
| | T374F | TVFVAYHRDSYENKTMQLMPDDYSNTHSTRYVTVKDQWHSRGST |
| | Cpack3-long: V677T, F678N, | WLYRETCNLNCMVTITTARSKYPYHFFATSTGDVVDISPFYNGT |
| | D679N, L680E, E681N, | NRNASYFGENADKFFIFPNYTIVSDFGRPNSALETHRLVAFLER |
| | E682S, R685Q, E686L, | ADSVISWDIQDEKNVTCQLTFWEASERTIRSEAEDSYHFISAKM |
| | N688E, Y690F, R693V, | TAFFLSKKQEVNMSDSALDCVRDEAINKLQQIFNTSYNQTYEKY |
| | V694L, K695D, D699K, | GNVSVFETTGGLVVFWQGIKQKSIVELERLANRSSLNLTHNRTK |
| | K700A, V701L, V702Q | RSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDFRVLELYSQKELRSSNTNN |
| | | ENSIMQLFESFKQVLDYVEKALQ |
| SEQ ID | gB-Towne-ext-N- | GAAVSSSSTRGTSATHSHHSSHTTSAAHSRSGSVSQRVTSSQTV |
| NO: 118 | term_Cpack4-short_dII- | SHGVNETIYNTTLKYGDVVGVNITKYPYRVCSMAQGIDLIRFER |
| | dIII-pack1 | NIVCTSMKPINEDLDEGIMVVYKRNIVAHTFKVRVYQKVLTERR |
| | dII-dIII-pack1: S367I, | SYAYIHTTYLLGSNTEYVAPPMWEIHHINSHSQCYSSYSRVIAG |
| | T374F | TVFVAYHRDSYENKTMQLMPDDYSNTHSTRYVTVKDQWHSRGST |
| | Cpack4-short: V677T, | WLYRETCNLNCMVTITTARSKYPYHFFATSTGDVVDISPFYNGT |
| | F678N, D679H, L680E, | NRNASYFGENADKFFIFPNYTIVSDFGRPNSALETHRLVAFLER |
| | E681N, E682S, M684S, | ADSVISWDIQDEKNVTCQLIFWEASERTIRSEAEDSYHFISAKM |
| | R685Q, E686L, F687A, | TAFFLSKKQEVNMSDSALDCVRDEAINKLQQIFNTSYNQTYEKY |
| | N688E, Y690F, Q692S, | GNVSVFETTGGLVVFWQGIKQKSLVELERLANRSSLNLTHNRTK |
| | R693V, V694L, K695D, Y696R | RSTDGNNATHLSNMESVHNLVYAQLQFTYDTLRGYINRALAQIA |
| | | EAWCVDQRRILEVFKELSKINPSAILSAIYNKPIAAREMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLFKR |
| | | MIDLSSISTVDSMIALDIDPLENTDERVLELYSQKELRSSNINH |
| | | ENSISQLAESFKSVLDRV |
| SEQ ID | gB-Towne-ext-N- | GAAVSSSSTRGTSATHSHHSSHTTSAAHSRSGSVSQRVTSSQTV |
| NO: 119 | term_Cpack4-long_dII-dIII- | SHGVNETIYNTTLKYGDVVGVNTTKYPYRVCSMAQGTDLIRFER |
| | pack1 | NIVCTSMKPINEDLDEGIMVVYKRNIVAHTEKVRVYQKVLTERR |
| | dII-dIII-pack1: S367I, | SYAYIHTTYLLGSNTEYVAPPMWEIHHINSHSQCYSSYSRVIAG |
| | T374F | TVFVAYHRDSYENKTMQLMPDDYSNTHSTRYVTVKDQWHSRGST |
| | Cpack4-long: V677T, F678N, | WLYRETCNLNCMVTITTARSKYPYHFFATSTGDVVDISPFYNGT |
| | D679H, L680E, E681N, | NRNASYFGENADKFFIFPNYTIVSDFGRPNSALETHRLVAFLER |
| | E682S, M684S, R685Q, | ADSVISWDIQDEKNVTCQLTFWEASERTIRSEAEDSYHFISAKM |
| | E686L, F687A, N688E, | TAFFLSKKQEVNMSDSALDCVRDEAINKLQQIFNTSYNQTYEKY |
| | Y690F, Q692S, R693V, | GNVSVFETTGGLVVFWQGIKQKSIVELERLANRSSLNLTHNRTK |
| | V694L, K695D, Y696R, | RSTDGNNATHLSNMESVHNLVYAQLQFTYDTLRGYINRALAQIA |
| | D699K, K700A, V701L, V702E | EAWCVDQRRTLEVFKELSKINPSAILSAIYNKPIAAREMGDVLG |
| | | LASCVTINQTSVKVLRDMNVKESPGRCYSRPVVIENFANSSYVQ |
| | | YGQLGEDNEILLGNHRTEECQLPSLKIFIAGNSAYEYVDYLFKR |
| | | MIDLSSISTVDSMIALDIDPLENTDERVLELYSQKELRSSNTNH |
| | | ENSISQLAESFKSVLDRVEKALE |
| SEQ ID | gB-Towne-ext-N- | GAAVSSSSTRGTSATHSHASSHTTSAAHSRSGSVSQRVTSSQTV |
| NO: 120 | term_Cpack1-short_dI-dIV- | SHGVNETIYNTTLKYGDVVGVNTTKYPYRVCSMAQGTDLIRFER |
| | dV-pack1 | NIVCTSMKPINEDLDEGIMVVYKRNIVAHTFKVRVYQKVLTERR |
| | Cpack1-short: E686L, | SYAYIHTTYLLGSNTEYVAPPMWEIHHINSHSQCYSSYSRVIAG |
| | Y690F, R693V, V694L | TVEVAYHRDSYENKTMQLMPDDFSNTHSTRYVTVKDQWHSRGST |
| | dI-dIV-dV-pack1: Y218F, | WLYRETCNLNCMVTITTARSKYPYHFFATSTGDVVDISPFYNGT |
| | Q591F, S668A | NRNASYFGENADKFFIFPNYTIVSDFGRPNSALETHRLVAFLER |
| | | ADSVISWDIQDEKNVTCQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCVTINQTSVKVLRDMNVKESPGRCYSRPVVIENFANSSYVF |
| | | YGQLGEDNEILLGNHRTEECQLPSLKIFIAGNSAYEYVDYLFKR |
| | | MIDLSSISTVDSMIALDIDPLENTDFRVLELYAQKELRSSNVFD |
| | | LEEIMRLENSFKQVLKYV |
| SEQ ID | gB-Towne-ext-N- | GAAVSSSSTRGTSATHSHHSSHTTSAAHSRSGSVSQRVTSSQTV |
| NO: 121 | term_Cpack1-long_dI-dIV- | SHGVNETIYNTTLKYGDVVGVNTTKYPYRVCSMAQGIDLIRFER |
| | dV-pack1 | NIVCTSMKPINEDLDEGIMVVYKRNIVAHTFKVRVYQKVLTERR |
| | Cpack1-long: E686L, Y690F, | SYAYIHTTYLLGSNTEYVAPPMWEIHHINSHSQCYSSYSRVIAG |
| | R693V, V694L, K700A, V702Q | TVFVAYHRDSYENKTMQLMPDDESNTHSTRYVTVKDQWHSRGST |
| | dI-dIV-dV-pack1: Y218F, | WLYRETCNLNCMVTITTARSKYPYHFFATSTGDVVDISPFYNGT |
| | Q591F, S668A | NRNASYFGENADKFFIFPNYTIVSDFGRPNSALETHRLVAFLER |
| | | ADSVISWDIQDEKNVTCQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIENFANSSYVF |
| | | YGQLGEDNEILLGNHRTEECQLPSLKIFIAGNSAYEYVDYLFKR |
| | | MIDLSSISTVDSMIALDIDPLENTDERVLELYAQKELRSSNVFD |
| | | LEEIMRLFNSFKQVLKYVEDAVQ |
| SEQ ID | gB-Towne-ext-N- | GAAVSSSSTRGTSATHSHHSSHTTSAAHSRSGSVSQRVTSSQTV |
| NO: 122 | term_Cpack1-short_Disulf1 | SHGVNETIYNTTLKYGDVVGVNTTKYPYRVCSMAQGTDLIRFER |
| | Cpack1-short: E686L, | NIVCTSMKPINEDLDEGIMVVYKRNIVAHTFKVRVYQKVLTERR |
| | Y690F, R693V, V694L | SYAYINTTYLLGSNTEYVAPPMWEIHHINSHSQCYSSYSRVIAG |
| | Disulf1: D217C, S587C | TVEVAYHRDSYENKTMQLMPDCYSNTHSTRYVTVKDQWHSRGST |
| | | WLYRETCNLNCMVTITTARSKYPYHFFATSTGDVVDISPFYNGT |
| | | NRNASYFGENADKFFIFPNYTIVSDFGRPNSALETHRLVAFLER |
| | | ADSVISWDIQDEKNVTCQLIFWEASERTIRSEAEDSYHESSAKM |
| | | TATFLSKKQEVNMSDSALDCVRDEAINKLQQIFNTSYNQTYEKY |
| | | GNVSVFETTGGLVVFWQGIKQKSIVELERLANRSSLNLTHNRTK |
| | | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIFNFANCSYVQ |
| | | YGQLGEDNEILLGNHRTEECQLPSLKIFIAGNSAYEYVDYLFKR |
| | | MIDLSSISTVDSMIALDIDPLENTDERVLELYSQKELRSSNVED |
| | | LEEIMRLENSFKQVLKYV |
| SEQ ID | gB-Towne-ext-N- | GAAVSSSSTRGTSATHSHHSSHTTSAAHSRSGSVSQRVTSSQTV |
| NO: 123 | term_Cpack1-long_Disulf1 | SHGVNETIYNTTLKYGDVVGVNTTKYPYRVCSMAQGTDLIRFER |
| | Cpack1-long: E686L, Y690F, | NIVCTSMKPINEDLDEGIMVVYKRNIVAHTFKVRVYQKVLTERR |
| | R693V, V694L, K700A, V702Q | SYAYIHTTYLLGSNTEYVAPPMWEIHHINSHSQCYSSYSRVIAG |
| | Disulf1: D217C, S587C | TVEVAYHRDSYENKTMQLMPDCYSNTHSTRYVTVKDQWHSRGST |
| | | WLYRETCNLNCMVTITTARSKYPYHFFATSTGDVVDISPFYNGT |
| | | NRNASYFGENADKFFIFPNYTIVSDFGRPNSALETHRLVAFLER |
| | | ADSVISWDIQDEKNVICQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSIVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCVTINQTSVKVLRDMNVKESPGRCYSRPVVIENFANCSYVQ |
| | | YGQLGEDNEILLGNHRTEECQLPSLKIFIAGNSAYEYVDYLEKR |
| | | MIDLSSISTVDSMIALDIDPLENTDFRVLELYSQKELRSSNVFD |
| | | LEEIMRLFNSFKQVLKYVEDAVQ |
| SEQ ID | gB-Towne-ext-N- | GAAVSSSSTRGTSATHSHHSSHTTSAAHSRSGSVSQRVTSSQTV |
| NO: 124 | term_Cpack1-short_Disulf2 | SHGVNETIYNTTLKYGDVVGVNTTKYPYRVCSMAQGIDLIRFER |
| | Cpack1-short: E686L, | NIVCTSMKPINEDLDEGIMVVYKRNIVAHTFKVRVYQKVLTERR |
| | Y690F, R693V, V694L | SYAYIHTTYLLGSNTEYVAPPMWEIHHINSHSQCYSSYSRVIAG |
| | Disulf2: D217C, Y589C | TVFVAYHRDSYENKTMQLMPDCYSNTHSTRYVTVKDQWHSRGST |
| | | WLYRETCNLNCMVTITTARSKYPYHFFATSTGDVVDISPFYNGT |
| | | NRNASYFGENADKFFIFPNYTIVSDFGRPNSALETHRLVAFLER |
| | | ADSVISWDIQDEKNVICQLTFWEASERTIRSEAEDSYHESSAKM |
| | | TATFLSKKQEVNMSDSALDCVRDEAINKLQQIFNTSYNQTYEKY |
| | | GNVSVFETTGGLVVFWQGIKQKSLVELERLANRSSINLTHNRTK |
| | | RSTDGNNATHLSNMESVHNLVYAQLQFTYDTERGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIENFANSSCVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVED |
| | | LEEIMRLENSFKQVLKYV |
| SEQ ID | gB-Towne-ext-N- | GAAVSSSSTRGTSATHSHHSSHTTSAAHSRSGSVSQRVTSSQTV |
| NO: 125 | term_Cpack1-long_Disulf2 | SHGVNETIYNTTLKYGDVVGVNTTKYPYRVCSMAQGTDLIRFER |
| | Cpack1-long: E686L, Y690F, | NIVCTSMKPINEDLDEGIMVVYKRNIVAHTFKVRVYQKVLTERR |
| | R693V, V694L, K700A, V702Q | SYAYIHTTYLLGSNTEYVAPPMWEIHHINSHSQCYSSYSRVIAG |
| | Disulf2: D217C, Y589C | TVFVAYHRDSYENKTMQLMPDCYSNTHSTRYVTVKDQWHSRGST |
| | | WLYRETCNLNCMVTITTARSKYPYHFFATSTGDVVDISPFYNGT |
| | | NRNASYFGENADKFFIFPNYTIVSDFGRPNSALETHRLVAFLER |
| | | ADSVISWDIQDEKNVTCQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSIVELERLANRSSLNLTHNRTK |
| | | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIFNFANSSCVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVED |
| | | LEEIMRLFNSFKQVLKYVEDAVQ |
| SEQ ID | gB-Towne-ext-N- | GAAVSSSSTRGTSATHSHHSSHTTSAAHSRSGSVSQRVTSSQTV |
| NO: 126 | term_Cpack1-short_dI-dIV- | SHGVNETIYNTTLKYGDVVGVNTTKYPYRVCSMAQGTDLIRFER |
| | dV-pack1_Disulf1 | NIVCTSMKPINEDLDEGIMVVYKRNIVAHTFKVRVYQKVLTERR |
| | Cpack1-short: E686L, | SYAYIHTTYLLGSNTEYVAPPMWEIHHINSHSQCYSSYSRVIAG |
| | Y690F, R693V, V694L | TVEVAYHRDSYENKTMQLMPDCFSNTHSTRYVTVKDQWHSRGST |
| | dI-dIV-dV-pack1: Y218F, | WLYRETCNLNCMVTITTARSKYPYHFFATSTGDVVDISPFYNGT |
| | Q591F, S668A | NRNASYFGENADKFFIFPNYTIVSDFGRPNSALETHRLVAFLER |
| | Disulf1: D217C, S587C | ADSVISWDIQDEKNVTCQLTFWEASERTIRSEAEDSYHESSAKM |
| | | TATFLSKKQEVNMSDSALDCVRDEAINKLQQIFNTSYNQTYEKY |
| | | GNVSVFETTGGLVVFWQGIKQKSIVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCVTINQTSVKVLRDMNVKESPGRCYSRPVVIENFANCSYVF |
| | | YGQLGEDNEILLGNHRTEECQLPSLKIFIAGNSAYEYVDYLFKR |
| | | MIDLSSISTVDSMIALDIDPLENTDERVLELYAQKELRSSNVED |
| | | LEEIMRLENSFKQVLKYV |
| SEQ ID | gB-Towne-ext-N- | GAAVSSSSTRGTSATHSHHSSHTTSAAHSRSGSVSQRVTSSQTV |
| NO: 127 | term Cpacki-long_dI-dIV- | SHGVNETIYNTTLKYGDVVGVNITKYPYRVCSMAQGTDLIRFER |
| | dV-pack1_Disulf1 | NIVCTSMKPINEDLDEGIMVVYKRNIVAHTFKVRVYQKVLTERR |
| | Cpack1-long: E686L, Y690F, | SYAYIHTTYLLGSNTEYVAPPMWEIHHINSHSQCYSSYSRVIAG |
| | R693V, V694L, K700A, V702Q | TVFVAYHRDSYENKTMQLMPDCFSNTHSTRYVTVKDQWHSRGST |
| | dI-dIV-dV-pack1: Y218F, | WLYRETCNLNCMVTITTARSKYPYHFFATSTGDVVDISPFYNGT |
| | Q591F, S668A | NRNASYFGENADKFFIFPNYTIVSDFGRPNSALETHRLVAFLER |
| | Disulf1: D217C, S587C | ADSVISWDIQDEKNVTCQLTFWEASERTIRSEAEDSYHESSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCVTINQTSVKVLRDMNVKESPGRCYSRPVVIENFANCSYVE |
| | | YGQLGEDNEILLGNHRTEECQLPSLKIFIAGNSAYEYVDYLEKR |
| | | MIDLSSISTVDSMIALDIDPLENTDERVLELYAQKELRSSNVFD |
| | | LEEIMRLFNSFKQVLKYVEDAVQ |
| SEQ ID | gB-Towne-ext-N- | GAAVSSSSTRGTSATHSHHSSHTTSAAHSRSGSVSQRVTSSQTV |
| NO: 128 | term_Cpack1-short_dI-dIV- | SHGVNETIYNTTLKYGDVVGVNITKYPYRVCSMAQGTDLIRFER |
| | dV-pack1_Disulf2 | NIVCTSMKPINEDLDEGIMVVYKRNIVAHTEKVRVYQKVLTERR |
| | Cpack1-short: E686L, | SYAYIHTTYLLGSNTEYVAPPMWEIHHINSHSQCYSSYSRVIAG |
| | Y690F, R693V, V694L | TVEVAYHRDSYENKTMQLMPDCFSNTHSTRYVTVKDQWHSRGST |
| | dI-dIV-dV-pack1: Y218F, | WLYRETCNLNCMVTITTARSKYPYHFFATSTGDVVDISPFYNGT |
| | Q591F, S668A | NRNASYFGENADKFFIFPNYTIVSDFGRPNSALETHRLVAFLER |
| | Disulf2: D217C, Y589C | ADSVISWDIQDEKNVTCQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSLVELERLANRSSINLTHNRTK |
| | | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIENFANSSCVE |
| | | YGQLGEDNEILLGNHRTEECQLPSLKIFIAGNSAYEYVDYLFKR |
| | | MIDLSSISTVDSMIALDIDPLENTDERVLELYAQKELRSSNVED |
| | | LEEIMRLFNSFKQVLKYV |
| SEQ ID | gB-Towne-ext-N- | GAAVSSSSTRGTSATHSHHSSHTTSAAHSRSGSVSQRVTSSQTV |
| NO: 129 | term_Cpack1-long_dI-dIV- | SHGVNETIYNTTLKYGDVVGVNTTKYPYRVCSMAQGTDLIRFER |
| | dV-pack1_Disulf2 | NIVCTSMKPINEDLDEGIMVVYKRNIVAHTFKVRVYQKVLTERR |
| | Cpack1-long: E686L, Y690F, | SYAYINTTYLLGSNTEYVAPPMWEIHHINSHSQCYSSYSRVIAG |
| | R693V, V694L, K700A, V702Q | TVEVAYHRDSYENKTMQLMPDCFSNTHSTRYVTVKDQWHSRGST |
| | dI-dIV-dV-pack1: Y218F, | WLYRETCNINCMVTITTARSKYPYHFFATSTGDVVDISPFYNGT |
| | Q591F, S668A | NRNASYFGENADKFFIFPNYTIVSDFGRPNSALETHRLVAFLER |
| | Disulf2: D217C, Y589C | ADSVISWDIQDEKNVICQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSIVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCVTINQTSVKVLRDMNVKESPGRCYSRPVVIENFANSSCVF |
| | | YGQLGEDNEILLGNHRTEECQLPSLKIFIAGNSAYEYVDYLFKR |
| | | MIDLSSISTVDSMIALDIDPLENTDFRVLELYAQKELRSSNVFD |
| | | LEEIMPLENSFKQVLKYVEDAVQ |
| SEQ ID | gB-Towne-ext-N- | GAAVSSSSTRGTSATHSHHSSHTTSAAHSRSGSVSQRVTSSQTV |
| NO: 130 | term_Cpack1-short_dI-dIV- | SHGVNETIYNTTLKYGDVVGVNITKYPYRVCSMAQGTDLIRFER |
| | dV-pack1_N165glyc | NIVCTSMKPINEDLDEGIMVVYKRNIVAHTFKVRVYQKVLTERR |
| | Cpack1-short: E686L, | SYAYIHTTYLLGSNTTYVAPPMWEIHHINSHSQCYSSYSRVIAG |
| | Y690F, R693V, V694L | TVFVAYHRDSYENKTMQLMPDDESNTHSTRYVTVKDQWHSRGST |
| | dI-dIV-dV-pack1: Y218F, | WLYRETCNLNCMVTITTARSKYPYHFFATSTGDVVDISPFYNGT |
| | Q591F, S668A | NRNASYFGENADKFFIFPNYTIVSDFGRPNSALETHRLVAFLER |
| | N165glyc: E167T | ADSVISWDIQDEKNVTCQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTERGYINRALAQIA |
| | | EAWCVDQRRILEVFKELSKINPSAILSAIYNKPIAARFMGDVLG |
| | | LASCVTINQTSVKVLRDMNVKESPGRCYSRPVVIENFANSSYVE |
| | | YGQLGEDNEILLGNHRTEECQLPSLKIFIAGNSAYEYVDYLEKR |
| | | MIDLSSISTVDSMIALDIDPLENTDERVLELYAQKELRSSNVED |
| | | LEEIMRLENSFKQVLKYV |
| SEQ ID | gB-Towne-ext-N- | GAAVSSSSTRGTSATHSHHSSHTTSAAHSRSGSVSQRVTSSQTV |
| NO: 131 | term_Cpack1-long_dI-dIV- | SHGVNETIYNTTLKYGDVVGVNTTKYPYRVCSMAQGTDLIRFER |
| | dV-pack1_N165glyc | NIVCTSMKPINEDLDEGIMVVYKRNIVAHTEKVRVYQKVLTERR |
| | Cpack1-long: E686L, Y690F, | SYAYIHTTYLLGSNTTYVAPPMWEIHHINSHSQCYSSYSRVIAG |
| | R693V, V694L, K700A, V702Q | TVFVAYHRDSYENKTMQLMPDDESNTHSTRYVTVKDQWHSRGST |
| | dI-dIV-dV-pack1: Y218F, | WLYRETCNLNCMVTITTARSKYPYHFFATSTGDVVDISPFYNGT |
| | Q591F, S668A | NRNASYFGENADKFFIFPNYTIVSDEGRPNSALETHRLVAFLER |
| | N165glyc: E167T | ADSVISWDIQDEKNVTCQLTFWEASERTIRSEAEDSYHESSAKM |
| | | TATFLSKKQEVNMSDSALDCVRDEAINKLQQIFNTSYNQTYEKY |
| | | GNVSVFETTGGLVVFWQGIKQKSIVELERLANRSSLNLTHNRTK |
| | | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIENFANSSYVE |
| | | YGQLGEDNEILLGNHRTEECQLPSLKIFIAGNSAYEYVDYLFKR |
| | | MIDLSSISTVDSMIALDIDPLENTDERVLELYAQKELRSSNVFD |
| | | LEEIMRLFNSFKQVLKYVEDAVQ |
| SEQ ID | gB-Towne-ext-N- | GAAVSSSSTRGTSATHSHHSSHTTSAAHSRSGSVSQRVTSSQTV |
| NO: 132 | term_Cpack1-short_dII- | SHGVNETIYNTTLKYGDVVGVNTTKYPYRVCSMAQGTDLIRFER |
| | dIII-pack1_dI-dIV-dV-pack1 | NIVCTSMKPINEDLDEGIMVVYKRNIVAHTFKVRVYQKVLTFRR |
| | dII-dIII-pack1: S367I, | SYAYINTTYLLGSNTEYVAPPMWEIHHINSHSQCYSSYSRVIAG |
| | T374F | TVFVAYHRDSYENKTMQLMPDDESNTHSTRYVTVKDQWHSRGST |
| | Cpack1-short: E686L, | WLYRETCNLNCMVTITTARSKYPYHFFATSTGDVVDISPFYNGT |
| | Y690F, R693V, V694L | NRNASYFGENADKFFIFPNYTIVSDFGRPNSALETHRLVAFLER |
| | dI-dIV-dV-pack1: Y218F, | ADSVISWDIQDEKNVTCQLIFWEASERTIRSEAEDSYHFISAKM |
| | Q591F, S668A | TAFFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIFNFANSSYVE |
| | | YGQLGEDNEILLGNHRTEECQLPSLKIFIAGNSAYEYVDYLFKR |
| | | MIDLSSISTVDSMIALDIDPLENTDFRVLELYAQKELRSSNVFD |
| | | LEEIMRLENSFKQVLKYV |
| SEQ ID | gB-Towne-ext-N- | GAAVSSSSTRGTSATHSHHSSHTTSAAHSRSGSVSQRVTSSQTV |
| NO: 133 | term_Cpack1-long_dII-dIII- | SHGVNETIYNTTLKYGDVVGVNTTKYPYRVCSMAQGTDLIRFER |
| | pack1_dI-dIV-dV-pack1 | NIVCTSMKPINEDLDEGIMVVYKRNIVAHTFKVRVYQKVLTERR |
| | dII-dIII-pack1: S367I, | SYAYIHTTYLLGSNTEYVAPPMWEIHHINSHSQCYSSYSRVIAG |
| | T374F | TVFVAYHRDSYENKTMQLMPDDFSNTHSTRYVTVKDQWHSRGST |
| | Cpack1-long: E686L, Y690F, | WLYRETCNLNCMVTITTARSKYPYHFFATSTGDVVDISPFYNGT |
| | R693V, V694L, K700A, V702Q | NRNASYFGENADKFFIFPNYTIVSDFGRPNSALETHRLVAFLER |
| | dI-dIV-dV-pack1: Y218F, | ADSVISWDIQDEKNVTCQLTFWEASERTIRSEAEDSYHFISAKM |
| | Q591F, S668A | TAFFLSKKQEVNMSDSALDCVRDEAINKLQQIFNTSYNQTYEKY |
| | | GNVSVFETTGGLVVFWQGIKQKSIVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCVTINQTSVKVLRDMNVKESPGRCYSRPVVIENFANSSYVE |
| | | YGQLGEDNEILLGNHRTEECQLPSLKIFIAGNSAYEYVDYLEKR |
| | | MIDLSSISTVDSMIALDIDPLENTDFRVLELYAQKELRSSNVFD |
| | | LEEIMRLFNSFKQVLKYVEDAVQ |
| SEQ ID | gB-Towne-ext-N- | GAAVSSSSTRGTSATHSHHSSHTTSAAHSRSGSVSQRVTSSQTV |
| NO: 134 | term_Cpack1-short_dII- | SHGVNETIYNTTLKYGDVVGVNTTKYPYRVCSMAQGTDLIRFER |
| | dIII-pack1_Disulf1 | NIVCTSMKPINEDLDEGIMVVYKRNIVAHTFKVRVYQKVLTERR |
| | dII-dIII-pack1: S367I, | SYAYIHTTYLLGSNTEYVAPPMWEIHHINSHSQCYSSYSRVIAG |
| | T374F | TVFVAYHRDSYENKTMQLMPDCYSNTHSTRYVTVKDQWHSRGST |
| | Cpack1-short: E686L, | WLYRETCNLNCMVTITTARSKYPYHFFATSTGDVVDISPFYNGT |
| | Y690F, R693V, V694L | NRNASYFGENADKFFIFPNYTIVSDFGRPNSALETHRLVAFLER |
| | Disulf1: D217C, S587C | ADSVISWDIQDEKNVTCQLIFWEASERTIRSEAEDSYHFISAKM |
| | | TAFFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVFKELSKINPSAILSAIYNKPIAARFMGDVLG |
| | | LASCVTINQTSVKVLRDMNVKESPGRCYSRPVVIENFANCSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVED |
| | | LEEIMRLENSFKQVLKYV |
| SEQ ID | gB-Towne-ext-N- | GAAVSSSSTRGTSATHSHHSSHTTSAAHSRSGSVSQRVTSSQTV |
| NO: 135 | term_Cpack1-long_dII-dIII- | SHGVNETIYNTTLKYGDVVGVNTTKYPYRVCSMAQGTDLIRFER |
| | pack1_Disulf1 | NIVCTSMKPINEDLDEGIMVVYKRNIVAHTEKVRVYQKVLTERR |
| | dII-dIII-pack1: S367I, | SYAYIHTTYLLGSNTEYVAPPMWEIHHINSHSQCYSSYSRVIAG |
| | T374F | TVEVAYHRDSYENKTMQLMPDCYSNTHSTRYVTVKDQWHSRGST |
| | Cpack1-long: E686L, Y690F, | WLYRETCNLNCMVTITTARSKYPYHFFATSIGDVVDISPFYNGT |
| | R693V, V694L, K700A, V702Q | NRNASYFGENADKFFIFPNYTIVSDFGRPNSALETHRLVAFLER |
| | Disulf1: D217C, 3587C | ADSVISWDIQDEKNVICQLTFWEASERTIRSEAEDSYHFISAKM |
| | | TAFFLSKKQEVNMSDSALDCVRDEAINKLQQIFNTSYNQTYEKY |
| | | GNVSVFETTGGLVVFWQGIKQKSIVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCVTINQTSVKVLRDMNVKESPGRCYSRPVVIENFANCSYVQ |
| | | YGQLGEDNEILLGNHRTEECQLPSLKIFIAGNSAYEYVDYLFKR |
| | | MIDLSSISTVDSMIALDIDPLENTDFRVLELYSQKELRSSNVED |
| | | LEEIMRLENSFKQVLKYVEDAVQ |
| SEQ ID | gB-Towne-ext-N- | GAAVSSSSTRGTSATHSHHSSHTTSAAHSRSGSVSQRVTSSQTV |
| NO: 136 | term_Cpack1-short_dII- | SHGVNETIYNTTLKYGDVVGVNTTKYPYRVCSMAQGTDLIRFER |
| | dIII-pack1_Disulf2 | NIVCTSMKPINEDLDEGIMVVYKRNIVAHTFKVRVYQKVLTERR |
| | dII-dIII-pack1: S367I, | SYAYIHTTYLLGSNTEYVAPPMWEIHHINSHSQCYSSYSRVIAG |
| | T374F | TVFVAYHRDSYENKTMQLMPDCYSNTHSTRYVTVKDQWHSRGST |
| | Cpack1-short: E686L, | WLYRETCNLNCMVTITTARSKYPYHFFATSTGDVVDISPFYNGT |
| | Y690F, R693V, V694L | NRNASYFGENADKFFIFPNYTIVSDFGRPNSALETHRLVAFLER |
| | Disulf2: D217C, Y589C | ADSVISWDIQDEKNVICQLTFWEASERTIRSEAEDSYHFISAKM |
| | | TAFFLSKKQEVNMSDSALDCVRDEAINKLQQIFNTSYNQTYEKY |
| | | GNVSVFETTGGLVVFWQGIKQKSIVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCVTINQTSVKVLRDMNVKESPGRCYSRPVVIENFANSSCVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVFD |
| | | LEEIMRLFNSFKQVLKYV |
| SEQ ID | gB-Towne-ext-N- | GAAVSSSSTRGTSATHSHHSSHTTSAAHSRSGSVSQRVTSSQTV |
| NO: 137 | term_Cpack1-long_dII-dIII- | SHGVNETIYNTTLKYGDVVGVNTTKYPYRVCSMAQGTDLIRFER |
| | pack1_Disulf2 | NIVCTSMKPINEDLDEGIMVVYKRNIVAHTFKVRVYQKVLTERR |
| | dII-dIII-pack1: S367I, | SYAYIHTTYLLGSNTEYVAPPMWEIHHINSHSQCYSSYSRVIAG |
| | T374F | TVFVAYHRDSYENKTMQLMPDCYSNTHSTRYVTVKDQWHSRGST |
| | Cpack1-long: E686L, Y690F, | WLYRETCNLNCMVTITTARSKYPYHFFATSTGDVVDISPFYNGT |
| | R693V, V694L, K700A, V702Q | NRNASYFGENADKFFIFPNYTIVSDFGRPNSALETHRLVAFLER |
| | Disulf2: D217C, Y589C | ADSVISWDIQDEKNVTCQLIFWEASERTIRSEAEDSYHFISAKM |
| | | TAFFLSKKQEVNMSDSALDCVRDEAINKLQQIFNTSYNQTYEKY |
| | | GNVSVFETTGGLVVFWQGIKQKSLVELERLANRSSINLTHNRTK |
| | | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCVTINQTSVKVLRDMNVKESPGRCYSRPVVIENFANSSCVQ |
| | | YGQLGEDNEILLGNHRTEECQLPSLKIFIAGNSAYEYVDYLFKR |
| | | MIDLSSISTVDSMIALDIDPLENTDERVLELYSQKELRSSNVED |
| | | LEEIMRLFNSFKQVLKYVEDAVQ |
| SEQ ID | gB-Towne-ext-N- | GAAVSSSSTRGTSATHSHHSSHTTSAAHSRSGSVSQRVTSSQTV |
| NO: 138 | term_Cpack1-short_dII- | SHGVNETIYNTTLKYGDVVGVNTTKYPYRVCSMAQGTDLIRFER |
| | dIII-pack1_dI-dIV-dV- | NIVCTSMKPINEDLDEGIMVVYKRNIVAHTEKVRVYQKVLTERR |
| | pack1_Disulf1 | SYAYIRTTYLLGSNTEYVAPPMWEIHHINSHSQCYSSYSRVIAG |
| | dII-dIII-pack1: S367I, | TVEVAYHRDSYENKTMQLMPDCFSNTHSTRYVTVKDQWHSRGST |
| | T374F | WLYRETCNLNCMVTITTARSKYPYHFFATSTGDVVDISPFYNGT |
| | Cpack1-short: E686L, | NRNASYFGENADKFFIFPNYTIVSDFGRPNSALETHRLVAFLER |
| | Y690F, R693V, V694L | ADSVISWDIQDEKNVTCQLTFWEASERTIRSEAEDSYHFISAKM |
| | dI-dIV-dV-pack1: Y218F, | TAFFLSKKQEVNMSDSALDCVRDEAINKLQQIFNTSYNQTYEKY |
| | Q591F, S668A | GNVSVFETTGGLVVFWQGIKQKSLVELERLANRSSLNLTHNRTK |
| | Disulf1: D217C, S587C | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIFNFANCSYVE |
| | | YGQLGEDNEILLGNHRTEECQLPSLKIFIAGNSAYEYVDYLFKR |
| | | MIDLSSISTVDSMIALDIDPLENTDERVLELYAQKELRSSNVED |
| | | LEEIMRLFNSFKQVLKYV |
| SEQ ID | gB-Towne-ext-N- | GAAVSSSSTRGTSATHSHASSHITSAAHSRSGSVSQRVTSSQTV |
| NO: 139 | term_Cpack1-long_dII-dIII- | SHGVNETIYNTTLKYGDVVGVNTTKYPYRVCSMAQGTDLIRFER |
| | pack1_dI-dIV-dV- | NIVCTSMKPINEDLDEGIMVVYKRNIVAHTFKVRVYQKVLTERR |
| | pack1_Disulf1 | SYAYIHTTYLLGSNTEYVAPPMWEIHHINSHSQCYSSYSRVIAG |
| | dII-dIII-pack1: S367I, | TVEVAYHRDSYENKTMQLMPDCFSNTHSTRYVTVKDQWHSRGST |
| | T374F | WLYRETCNINCMVTITTARSKYPYHFFATSTGDVVDISPFYNGT |
| | Cpack1-long: E686L, Y690F, | NRNASYFGENADKFFIFPNYTIVSDFGRPNSALETHRLVAFLER |
| | R693V, V694L, K700A, V702Q | ADSVISWDIQDEKNVTCQLTFWEASERTIRSEAEDSYHFISAKM |
| | dI-dIV-dV-pack1: Y218F, | TAFFLSKKQEVNMSDSALDCVRDEAINKLQQIFNTSYNQTYEKY |
| | Q591F, S668A | GNVSVFETTGGLVVFWQGIKQKSLVELERLANRSSLNLTHNRTK |
| | Disulf1: D217C, S587C | RSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCVTINQTSVKVLRDMNVKESPGRCYSRPVVIFNFANCSYVF |
| | | YGQLGEDNEILLGNHRTEECQLPSLKIFIAGNSAYEYVDYLFKR |
| | | MIDLSSISTVDSMIALDIDPLENTDFRVLELYAQKELRSSNVFD |
| | | LEEIMPLENSFKQVLKYVEDAVQ |
| SEQ ID | gB-Towne-ext-N- | GAAVSSSSTRGTSATHSHHSSHTTSAAHSRSGSVSQRVTSSQTV |
| NO: 140 | term_Cpack1-short_dII- | SHGVNETIYNTTLKYGDVVGVNTTKYPYRVCSMAQGTDLIRFER |
| | dIII-pack1_dI-dIV-dV- | NIVCTSMKPINEDLDEGIMVVYKRNIVAHTFKVRVYQKVLTERR |
| | pack1_Disulf2 | SYAYIHTTYLLGSNTEYVAPPMWEIHHINSHSQCYSSYSRVIAG |
| | dII-dIII-pack1: S367I, | TVFVAYHRDSYENKTMQLMPDCFSNTHSTRYVTVKDQWHSRGST |
| | T374F | WLYRETCNLNCMVTITTARSKYPYHFFATSTGDVVDISPFYNGT |
| | Cpack1-short: E686L, | NRNASYFGENADKFFIFPNYTIVSDEGRPNSALETHRLVAFLER |
| | Y690F, R693V, V694L | ADSVISWDIQDEKNVTCQLTFWEASERTIRSEAEDSYHFISAKM |
| | dI-dIV-dV-pack1: Y218F, | TAFFLSKKQEVNMSDSALDCVRDEAINKLQQIFNTSYNQTYEKY |
| | Q591F, S668A | GNVSVFETTGGLVVFWQGIKQKSLVELERLANRSSLNLTHNRTK |
| | Disulf2: D217C, Y589C | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIENFANSSCVE |
| | | YGQLGEDNEILLGNHRTEECQLPSLKIFIAGNSAYEYVDYLFKR |
| | | MIDLSSISTVDSMIALDIDPLENTDERVLELYAQKELRSSNVFD |
| | | LEEIMRLFNSFKQVLKYV |
| SEQ ID | gB-Towne-ext-N- | GAAVSSSSTRGTSATHSHHSSHTTSAAHSRSGSVSQRVTSSQTV |
| NO: 141 | term_Cpack1-long_dII-dIII- | SHGVNETIYNTTLKYGDVVGVNTTKYPYRVCSMAQGTDLIRFER |
| | pack1_dI-dIV-dV- | NIVCTSMKPINEDLDEGIMVVYKRNIVAHTFKVRVYQKVLTERR |
| | pack1_Disulf2 | SYAYIHTTYLLGSNTEYVAPPMWEIHHINSHSQCYSSYSRVIAG |
| | dII-dIII-pack1: S367I, | TVFVAYHRDSYENKTMQLMPDCFSNTHSTRYVTVKDQWHSRGST |
| | T374F | WLYRETCNLNCMVTITTARSKYPYHFFATSTGDVVDISPFYNGT |
| | Cpack1-long: E686L, Y690F, | NRNASYFGENADKFFIFPNYTIVSDFGRPNSALETHRLVAFLER |
| | R693V, V694D, K700A, V702Q | ADSVISWDIQDEKNVICQLIFWEASERTIRSEAEDSYHFISAKM |
| | dI-dIV-dV-pack1: Y218F, | TAFFLSKKQEVNMSDSALDCVRDEAINKLQQIFNTSYNQTYEKY |
| | Q591F, S668A | GNVSVFETTGGLVVFWQGIKQKSLVELERLANRSSLNLTHNRTK |
| | Disulf2: D217C, Y589C | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCVTINQTSVKVLRDMNVKESPGRCYSRPVVIFNFANSSCVE |
| | | YGQLGEDNEILLGNHRTEECQLPSLKIFIAGNSAYEYVDYLFKR |
| | | MIDLSSISTVDSMIALDIDPLENTDERVLELYAQKELRSSNVED |
| | | LEEIMRLENSFKQVLKYVEDAVQ |
| SEQ ID | gB-Towne-ext-N- | GAAVSSSSTRGTSATHSHHSSHTTSAAHSRSGSVSQRVTSSQTV |
| NO: 142 | term_Cpack1-short_dII- | SHGVNETIYNTTLKYGDVVGVNTTKYPYRVCSMAQGTDLIRFER |
| | dIII-pack1_dI-dIV-dV- | NIVCTSMKPINEDLDEGIMVVYKRNIVAHTFKVRVYQKVLTERR |
| | pack1_N165glyc | SYAYINTTYLLGSNTTYVAPPMWEIHHINSHSQCYSSYSRVIAG |
| | dII-dIII-pack1: S367I, | TVFVAYHRDSYENKTMQLMPDDFSNTHSTRYVTVKDQWHSRGST |
| | T374F | WLYRETCNLNCMVTITTARSKYPYHFFATSTGDVVDISPFYNGT |
| | Cpack1-short: E686L, | NRNASYFGENADKFFIFPNYTIVSDFGRPNSALETHRLVAFLER |
| | Y690F, R693V, V694L | ADSVISWDIQDEKNVICQLTFWEASERTIRSEAEDSYHFISAKM |
| | dI-dIV-dV-pack1: Y218F, | TAFFLSKKQEVNMSDSALDCVRDEAINKLQQIFNTSYNQTYEKY |
| | Q591F, S668A | GNVSVFETTGGLVVFWQGIKQKSIVELERLANRSSLNLTHNRTK |
| | N165glyc: E167T | RSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCVTINQTSVKVLRDMNVKESPGRCYSRPVVIENFANSSYVF |
| | | YGQLGEDNEILLGNHRTEECQLPSLKIFIAGNSAYEYVDYLEKR |
| | | MIDLSSISTVDSMIALDIDPLENTDFRVLELYAQKELRSSNVFD |
| | | LEEIMRLFNSFKQVLKYV |
| SEQ ID | gB-Towne-ext-N- | GAAVSSSSTRGTSATHSHHSSHTTSAAHSRSGSVSQRVTSSQTV |
| NO: 143 | term_Cpack1-long_dII-dIII- | SHGVNETIYNTTLKYGDVVGVNTTKYPYRVCSMAQGIDLIRFER |
| | pack1_dI-dIV-dV- | NIVCTSMKPINEDLDEGIMVVYKRNIVAHTFKVRVYQKVLTERR |
| | pack1_N165glyc | SYAYIHTTYLLGSNTTYVAPPMWEIHHINSHSQCYSSYSRVIAG |
| | dII-dIII-pack1: S367I, | TVFVAYHRDSYENKTMQLMPDDESNTHSTRYVTVKDQWHSRGST |
| | T374F | WLYRETCNLNCMVTITTARSKYPYHFFATSTGDVVDISPFYNGT |
| | Cpack1-long: E686L, Y690F, | NRNASYFGENADKFFIFPNYTIVSDFGRPNSALETHRLVAFLER |
| | R693V, V694L, K700A, V702Q | ADSVISWDIQDEKNVICQLTFWEASERTIRSEAEDSYHFISAKM |
| | dI-dIV-dV-pack1: Y218F, | TAFFLSKKQEVNMSDSALDCVRDEAINKLQQIFNTSYNQTYEKY |
| | Q591F, S668A | GNVSVFETTGGLVVFWQGIKQKSLVELERLANRSSLNLTHNRTK |
| | N165glyc: E167T | RSTDGNNATHLSNMESVHNLVYAQLQFTYDTLRGYINRALAQIA |
| | | EAWCVDQRRTLEVFKELSKINPSAILSAIYNKPIAARFMGDVLG |
| | | LASCVTINQTSVKVLRDMNVKESPGRCYSRPVVIENFANSSYVE |
| | | YGQLGEDNEILLGNHRTEECQLPSLKIFIAGNSAYEYVDYLEKR |
| | | MIDLSSISTVDSMIALDIDPLENTDERVLELYAQKELRSSNVED |
| | | LEEIMRLENSFKQVLKYVEDAVQ |
| SEQ ID | gB-Towne-ext-N- | GAAVSSSSTRGTSATHSHHSSHTTSAAHSRSGSVSQRVTSSQTV |
| NO: 144 | term_Cpack1-short_dI-dIV- | SHGVNETIYNTTLKYGDVVGVNTTKYPYRVCSMAQGTDLIRFER |
| | dV-pack2 | NIVCTSMKPINEDLDEGIMVVYKRNIVAHTFKVRVYQKVLTERR |
| | Cpack1-short: E686L, | SYAYIHTTYLLGSNTEYVAPPMWEIHHINSHSQCYSSYSRVIAG |
| | Y690F, R693V, V694L | TVFVAYHRDSYENKTMQLMPDDYSSTHSTRYVTVKDQWHSRGST |
| | dI-dIV-dV-pack2: N220S, | WLYRETCNLNCMVTITTARSKYPYHFFATSTGDVVDISPFYNGT |
| | V552L | NRNASYFGENADKFFIFPNYTIVSDFGRPNSALETHRLVAFLER |
| | | ADSVISWDIQDEKNVICQLTFWEASERTIRSEAEDSYHESSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCLTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVED |
| | | LEEIMRLFNSFKQVLKYV |
| SEQ ID | gB-Towne-ext-N- | GAAVSSSSTRGTSATHSHHSSHTTSAAHSRSGSVSQRVTSSQTV |
| NO: 145 | term_Cpack1-long_dI-dIV- | SHGVNETIYNTTLKYGDVVGVNTTKYPYRVCSMAQGTDLIRFER |
| | dV-pack2 | NIVCTSMKPINEDLDEGIMVVYKRNIVAHTFKVRVYQKVLTERR |
| | Cpack1-long: E686L, Y690F, | SYAYIHTTYLLGSNTEYVAPPMWEIHHINSHSQCYSSYSRVIAG |
| | R693V, V694L, K700A, V702Q | TVEVAYHRDSYENKIMQLMPDDYSSTHSTRYVTVKDQWHSRGST |
| | dI-dIV-dV-pack2: N220S, | WLYRETCNLNCMVTITTARSKYPYHFFATSTGDVVDISPFYNGT |
| | V552L | NRNASYFGENADKFFIFPNYTIVSDFGRPNSALETHRLVAFLER |
| | | ADSVISWDIQDEKNVICQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSIVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCLTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDFRVLELYSQKELRSSNVED |
| | | LEEIMPLENSFKQVLKYVEDAVQ |
| SEQ ID | gB-Towne-ext-N- | GAAVSSSSTRGTSATHSHHSSHITSAAHSRSGSVSQRVTSSQTV |
| NO: 146 | term_Cpackl-short_dI-dIV- | SHGVNETIYNTTLKYGDVVGVNTTKYPYRVCSMAQGTDLIRFER |
| | dV-pack3 | NIVCTSMKPINEDLDEGIMVVYKRNIVAHTFKVRVYQKVLTERR |
| | Cpack1-short: E686L, | SYAYIHTTYLLGSNTEYVAPPMWEIHHINSHSQCYSSYSRVIAG |
| | Y690F, R693V, V694L | TVFVAYHRDSYENKTMQLMPDDESSTHSTRYVTVKDQWHSRGST |
| | dI-dIV-dV-pack3: Y218F, | WLYRETCNLNCMVTITTARSKYPYHFFATSTGDVVDISPFYNGT |
| | N220S, V552L, Q591F, S668A | NRNASYFGENADKFFIFPNYTIVSDFGRPNSALETHRLVAFLER |
| | | ADSVISWDIQDEKNVTCQLTFWEASERTIRSEAEDSYHESSAKM |
| | | TATFLSKKQEVNMSDSALDCVRDEAINKLQQIFNTSYNQTYEKY |
| | | GNVSVFETTGGLVVFWQGIKQKSIVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCLTINQTSVKVLRDMNVKESPGRCYSRPVVIENFANSSYVE |
| | | YGQLGEDNEILLGNHRTEECQLPSLKIFIAGNSAYEYVDYLEKR |
| | | MIDLSSISTVDSMIALDIDPLENTDERVLELYAQKELRSSNVFD |
| | | LEEIMRLFNSFKQVLKYV |
| SEQ ID | gB-Towne-ext-N- | GAAVSSSSTRGTSATHSHHSSHTTSAAHSRSGSVSQRVTSSQTV |
| NO: 147 | term_Cpack1-long_dI-dIV- | SHGVNETIYNTTLKYGDVVGVNTTKYPYRVCSMAQGTDLIRFER |
| | dV-pack3 | NIVCTSMKPINEDLDEGIMVVYKRNIVAHTEKVRVYQKVLTERR |
| | Cpack1-long: E686L, Y690F, | SYAYIHTTYLLGSNTEYVAPPMWEIHHINSHSQCYSSYSRVIAG |
| | R693V, V694L, K700A, V702Q | TVEVAYHRDSYENKTMQLMPDDESSTHSTRYVTVKDQWHSRGST |
| | dI-dIV-dV-pack3: Y218F, | WLYRETCNLNCMVTITTARSKYPYHFFATSTGDVVDISPFYNGT |
| | N220S, V552L, Q591F, S668A | NRNASYFGENADKFFIFPNYTIVSDFGRPNSALETHRLVAFLER |
| | | ADSVISWDIQDEKNVTCQLTFWEASERTIRSEAEDSYHESSAKM |
| | | TATFLSKKQEVNMSDSALDCVRDEAINKLQQIFNTSYNQTYEKY |
| | | GNVSVFETTGGLVVFWQGIKQKSIVELERLANRSSENLTHNRTK |
| | | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCLTINQTSVKVLRDMNVKESPGRCYSRPVVIENFANSSYVE |
| | | YGQLGEDNEILLGNHRTEECQLPSLKIFIAGNSAYEYVDYLFKR |
| | | MIDLSSISTVDSMIALDIDPLENTDERVLELYAQKELRSSNVED |
| | | LEEIMRLENSFKQVLKYVEDAVQ |
| SEQ ID | gB-Towne-ext-N- | GAAVSSSSTRGTSATHSHHSSHTTSAAHSRSGSVSQRVTSSQTV |
| NO: 148 | term_Cpack1-short_dII- | SHGVNETIYNTTLKYGDVVGVNITKYPYRVCSMAQGIDLIRFER |
| | dIII-pack1_dI-dIV-dV-pack2 | NIVCTSMKPINEDLDEGIMVVYKRNIVAHTFKVRVYQKVLTERR |
| | dII-dIII-pack1: S367I, | SYAYIHTTYLLGSNTEYVAPPMWEIHHINSHSQCYSSYSRVIAG |
| | T374F | TVEVAYHRDSYENKTMQLMPDDYSSTRSTRYVTVKDQWHSRGST |
| | Cpack1-short: E686L, | WLYRETCNINCMVTITTARSKYPYHFFATSTGDVVDISPFYNGT |
| | Y690F, R693V, V694L | NRNASYFGENADKFFIFPNYTIVSDFGRPNSALETHRLVAFLER |
| | dI-dIV-dV-pack2: N220S, | ADSVISWDIQDEKNVICQLTFWEASERTIRSEAEDSYHFISAKM |
| | V552L | TAFFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCLTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDFRVLELYSQKELRSSNVFD |
| | | LEEIMPLENSFKQVLKYV |
| SEQ ID | gB-Towne-ext-N- | GAAVSSSSTRGTSATHSHHSSHTTSAAHSRSGSVSQRVTSSQTV |
| NO: 149 | term_Cpack1-long_dII-dIII- | SHGVNETIYNTTLKYGDVVGVNITKYPYRVCSMAQGTDLIRFER |
| | pack1_dI-dIV-dV-pack2 | NIVCTSMKPINEDLDEGIMVVYKRNIVAHTFKVRVYQKVLTERR |
| | dII-dIII-pack1: S367I, | SYAYIHTTYLLGSNTEYVAPPMWEIHHINSHSQCYSSYSRVIAG |
| | T374F | TVFVAYHRDSYENKTMQLMPDDYSSTHSTRYVTVKDQWHSRGST |
| | Cpack1-long: E686L, Y690F, | WLYRETCNLNCMVTITTARSKYPYHFFATSTGDVVDISPFYNGT |
| | R693V, V694L, K700A, V702Q | NRNASYFGENADKFFIFPNYTIVSDFGRPNSALETHRLVAFLER |
| | dI-dIV-dV-pack2: N220S, | ADSVISWDIQDEKNVICQLTFWEASERTIRSEAEDSYHFISAKM |
| | V552L | TAFFLSKKQEVNMSDSALDCVRDEAINKLQQIFNTSYNQTYEKY |
| | | GNVSVFETTGGLVVFWQGIKQKSLVELERLANRSSLNLTANRTK |
| | | RSTDGNNATHLSNMESVHNLVYAQLQFTYDTERGYINRALAQIA |
| | | EAWCVDQRRTLEVEKELSKINPSAILSAIYNKPIAAREMGDVLG |
| | | LASCLTINQTSVKVLRDMNVKESPGRCYSRPVVIENFANSSYVQ |
| | | YGQLGEDNEILLGNHRTEECQLPSLKIFIAGNSAYEYVDYLEKR |
| | | MIDLSSISTVDSMIALDIDPLENTDERVLELYSQKELRSSNVED |
| | | LEEIMRLENSFKQVLKYVEDAVQ |
| SEQ ID | gB-Towne-ext-N- | GAAVSSSSTRGTSATHSHHSSHTTSAAHSRSGSVSQRVTSSQTV |
| NO : 150 | term_Cpack1-short_dII- | SHGVNETIYNTTLKYGDVVGVNTTKYPYRVCSMAQGTDLIRFER |
| | dIII-pack1_dI-dIV-dV-pack3 | NIVCTSMKPINEDLDEGIMVVYKRNIVAHTFKVRVYQKVLTERR |
| | dII-dIII-pack1: S367I, | SYAYIHTTYLLGSNTEYVAPPMWEIHHINSHSQCYSSYSRVIAG |
| | T374F | TVFVAYHRDSYENKTMQLMPDDFSSTHSTRYVTVKDQWHSRGST |
| | Cpack1-short: E686L, | WLYRETCNLNCMVTITTARSKYPYHFFATSTGDVVDISPFYNGT |
| | Y690F, R693V, V694L | NRNASYFGENADKFFIFPNYTIVSDFGRPNSALETHRLVAFLER |
| | dI-dIV-dV-pack3: Y218F, | ADSVISWDIQDEKNVTCQLTFWEASERTIRSEAEDSYHFISAKM |
| | N220S, V552L, Q591F, S668A | TAFFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCLTINQTSVKVLRDMNVKESPGRCYSRPVVIENFANSSYVE |
| | | YGQLGEDNEILLGNHRTEECQLPSLKIFIAGNSAYEYVDYLFKR |
| | | MIDLSSISTVDSMIALDIDPLENTDERVLELYAQKELRSSNVFD |
| | | LEEIMRLFNSFKQVLKYV |
| SEQ ID | gB-Towne-ext-N- | GAAVSSSSTRGTSATHSHHSSHTTSAAHSRSGSVSQRVTSSQTV |
| NO: 151 | term_Cpack1-long_dII-dIII- | SHGVNETIYNTTLKYGDVVGVNTTKYPYRVCSMAQGTDLIRFER |
| | pack1_dI-dIV-dV-pack3 | NIVCTSMKPINEDLDEGIMVVYKRNIVAHTFKVRVYQKVLTFRR |
| | dII-dIII-pack1: S367I, | SYAYINTTYLLGSNTEYVAPPMWEIHHINSHSQCYSSYSRVIAG |
| | T374F | TVEVAYHRDSYENKTMQLMPDDESSTHSTRYVTVKDQWHSRGST |
| | Cpack1-long: E686L, Y690F, | WLYRETCNLNCMVTITTARSKYPYHFFATSTGDVVDISPFYNGT |
| | R693V, V694L, K700A, V702Q | NRNASYFGENADKFFIFPNYTIVSDFGRPNSALETHRLVAFLER |
| | dI-dIV-dV-pack3: Y218F, | ADSVISWDIQDEKNVTCQLIFWEASERTIRSEAEDSYHFISAKM |
| | N220S, V552L, Q591F, S668A | TAFFLSKKQEVNMSDSALDCVRDEAINKLQQIFNTSYNQTYEKY |
| | | GNVSVFETTGGLVVFWQGIKQKSIVELERLANRSSLNLTHNRIK |
| | | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCLTINQTSVKVLRDMNVKESPGRCYSRPVVIENFANSSYVE |
| | | YGQLGEDNEILLGNHRTEECQLPSLKIFIAGNSAYEYVDYLFKR |
| | | MIDLSSISTVDSMIALDIDPLENTDERVLELYAQKELRSSNVFD |
| | | LEEIMRLENSFKQVLKYVEDAVQ |
| SEQ ID | gB-Towne-ext-N- | GAAVSSSSTRGTSATHSHHSSHTTSAAHSRSGSVSQRVTSSQTV |
| NO: 152 | term_Cpack1-short_dI-dIV- | SHGVNETIYNTTLKYGDVVGVNTTKYPYRVCSMAQGTDLIRFER |
| | dV-pack3_Disulf1 | NIVCTSMKPINEDLDEGIMVVYKRNIVAHTFKVRVYQKVLTERR |
| | Cpack1-short: E686L, | SYAYIHTTYLLGSNTEYVAPPMWEIHHINSHSQCYSSYSRVIAG |
| | Y690F, R693V, V694L | TVFVAYHRDSYENKTMQLMPDCFSSTHSTRYVTVKDQWHSRGST |
| | dI-dIV-dV-pack3: Y218F, | WLYRETCNLNCMVTITTARSKYPYHFFATSTGDVVDISPFYNGT |
| | N220S, V552L, Q591F, S668A | NRNASYFGENADKFFIFPNYTIVSDFGRPNSALETHRLVAFLER |
| | Disulf1: D217C, S587C | ADSVISWDIQDEKNVTCQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCLTINQTSVKVLRDMNVKESPGRCYSRPVVIENFANCSYVE |
| | | YGQLGEDNEILLGNHRTEECQLPSLKIFIAGNSAYEYVDYLEKR |
| | | MIDLSSISTVDSMIALDIDPLENTDERVLELYAQKELRSSNVFD |
| | | LEEIMRLFNSFKQVLKYV |
| SEQ ID | GB-Towne-ext-N- | GAAVSSSSTRGTSATHSHHSSHTTSAAHSRSGSVSQRVTSSQTV |
| NO: 153 | term_Cpack1-long_dI-dIV- | SHGVNETIYNTTLKYGDVVGVNTTKYPYRVCSMAQGTDLIRFER |
| | dV-pack3_Disulf1 | NIVCTSMKPINEDLDEGIMVVYKRNIVAHTFKVRVYQKVLTERR |
| | Cpack1-long: E686L, Y690F, | SYAYIHTTYLLGSNTEYVAPPMWEIHHINSHSQCYSSYSRVIAG |
| | R693V, V694L, K700A, V702Q | TVEVAYHRDSYENKTMQLMPDCFSSTHSTRYVTVKDQWHSRGST |
| | dI-dIV-dV-pack3: Y218F, | WLYRETCNLNCMVTITTARSKYPYHFFATSTGDVVDISPFYNGT |
| | N220S, V552L, Q591F, S668A | NRNASYFGENADKFFIFPNYTIVSDFGRPNSALETHRLVAFLER |
| | Disulf1: D217C, S587C | ADSVISWDIQDEKNVTCQLIFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVFKELSKINPSAILSAIYNKPIAARFMGDVLG |
| | | LASCLTINQTSVKVLRDMNVKESPGRCYSRPVVIENFANCSYVE |
| | | YGQLGEDNEILLGNHRTEECQLPSLKIFIAGNSAYEYVDYLEKR |
| | | MIDLSSISTVDSMIALDIDPLENTDERVLELYAQKELRSSNVED |
| | | LEEIMRLFNSFKQVLKYVEDAVQ |
| SEQ ID | gB-Towne-ext-N- | GAAVSSSSTRGTSATHSHHSSHTTSAAHSRSGSVSQRVTSSQTV |
| NO: 154 | term_Cpack1-short_dII- | SHGVNETIYNTTLKYGDVVGVNTTKYPYRVCSMAQGTDLIRFER |
| | dIII-pack1_dI-dIV-dV- | NIVCTSMKPINEDLDEGIMVVYKRNIVAHTFKVRVYQKVLTERR |
| | pack3_Disulf1 | SYAYIHTTYLLGSNTEYVAPPMWEIHHINSHSQCYSSYSRVIAG |
| | Cpack1-short: E686L, | TVFVAYHRDSYENKTMQLMPDCFSSTHSTRYVTVKDQWHSRGST |
| | Y690F, R693V, V694L | WLYRETCNLNCMVTITTARSKYPYHFFATSIGDVVDISPFYNGT |
| | dII-dIII-pack1: S367I, | NRNASYFGENADKFFIFPNYTIVSDFGRPNSALETHRLVAFLER |
| | T374F | ADSVISWDIQDEKNVICQLTFWEASERTIRSEAEDSYHFISAKM |
| | dI-dIV-dV-pack3: Y218F, | TAFFLSKKQEVNMSDSALDCVRDEAINKLQQIFNTSYNQTYEKY |
| | N220S, V552L, Q591F, S668A | GNVSVFETTGGLVVFWQGIKQKSIVELERLANRSSLNLTHNRTK |
| | Disulf1: D217C, S587C | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCLTINQTSVKVLRDMNVKESPGRCYSRPVVIENFANCSYVF |
| | | YGQLGEDNEILLGNHRTEECQLPSLKIFIAGNSAYEYVDYLEKR |
| | | MIDLSSISTVDSMIALDIDPLENTDERVLELYAQKELRSSNVFD |
| | | LEEIMRLENSFKQVLKYV |
| SEQ ID | gB-Towne-ext-N- | GAAVSSSSTRGTSATHSHHSSHETSAAHSRSGSVSQRVTSSQTV |
| NO: 155 | term_Cpack1-long_dII-dIII- | SHGVNETIYNTTLKYGDVVGVNITKYPYRVCSMAQGTDLIRFER |
| | pack1_dI-dIV-dV- | NIVCTSMKPINEDLDEGIMVVYKRNIVAHTFKVRVYQKVLTERR |
| | pack3_Disulf1 | SYAYIHTTYLLGSNTEYVAPPMWEIHHINSHSQCYSSYSRVIAG |
| | Cpack1-long: E686L, Y690F, | TVFVAYHRDSYENKTMQLMPDCFSSTHSTRYVTVKDQWHSRGST |
| | R693V, V694L, K700A, V702Q | WLYRETCNLNCMVTITTARSKYPYHFFATSTGDVVDISPFYNGT |
| | dII-dIII-pack1: S367I, | NRNASYFGENADKFFIFPNYTIVSDFGRPNSALETHRLVAFLER |
| | T374F | ADSVISWDIQDEKNVTCQLTFWEASERTIRSEAEDSYHFISAKM |
| | dI-dIV-dV-pack3: Y218F, | TAFFLSKKQEVNMSDSALDCVRDEAINKLQQIFNTSYNQTYEKY |
| | N220S, V552L, Q591F, S668A | GNVSVFETTGGLVVFWQGIKQKSIVELERLANRSSLNLTHNRTK |
| | Disulf1: D217C, S587C | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCLTINQTSVKVLRDMNVKESPGRCYSRPVVIENFANCSYVE |
| | | YGQLGEDNEILLGNHRTEECQLPSLKIFIAGNSAYEYVDYLEKR |
| | | MIDLSSISTVDSMIALDIDPLENTDERVLELYAQKELRSSNVFD |
| | | LEEIMRLFNSFKQVLKYVEDAVQ |
| SEQ ID | gB-Towne-ext-N- | GAAVSSSSTRGTSATHSHHSSHTTSAAHSRSGSVSQRVTSSQTV |
| NO: 156 | term_Cpack1-short_dII- | SHGVNETIYNTTLKYGDVVGVNTTKYPYRVCSMAQGTDLIRFER |
| | dIII-pack1_dI-dIV-dV- | NIVCTSMKPINEDLDEGIMVVYKRNIVAHTFKVRVYQKVLTERR |
| | pack4_Disulf1 | SYAYIHTTYLLGSNTEYVAPPMWEIHHINSHSQCYSSYSRVIAG |
| | Cpack1-short: E686L, | TVFVAYHRDSYENKTMQLMPDCFSSTHSTRYVTVKDQWHSRGST |
| | Y690F, R693V, V694L | WLYRETCNLNCMVTITTARSKYPYHFFATSTGDVVDISPFYNGT |
| | dII-dIII-pack1: S367I, | NRNASYFGENADKFFIFPNYTIVSDFGRPNSALETHRLVAFLER |
| | T374F | ADSVISWDIQDEKNVTCQLTFWEASERTIRSEAEDSYHFISAKM |
| | dI-dIV-dV-pack4: Y218F, | TAFFLSKKQEVNMSDSALDCVRDEAINKLQQIFNTSYNQTYEKY |
| | N220S, V552L, Q591Y, S668A | GNVSVFETTGGLVVFWQGIKQKSLVELERLANRSSINLTHNRTK |
| | Disulf1: D217C, S587C | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCLTINQTSVKVLRDMNVKESPGRCYSRPVVIENFANCSYVY |
| | | YGQLGEDNEILLGNHRTEECQLPSLKIFIAGNSAYEYVDYLFKR |
| | | MIDLSSISTVDSMIALDIDPLENTDFRVLELYAQKELRSSNVED |
| | | LEEIMRLENSFKQVLKYV |
| SEQ ID | gB-Towne-ext-N- | GAAVSSSSTRGTSATHSHHSSHTTSAAHSRSGSVSQRVTSSQTV |
| NO: 157 | term_Cpack1-long_dII-dIII- | SHGVNETIYNTTLKYGDVVGVNITKYPYRVCSMAQGTDLIRFER |
| | pack1_dI-dIV-dV- | NIVCTSMKPINEDLDEGIMVVYKRNIVAHTEKVRVYQKVLTERR |
| | pack4_Disulf1 | SYAYIHTTYLLGSNTEYVAPPMWEIHHINSHSQCYSSYSRVIAG |
| | Cpack1-long: E686L, Y690F, | TVEVAYHRDSYENKTMQLMPDCFSSTHSTRYVTVKDQWHSRGST |
| | R693V, V694L, K700A, V702Q | WLYRETCNLNCMVTITTARSKYPYHFFATSTGDVVDISPFYNGT |
| | dII-dIII-pack1: S367I, | NRNASYFGENADKFFIFPNYTIVSDFGRPNSALETHRLVAFLER |
| | T374F | ADSVISWDIQDEKNVTCQLTFWEASERTIRSEAEDSYHFISAKM |
| | dI-dIV-dV-pack4: Y218F, | TAFFLSKKQEVNMSDSALDCVRDEAINKLQQIFNTSYNQTYEKY |
| | N220S, V552L, Q591Y, S668A | GNVSVFETTGGLVVFWQGIKQKSLVELERLANRSSLNLTHNRTK |
| | Disulf1: D217C, S587C | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCLTINQTSVKVLRDMNVKESPGRCYSRPVVIENFANCSYVY |
| | | YGQLGEDNEILLGNHRTEECQLPSLKIFIAGNSAYEYVDYLFKR |
| | | MIDLSSISTVDSMIALDIDPLENTDFRVLELYAQKELRSSNVED |
| | | LEEIMRLFNSFKQVLKYVEDAVQ |
| SEQ ID | gB-Towne-ext-N- | GAAVSSSSTRGTSATHSHASSHTTSAAHSRSGSVSQRVTSSQTV |
| NO: 158 | term_Cpack1-short_dII- | SHGVNETIYNTTLKYGDVVGVNTTKYPYRVCSMAQGTDLIRFER |
| | dIII-pack1_dI-dIV-dV- | NIVCTSMKPINEDLDEGIMVVYKRNIVAHTFKVRVYQKVLTERR |
| | pack5_Disulf1 | SYAYINTTYLLGSNTEYVAPPMWEIHHINSHSQCYSSYSRVIAG |
| | Cpack1-short: E686L, | TVEVAYHRDSYENKTMQLMPDCYSSTHSTRYVTVKDQWHSRGST |
| | Y690F, R693V, V694L | WLYRETCNLNCMVTITTARSKYPYHFFATSTGDVVDISPFYNGT |
| | dII-dIII-pack1: S367I, | NRNASYFGENADKFFIFPNYTIVSDFGRPNSALETHRLVAFLER |
| | T374F | ADSVISWDIQDEKNVICQLTFWEASERTIRSEAEDSYHFISAKM |
| | dI-dIV-dV-pack5: N220S, | TAFFLSKKQEVNMSDSALDCVRDEAINKLQQIFNTSYNQTYEKY |
| | V552L, Q591Y | GNVSVFETTGGLVVFWQGIKQKSLVELERLANRSSLNLTHNRTK |
| | Disulf1: D217C, S587C | RSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCLTINQTSVKVLRDMNVKESPGRCYSRPVVIENFANCSYVY |
| | | YGQLGEDNEILLGNHRTEECQLPSLKIFIAGNSAYEYVDYLFKR |
| | | MIDLSSISTVDSMIALDIDPLENTDFRVLELYSQKELRSSNVFD |
| | | LEEIMRLENSFKQVLKYV |
| SEQ ID | gB-Towne-ext-N- | GAAVSSSSTRGTSATHSHHSSHTTSAAHSRSGSVSQRVTSSQTV |
| NO: 159 | term_Cpack1-long_dII-dIII- | SHGVNETIYNTTLKYGDVVGVNTTKYPYRVCSMAQGTDLIRFER |
| | pack1_dI-dIV-dV- | NIVCTSMKPINEDLDEGIMVVYKRNIVAHTEKVRVYQKVLTFRR |
| | pack5_Disulf1 | SYAYIHTTYLLGSNTEYVAPPMWEIHHINSHSQCYSSYSRVIAG |
| | Cpack1-long: E686L, Y690F, | TVFVAYHRDSYENKTMQLMPDCYSSTHSTRYVTVKDQWHSRGST |
| | R693V, V694D, K700A, V7020 | WLYRETCNLNCMVTITTARSKYPYHFFATSTGDVVDISPFYNGT |
| | dII-dIII-pack1: S367I, | NRNASYFGENADKFFIFPNYTIVSDFGRPNSALETHRLVAFLER |
| | T374F | ADSVISWDIQDEKNVTCQLTFWEASERTIRSEAEDSYHFISAKM |
| | dI-dIV-dV-pack5: N220S, | TAFFLSKKQEVNMSDSALDCVRDEAINKLQQIFNTSYNQTYEKY |
| | V552L, Q591Y | GNVSVFETTGGLVVFWQGIKQKSIVELERLANRSSLNLTHNRTK |
| | Disulf1: D217C, S587C | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCLTINQTSVKVLRDMNVKESPGRCYSRPVVIENFANCSYVY |
| | | YGQLGEDNEILLGNHRTEECQLPSLKIFIAGNSAYEYVDYLEKR |
| | | MIDLSSISTVDSMIALDIDPLENTDERVLELYSQKELRSSNVED |
| | | LEEIMRLFNSFKQVLKYVEDAVQ |
| SEQ ID | gB-Towne-ext-N- | GAAVSSSSTRGTSATHSHHSSHTTSAAHSRSGSVSQRVTSSQTV |
| NO: 160 | term_Cpack1-short_dII- | SHGVNETIYNTTLKYGDVVGVNTTKYPYRVCSMAQGTDLIRFER |
| | dIII-pack1_dI-dIV-dV- | NIVCTSMKPINEDLDEGIMVVYKRNIVAHTFKVRVYQKVLTERR |
| | pack6_Disulf1 | SYAYIHTTYLLGSNTEYVAPPMWEIHHINSHSQCYSSYSRVIAG |
| | Cpack1-short: E686L, | TVEVAYHRDSYENKTMQLMPDCYSSTHSTRYVTVKDQWHSRGST |
| | Y690F, R693V, V694L | WLYRETCNLNCMVTITTARSKYPYHFFATSTGDVVDISPFYNGT |
| | dII-dIII-pack1: S367I, | NRNASYFGENADKFFIFPNYTIVSDFGRPNSALETHRLVAFLER |
| | T374F | ADSVISWDIQDEKNVTCQLIFWEASERTIRSEAEDSYHFISAKM |
| | dI-dIV-dV-pack6: N220S, | TAFFLSKKQEVNMSDSALDCVRDEAINKLQQIFNTSYNQTYEKY |
| | V552L, Q591F | GNVSVFETTGGLVVFWQGIKQKSIVELERLANRSSLNLTHNRTK |
| | Disulf1: D217C, S587C | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCLTINQTSVKVLRDMNVKESPGRCYSRPVVIENFANCSYVE |
| | | YGQLGEDNEILLGNHRTEECQLPSLKIFIAGNSAYEYVDYLFKR |
| | | MIDLSSISTVDSMIALDIDPLENTDERVLELYSQKELRSSNVED |
| | | LEEIMRLFNSFKQVLKYV |
| SEQ ID | gB-Towne-ext-N- | GAAVSSSSTRGTSATHSHHSSHTTSAAHSRSGSVSQRVTSSQTV |
| NO: 161 | term_Cpack1-long_dII-dIII- | SHGVNETIYNTTLKYGDVVGVNTTKYPYRVCSMAQGTDLIRFER |
| | pack1_dI-dIV-dV- | NIVCTSMKPINEDLDEGIMVVYKRNIVAHTFKVRVYQKVLTERR |
| | pack6_Disulf1 | SYAYIHTTYLLGSNTEYVAPPMWEIHHINSHSQCYSSYSRVIAG |
| | Cpack1-long: E686L, Y690F, | TVFVAYHRDSYENKTMQLMPDCYSSTHSTRYVTVKDQWHSRGST |
| | R693V, V694L, K700A, V702Q | WLYRETCNINCMVTITTARSKYPYHFFATSTGDVVDISPFYNGT |
| | dII-dIII-pack1: S367I, | NRNASYFGENADKFFIFPNYTIVSDFGRPNSALETHRLVAFLER |
| | T374F | ADSVISWDIQDEKNVICQLTFWEASERTIRSEAEDSYHFISAKM |
| | dI-dIV-dV-pack6: N220S, | TAFFLSKKQEVNMSDSALDCVRDEAINKLQQIFNTSYNQTYEKY |
| | V5521, Q591F | GNVSVFETTGGLVVFWQGIKQKSIVELERLANRSSLNLTHNRTK |
| | Disulf1: D217C, S587C | RSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCLTINQTSVKVLRDMNVKESPGRCYSRPVVIENFANCSYVE |
| | | YGQLGEDNEILLGNHRTEECQLPSLKIFIAGNSAYEYVDYLEKR |
| | | MIDLSSISTVDSMIALDIDPLENTDFRVLELYSQKELRSSNVFD |
| | | LEEIMRLFNSFKQVLKYVEDAVQ |
| SEQ ID | GB-Towne-ext-N- | GAAVSSSSTRGTSATHSHHSSHTTSAAHSRSGSVSQRVTSSQTV |
| NO: 162 | term_Cpack1-short_dI-dIV- | SHGVNETIYNTTLKYGDVVGVNTTKYPYRVCSMAQGIDLIRFER |
| | dV-pack4_Disulf1 | NIVCTSMKPINEDLDEGIMVVYKRNIVAHTFKVRVYQKVLTERR |
| | Cpack1-short: E686L, | SYAYIHTTYLLGSNTEYVAPPMWEIHHINSHSQCYSSYSRVIAG |
| | Y690F, R693V, V694L | TVEVAYHRDSYENKTMQLMPDCFSSTHSTRYVTVKDQWHSRGST |
| | dI-dIV-dV-pack4: Y218F, | WLYRETCNLNCMVTITTARSKYPYHFFATSTGDVVDISPFYNGT |
| | N220S, V552L, Q591Y, S668A | NRNASYFGENADKFFIFPNYTIVSDFGRPNSALETHRLVAFLER |
| | Disulf1: D217C, S587C | ADSVISWDIQDEKNVICQLIFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCLTINQTSVKVLRDMNVKESPGRCYSRPVVIENFANCSYVY |
| | | YGQLGEDNEILLGNHRTEECQLPSLKIFIAGNSAYEYVDYLEKR |
| | | MIDLSSISTVDSMIALDIDPLENTDERVLELYAQKELRSSNVFD |
| | | LEEIMRLENSFKQVLKYV |
| SEQ ID | gB-Towne-ext-N- | GAAVSSSSTRGTSATHSHHSSHTTSAAHSRSGSVSQRVTSSQTV |
| NO: 163 | term_Cpack1-long_dI-dIV- | SHGVNETIYNTTLKYGDVVGVNTTKYPYRVCSMAQGTDLIRFER |
| | dV-pack4_Disulf1 | NIVCTSMKPINEDLDEGIMVVYKRNIVAHTFKVRVYQKVLTFRR |
| | Cpack1-long: E686L, Y690F, | SYAYIHTTYLLGSNTEYVAPPMWEIHHINSHSQCYSSYSRVIAG |
| | R693V, V694L, K700A, V702Q | TVFVAYHRDSYENKTMQLMPDCFSSTHSTRYVTVKDQWHSRGST |
| | dI-dIV-dV-pack4: Y218F, | WLYRETCNLNCMVTITTARSKYPYHFFATSTGDVVDISPFYNGT |
| | N220S, V552L, Q591Y, S668A | NRNASYFGENADKFFIFPNYTIVSDFGRPNSALETHRLVAFLER |
| | Disulf1: D217C, S587C | ADSVISWDIQDEKNVTCQLTFWEASERTIRSEAEDSYHESSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCLTINQTSVKVLRDMNVKESPGRCYSRPVVIENFANCSYVY |
| | | YGQLGEDNEILLGNHRTEECQLPSLKIFIAGNSAYEYVDYLEKR |
| | | MIDLSSISTVDSMIALDIDPLENTDERVLELYAQKELRSSNVED |
| | | LEEIMRLFNSFKQVLKYVEDAVQ |
| SEQ ID | gB-Towne-ext-N- | GAAVSSSSTRGTSATHSHHSSHTTSAAHSRSGSVSQRVTSSQTV |
| NO: 164 | term_Cpack1-short_dI-dIV- | SHGVNETIYNTTLKYGDVVGVNTTKYPYRVCSMAQGTDLIRFER |
| | dV-pack5_Disulf1 | NIVCTSMKPINEDLDEGIMVVYKRNIVAHTFKVRVYQKVLTERR |
| | Cpack1-short: E686L, | SYAYIHTTYLLGSNTEYVAPPMWEIHHINSHSQCYSSYSRVIAG |
| | Y690F, R693V, V694L | TVEVAYHRDSYENKTMQLMPDCYSSTHSTRYVTVKDQWHSRGST |
| | dI-dIV-dV-pack5: N220S, | WLYRETCNLNCMVTITTARSKYPYHFFATSIGDVVDISPFYNGT |
| | V552L, Q591Y | NRNASYFGENADKFFIFPNYTIVSDFGRPNSALETHRLVAFLER |
| | Disulf1: D217C, S587C | ADSVISWDIQDEKNVICQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAAREMGDVLG |
| | | LASCLTINQTSVKVLRDMNVKESPGRCYSRPVVIENFANCSYVY |
| | | YGQLGEDNEILLGNHRTEECQLPSLKIFIAGNSAYEYVDYLEKR |
| | | MIDLSSISTVDSMIALDIDPLENTDERVLELYSQKELRSSNVED |
| | | LEEIMRLENSFKQVLKYV |
| SEQ ID | gB-Towne-ext-N- | GAAVSSSSTRGTSATHSHHSSHTTSAAHSRSGSVSQRVTSSQTV |
| NO: 165 | term_Cpack1-long_dI-dIV- | SHGVNETIYNTTLKYGDVVGVNTTKYPYRVCSMAQGTDLIRFER |
| | dV-pack5_Disulf1 | NIVCTSMKPINEDLDEGIMVVYKRNIVAHTFKVRVYQKVLTERR |
| | Cpack1-long: E686L, Y690F, | SYAYIHTTYLLGSNTEYVAPPMWEIHHINSHSQCYSSYSRVIAG |
| | R693V, V694L, K700A, V702Q | TVFVAYHRDSYENKTMQLMPDCYSSTHSTRYVTVKDQWHSRGST |
| | dI-dIV-dV-pack5: N220S, | WLYRETCNLNCMVTITTARSKYPYHFFATSTGDVVDISPFYNGT |
| | V552L, Q591Y | NRNASYFGENADKFFIFPNYTIVSDFGRPNSALETHRLVAFLER |
| | Disulf1: D217C, S587C | ADSVISWDIQDEKNVICQLTFWEASERTIRSEAEDSYHESSAKM |
| | | TATFLSKKQEVNMSDSALDCVRDEAINKLQQIFNTSYNQTYEKY |
| | | GNVSVFETTGGLVVFWQGIKQKSIVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCLTINQTSVKVLRDMNVKESPGRCYSRPVVIENFANCSYVY |
| | | YGQLGEDNEILLGNHRTEECQLPSLKIFIAGNSAYEYVDYLEKR |
| | | MIDLSSISTVDSMIALDIDPLENTDFRVLELYSQKELRSSNVFD |
| | | LEEIMRLFNSFKQVLKYVEDAVQ |
| SEQ ID | GB-Towne-ext-N- | GAAVSSSSTRGTSATHSHHSSHTTSAAHSRSGSVSQRVTSSQTV |
| NO: 166 | term_Cpack1-short_dI-dIV- | SHGVNETIYNTTLKYGDVVGVNITKYPYRVCSMAQGTDLIRFER |
| | dV-pack6_Disulf1 | NIVCTSMKPINEDLDEGIMVVYKRNIVAHTEKVRVYQKVLTERR |
| | Cpack1-short: E686L, | SYAYIHTTYLLGSNTEYVAPPMWEIHHINSHSQCYSSYSRVIAG |
| | Y690F, R693V, V694L | TVFVAYHRDSYENKTMQLMPDCYSSTHSTRYVTVKDQWHSRGST |
| | dI-dIV-dV-pack6: N220S, | WLYRETCNLNCMVTITTARSKYPYHFFATSTGDVVDISPFYNGT |
| | V5521, Q591F | NRNASYFGENADKFFIFPNYTIVSDFGRPNSALETHRLVAFLER |
| | Disulf1: D217C, S587C | ADSVISWDIQDEKNVTCQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSIVELERLANRSSLNLTHNRTK |
| | | RSTDGNNATHLSNMESVHNLVYAQLQFTYDTLRGYINRALAQIA |
| | | EAWCVDQRRTLEVFKELSKINPSAILSAIYNKPIAAREMGDVLG |
| | | LASCLTINQTSVKVLRDMNVKESPGRCYSRPVVIENFANCSYVE |
| | | YGQLGEDNEILLGNHRTEECQLPSLKIFIAGNSAYEYVDYLFKR |
| | | MIDLSSISTVDSMIALDIDPLENTDERVLELYSQKELRSSNVED |
| | | LEEIMRLFNSFKQVLKYV |
| SEQ ID | gB-Towne-ext-N- | GAAVSSSSTRGTSATHSHHSSHTTSAAHSRSGSVSQRVTSSQTV |
| NO: 167 | term_Cpack1-long_dI-dIV- | SHGVNETIYNTTLKYGDVVGVNTTKYPYRVCSMAQGTDLIRFER |
| | dV-pack6_Disulf1 | NIVCTSMKPINEDLDEGIMVVYKRNIVAHTFKVRVYQKVLTERR |
| | Cpack1-long: E686L, Y690F, | SYAYIHTTYLLGSNTEYVAPPMWEIHHINSHSQCYSSYSRVIAG |
| | R693V, V694L, K700A, V702Q | TVEVAYHRDSYENKTMQLMPDCYSSTHSTRYVTVKDQWHSRGST |
| | dI-dIV-dV-pack6: N220S, | WLYRETCNLNCMVTITTARSKYPYHFFATSTGDVVDISPFYNGT |
| | V552L, Q591F | NRNASYFGENADKFFIFPNYTIVSDFGRPNSALETHRLVAFLER |
| | Disulf1: D217C, S587C | ADSVISWDIQDEKNVICQLTFWEASERTIRSEAEDSYHFSSAKM |
| | | TATFLSKKQEVNMSDSALDCVRDEAINKLQQIFNTSYNQTYEKY |
| | | GNVSVFETTGGLVVFWQGIKQKSLVELERLANRSSLNLTHNRTK |
| | | RSTDGNNATHLSNMESVHNLVYAQLQFTYDTLRGYINRALAQIA |
| | | EAWCVDQRRTLEVEKELSKINPSAILSAIYNKPIAARFMGDVLG |
| | | LASCLTINQTSVKVLRDMNVKESPGRCYSRPVVIFNFANCSYVF |
| | | YGQLGEDNEILLGNHRTEECQLPSLKIFIAGNSAYEYVDYLFKR |
| | | MIDLSSISTVDSMIALDIDPLENTDFRVLELYSQKELRSSNVFD |
| | | LEEIMRLFNSFKQVLKYVEDAVQ |

### Timerization Domain

In some embodiments, the encoded polypeptide includes a heterologous trimerization domain. In some embodiments, the trimerization domain is at the C-terminus of the viral protein monomer. In some embodiments, the trimerization domain is a Foldon. The sequence of a representative FoldOn is as follows:

(SEQ ID NO: 308)
GYIPEAPRDGQAYVRKDGEWVLLSTF

In some embodiments, the trimerization domain is a GCN4 coiled-coil domain. The sequence of a representative GCN4 coiled-coil domain is as follows:

(SEQ ID NO: 309)
IEDKIEEILSKIYHIENEIARIKKLI

In some embodiments, the assembly domain is expressed as part of a fusion protein that comprises a viral protein monomer, a linker, and the assembly domain. Illustrative sequences of fusion proteins are provided in Table 10. In each case, in further embodiments, the assembly domain is any one of the assembly domains provided in Table 10, or another assembly domain.

TABLE 10

| | | |
|---|---|---|
| SEQ ID | gB-Towne_Bg-KOs_dII- | YRVCSMAQGTDLIRFERNIVCTSMKPINEDLDEGIMVVYKRNIVAHTEK |
| NO: 168 | dIII-pack1 | VRVYQKVLTFRRSYAYHRTTYLLGSNTEYVAPPMWEIHHINSHSQCYSS |
| | Bg-KOs: I156H, H157R, | YSRVIAGTVFVAYHRDSYENKTMQLMPDDYSNTHSTRYVTVKDQWHSRG |
| | W240N, Y242T, C246S, | STNLTRETSNLNCMVTITTARSKYPYHFFATSTGDVVDISPFYNGTNRN |
| | R457S, R460S | ASYFGENADKFFIFPNYTIVSDFGRPNSALETHRLVAFLERADSVISWD |
| | dII-dIII-pack1: S367I, | IQDEKNVTCQLTFWEASERTIRSEAEDSYHFISAKMTAFFLSKKQEVNM |
| | T374F | SDSALDCVRDEAINKLQQIFNTSYNQTYEKYGNVSVFETTGGLVVFWQG |
| | | IKQKSLVELERLANRSSLNLTHNSTKSSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNVF |
| | | DLEEIMREENSYKQRVKYV |
| SEQ ID | gB-Towne_Bg- | YRVCSMAQGTDLIRFERNIVCTSMKPINEDLDEGIMVVYKRNIVAHTFK |
| NO: 169 | KOs_Cpack1-short | VRVYQKVLIFRRSYAYHRTTYLLGSNTEYVAPPMWEIHHINSHSQCYSS |
| | Bg-KOs: I156H, H157R, | YSRVIAGTVFVAYHRDSYENKTMQLMPDDYSNTHSTRYVTVKDQWHSRG |
| | W240N, Y242T, C246S, | SINLTRETSNLNCMVTITTARSKYPYHFFATSTGDVVDISPFYNGTNRN |
| | R457S, R460S | ASYFGENADKFFIFPNYTIVSDFGRPNSALETHRLVAFLERADSVISWD |
| | Cpack1-short: E686L, | IQDEKNVTCQLTEWEASERTIRSEAEDSYHFSSAKMTATFLSKKQEVNM |
| | Y690F, R693V, V694L | SDSALDCVRDEAINKLQQIFNTSYNQTYEKYGNVSVFETTGGLVVFWQG |
| | | IKQKSIVELERLANRSSLNLTHNSTKSSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNVE |
| | | DLEEIMRLENSFKQVLKYV |
| SEQ ID | gB-Towne_Bg- | YRVCSMAQGTDLIRFERNIVCTSMKPINEDLDEGIMVVYKRNIVAHTFK |
| NO: 170 | KOs_Cpack1-long | VRVYQKVLTFRRSYAYHRTTYLLGSNTEYVAPPMWEIHHINSHSQCYSS |
| | Bg-KOs: I156H, H157R, | YSRVIAGTVFVAYHRDSYENKTMQLMPDDYSNTHSTRYVTVKDQWHSRG |
| | W240N, Y242T, C246S, | SINLTRETSNLNCMVTITTARSKYPYHFFATSTGDVVDISPFYNGTNRN |
| | R457S, R460S | ASYFGENADKFFIFPNYTIVSDFGRPNSALETHRLVAFLERADSVISWD |
| | Cpack1-long: E686L, | IQDEKNVTCQLTEWEASERTIRSEAEDSYHFSSAKMTATFLSKKQEVNM |
| | Y690F, R693V, V694L, | SDSALDCVRDEAINKLQQIFNTSYNQTYEKYGNVSVFETTGGLVVFWQG |
| | K700A, V702Q | IKQKSLVELERLANRSSLNLTANSTKSSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNVE |
| | | DLEEIMRLENSFKQVLKYVEDAVQ |
| SEQ ID | gB-Towne_Bg- | YRVCSMAQGTDLIRFERNIVCTSMKPINEDLDEGIMVVYKRNIVAHTEK |
| NO: 171 | KOs_Cpack2-short | VRVYQKVLTFRRSYAYHRTTYLLGSNTEYVAPPMWEIHHINSHSQCYSS |
| | Bg-KOs: I156H, H157R, | YSRVIAGTVFVAYHRDSYENKTMQLMPDDYSNTHSTRYVTVKDQWHSRG |
| | W240N, Y242T, C246S, | STNLTRETSNLNCMVTITTARSKYPYHFFATSTGDVVDISPFYNGTNRN |
| | R457S, R460S | ASYFGENADKFFIFPNYTIVSDFGRPNSALETHRLVAFLERADSVISWD |
| | Cpack2-short: D679N, | IQDEKNVTCQLTFWEASERTIRSEAEDSYHFSSAKMTATFLSKKQEVNM |
| | L680E, E681N, R685Q, | SDSALDCVRDEAINKLQQIFNTSYNQTYEKYGNVSVFETTGGLVVFWQG |
| | E686L, Y690F, R693V, | IKQKSLVELERLANRSSLNLTHNSTKSSTDGNNATHLSNMESVHNLVYA |
| | V694L, K695D | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNVF |
| | | NENEIMQLENSFKQVLDYV |
| SEQ ID | gB-Towne_Bg- | YRVCSMAQGTDLIRFERNIVCTSMKPINEDLDEGIMVVYKRNIVAHTEK |
| NO: 172 | KOs_Cpack2-long | VRVYQKVLTFRRSYAYHRTTYLLGSNTEYVAPPMWEIHHINSHSQCYSS |
| | Bg-KOs: I156H, H157R, | YSRVIAGTVFVAYHRDSYENKTMQLMPDDYSNTHSTRYVTVKDQWHSRG |
| | W240N, Y242T, C246S, | SINLTRETSNLNCMVTITTARSKYPYHFFATSTGDVVDISPFYNGTNRN |
| | R457S, R460S | ASYFGENADKFFIFPNYTIVSDFGRPNSALETHRLVAFLERADSVISWD |
| | Cpack2-long: D679N, | IQDEKNVTCQLTEWEASERTIRSEAEDSYHFSSAKMTATELSKKQEVNM |
| | L680E, E681N, R685Q, | SDSALDCVRDEAINKLQQIFNTSYNQTYEKYGNVSVFETTGGLVVFWQG |
| | E686L, Y690F, R693V, | IKQKSIVELERLANRSSLNLTANSTKSSTDGNNATHLSNMESVHNLVYA |
| | V694L, K695D, K700A, | QLQFTYDTLRGYINRALAQIAEAWCVDQRRTLEVFKELSKINPSAILSA |
| | V701L, V702Q | IYNKPIAARFMGDVLGLASCVTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNVE |
| | | NENEIMQLENSFKQVLDYVEDALQ |
| SEQ ID | gB-Towne_Bg- | YRVCSMAQGTDLIRFERNIVCTSMKPINEDLDEGIMVVYKRNIVAHTEK |
| NO: 173 | KOs_Cpack3-short | VRVYQKVLTFRRSYAYHRTTYLLGSNTEYVAPPMWEIHHINSHSQCYSS |
| | Bg-KOs: I156H, H157R, | YSRVIAGTVFVAYHRDSYENKTMQLMPDDYSNTHSTRYVTVKDQWHSRG |
| | W240N, Y242T, C246S, | STNLTRETSNINCMVTITTARSKYPYHFFATSTGDVVDISPFYNGTNRN |
| | R457S, R460S | ASYFGENADKFFIFPNYTIVSDEGRPNSALETHRLVAFLERADSVISWD |
| | Cpack3-short: V677T, | IQDEKNVTCQLTFWEASERTIRSEAEDSYHFSSAKMTATFLSKKQEVNM |
| | F678N, D679N, L680E, | SDSALDCVRDEAINKLQQIFNTSYNQTYEKYGNVSVFETTGGLVVFWQG |
| | E681N, E682S, R685Q, | IKQKSLVELERLANRSSLNLTHNSTKSSTDGNNATHLSNMESVHNLVYA |
| | E686L, N688E, Y690F, | QLQFTYDTLRGYINRALAQIAEAWCVDQRRTLEVFKELSKINPSAILSA |
| | R693V, V694L, K695D | IYNKPIAARFMGDVLGLASCVTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNTN |
| | | NENSIMQLFESFKQVLDYV |
| SEQ ID | gB-Towne_Bg- | YRVCSMAQGTDLIRFERNIVCTSMKPINEDLDEGIMVVYKRNIVAHTFK |
| NO: 174 | KOs_Cpack3-long | VRVYQKVLTFRRSYAYHRITYLLGSNTEYVAPPMWEIHHINSHSQCYSS |
| | Bg-KOs: I156H, H157R, | YSRVIAGTVFVAYHRDSYENKTMQLMPDDYSNTHSTRYVTVKDQWHSRG |
| | W240N, Y242T, C246S, | STNLTRETSNLNCMVTITTARSKYPYHFFATSTGDVVDISPFYNGTNRN |
| | R457S, R460S | ASYFGENADKFFIFPNYTIVSDEGRPNSALETHRLVAFLERADSVISWD |
| | Cpack3-long: V677T, | IQDEKNVICQLTFWEASERTIRSEAEDSYHFSSAKMTATFLSKKQEVNM |
| | F678N, D679N, L680E, | SDSALDCVRDEAINKLQQIFNTSYNQTYEKYGNVSVFETTGGLVVFWQG |
| | E681N, E682S, R685Q, | IKQKSLVELERLANRSSLNLTHNSTKSSTDGNNATHLSNMESVHNLVYA |
| | E686L, N688E, Y690F, | QLQFTYDTERGYINRALAQIAEAWCVDQRRTLEVEKELSKINPSAILSA |
| | R693V, V694L, K695D, | IYNKPIAARFMGDVLGLASCVTINQTSVKVLRDMNVKESPGRCYSRPVV |
| | D699K, K700A, V701L, | IFNFANSSYVQYGQLGEDNEILLGNHRTEECQLPSLKIFIAGNSAYEYV |
| | V702Q | DYLFKRMIDLSSISTVDSMIALDIDPLENTDFRVLELYSQKELRSSNIN |
| | | NENSIMQLFESFKQVLDYVEKALQ |
| SEQ ID | gB-Towne_Bg- | YRVCSMAQGTDLIRFERNIVCTSMKPINEDLDEGIMVVYKRNIVAHTFK |
| NO: 175 | KOs_Cpack4-short | VRVYQKVLTFRRSYAYHRTTYLLGSNTEYVAPPMWEIHHINSHSQCYSS |
| | Bg-KOs: I156H, H157R, | YSRVIAGTVFVAYHRDSYENKTMQLMPDDYSNTHSTRYVIVKDQWHSRG |
| | W240N, Y242T, C246S, | STNLTRETSNLNCMVTITTARSKYPYHFFATSTGDVVDISPFYNGTNRN |
| | R457S, R460S | ASYFGENADKFFIFPNYTIVSDFGRPNSALETHRLVAFLERADSVISWD |
| | Cpack4-short: V677T, | IQDEKNVTCQLIFWEASERTIRSEAEDSYHFSSAKMTATFLSKKQEVNM |
| | F678N, D679H, L680E, | SDSALDCVRDEAINKLQQIFNTSYNQTYEKYGNVSVFETTGGLVVFWQG |
| | E681N, E682S, M684S, | IKQKSLVELERLANRSSLNLTHNSTKSSTDGNNATHLSNMESVHNLVYA |
| | R685Q, E686L, F687A, | QLQFTYDTLRGYINRALAQIAEAWCVDQRRTLEVFKELSKINPSAILSA |
| | N688E, Y690F, Q692S, | IYNKPIAARFMGDVLGLASCVTINQTSVKVLRDMNVKESPGRCYSRPVV |
| | R693V, V694L, K695D, | IFNFANSSYVQYGQLGEDNEILLGNHRTEECQLPSLKIFIAGNSAYEYV |
| | Y696R | DYLFKRMIDLSSISTVDSMIALDIDPLENTDERVLELYSQKELRSSNTN |
| | | HENSISQLAESFKSVLDRV |
| SEQ ID | gB-Towne_Bg- | YRVCSMAQGTDLIRFERNIVCTSMKPINEDLDEGIMVVYKRNIVAHTEK |
| NO: 176 | KOs_Cpack4-long | VRVYQKVLTFRRSYAYHRTTYLLGSNTEYVAPPMWEIHHINSHSQCYSS |
| | Bg-KOs: I156H, H157R, | YSRVIAGTVFVAYHRDSYENKTMQLMPDDYSNTHSTRYVTVKDQWHSRG |
| | W240N, Y242T, C246S, | STNLIRETSNLNCMVTITTARSKYPYHFFATSTGDVVDISPFYNGTNRN |
| | R457S, R460S | ASYFGENADKFFIFPNYTIVSDEGRPNSALETHRLVAFLERADSVISWD |
| | Cpack4-long: V677T, | IQDEKNVICQLTFWEASERTIRSEAEDSYHFSSAKMIATFLSKKQEVNM |
| | F678N, D679H, L680E, | SDSALDCVRDEAINKLQQIFNTSYNQTYEKYGNVSVFETTGGLVVFWQG |
| | E681N, E682S, M684S, | IKQKSLVELERLANRSSLNLTHNSTKSSTDGNNATHLSNMESVHNLVYA |
| | R685Q, E686L, F687A, | QLQFTYDTLRGYINRALAQIAEAWCVDQRRTLEVFKELSKINPSAILSA |
| | N688E, Y690F, Q692S, | IYNKPIAARFMGDVLGLASCVTINQTSVKVLRDMNVKESPGRCYSRPVV |
| | R693V, V694L, K695D, | IFNFANSSYVQYGQLGEDNEILLGNHRTEECQLPSLKIFIAGNSAYEYV |
| | Y696R, D699K, K700A, | DYLFKRMIDLSSISTVDSMIALDIDPLENTDERVLELYSQKELRSSNIN |
| | V701L, V702E | HENSISQLAESFKSVLDRVEKALE |
| SEQ ID | gB-Towne_Bg- | YRVCSMAQGTDLIRFERNIVCTSMKPINEDLDEGIMVVYKRNIVAHTFK |
| NO: 177 | KOs_Cpack1-short_dII- | VRVYQKVLTFRRSYAYHRTTYLLGSNTEYVAPPMWEIHHINSHSQCYSS |
| | dIII-pack1 | YSRVIAGTVFVAYHRDSYENKIMQLMPDDYSNTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S, | ASYFGENADKFFIFPNYTIVSDEGRPNSALETHRLVAFLERADSVISWD |
| | R457S, R460S | IQDEKNVTCQLTFWEASERTIRSEAEDSYHFISAKMTAFFLSKKQEVNM |
| | dII-dIII-pack1: S367I, | SDSALDCVRDEAINKLQQIFNTSYNQTYEKYGNVSVFETTGGLVVFWQG |
| | T374F | IKQKSIVELERLANRSSLNLTHNSTKSSTDGNNATHLSNMESVHNLVYA |
| | Cpack1-short: E686L, | QLQFTYDTLRGYINRALAQIAEAWCVDQRRTLEVFKELSKINPSAILSA |
| | Y690F, R693V, V694L | IYNKPIAARFMGDVLGLASCVTINQTSVKVERDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDFRVLELYSQKELRSSNVF |
| | | DLEEIMRLENSFKQVLKYV |
| SEQ ID | gB-Towne_Bg- | YRVCSMAQGTDLIRFERNIVCTSMKPINEDLDEGIMVVYKRNIVAHTEK |
| NO: 178 | KOs_Cpack1-long_dII- | VRVYQKVLTFRRSYAYHRTTYLLGSNTEYVAPPMWEIHHINSHSQCYSS |
| | dIII-pack1 | YSRVIAGTVFVAYHRDSYENKTMQLMPDDYSNTHSTRYVTVKDQWHSRG |
| | Bg-KOs: I156H, H157R, | SINLTRETSNLNCMVTITTARSKYPYHFFATSTGDVVDISPFYNGTNRN |
| | W240N, Y242T, C246S, | ASYFGENADKFFIFPNYTIVSDEGRENSALETARLVAFLERADSVISWD |
| | R457S, R460S | IQDEKNVICQLTFWEASERTIRSEAEDSYHFISAKMTAFFLSKKQEVNM |
| | dII-dIII-pack1: S367I, | SDSALDCVRDEAINKLQQIFNTSYNQTYEKYGNVSVFETTGGLVVFWQG |
| | T374F | IKQKSLVELERLANRSSLNLTHNSTKSSTDGNNATHLSNMESVHNLVYA |
| | Cpack1-long: E686L, | QLQFTYDTLRGYINRALAQIAEAWCVDQRRTLEVFKELSKINPSAILSA |
| | Y690F, R693V, V694L, | IYNKPIAARFMGDVLGLASCVTINQTSVKVLRDMNVKESPGRCYSRPVV |
| | K700A, V702Q | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNVF |
| | | DLEEIMRLENSFKQVLKYVEDAVQ |
| SEQ ID | gB-Towne_Bg- | YRVCSMAQGTDLIRFERNIVCTSMKPINEDLDEGIMVVYKRNIVAHTEK |
| NO: 179 | KOs_Cpack2-short_dII- | VRVYQKVLIFRRSYAYHRTTYLLGSNTEYVAPPMWEIHHINSHSQCYSS |
| | dIII-pack1 | YSRVIAGTVFVAYHRDSYENKTMQLMPDDYSNTHSTRYVTVKDQWHSRG |
| | Bg-KOs: I156H, H157R, | SINLTRETSNLNCMVTITTARSKYPYHFFATSTGDVVDISPFYNGTNRN |
| | W240N, Y242T, C246S, | ASYFGENADKFFIFPNYTIVSDEGRENSALETHRLVAFLERADSVISWD |
| | R457S, R460S | IQDEKNVTCQLTEWEASERTIRSEAEDSYHFISAKMTAFFLSKKQEVNM |
| | dII-dIII-pack1: S367I, | SDSALDCVRDEAINKLQQIFNTSYNQTYEKYGNVSVFETTGGLVVFWQG |
| | T374F | IKQKSLVELERLANRSSLNLTANSTKSSTDGNNATHLSNMESVHNLVYA |
| | Cpack2-short: D679N, | QLQFTYDTERGYINRALAQIAEAWCVDQRRTLEVFKELSKINPSAILSA |
| | L680E, E681N, R685Q, | IYNKPIAARFMGDVLGLASCVTINQTSVKVLRDMNVKESPGRCYSRPVV |
| | E686L, Y690F, R693V, | IFNFANSSYVQYGQLGEDNEILLGNHRTEECQLPSLKIFIAGNSAYEYV |
| | V694L, K695D | DYLFKRMIDLSSISTVDSMIALDIDPLENTDERVLELYSQKELRSSNVE |
| | | NENEIMQLENSFKQVLDYV |
| SEQ ID | gB-Towne_Bg- | YRVCSMAQGTDLIRFERNIVCTSMKPINEDLDEGIMVVYKRNIVAHTEK |
| NO: 180 | KOs_Cpack2-long_dII- | VRVYQKVLTFRRSYAYHRTTYLLGSNTEYVAPPMWEIHHINSHSQCYSS |
| | dIII-pack1 | YSRVIAGTVFVAYHRDSYENKTMQLMPDDYSNTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S, | ASYFGENADKFFIFPNYTIVSDEGRPNSALETHRLVAFLERADSVISWD |
| | R457S, R460S | IQDEKNVICQLTFWEASERTIRSEAEDSYHFISAKMTAFFLSKKQEVNM |
| | dII-dIII-pack1: S367I, | SDSALDCVRDEAINKLQQIFNTSYNQTYEKYGNVSVFETTGGLVVFWQG |
| | T374F | IKQKSLVELERLANRSSLNLTHNSTKSSIDGNNATHLSNMESVHNLVYA |
| | Cpack2-long: D679N, | QLQFTYDELRGYINRALAQIAEAWCVDQRRTLEVFKELSKINPSAILSA |
| | L680E, E681N, R685Q, | IYNKPIAARFMGDVLGLASCVTINQTSVKVLRDMNVKESPGRCYSRPVV |
| | E686L, Y690F, R693V, | IFNFANSSYVQYGQLGEDNEILLGNHRTEECQLPSLKIFIAGNSAYEYV |
| | V694L, K695D, K700A, | DYLFKRMIDLSSISTVDSMIALDIDPLENTDERVLELYSQKELRSSNVF |
| | V701L, V702Q | NENEIMQLENSFKQVLDYVEDALQ |
| SEQ ID | gB-Towne_Bg- | YRVCSMAQGTDLIRFERNIVCISMKPINEDLDEGIMVVYKRNIVAHTEK |
| NO: 181 | KOs_Cpack3-short_dII- | VRVYQKVLTFRRSYAYHRTTYLLGSNTEYVAPPMWEIHHINSHSQCYSS |
| | dIII-pack1 | YSRVIAGTVFVAYHRDSYENKIMQLMPDDYSNTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S, | ASYFGENADKFFIFPNYTIVSDEGRPNSALETHRLVAFLERADSVISWD |
| | R457S, R460S | IQDEKNVTCQLTFWEASERTIRSEAEDSYHFISAKMTAFELSKKQEVNM |
| | dII-dIII-pack1: S367I, | SDSALDCVRDEAINKLQQIFNTSYNQTYEKYGNVSVFETTGGLVVFWQG |
| | T374F | IKQKSIVELERLANRSSLNLTHNSTKSSTDGNNATHLSNMESVHNLVYA |
| | Cpack3-short: V677T, | QLQFTYDTLRGYINRALAQIAEAWCVDQRRTLEVFKELSKINPSAILSA |
| | F678N, D679N, L680E, | IYNKPIAARFMGDVLGLASCVTINQTSVKVERDMNVKESPGRCYSRPVV |
| | E681N, E682S, R685Q, | IFNFANSSYVQYGQLGEDNEILLGNHRTEECQLPSLKIFIAGNSAYEYV |
| | E686L, N688E, Y690F, | DYLFKRMIDLSSISTVDSMIALDIDPLENTDERVLELYSQKELRSSNTN |
| | R693V, V694L, K695D | NENSIMQLFESFKQVLDYV |
| SEQ ID | gB-Towne_Bg- | YRVCSMAQGTDLIRFERNIVCTSMKPINEDLDEGIMVVYKRNIVAHTEK |
| NO: 182 | KOs_Cpack3-long_dII- | VRVYQKVLTFRRSYAYHRTTYLLGSNTEYVAPPMWEIHHINSHSQCYSS |
| | dIII-pack1 | YSRVIAGTVFVAYHRDSYENKIMQLMPDDYSNTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S, | ASYFGENADKFFIFPNYTIVSDFGRPNSALETHRLVAFLERADSVISWD |
| | R457S, R460S | IQDEKNVTCQLTFWEASERTIRSEAEDSYHFISAKMTAFFLSKKQEVNM |
| | dII-dIII-pack1: S367I, | SDSALDCVRDEAINKLQQIFNTSYNQTYEKYGNVSVFETTGGLVVFWQG |
| | T374F | IKQKSLVELERLANRSSLNLTHNSTKSSTDGNNATHLSNMESVHNLVYA |
| | Cpack3-long: V677T, | QLQFTYDTERGYINRALAQIAEAWCVDQRRTLEVFKELSKINPSAILSA |
| | F678N, D679N, L680E, | IYNKPIAARFMGDVLGLASCVTINQTSVKVLRDMNVKESPGRCYSRPVV |
| | E681N, E682S, R6850, | IFNFANSSYVQYGQLGEDNEILLGNHRTEECQLPSLKIFIAGNSAYEYV |
| | E686L, N688E, Y690F, | DYLFKRMIDLSSISTVDSMIALDIDPLENTDERVLELYSQKELRSSNTN |
| | R693V, V694L, K695D, | NENSIMQLFESFKQVLDYVEKALQ |
| | D699K, K700A, V701L, | |
| | V702Q | |
| SEQ ID | gB-Towne_Bg- | YRVCSMAQGTDLIRFERNIVCTSMKPINEDLDEGIMVVYKRNIVAHTEK |
| NO: 183 | KOs_Cpack4-short_dII- | VRVYQKVLIFRRSYAYHRITYLLGSNTEYVAPPMWEIHHINSHSQCYSS |
| | dIII-pack1 | YSRVIAGTVFVAYHRDSYENKTMQLMPDDYSNTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S, | ASYFGENADKFFIFPNYTIVSDFGRPNSALETARLVAFLERADSVISWD |
| | R457S, R460S | IQDEKNVICQLTFWEASERTIRSEAEDSYHFISAKMTAFELSKKQEVNM |
| | dII-dIII-pack1: 3367I, | SDSALDCVRDEAINKLQQIFNTSYNQTYEKYGNVSVFETTGGLVVFWQG |
| | T374F | IKQKSLVELERLANRSSLNLTHNSTKSSTDGNNATHLSNMESVHNLVYA |
| | Cpack4-short: V677T, | QLQFTYDTLRGYINRALAQIAEAWCVDQRRTLEVFKELSKINPSAILSA |
| | F678N, D679H, L680E, | IYNKPIAARFMGDVLGLASCVTINQTSVKVLRDMNVKESPGRCYSRPVV |
| | E681N, E682S, M684S, | IFNFANSSYVQYGQLGEDNEILLGNHRTEECQLPSLKIFIAGNSAYEYV |
| | R685Q, E686L, F687A, | DYLFKRMIDLSSISTVDSMIALDIDPLENTDERVLELYSQKELRSSNIN |
| | N688E, Y690F, Q692S, | HENSISQLAESFKSVLDRV |
| | R693V, V694L, K695D, | |
| | Y696R | |
| SEQ ID | gB-Towne_Bg- | YRVCSMAQGTDLIRFERNIVCTSMKPINEDLDEGIMVVYKRNIVAHTEK |
| NO: 184 | KOs_Cpack4-long_dII- | VRVYQKVLTFRRSYAYHRTTYLLGSNTEYVAPPMWEIHHINSHSQCYSS |
| | dIII-pack1 | YSRVIAGTVFVAYHRDSYENKTMQLMPDDYSNTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S, | ASYFGENADKFFIFPNYTIVSDEGRPNSALETHRLVAFLERADSVISWD |
| | R457S, R460S | IQDEKNVICQLTFWEASERTIRSEAEDSYHFISAKMTAFFLSKKQEVNM |
| | dII-dIII-pack1: S367I, | SDSALDCVRDEAINKLQQIFNTSYNQTYEKYGNVSVFETTGGLVVFWQG |
| | T374F | IKQKSLVELERLANRSSLNLTHNSTKSSTDGNNATHLSNMESVHNLVYA |
| | Cpack4-long: V677T, | QLQFTYDTLRGYINRALAQIAEAWCVDQRRTLEVFKELSKINPSAILSA |
| | F678N, D679H, L680E, | IYNKPIAARFMGDVLGLASCVTINQTSVKVLRDMNVKESPGRCYSRPVV |
| | E681N, E682S, M684S, | IFNFANSSYVQYGQLGEDNEILLGNHRTEECQLPSLKIFIAGNSAYEYV |
| | R685Q, E686L, F687A, | DYLFKRMIDLSSISTVDSMIALDIDPLENTDERVLELYSQKELRSSNIN |
| | N688E, Y690F, Q692S, | HENSISQLAESFKSVLDRVEKALE |
| | R693V, V694L, K695D, | |
| | Y696R, D699K, K700A, | |
| | V701L, V702E | |
| SEQ ID | gB-Towne_Bg- | YRVCSMAQGTDLIRFERNIVCTSMKPINEDLDEGIMVVYKRNIVAHTEK |
| NO: 185 | KOs_Cpack1-short_dI- | VRVYQKVLTFRRSYAYHRTTYLLGSNTEYVAPPMWEIHHINSHSQCYSS |
| | dIV-dV-pack1 | YSRVIAGTVFVAYHRDSYENKTMQLMPDDESNTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S, | ASYFGENADKFFIFPNYTIVSDEGRPNSALETHRLVAFLERADSVISWD |
| | R457S, R460S | IQDEKNVICQLTFWEASERTIRSEAEDSYHFSSAKMTATFLSKKQEVNM |
| | Cpack1-short: E686L, | SDSALDCVRDEAINKLQQIFNTSYNQTYEKYGNVSVFETTGGLVVFWQG |
| | Y690F, R693V, V694L | IKQKSIVELERLANRSSLNLTHNSTKSSTDGNNATHLSNMESVHNLVYA |
| | dI-dIV-dV-pack1: | QLQFTYDTLRGYINRALAQIAEAWCVDQRRTLEVFKELSKINPSAILSA |
| | Y218F, Q591F, S668A | IYNKPIAARFMGDVLGLASCVTINQTSVKVLRDMNVKESPGRCYSRPVV |
| | | IFNFANSSYVFYGQLGEDNEILLGNHRTEECQLPSLKIFIAGNSAYEYV |
| | | DYLFKRMIDLSSISTVDSMIALDIDPLENTDERVLELYAQKELRSSNVF |
| | | DLEEIMRLENSFKQVLKYV |
| SEQ ID | gB-Towne_Bg- | YRVCSMAQGTDLIRFERNIVCTSMKPINEDLDEGIMVVYKRNIVAHTEK |
| NO: 186 | KOs_Cpackl-long_dI- | VRVYQKVLTFRRSYAYHRTTYLLGSNTEYVAPPMWEIHHINSHSQCYSS |
| | dIV-dV-pack1 | YSRVIAGTVFVAYHRDSYENKTMQLMPDDFSNTHSTRYVTVKDQWHSRG |
| | Bg-KOs: I156H, H157R, | SINLTRETSNLNCMVTITTARSKYPYHFFATSTGDVVDISPFYNGTNRN |
| | W240N, Y242T, C246S, | ASYFGENADKFFIFPNYTIVSDEGRPNSALETHRLVAFLERADSVISWD |
| | R457S, R460S | IQDEKNVTCQLTFWEASERTIRSEAEDSYHFSSAKMTATFLSKKQEVNM |
| | Cpack1-long: E686L, | SDSALDCVRDEAINKLQQIFNTSYNQTYEKYGNVSVFETTGGLVVFWQG |
| | Y690F, R693V, V694L, | IKQKSLVELERLANRSSLNLTHNSTKSSTDGNNATHLSNMESVHNLVYA |
| | K700A, V702Q | QLQFTYDTLRGYINRALAQIAEAWCVDQRRTLEVFKELSKINPSAILSA |
| | dI-dIV-dV-pack1: | IYNKPIAARFMGDVLGLASCVTINQTSVKVLRDMNVKESPGRCYSRPVV |
| | Y218F, Q591F, S668A | IFNFANSSYVFYGQLGEDNEILLGNHRTEECQLPSLKIFIAGNSAYEYV |
| | | DYLFKRMIDLSSISTVDSMIALDIDPLENTDERVLELYAQKELRSSNVE |
| | | DLEEIMRLENSFKQVLKYVEDAVQ |
| SEQ ID | gB-Towne_Bg- | YRVCSMAQGTDLIRFERNIVCTSMKPINEDLDEGIMVVYKRNIVAHTEK |
| NO: 187 | KOs_Cpack1- | VRVYQKVLTFRRSYAYHRTTYLLGSNTEYVAPPMWEIHHINSHSQCYSS |
| | short_Disulf1 | YSRVIAGTVFVAYHRDSYENKTMQLMPDCYSNTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S, | ASYFGENADKFFIFPNYTIVSDEGRPNSALETHRLVAFLERADSVISWD |
| | R457S, R460S | IQDEKNVICQLTFWEASERTIRSEAEDSYHFSSAKMTATFLSKKQEVNM |
| | Cpack1-short: E686L, | SDSALDCVRDEAINKLQQIFNTSYNQTYEKYGNVSVFETTGGLVVFWQG |
| | Y690F, R693V, V694L | IKQKSLVELERLANRSSLNLTHNSTKSSTDGNNATHLSNMESVHNLVYA |
| | Disulf1: D217C, S587C | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANCSYVQYGQLGEDNEILLGNHRTEECQLPSLKIFIAGNSAYEYV |
| | | DYLFKRMIDLSSISTVDSMIALDIDPLENTDFRVLELYSQKELRSSNVF |
| | | DLEEIMRLENSFKQVLKYV |
| SEQ ID | gB-Towne_Bg- | YRVCSMAQGTDLIRFERNIVCTSMKPINEDLDEGIMVVYKRNIVAHTEK |
| NO: 188 | KOs_Cpack1- | VRVYQKVLTFRRSYAYHRTTYLLGSNTEYVAPPMWEIHHINSHSQCYSS |
| | long_Disulf1 | YSRVIAGTVFVAYHRDSYENKTMQLMPDCYSNTHSTRYVTVKDQWHSRG |
| | Bg-KOs: I156H, H157R, | SINLTRETSNLNCMVTITTARSKYPYHFFATSTGDVVDISPFYNGTNRN |
| | W240N, Y242T, C246S, | ASYFGENADKFFIFPNYTIVSDEGRPNSALETHRLVAFLERADSVISWD |
| | R457S, R460S | IQDEKNVTCQLTFWEASERTIRSEAEDSYHFSSAKMTATFLSKKQEVNM |
| | Cpack1-long: E686L, | SDSALDCVRDEAINKLQQIFNTSYNQTYEKYGNVSVFETTGGLVVFWQG |
| | Y690F, R693V, V694L, | IKQKSLVELERLANRSSLNLTHNSTKSSTDGNNATHLSNMESVHNLVYA |
| | K700A, V702Q | QLQFTYDTLRGYINRALAQIAEAWCVDQRRTLEVEKELSKINPSAILSA |
| | Disulf1: D217C, S587C | IYNKPIAARFMGDVLGLASCVTINQTSVKVLRDMNVKESPGRCYSRPVV |
| | | IFNFANCSYVQYGQLGEDNEILLGNHRTEECQLPSLKIFIAGNSAYEYV |
| | | DYLFKRMIDLSSISTVDSMIALDIDPLENTDFRVLELYSQKELRSSNVF |
| | | DLEEIMRLENSFKQVLKYVEDAVQ |
| SEQ ID | gB-Towne_Bg- | YRVCSMAQGTDLIRFERNIVCTSMKPINEDLDEGIMVVYKRNIVAHTFK |
| NO: 189 | KOs_Cpack1- | VRVYQKVLTFRRSYAYHRTTYLLGSNTEYVAPPMWEIHHINSHSQCYSS |
| | short_Disulf2 | YSRVIAGTVFVAYHRDSYENKIMQLMPDCYSNTHSTRYVTVKDQWHSRG |
| | Bg-KOs: I156H, H157R; | STNLTRETSNLNCMVTITTARSKYPYHFFATSTGDVVDISPFYNGINRN |
| | W240N, Y242T, C246S, | ASYFGENADKFFIFPNYTIVSDEGRPNSALETHRLVAFLERADSVISWD |
| | R457S, R460S | IQDEKNVTCQLIFWEASERTIRSEAEDSYHFSSAKMTATFLSKKQEVNM |
| | Cpack1-short: E686L, | SDSALDCVRDEAINKLQQIFNTSYNQTYEKYGNVSVFETTGGLVVFWQG |
| | Y690F, R693V, V694L | IKQKSIVELERLANRSSLNLTHNSTKSSTDGNNATHLSNMESVHNLVYA |
| | Disulf2: D217C, Y589C | QLQFTYDTLRGYINRALAQIAEAWCVDQRRTLEVFKELSKINPSAILSA |
| | | IYNKPIAARFMGDVLGLASCVTINQTSVKVERDMNVKESPGRCYSRPVV |
| | | IFNFANSSCVQYGQLGEDNEILLGNHRTEECQLPSLKIFIAGNSAYEYV |
| | | DYLFKRMIDLSSISTVDSMIALDIDPLENTDFRVLELYSQKELRSSNVF |
| | | DLEEIMRLENSFKQVLKYV |
| SEQ ID | gB-Towne_Bg- | YRVCSMAQGTDLIRFERNIVCTSMKPINEDLDEGIMVVYKRNIVAHTEK |
| NO: 190 | KOs_Cpack1- | VRVYQKVLTFRRSYAYHRTTYLLGSNTEYVAPPMWEIHHINSHSQCYSS |
| | long_Disulf2 | YSRVIAGTVFVAYHRDSYENKTMQLMPDCYSNTHSTRYVTVKDQWHSRG |
| | Bg-KOs: I156H, H157R, | SINLTRETSNLNCMVTITTARSKYPYHFFATSTGDVVDISPFYNGTNRN |
| | W240N, Y242T, C246S, | ASYFGENADKFFIFPNYTIVSDEGRPNSALETHRLVAFLERADSVISWD |
| | R457S, R460S | IQDEKNVICQLTFWEASERTIRSEAEDSYHFSSAKMTATFLSKKQEVNM |
| | Cpack1-long: E686L, | SDSALDCVRDEAINKLQQIFNTSYNQTYEKYGNVSVFETTGGLVVFWQG |
| | Y690F, R693V, V694L, | IKQKSLVELERLANRSSLNLTHNSTKSSTDGNNATHLSNMESVHNLVYA |
| | K700A, V702Q | QLQFTYDTLRGYINRALAQIAEAWCVDQRRTLEVFKELSKINPSAILSA |
| | Disulf2: D217C, Y589C | IYNKPIAARFMGDVLGLASCVTINQTSVKVLRDMNVKESPGRCYSRPVV |
| | | IFNFANSSCVQYGQLGEDNEILLGNHRTEECQLPSLKIFIAGNSAYEYV |
| | | DYLFKRMIDLSSISTVDSMIALDIDPLENTDFRVLELYSQKELRSSNVF |
| | | DLEEIMRLENSFKQVLKYVEDAVQ |
| SEQ ID | gB-Towne_Bg- | YRVCSMAQGTDLIRFERNIVCTSMKPINEDLDEGIMVVYKRNIVAHTFK |
| NO: 191 | KOs_Cpack1-short_dI- | VRVYQKVLTFRRSYAYHRTTYLLGSNTEYVAPPMWEIHHINSHSQCYSS |
| | dIV-dV-pack1_Disulf1 | YSRVIAGTVFVAYHRDSYENKTMQLMPDCFSNTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S, | ASYFGENADKFFIFPNYTIVSDFGRENSALETHRLVAFLERADSVISWD |
| | R457S, R460S | IQDEKNVTCQLTEWEASERTIRSEAEDSYHFSSAKMTATFLSKKQEVNM |
| | Cpack1-short: E686L, | SDSALDCVRDEAINKLQQIFNTSYNQTYEKYGNVSVFETTGGLVVFWQG |
| | Y690F, R693V, V694L | IKQKSLVELERLANRSSLNLTHNSTKSSTDGNNATHLSNMESVHNLVYA |
| | dI-dIV-dV-pack1: | QLQFTYDTERGYINRALAQIAEAWCVDQRRTLEVFKELSKINPSAILSA |
| | Y218F, Q591F, S668A | IYNKPIAARFMGDVLGLASCVTINQTSVKVLRDMNVKESPGRCYSRPVV |
| | Disulf1: D217C, S587C | IFNFANCSYVFYGQLGEDNEILLGNHRTEECQLPSLKIFIAGNSAYEYV |
| | | DYLFKRMIDLSSISTVDSMIALDIDPLENTDERVLELYAQKELRSSNVE |
| | | DLEEIMRLENSFKQVLKYV |
| SEQ ID | gB-Towne_Bg- | YRVCSMAQGTDLIRFERNIVCTSMKPINEDLDEGIMVVYKRNIVAHTEK |
| NO: 192 | KOs_Cpack1-long_dI- | VRVYQKVLTFRRSYAYHRTTYLLGSNTEYVAPPMWEIHHINSHSQCYSS |
| | dIV-dV-pack1_Disulf1 | YSRVIAGTVFVAYHRDSYENKTMQLMPDCFSNTHSTRYVTVKDQWHSRG |
| | Bg-KOs: I156H, H157R, | STNLTRETSNLNCMVTITTARSKYPYHFFATSTGDVVDISPFYNGINRN |
| | W240N, Y242T, C246S, | ASYFGENADKFFIFPNYTIVSDEGRPNSALETHRLVAFLERADSVISWD |
| | R457S, R460S | IQDEKNVTCQLTFWEASERTIRSEAEDSYHFSSAKMTATFLSKKQEVNM |
| | Cpack1-long: E686L, | SDSALDCVRDEAINKLQQIFNTSYNQTYEKYGNVSVFETTGGLVVFWQG |
| | Y690F, R693V, V694L, | IKQKSLVELERLANRSSLNLTHNSTKSSTDGNNATHLSNMESVHNLVYA |
| | K700A, V702Q | QLQFTYDTLRGYINPALAQIAEAWCVDQRRTLEVFKELSKINPSAILSA |
| | dI-dIV-dV-pack1: | IYNKPIAARFMGDVLGLASCVTINQTSVKVLRDMNVKESPGRCYSRPVV |
| | Y218F, Q591F, S668A | IFNFANCSYVFYGQLGEDNEILLGNHRTEECQLPSLKIFIAGNSAYEYV |
| | Disulf1: D217C, S587C | DYLFKRMIDLSSISTVDSMIALDIDPLENTDERVLELYAQKELRSSNVF |
| | | DLEEIMRLENSFKQVLKYVEDAVQ |
| SEQ ID | gB-Towne_Bg- | YRVCSMAQGTDLIRFERNIVCTSMKPINEDLDEGIMVVYKRNIVAHTEK |
| NO: 193 | KOs_Cpack1-short_dI- | VRVYQKVLTFRRSYAYHRTTYLLGSNTEYVAPPMWEIHHINSHSQCYSS |
| | dIV-dV-pack1 Disulf2 | YSRVIAGTVFVAYHRDSYENKTMQLMPDCFSNTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S, | ASYFGENADKFFIFPNYTIVSDFGRPNSALETHRLVAFLERADSVISWD |
| | R457S, R460S | IQDEKNVICQLTFWEASERTIRSEAEDSYHFSSAKMTATFLSKKQEVNM |
| | Cpack1-short: E686L, | SDSALDCVRDEAINKLQQIFNTSYNQTYEKYGNVSVFETTGGLVVFWQG |
| | Y690F, R693V, V694L | IKQKSLVELERLANRSSLNLTHNSTKSSTDGNNATHLSNMESVHNLVYA |
| | dI-dIV-dV-pack1: | QLQFTYDTLRGYINRALAQIAEAWCVDQRRTLEVFKELSKINPSAILSA |
| | Y218F, Q591F, S668A | IYNKPIAARFMGDVLGLASCVTINQTSVKVLRDMNVKESPGRCYSRPVV |
| | Disulf2: D217C, Y589C | IFNFANSSCVFYGQLGEDNEILLGNARTEECQLPSLKIFIAGNSAYEYV |
| | | DYLFKRMIDLSSISTVDSMIALDIDPLENTDFRVLELYAQKELRSSNVF |
| | | DLEEIMRLENSFKQVLKYV |
| SEQ ID | gB-Towne_Bg- | YRVCSMAQGTDLIRFERNIVCTSMKPINEDLDEGIMVVYKRNIVAHTEK |
| NO: 194 | KOs_Cpack1-long_dI- | VRVYQKVLTFRRSYAYHRTTYLLGSNTEYVAPPMWEIHHINSHSQCYSS |
| | dIV-dV-pack1_Disulf2 | YSRVIAGTVFVAYHRDSYENKTMQLMPDCFSNTHSTRYVTVKDQWHSRG |
| | Bg-KOs: I156H, H157R, | SINLTRETSNLNCMVTITTARSKYPYHFFATSTGDVVDISPFYNGTNRN |
| | W240N, Y242T, C246S, | ASYFGENADKFFIFPNYTIVSDFGRPNSALETHRLVAFLERADSVISWD |
| | R457S, R460S | IQDEKNVTCQLTFWEASERTIRSEAEDSYHFSSAKMTATFLSKKQEVNM |
| | Cpack1-long: E686L, | SDSALDCVRDEAINKLQQIFNTSYNQTYEKYGNVSVFETTGGLVVFWQG |
| | Y690F, R693V, V694L, | IKQKSLVELERLANRSSLNLTANSTKSSTDGNNATHLSNMESVHNLVYA |
| | K700A, V702Q | QLQFTYDTERGYINRALAQIAEAWCVDQRRTLEVEKELSKINPSAILSA |
| | dI-dIV-dV-pack1: | IYNKPIAARFMGDVLGLASCVTINQTSVKVLRDMNVKESPGRCYSRPVV |
| | Y218F, Q591F, S668A | IFNFANSSCVFYGQLGEDNEILLGNHRTEECQLPSLKIFIAGNSAYEYV |
| | Disulf2: D217C, Y589C | DYLFKRMIDLSSISTVDSMIALDIDPLENTDERVLELYAQKELRSSNVE |
| | | DLEEIMRLENSFKQVLKYVEDAVQ |
| SEQ ID | gB-Towne_Bg- | YRVCSMAQGTDLIRFERNIVCTSMKPINEDLDEGIMVVYKRNIVAHTEK |
| NO: 195 | KOs_Cpack1-short_dI- | VRVYQKVLTFRRSYAYHRTTYLLGSNITYVAPPMWEIHHINSHSQCYSS |
| | dIV-dV-pack1_N165glyc | YSRVIAGTVFVAYHRDSYENKIMQLMPDDESNTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S | ASYFGENADKFFIFPNYTIVSDEGRPNSALETHRLVAFLERADSVISWD |
| | R457S, R460S | IQDEKNVICQLTFWEASERTIRSEAEDSYHFSSAKMTATFLSKKQEVNM |
| | Cpack1-short: E686L, | SDSALDCVRDEAINKLQQIFNTSYNQTYEKYGNVSVFETTGGLVVFWQG |
| | Y690F, R693V, V694L | IKQKSLVELERLANRSSLNLTHNSTKSSTDGNNATHLSNMESVHNLVYA |
| | dI-dIV-dV-pack1: | QLQFTYDTLRGYINRALAQIAEAWCVDQRRTLEVFKELSKINPSAILSA |
| | Y218F, Q591F, S668A | IYNKPIAARFMGDVLGLASCVTINQTSVKVLRDMNVKESPGRCYSRPVV |
| | N165glyc: E167T | IFNFANSSYVFYGQLGEDNEILLGNHRTEECQLPSLKIFIAGNSAYEYV |
| | | DYLFKRMIDLSSISTVDSMIALDIDPLENTDERVLELYAQKELRSSNVF |
| | | DLEEIMRLENSFKQVLKYV |
| SEQ ID | gB-Towne_Bg- | YRVCSMAQGTDLIRFERNIVCTSMKPINEDLDEGIMVVYKRNIVAHTFK |
| NO: 196 | KOs_Cpackl-long_dI- | VRVYQKVLIFRRSYAYHRTTYLLGSNTTYVAPPMWEIHHINSHSQCYSS |
| | dIV-dV-pack1_N165glyc | YSRVIAGTVFVAYHRDSYENKTMQLMPDDESNTHSTRYVTVKDQWHSRG |
| | Bg-KOs: I156H, H157R, | SINLTRETSNLNCMVTITTARSKYPYHFFATSTGDVVDISPFYNGTNRN |
| | W240N, Y242T, C246S, | ASYFGENADKFFIFPNYTIVSDFGRPNSALETHRLVAFLERADSVISWD |
| | R457S, R460S | IQDEKNVTCQLTEWEASERTIRSEAEDSYHFSSAKMTATELSKKQEVNM |
| | Cpack1-long: E686L, | SDSALDCVRDEAINKLQQIFNTSYNQTYEKYGNVSVFETTGGLVVFWQG |
| | Y690F, R693V, V694L, | IKQKSLVELERLANRSSLNLTANSTKSSTDGNNATHLSNMESVHNLVYA |
| | K700A, V702Q | QLQFTYDTLRGYINRALAQIAEAWCVDQRRTLEVFKELSKINPSAILSA |
| | dI-dIV-dV-pack1: | IYNKPIAARFMGDVLGLASCVTINQTSVKVLRDMNVKESPGRCYSRPVV |
| | Y218F, Q591F, S668A | IFNFANSSYVFYGQLGEDNEILLGNHRTEECQLPSLKIFIAGNSAYEYV |
| | N165glyc: E167T | DYLFKRMIDLSSISTVDSMIALDIDPLENTDERVLELYAQKELRSSNVF |
| | | DLEEIMRLENSFKQVLKYVEDAVQ |
| SEQ ID | gB-Towne_Bg- | YRVCSMAQGTDLIRFERNIVCTSMKPINEDLDEGIMVVYKRNIVAHTFK |
| NO: 197 | KOs_Cpack1-short_dII- | VRVYQKVLTFRRSYAYHRTTYLLGSNTEYVAPPMWEIHHINSHSQCYSS |
| | dIII-pack1_dI-dIV-dV- | YSRVIAGTVFVAYHRDSYENKTMQLMPDDFSNTHSTRYVTVKDQWHSRG |
| | pack1 | STNLTRETSNLNCMVTITTARSKYPYHFFATSTGDVVDISPFYNGTNRN |
| | Bg-KOs: I156H, H157R, | ASYFGENADKFFIFPNYTIVSDEGRPNSALETHRLVAFLERADSVISWD |
| | W240N, Y242T, C246S, | IQDEKNVTCQLTFWEASERTIRSEAEDSYHFISAKMTAFFLSKKQEVNM |
| | R457S, R460S | SDSALDCVRDEAINKLQQIFNTSYNQTYEKYGNVSVFETTGGLVVFWQG |
| | dII-dIII-pack1: S367I, | IKQKSLVELERLANRSSLNLTHNSTKSSTDGNNATHLSNMESVHNLVYA |
| | T374F | QLQFTYDTLRGYINRALAQIAEAWCVDQRRTLEVFKELSKINPSAILSA |
| | Cpack1-short: E686L, | IYNKPIAARFMGDVLGLASCVTINQTSVKVLRDMNVKESPGRCYSRPVV |
| | Y690F, R693V, V694L | IFNFANSSYVFYGQLGEDNEILLGNHRTEECQLPSLKIFIAGNSAYEYV |
| | dI-dIV-dV-pack1: | DYLFKRMIDLSSISTVDSMIALDIDPLENTDFRVLELYAQKELRSSNVF |
| | Y218F, Q591F, S668A | DLEEIMRLENSFKQVLKYV |
| SEQ ID | gB-Towne_Bg- | YRVCSMAQGTDLIRFERNIVCTSMKPINEDLDEGIMVVYKRNIVAHTEK |
| NO: 198 | KOs_Cpack1-long_dII- | VRVYQKVLTFRRSYAYHRITYLLGSNTEYVAPPMWEIHHINSHSQCYSS |
| | dIII-pack1_dI-dIV-dV- | YSRVIAGTVFVAYHRDSYENKTMQLMPDDFSNTHSTRYVTVKDQWHSRG |
| | pack1 | STNLTRETSNLNCMVTITTARSKYPYHFFATSTGDVVDISPFYNGTNRN |
| | Bg-KOs: I156H, H157R, | ASYFGENADKFFIFPNYTIVSDEGRPNSALETHRLVAFLERADSVISWD |
| | W240N, Y242T, C246S, | IQDEKNVTCQLTFWEASERTIRSEAEDSYHFISAKMTAFFLSKKQEVNM |
| | R457S, R460S | SDSALDCVRDEAINKLQQIFNTSYNQTYEKYGNVSVFETTGGLVVFWQG |
| | dII-dIII-pack1: S367I, | IKQKSLVELERLANRSSLNLTHNSTKSSTDGNNATHLSNMESVHNLVYA |
| | T374F | QLQFTYDTLRGYINRALAQIAEAWCVDQRRTLEVFKELSKINPSAILSA |
| | Cpack1-long: E686L, | IYNKPIAARFMGDVLGLASCVTINQTSVKVLRDMNVKESPGRCYSRPVV |
| | Y690F, R693V, V694L, | IFNFANSSYVFYGQLGEDNEILLGNHRTEECQLPSLKIFIAGNSAYEYV |
| | K700A, V702Q | DYLFKRMIDLSSISTVDSMIALDIDPLENTDERVLELYAQKELRSSNVF |
| | dI-dIV-dV-pack1: | DLEEIMRLENSFKQVLKYVEDAVQ |
| | Y218F, Q591F, S668A | |
| SEQ ID | gB-Towne_Bg- | YRVCSMAQGTDLIRFERNIVCTSMKPINEDLDEGIMVVYKRNIVAHTEK |
| NO: 199 | KOs_Cpack1-short_dII- | VRVYQKVLTFRRSYAYHRTTYLLGSNTEYVAPPMWEIHHINSHSQCYSS |
| | dIII-pack1_Disulf1 | YSRVIAGIVEVAYHRDSYENKTMQLMPDCYSNTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S, | ASYFGENADKFFIFPNYTIVSDFGRPNSALETHRLVAFLERADSVISWD |
| | R457S, R460S | IQDEKNVTCQLTFWEASERTIRSEAEDSYHFISAKMTAFFLSKKQEVNM |
| | dII-dIII-pack1: S367I, | SDSALDCVRDEAINKLQQIFNTSYNQTYEKYGNVSVFETTGGLVVFWQG |
| | T374F | IKQKSLVELERLANRSSLNLTHNSTKSSTDGNNATHLSNMESVHNLVYA |
| | Cpack1-short: E686L, | QLQFTYDTLRGYINRALAQIAEAWCVDQRRTLEVFKELSKINPSAILSA |
| | Y690F, R693V, V694L | IYNKPIAARFMGDVLGLASCVTINQTSVKVLRDMNVKESPGRCYSRPVV |
| | Disulf1: D217C, S587C | IFNFANCSYVQYGQLGEDNEILLGNHRTEECQLPSLKIFIAGNSAYEYV |
| | | DYLFKRMIDLSSISTVDSMIALDIDPLENTDERVLELYSQKELRSSNVE |
| | | DLEEIMRLENSFKQVLKYV |
| SEQ ID | gB-Towne_Bg- | YRVCSMAQGTDLIRFERNIVCTSMKPINEDLDEGIMVVYKRNIVAHTEK |
| NO: 200 | Kos Cpack1-long_dII- | VRVYQKVLTFRRSYAYHRTTYLLGSNTEYVAPPMWEIHHINSHSQCYSS |
| | dIII-pack1_Disulf1 | YSRVIAGTVFVAYHRDSYENKTMQLMPDCYSNTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S, | ASYFGENADKFFIFPNYTIVSDEGRPNSALETHRLVAFLERADSVISWD |
| | R457S, R460S | IQDEKNVTCQLTFWEASERTIRSEAEDSYHFISAKMTAFFLSKKQEVNM |
| | dII-dIII-pack1: S367I, | SDSALDCVRDEAINKLQQIFNTSYNQTYEKYGNVSVFETTGGLVVFWQG |
| | T374F | IKQKSIVELERLANRSSLNLTHNSTKSSTDGNNATHLSNMESVHNLVYA |
| | Cpack1-long: E686L, | QLQFTYDTERGYINRALAQIAEAWCVDQRRTLEVFKELSKINPSAILSA |
| | Y690F, R693V, V694L, | IYNKPIAARFMGDVLGLASCVTINQTSVKVLRDMNVKESPGRCYSRPVV |
| | K700A, V702Q | IFNFANCSYVQYGQLGEDNEILLGNHRTEECQLPSLKIFIAGNSAYEYV |
| | | DYLFKRMIDLSSISTVDSMIALDIDPLENTDERVLELYSQKELRSSNVE |
| | Disulf1: D217C, S587C | DLEEIMRLENSFKQVLKYVEDAVQ |
| SEQ ID | gB-Towne_Bg- | YRVCSMAQGTDLIRFERNIVCTSMKPINEDLDEGIMVVYKRNIVAHTEK |
| NO: 201 | KOs_Cpack1-short_dII- | VRVYQKVLTFRRSYAYHRITYLLGSNTEYVAPPMWEIHHINSHSQCYSS |
| | dIII-pack1_Disulf2 | YSRVIAGTVFVAYHRDSYENKTMQLMPDCYSNTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S, | ASYFGENADKFFIFPNYTIVSDEGRPNSALETHRLVAFLERADSVISWD |
| | R457S, R460S | IQDEKNVTCQLTFWEASERTIRSEAEDSYHFISAKMTAFFLSKKQEVNM |
| | dII-dIII-pack1: S367I, | SDSALDCVRDEAINKLQQIFNTSYNQTYEKYGNVSVFETTGGLVVFWQG |
| | T374F | IKQKSLVELERLANRSSLNLTHNSTKSSTDGNNATHLSNMESVHNLVYA |
| | Cpack1-short: E686L, | QLQFTYDTERGYINRALAQIAEAWCVDQRRTLEVEKELSKINPSAILSA |
| | Y690F, R693V, V694L | IYNKPIAARFMGDVLGLASCVTINQTSVKVLRDMNVKESPGRCYSRPVV |
| | Disulf2: D217C, Y589C | IFNFANSSCVQYGQLGEDNEILLGNHRTEECQLPSLKIFIAGNSAYEYV |
| | | DYLFKRMIDLSSISTVDSMIALDIDPLENTDFRVLELYSQKELRSSNVF |
| | | DLEEIMRLENSFKQVLKYV |
| SEQ ID | gB-Towne_Bg- | YRVCSMAQGTDLIRFERNIVCTSMKPINEDLDEGIMVVYKRNIVAHTFK |
| NO: 202 | KOs_Cpack1-long_dII- | VRVYQKVLTFRRSYAYHRTTYLLGSNTEYVAPPMWEIHHINSHSQCYSS |
| | dIII-pack1_Disulf2 | YSRVIAGTVEVAYHRDSYENKIMQLMPDCYSNTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S, | ASYFGENADKFFIFPNYTIVSDEGRPNSALETHRLVAFLERADSVISWD |
| | R457S, R460S | IQDEKNVICQLTFWEASERTIRSEAEDSYHFISAKMTAFFLSKKQEVNM |
| | dII-dIII-pack1: S367I, | SDSALDCVRDEAINKLQQIFNTSYNQTYEKYGNVSVFETTGGLVVFWQG |
| | T374F | IKQKSLVELERLANRSSLNLTHNSTKSSTDGNNATHLSNMESVHNLVYA |
| | Cpack1-long: E686L, | QLQFTYDTLRGYINRALAQIAEAWCVDQRRTLEVFKELSKINPSAILSA |
| | Y690F, R693V, V694L, | IYNKPIAARFMGDVLGLASCVTINQTSVKVLRDMNVKESPGRCYSRPVV |
| | K700A, V702Q | IFNFANSSCVQYGQLGEDNEILLGNHRTEECQLPSLKIFIAGNSAYEYV |
| | Disulf2: D217C, Y589C | DYLFKRMIDLSSISTVDSMIALDIDPLENTDERVLELYSQKELRSSNVF |
| | | DLEEIMRLENSFKQVLKYVEDAVQ |
| SEQ ID | gB-Towne_Bg- | YRVCSMAQGTDLIRFERNIVCISMKPINEDLDEGIMVVYKRNIVAHTFK |
| NO: 203 | KOs_Cpack1-short_dII- | VRVYQKVLTFRRSYAYHRTTYLLGSNTEYVAPPMWEIHHINSHSQCYSS |
| | dIII-pack1_dI-dIV-dV- | YSRVIAGTVFVAYHRDSYENKTMQLMPDCFSNTHSTRYVTVKDQWHSRG |
| | pack1_Disulf1 | SINLTRETSNLNCMVTITTARSKYPYHFFATSTGDVVDISPFYNGTNRN |
| | Bg-KOs: I156H, H157R, | ASYFGENADKFFIFPNYTIVSDFGRPNSALETHRLVAFLERADSVISWD |
| | W240N, Y242T, C246S, | IQDEKNVTCQLTEWEASERTIRSEAEDSYHFISAKMTAFFLSKKQEVNM |
| | R457S, R460S | SDSALDCVRDEAINKLQQIFNTSYNQTYEKYGNVSVFETTGGLVVFWQG |
| | dII-dIII-pack1: S367I, | IKQKSLVELERLANRSSLNLTANSTKSSTDGNNATHLSNMESVHNLVYA |
| | T374F | QLQFTYDTLRGYINRALAQIAEAWCVDQRRTLEVEKELSKINPSAILSA |
| | Cpack1-short: E686L, | IYNKPIAARFMGDVLGLASCVTINQTSVKVLRDMNVKESPGRCYSRPVV |
| | Y690F, R693V, V694L | IFNFANCSYVFYGQLGEDNEILLGNHRTEECQLPSLKIFIAGNSAYEYV |
| | dI-dIV-dV-pack1: | DYLFKRMIDLSSISTVDSMIALDIDPLENTDERVLELYAQKELRSSNVE |
| | Y218F, Q591F, S668A | DLEEIMRLENSFKQVLKYV |
| | Disulf1: D217C, S587C | |
| SEQ ID | gB-Towne_Bg- | YRVCSMAQGTDLIRFERNIVCTSMKPINEDLDEGIMVVYKRNIVAHTFK |
| NO: 204 | KOs_Cpack1-long_dII- | VRVYQKVLTFRRSYAYHRTTYLLGSNTEYVAPPMWEIHHINSHSQCYSS |
| | dIII-pack1_dI-dIV-dV- | YSRVIAGTVFVAYHRDSYENKTMQLMPDCFSNTHSTRYVTVKDQWHSRG |
| | pack1_Disulf1 | SINLTRETSNLNCMVTITTARSKYPYHFFATSTGDVVDISPFYNGTNRN |
| | Bg-KOs: I156H, H157R, | ASYFGENADKFFIFPNYTIVSDFGRPNSALETHRLVAFLERADSVISWD |
| | W240N, Y242T, C246S, | IQDEKNVTCQLTEWEASERTIRSEAEDSYHFISAKMTAFELSKKQEVNM |
| | R457S, R460S | SDSALDCVRDEAINKLQQIFNTSYNQTYEKYGNVSVFETTGGLVVFWQG |
| | dII-dIII-pack1: S367I, | IKQKSEVELERLANRSSINLTANSTKSSTDGNNATHLSNMESVHNLVYA |
| | T374F | QLQFTYDTLRGYINRALAQIAEAWCVDQRRTLEVFKELSKINPSAILSA |
| | Cpack1-long: B686L, | IYNKPIAARFMGDVLGLASCVTINQTSVKVLRDMNVKESPGRCYSRPVV |
| | Y690F, R693V, V694L, | IFNFANCSYVFYGQLGEDNEILLGNHRTEECQLPSLKIFIAGNSAYEYV |
| | K700A, V702Q | DYLFKRMIDLSSISTVDSMIALDIDPLENTDERVLELYAQKELRSSNVE |
| | dI-dIV-dV-pack1: | DLEEIMRLENSFKQVLKYVEDAVQ |
| | Y218F, Q591F, S668A | |
| | Disulf1: D217C, S587C | |
| SEQ ID | gB-Towne_Bg- | YRVCSMAQGTDLIRFERNIVCTSMKPINEDLDEGIMVVYKRNIVAHTFK |
| NO: 205 | KOs_Cpack1-short_dII- | VRVYQKVLIFRRSYAYHRTTYLLGSNTEYVAPPMWEIHHINSHSQCYSS |
| | dIII-pack1_dI-dIV-dV- | YSRVIAGTVFVAYHRDSYENKTMQLMPDCFSNTHSTRYVTVKDQWHSRG |
| | pack1_Disulf2 | SINLTRETSNLNCMVTITTARSKYPYHFFATSTGDVVDISPFYNGTNRN |
| | Bg-KOs: I156H, H157R, | ASYFGENADKFFIFPNYTIVSDEGRPNSALETHRLVAFLERADSVISWD |
| | W240N, Y242T, C246S, | IQDEKNVTCQLTEWEASERTIRSEAEDSYHFISAKMTAFFLSKKQEVNM |
| | R457S, R460S | SDSALDCVRDEAINKLQQIFNTSYNQTYEKYGNVSVFETTGGLVVEWQG |
| | dII-dIII-pack1: S367I, | IKQKSEVELERLANRSSINLTANSTKSSTDGNNATHLSNMESVHNLVYA |
| | T374F | QLQFTYDTLRGYINRALAQIAEAWCVDQRRTLEVFKELSKINPSAILSA |
| | Cpack1-short: E686L, | IYNKPIAARFMGDVLGLASCVTINQTSVKVLRDMNVKESPGRCYSRPVV |
| | Y690F, R693V, V694L | IFNFANSSCVFYGQLGEDNEILLGNHRTEECQLPSLKIFIAGNSAYEYV |
| | dI-dIV-dV-pack1: | DYLFKRMIDLSSISTVDSMIALDIDPLENTDERVLELYAQKELRSSNVE |
| | Y218F, Q591F, S668A | DLEEIMRLENSFKQVLKYV |
| | Disulf2: D217C, Y589C | |
| SEQ ID | gB-Towne_Bg- | YRVCSMAQGTDLIRFERNIVCTSMKPINEDLDEGIMVVYKRNIVAHTFK |
| NO: 206 | KOs_Cpack1-long_dII- | VRVYQKVLIFRRSYAYHRTTYLLGSNTEYVAPPMWEIHHINSHSQCYSS |
| | dIII-pack1_dI-dIV-dV- | YSRVIAGTVFVAYHRDSYENKTMQLMPDCFSNTHSTRYVTVKDQWHSRG |
| | pack1_Disulf2 | STNLTRETSNLNCMVTITTARSKYPYHFFATSTGDVVDISPFYNGTNRN |
| | Bg-KOs: I156H, H157R, | ASYFGENADKFFIFPNYTIVSDEGRPNSALETHRLVAFLERADSVISWD |
| | W240N, Y242T, C246S, | IQDEKNVTCQLTEWEASERTIRSEAEDSYHFISAKMTAFFLSKKQEVNM |
| | R457S, R460S | SDSALDCVRDEAINKLQQIFNTSYNQTYEKYGNVSVFETTGGLVVFWQG |
| | dII-dIII-pack1: S367I, | IKQKSLVELERLANRSSLNLTHNSTKSSTDGNNATHLSNMESVHNLVYA |
| | T374F | QLQFTYDTERGYINRALAQIAEAWCVDQRRTLEVFKELSKINPSAILSA |
| | Cpack1-long: E686L, | IYNKPIAARFMGDVLGLASCVTINQTSVKVLRDMNVKESPGRCYSRPVV |
| | Y690F, R693V, V694L, | IFNFANSSCVFYGQLGEDNEILLGNHRTEECQLPSLKIFIAGNSAYEYV |
| | K700A, V702Q | DYLFKRMIDLSSISTVDSMIALDIDPLENTDERVLELYAQKELRSSNVE |
| | dI-dIV-dV-pack1: | DLEEIMRLENSFKQVLKYVEDAVQ |
| | Y218F, Q591F, S668A | |
| | Disulf2: D217C, Y589C | |
| SEQ ID | gB-Towne_Bg- | YRVCSMAQGTDLIRFERNIVCTSMKPINEDLDEGIMVVYKRNIVAHTEK |
| NO: 207 | KOs_Cpack1-short_dII- | VRVYQKVLIFRRSYAYHRTTYLLGSNTTYVAPPMWEIHHINSHSQCYSS |
| | dIII-pack1_dI-dIV-dV- | YSRVIAGTVFVAYHRDSYENKTMQLMPDDESNTHSTRYVTVKDQWHSRG |
| | pack1_N165glyc | STNLTRETSNLNCMVTITTARSKYPYHFFATSTGDVVDISPFYNGTNRN |
| | Bg-KOs: I156H, H157R, | ASYFGENADKFFIFPNYTIVSDEGRPNSALETHRLVAFLERADSVISWD |
| | W240N, Y242T, C246S, | IQDEKNVTCQLTFWEASERTIRSEAEDSYHFISAKMTAFFLSKKQEVNM |
| | R457S, R460S | SDSALDCVRDEAINKLQQIFNTSYNQTYEKYGNVSVFETTGGLVVFWQG |
| | dII-dIII-pack1: S367I, | IKQKSLVELERLANRSSLNLTANSTKSSTDGNNATHLSNMESVHNLVYA |
| | T374F | QLQFTYDTERGYINRALAQIAEAWCVDQRRTLEVFKELSKINPSAILSA |
| | Cpack1-short: E686L, | IYNKPIAARFMGDVLGLASCVTINQTSVKVLRDMNVKESPGRCYSRPVV |
| | Y690F, R693V, V694L | IFNFANSSYVFYGQLGEDNEILLGNHRTEECQLPSLKIFIAGNSAYEYV |
| | dI-dIV-dV-pack1: | DYLFKRMIDLSSISTVDSMIALDIDPLENTDFRVLELYAQKELRSSNVE |
| | Y218F, Q591F, S668A | DLEEIMRLENSFKQVLKYV |
| | N165glyc: E167T | |
| SEQ ID | gB-Towne_Bg- | YRVCSMAQGTDLIRFERNIVCTSMKPINEDLDEGIMVVYKRNIVAHTFK |
| NO: 208 | KOs_Cpack1-long_dII- | VRVYQKVLIFRRSYAYHRTTYLLGSNTTYVAPPMWEIHHINSHSQCYSS |
| | dIII-pack1_dI-dIV-dV- | YSRVIAGTVFVAYHRDSYENKTMQLMPDDFSNTHSTRYVTVKDQWHSRG |
| | pack1_N165glyc | SINLTRETSNLNCMVTITTARSKYPYHFFATSTGDVVDISPFYNGTNRN |
| | Bg-KOs: I156H, H157R, | ASYFGENADKFFIFPNYTIVSDFGRPNSALETHRLVAFLERADSVISWD |
| | W240N, Y242T, C246S, | IQDEKNVTCQLTFWEASERTIRSEAEDSYHFISAKMTAFFLSKKQEVNM |
| | R457S, R460S | SDSALDCVRDEAINKLQQIFNTSYNQTYEKYGNVSVFETTGGLVVFWQG |
| | dII-dIII-pack1: S367I, | IKQKSIVELERLANRSSLNLTHNSTKSSTDGNNATHLSNMESVHNLVYA |
| | T374F | QLQFTYDTLRGYINRALAQIAEAWCVDQRRTLEVFKELSKINPSAILSA |
| | Cpackl-long: E686L, | IYNKPIAARFMGDVLGLASCVTINQTSVKVLRDMNVKESPGRCYSRPVV |
| | Y690F, R693V, V694L, | IFNFANSSYVFYGQLGEDNEILLGNHRTEECQLPSLKIFIAGNSAYEYV |
| | K700A, V702Q | DYLFKRMIDLSSISTVDSMIALDIDPLENTDERVLELYAQKELRSSNVE |
| | dI-dIV-dV-pack1: | DLEEIMRLENSFKQVLKYVEDAVQ |
| | Y218F, Q591F, S668A | |
| | N165glyc: E167T | |
| SEQ ID | gB-Towne_Bg- | YRVCSMAQGTDLIRFERNIVCTSMKPINEDLDEGIMVVYKRNIVAHTEK |
| NO: 209 | KOs_Cpack1-short_dI- | VRVYQKVLTFRRSYAYHRTTYLLGSNTEYVAPPMWEIHHINSHSQCYSS |
| | dIV-dV-pack2 | YSRVIAGTVFVAYHRDSYENKTMQLMPDDYSSTHSTRYVTVKDQWHSRG |
| | Bg-KOs: I156H, H157R, | STNLTRETSNLNCMVTITTARSKYPYHFFATSTGDVVDISPFYNGINRN |
| | W240N, Y242T, C246S, | ASYFGENADKFFIFPNYTIVSDFGRPNSALETHRLVAFLERADSVISWD |
| | R457S, R460S | IQDEKNVTCQLIFWEASERTIRSEAEDSYHFSSAKMTATFLSKKQEVNM |
| | Cpack1-short: E686L, | SDSALDCVRDEAINKLQQIFNTSYNQTYEKYGNVSVFETTGGLVVFWQG |
| | Y690F, R693V, V694L | IKQKSIVELERLANRSSLNLTHNSTKSSTDGNNATHLSNMESVHNLVYA |
| | dI-dIV-dV-pack2: | QLQFTYDTLRGYINRALAQIAEAWCVDQRRTLEVFKELSKINPSAILSA |
| | N220S, V552L | IYNKPIAARFMGDVLGLASCLTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNVF |
| | | DLEEIMRLENSFKQVLKYV |
| SEQ ID | gB-Towne_Bg- | YRVCSMAQGTDLIRFERNIVCTSMKPINEDLDEGIMVVYKRNIVAHTFK |
| NO: 210 | KOs_Cpack1-long_dI- | VRVYQKVLTFRRSYAYHRITYLLGSNTEYVAPPMWEIHHINSHSQCYSS |
| | dIV-dV-pack2 | YSRVIAGTVFVAYHRDSYENKTMQLMPDDYSSTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S, | ASYFGENADKFFIFPNYTIVSDFGRPNSALETHRLVAFLERADSVISWD |
| | R457S, R460S | IQDEKNVTCQLTFWEASERTIRSEAEDSYHFSSAKMTATFLSKKQEVNM |
| | Cpack1-long: E686L, | SDSALDCVRDEAINKLQQIFNTSYNQTYEKYGNVSVFETTGGLVVFWQG |
| | Y690F, R693V, V694L, | IKQKSLVELERLANRSSLNLTHNSTKSSTDGNNATHLSNMESVHNLVYA |
| | K700A, V702Q | QLQFTYDTERGYINRALAQIAEAWCVDQRRTLEVEKELSKINPSAILSA |
| | dI-dIV-dV-pack2: | IYNKPIAARFMGDVLGLASCLTINQTSVKVLRDMNVKESPGRCYSRPVV |
| | N220S, V552L | IFNFANSSYVQYGQLGEDNEILLGNHRTEECQLPSLKIFIAGNSAYEYV |
| | | DYLFKRMIDLSSISTVDSMIALDIDPLENTDFRVLELYSQKELRSSNVF |
| | | DLEEIMRLENSFKQVLKYVEDAVQ |
| SEQ ID | gB-Towne_Bg- | YRVCSMAQGTDLIRFERNIVCTSMKPINEDLDEGIMVVYKRNIVAHTEK |
| NO: 211 | KOs_Cpack1-short_dI- | VRVYQKVLTFRRSYAYHRTTYLLGSNTEYVAPPMWEIHHINSHSQCYSS |
| | dIV-dV-pack3 | YSRVIAGIVEVAYHRDSYENKIMQLMPDDESSTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S, | ASYFGENADKFFIFPNYTIVSDFGRPNSALETHRLVAFLERADSVISWD |
| | R457S, R460S | IQDEKNVTCQLTFWEASERTIRSEAEDSYHFSSAKMTATFLSKKQEVNM |
| | Cpack1-short: E686L, | SDSALDCVRDEAINKLQQIFNTSYNQTYEKYGNVSVFETTGGLVVFWQG |
| | Y690F, R693V, V694L | IKQKSLVELERLANRSSLNLTHNSTKSSTDGNNATHLSNMESVHNLVYA |
| | dI-dIV-dV-pack3: | QLQFTYDTLRGYINRALAQIAEAWCVDQRRTLEVFKELSKINPSAILSA |
| | Y218F, N220S, V552L, | IYNKPIAARFMGDVLGLASCLTINQTSVKVLRDMNVKESPGRCYSRPVV |
| | Q591F, S668A | IFNFANSSYVFYGQLGEDNEILLGNHRTEECQLPSLKIFIAGNSAYEYV |
| | | DYLFKRMIDLSSISTVDSMIALDIDPLENTDFRVLELYAQKELRSSNVE |
| | | DLEEIMRLENSFKQVLKYV |
| SEQ ID | gB-Towne_Bg- | YRVCSMAQGTDLIRFERNIVCTSMKPINEDLDEGIMVVYKRNIVAHTFK |
| NO: 212 | KOs_Cpack1-long_dI- | VRVYQKVLTFRRSYAYHRTTYLLGSNTEYVAPPMWEIHHINSHSQCYSS |
| | dIV-dV-pack3 | YSRVIAGTVFVAYHRDSYENKTMQLMPDDFSSTHSTRYVTVKDQWHSRG |
| | Bg-KOs: I156H, H157R, | SINLTRETSNLNCMVTITTARSKYPYHFFATSTGDVVDISPFYNGTNRN |
| | W240N, Y242T, C246S, | ASYFGENADKFFIFPNYTIVSDEGRENSALETHRLVAFLERADSVISWD |
| | R457S, R460S | IQDEKNVTCQLTFWEASERTIRSEAEDSYHFSSAKMTATFLSKKQEVNM |
| | Cpack1-long: E686L, | SDSALDCVRDEAINKLQQIFNTSYNQTYEKYGNVSVFETTGGLVVFWQG |
| | Y690F, R693V, V694L, | IKQKSLVELERLANRSSLNLTHNSTKSSTDGNNATHLSNMESVHNLVYA |
| | K700A, V702Q | QLQFTYDTERGYINRALAQIAEAWCVDQRRTLEVEKELSKINPSAILSA |
| | dI-dIV-dV-pack3: | IYNKPIAARFMGDVLGLASCLTINQTSVKVLRDMNVKESPGRCYSRPVV |
| | Y218F, N220S, V552L, | IFNFANSSYVFYGQLGEDNEILLGNHRTEECQLPSLKIFIAGNSAYEYV |
| | Q591F, S668A | DYLFKRMIDLSSISTVDSMIALDIDPLENTDFRVLELYAQKELRSSNVF |
| | | DLEEIMRLENSFKQVLKYVEDAVQ |
| SEQ ID | gB-Towne_Bg- | YRVCSMAQGTDLIRFERNIVCTSMKPINEDLDEGIMVVYKRNIVAHTEK |
| NO: 213 | KOs_Cpack1-short_dII- | VRVYQKVLTFRRSYAYHRTTYLLGSNTEYVAPPMWEIHHINSHSQCYSS |
| | dIII-pack1_dI-dIV-dV- | YSRVIAGIVEVAYHRDSYENKTMQLMPDDYSSTHSTRYVTVKDQWHSRG |
| | pack2 | STNLTRETSNLNCMVTITTARSKYPYHFFATSTGDVVDISPFYNGTNRN |
| | Bg-KOs: I156H, H157R, | ASYFGENADKFFIFPNYTIVSDFGRPNSALETHRLVAFLERADSVISWD |
| | W240N, Y242T, C246S, | IQDEKNVTCQLTFWEASERTIRSEAEDSYHFISAKMTAFFLSKKQEVNM |
| | R457S, R460S | SDSALDCVRDEAINKLQQIFNTSYNQTYEKYGNVSVFETTGGLVVFWQG |
| | dII-dIII-pack1: S367I, | IKQKSLVELERLANRSSLNLTHNSTKSSTDGNNATHLSNMESVHNLVYA |
| | T374F | QLQFTYDTLRGYINRALAQIAEAWCVDQRRTLEVFKELSKINPSAILSA |
| | Cpack1-short: E686L, | IYNKPIAARFMGDVLGLASCLTINQTSVKVLRDMNVKESPGRCYSRPVV |
| | Y690F, R693V, V694L | IFNFANSSYVQYGQLGEDNEILLGNHRTEECQLPSLKIFIAGNSAYEYV |
| | dI-dIV-dV-pack2: | DYLFKRMIDLSSISTVDSMIALDIDPLENTDERVLELYSQKELRSSNVF |
| | N220S, V552L | DLEEIMRLENSFKQVLKYV |
| SEQ ID | gB-Towne_Bg- | YRVCSMAQGTDLIRFERNIVCTSMKPINEDLDEGIMVVYKRNIVAHTEK |
| NO: 214 | KOs_Cpack1-long_dII- | VRVYQKVLTFRRSYAYHRTTYLLGSNTEYVAPPMWEIHHINSHSQCYSS |
| | dIII-pack1_dI-dIV-dV- | YSRVIAGTVFVAYHRDSYENKTMQLMPDDYSSTHSTRYVTVKDQWHSRG |
| | pack2 | STNLTRETSNLNCMVTITTARSKYPYHFFATSTGDVVDISPFYNGTNRN |
| | Bg-KOs: I156H, H157R, | ASYFGENADKFFIFPNYTIVSDEGRPNSALETHRLVAFLERADSVISWD |
| | W240N, Y242T, C246S, | IQDEKNVTCQLTEWEASERTIRSEAEDSYHFISAKMTAFFLSKKQEVNM |
| | R457S, R460S | SDSALDCVRDEAINKLQQIFNTSYNQTYEKYGNVSVFETTGGLVVEWQG |
| | dII-dIII-pack1: S367I, | IKQKSLVELERLANRSSINLTANSTKSSTDGNNATHLSNMESVHNLVYA |
| | T374F | QLQFTYDTLRGYINRALAQIAEAWCVDQRRTLEVFKELSKINPSAILSA |
| | Cpack1-long: E686L, | IYNKPIAARFMGDVLGLASCLTINQTSVKVLRDMNVKESPGRCYSRPVV |
| | Y690F, R693V, V694L, | IFNFANSSYVQYGQLGEDNEILLGNHRTEECQLPSLKIFIAGNSAYEYV |
| | K700A, V702Q | DYLFKRMIDLSSISTVDSMIALDIDPLENTDERVLELYSQKELRSSNVE |
| | dI-dIV-dV-pack2: | DLEEIMRLENSFKQVLKYVEDAVQ |
| | N220S, V552L | |
| SEQ ID | gB-Towne_Bg- | YRVCSMAQGTDLIRFERNIVCTSMKPINEDLDEGIMVVYKRNIVAHTEK |
| NO: 215 | KOs_Cpack1-short_dII- | VRVYQKVLTFRRSYAYHRTTYLLGSNTEYVAPPMWEIHHINSHSQCYSS |
| | dIII-pack1_dI-dIV-dV- | YSRVIAGTVFVAYHRDSYENKTMQLMPDDFSSTHSTRYVTVKDQWHSRG |
| | pack3 | SINLTRETSNLNCMVTITTARSKYPYHFFATSTGDVVDISPFYNGTNRN |
| | Bg-KOs: I156H, H157R, | ASYFGENADKFFIFPNYTIVSDFGRPNSALETHRLVAFLERADSVISWD |
| | W240N, Y242T, C246S, | IQDEKNVICQLTEWEASERTIRSEAEDSYHFISAKMTAFFLSKKQEVNM |
| | R457S, R460S | SDSALDCVRDEAINKLQQIFNTSYNQTYEKYGNVSVFETTGGLVVFWQG |
| | dII-dIII-pack1: S367I, | IKQKSLVELERLANRSSLNLTANSTKSSTDGNNATHLSNMESVHNLVYA. |
| | T374F | QLQFTYDTLRGYINRALAQIAEAWCVDQRRTLEVFKELSKINPSAILSA |
| | Cpack1-short: E686L, | IYNKPIAARFMGDVLGLASCLTINQTSVKVLRDMNVKESPGRCYSRPVV |
| | Y690F, R693V, V694L | IFNFANSSYVFYGQLGEDNEILLGNHRTEECQLPSLKIFIAGNSAYEYV |
| | dI-dIV-dV-pack3: | DYLFKRMIDLSSISTVDSMIALDIDPLENTDERVLELYAQKELRSSNVE |
| | Y218F, N220S, V552L, | DLEEIMRLENSFKQVLKYV |
| | Q591F, S668A | |
| SEQ ID | gB-Towne_Bg- | YRVCSMAQGTDLIRFERNIVCTSMKPINEDLDEGIMVVYKRNIVAHTEK |
| NO: 216 | KOs_Cpack1-long_dII- | VRVYQKVLTFRRSYAYHRTTYLLGSNTEYVAPPMWEIHHINSHSQCYSS |
| | dIII-pack1_dI-dIV-dV- | YSRVIAGTVFVAYHRDSYENKTMQLMPDDESSTHSTRYVTVKDQWHSRG |
| | pack3 | STNLTRETSNLNCMVTITTARSKYPYHFFATSTGDVVDISPFYNGTNRN |
| | Bg-KOs: I156H, H157R, | ASYFGENADKFFIFPNYTIVSDEGRPNSALETHRLVAFLERADSVISWD |
| | W240N, Y242T, C246S, | IQDEKNVTCQLTFWEASERTIRSEAEDSYHFISAKMTAFFLSKKQEVNM |
| | R457S, R460S | SDSALDCVRDEAINKLQQIFNTSYNQTYEKYGNVSVFETTGGLVVFWQG |
| | dII-dIII-pack1: S367I, | IKQKSLVELERLANRSSLNLTHNSTKSSTDGNNATHLSNMESVHNLVYA |
| | T374F | QLQFTYDTERGYINRALAQIAEAWCVDQRRTLEVFKELSKINPSAILSA |
| | Cpack1-long: E686L, | IYNKPIAARFMGDVLGLASCLTINQTSVKVLRDMNVKESPGRCYSRPVV |
| | Y690F, R693V, V694L, | IFNFANSSYVFYGQLGEDNEILLGNHRTEECQLPSLKIFIAGNSAYEYV |
| | K700A, V702Q | DYLFKRMIDLSSISTVDSMIALDIDPLENTDFRVLELYAQKELRSSNVF |
| | dI-dIV-dV-pack3: | DLEEIMRLENSFKQVLKYVEDAVQ |
| | Y218F, N220S, V552L, | |
| | Q591F, S668A | |
| SEQ ID | gB-Towne_Bg- | YRVCSMAQGTDLIRFERNIVCTSMKPINEDLDEGIMVVYKRNIVAHTEK |
| NO: 217 | KOs_Cpack1-short_dI- | VRVYQKVLTFRRSYAYHRTTYLLGSNTEYVAPPMWEIHHINSHSQCYSS |
| | dIV-dV-pack3_Disulf1 | YSRVIAGTVFVAYHRDSYENKTMQLMPDCFSSTHSTRYVTVKDQWHSRG |
| | Bg-KOs: I156H, H157R, | SINLTRETSNLNCMVTITTARSKYPYHFFATSTGDVVDISPFYNGTNRN |
| | W240N, Y242T, C246S, | ASYFGENADKFFIFPNYTIVSDFGRPNSALETHRLVAFLERADSVISWD |
| | R457S, R460S | IQDEKNVTCQLTEWEASERTIRSEAEDSYHFSSAKMTATELSKKQEVNM |
| | Cpack1-short: E686L, | SDSALDCVRDEAINKLQQIFNTSYNQTYEKYGNVSVFETTGGLVVFWQG |
| | Y690F, R693V, V694L | IKQKSLVELERLANRSSLNLTHNSTKSSTDGNNATHLSNMESVHNLVYA |
| | dI-dIV-dV-pack3: | QLQFTYDTLRGYINRALAQIAEAWCVDQRRTLEVFKELSKINPSAILSA |
| | Y218F, N220S, V552L, | IYNKPIAARFMGDVLGLASCLTINQTSVKVLRDMNVKESPGRCYSRPVV |
| | Q591F, S668A | IFNFANCSYVFYGQLGEDNEILLGNHRTEECQLPSLKIFIAGNSAYEYV |
| | Disulf1: D217C, S587C | DYLFKRMIDLSSISTVDSMIALDIDPLENTDFRVLELYAQKELRSSNVF |
| | | DLEEIMRLENSFKQVLKYV |
| SEQ ID | gB-Towne_Bg- | YRVCSMAQGTDLIRFERNIVCTSMKPINEDLDEGIMVVYKRNIVAHTEK |
| NO: 218 | KOs_Cpack1-long_dI- | VRVYQKVLTFRRSYAYHRTTYLLGSNTEYVAPPMWEIHHINSHSQCYSS |
| | dIV-dV-pack3_Disulf1 | YSRVIAGTVFVAYHRDSYENKTMQLMPDCFSSTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S, | ASYFGENADKFFIFPNYTIVSDEGRENSALETHRLVAFLERADSVISWD |
| | R457S, R460S | IQDEKNVTCQLTFWEASERTIRSEAEDSYHFSSAKMTATFLSKKQEVNM |
| | Cpack1-long: E686L, | SDSALDCVRDEAINKLQQIFNTSYNQTYEKYGNVSVFETTGGLVVFWQG |
| | Y690F, R693V, V694L, | IKQKSLVELERLANRSSLNLTHNSTKSSTDGNNATHLSNMESVHNLVYA |
| | K700A, V702Q | QLQFTYDTLRGYINRALAQIAEAWCVDQRRTLEVFKELSKINPSAILSA |
| | dI-dIV-dV-pack3: | IYNKPIAARFMGDVLGLASCLTINQTSVKVLRDMNVKESPGRCYSRPVV |
| | Y218F, N220S, V552L, | IFNFANCSYVFYGQLGEDNEILLGNHRTEECQLPSLKIFIAGNSAYEYV |
| | Q591F, S668A | DYLFKRMIDLSSISTVDSMIALDIDPLENTDERVLELYAQKELRSSNVF |
| | Disulf1: D217C, S587C | DLEEIMRLENSFKQVLKYVEDAVQ |
| SEQ ID | gB-Towne_Bg- | YRVCSMAQGTDLIRFERNIVCTSMKPINEDLDEGIMVVYKRNIVAHTEK |
| NO: 219 | KOs_Cpack1-short_dII- | VRVYQKVLTFRRSYAYHRTTYLLGSNTEYVAPPMWEIHHINSHSQCYSS |
| | dIII-pack1_dI-dIV-dV- | YSRVIAGIVEVAYHRDSYENKTMQLMPDCFSSTHSTRYVTVKDQWHSRG |
| | pack3_Disulf1 | STNLTRETSNLNCMVTITTARSKYPYHFFATSTGDVVDISPFYNGTNRN |
| | Bg-KOs: I156H, H157R, | ASYFGENADKFFIFPNYTIVSDFGRPNSALETHRLVAFLERADSVISWD |
| | W240N, Y242T, C246S, | IQDEKNVTCQLTEWEASERTIRSEAEDSYHFISAKMTAFELSKKQEVNM |
| | R457S, R460S | SDSALDCVRDEAINKLQQIFNTSYNQTYEKYGNVSVFETTGGLVVFWQG |
| | Cpack1-short: E686L, | IKQKSLVELERLANRSSLNLTHNSTKSSTDGNNATHLSNMESVHNLVYA |
| | Y690F, R693V, V694L | QLQFTYDTLRGYINRALAQIAEAWCVDQRRTLEVFKELSKINPSAILSA |
| | dII-dIII-pack1: S367I, | IYNKPIAARFMGDVLGLASCLTINQTSVKVLRDMNVKESPGRCYSRPVV |
| | T374F | IFNFANCSYVFYGQLGEDNEILLGNHRTEECQLPSLKIFIAGNSAYEYV |
| | dI-dIV-dV-pack3: | DYLFKRMIDLSSISTVDSMIALDIDPLENTDERVLELYAQKELRSSNVE |
| | Y218F, N220S, V552L, | DLEEIMRLENSFKQVLKYV |
| | Q591F, S668A | |
| | Disulf1: D217C, S587C | |
| SEQ ID | gB-Towne_Bg- | YRVCSMAQGTDLIRFERNIVCTSMKPINEDLDEGIMVVYKRNIVAHTEK |
| NO: 220 | KOs_Cpack1-long_dII- | VRVYQKVLTFRRSYAYHRTTYLLGSNTEYVAPPMWEIHHINSHSQCYSS |
| | dIII-pack1_dI-dIV-dV- | YSRVIAGTVFVAYHRDSYENKTMQLMPDCFSSTHSTRYVTVKDQWHSRG |
| | pack3_Disulf1 | STNLTRETSNLNCMVTITTARSKYPYHFFATSTGDVVDISPFYNGINRN |
| | Bg-KOs: I156H, H157R, | ASYFGENADKFFIFPNYTIVSDFGRPNSALETHRLVAFLERADSVISWD |
| | W240N, Y242T, C246S, | IQDEKNVICQLTFWEASERTIRSEAEDSYHFISAKMTAFFLSKKQEVNM |
| | R457S, R460S | SDSALDCVRDEAINKLQQIFNTSYNQTYEKYGNVSVFETTGGLVVFWQG |
| | Cpack1-long: E686L, | IKQKSLVELERLANRSSLNLTHNSTKSSTDGNNATHLSNMESVHNLVYA |
| | Y690F, R693V, V694L, | QLQFTYDTLRGYINRALAQIAEAWCVDQRRTLEVFKELSKINPSAILSA |
| | K700A, V702Q | IYNKPIAARFMGDVLGLASCLTINQTSVKVLRDMNVKESPGRCYSRPVV |
| | dII-dIII-pack1: S367I, | IFNFANCSYVFYGQLGEDNEILLGNHRTEECQLPSLKIFIAGNSAYEYV |
| | T374F | DYLFKRMIDLSSISTVDSMIALDIDPLENTDERVLELYAQKELRSSNVF |
| | dI-dIV-dV-pack3: | DLEEIMRLENSFKQVLKYVEDAVQ |
| | Y218F, N220S, V552L, | |
| | Q591F, S668A | |
| | Disulf1: D217C, S587C | |
| SEQ ID | gB-Towne_Bg- | YRVCSMAQGTDLIRFERNIVCTSMKPINEDLDEGIMVVYKRNIVAHTEK |
| NO: 221 | KOs_Cpack1-short_dII- | VRVYQKVLTFRRSYAYHRTTYLLGSNTEYVAPPMWEIHHINSHSQCYSS |
| | dIII-pack1_dI-dIV-dV- | YSRVIAGTVFVAYHRDSYENKTMQLMPDCFSSTHSTRYVTVKDQWHSRG |
| | pack4_Disulf1 | STNLTRETSNLNCMVTITTARSKYPYHFFATSTGDVVDISPFYNGINRN |
| | Bg-KOs: I156H, H157R, | ASYFGENADKFFIFPNYTIVSDFGRPNSALETHRLVAFLERADSVISWD |
| | W240N, Y242T, C246S, | IQDEKNVTCQLTFWEASERTIRSEAEDSYHFISAKMTAFFLSKKQEVNM |
| | R457S, R460S | SDSALDCVRDEAINKLQQIFNTSYNQTYEKYGNVSVFETTGGLVVFWQG |
| | Cpack1-short: E686L, | IKQKSLVELERLANRSSLNLTHNSTKSSTDGNNATHLSNMESVHNLVYA |
| | Y690F, R693V, V694L | QLQFTYDTLRGYINPALAQIAEAWCVDQRRTLEVFKELSKINPSAILSA |
| | dII-dIII-pack1: S367I, | IYNKPIAARFMGDVLGLASCLTINQTSVKVLRDMNVKESPGRCYSRPVV |
| | T374F | IFNFANCSYVYYGQLGEDNEILLGNHRTEECQLPSLKIFIAGNSAYEYV |
| | dI-dIV-dV-pack4: | DYLFKRMIDLSSISTVDSMIALDIDPLENTDERVLELYAQKELRSSNVF |
| | Y218F, N220S, V552L, | DLEEIMRLENSFKQVLKYV |
| | Q591Y, S668A | |
| | Disulf1: D217C, S587C | |
| SEQ ID | gB-Towne_Bg- | YRVCSMAQGTDLIRFERNIVCTSMKPINEDLDEGIMVVYKRNIVAHTEK |
| NO: 222 | KOs_Cpackl-long_dII- | VRVYQKVLTFRRSYAYHRTTYLLGSNTEYVAPPMWEIHHINSHSQCYSS |
| | dIII-pack1_dI-dIV-dV- | YSRVIAGTVFVAYHRDSYENKTMQLMPDCFSSTHSTRYVTVKDQWHSRG |
| | pack4_Disulf1 | STNLTRETSNLNCMVTITTARSKYPYHFFATSTGDVVDISPFYNGTNRN |
| | Bg-KOs: I156H, H157R, | ASYFGENADKFFIFPNYTIVSDEGRPNSALETHRLVAFLERADSVISWD |
| | W240N, Y242T, C246S, | IQDEKNVTCQLTFWEASERTIRSEAEDSYHFISAKMTAFFLSKKQEVNM |
| | R457S, R460S | SDSALDCVRDEAINKLQQIFNTSYNQTYEKYGNVSVFETTGGLVVFWQG |
| | Cpack1-long: S686L, | IKQKSLVELERLANRSSLNLTHNSTKSSTDGNNATHLSNMESVHNLVYA |
| | Y690F, R693V, V694L, | QLQFTYDTLRGYINRALAQIAEAWCVDQRRTLEVFKELSKINPSAILSA |
| | K700A, V702Q | IYNKPIAARFMGDVLGLASCLTINQTSVKVLRDMNVKESPGRCYSRPVV |
| | dII-dIII-pack1: S367I, | IFNFANCSYVYYGQLGEDNEILLGNHRTEECQLPSLKIFIAGNSAYEYV |
| | T374F | DYLFKRMIDLSSISTVDSMIALDIDPLENTDERVLELYAQKELRSSNVF |
| | dI-dIV-dV-pack4: | DLEEIMRLENSFKQVLKYVEDAVQ |
| | Y218F, N220S, V552L, | |
| | Q591Y, S668A | |
| | Disulf1: D217C, S587C | |
| SEQ ID | gB-Towne_Bg- | YRVCSMAQGTDLIRFERNIVCTSMKPINEDLDEGIMVVYKRNIVAHTEK |
| NO: 223 | KOs_Cpack1-short_dII- | VRVYQKVLTFRRSYAYHRTTYLLGSNTEYVAPPMWEIHHINSHSQCYSS |
| | dIII-pack1_dI-dIV-dV- | YSRVIAGIVEVAYHRDSYENKTMQLMPDCYSSTHSTRYVTVKDQWHSRG |
| | pack5_Disulf1 | SINLTRETSNLNCMVTITTARSKYPYHFFATSTGDVVDISPFYNGTNRN |
| | Bg-KOs: I156H, H157R, | ASYFGENADKFFIFPNYTIVSDEGRENSALETHRLVAFLERADSVISWD |
| | W240N, Y242T, C246S, | IQDEKNVTCQLTFWEASERTIRSEAEDSYHFISAKMTAFELSKKQEVNM |
| | R457S, R460S | SDSALDCVRDEAINKLQQIFNTSYNQTYEKYGNVSVFETTGGLVVFWQG |
| | Cpack1-short: E686L, | IKQKSLVELERLANRSSLNLTANSTKSSTDGNNATHLSNMESVHNLVYA |
| | Y690F, R693V, V694L | QLQFTYDTLRGYINRALAQIAEAWCVDQRRTLEVFKELSKINPSAILSA |
| | dII-dIII-pack1: S367I, | IYNKPIAARFMGDVLGLASCLTINQTSVKVLRDMNVKESPGRCYSRPVV |
| | T374F | IFNFANCSYVYYGQLGEDNEILLGNHRTEECQLPSLKIFIAGNSAYEYV |
| | dI-dIV-dV-pack5: | DYLFKRMIDLSSISTVDSMIALDIDPLENTDFRVLELYSQKELRSSNVF |
| | N220S, V552L, Q591Y | DLEEIMRLENSFKQVLKYV |
| | Disulf1: D217C, S587C | |
| SEQ ID | gB-Towne_Bg- | YRVCSMAQGTDLIRFERNIVCTSMKPINEDLDEGIMVVYKRNIVAHTEK |
| NO: 224 | KOs_Cpack1-long_dII- | VRVYQKVLTFRRSYAYHRTTYLLGSNTEYVAPPMWEIHHINSHSQCYSS |
| | dIII-pack1_dI-dIV-dV- | YSRVIAGTVFVAYHRDSYENKTMQLMPDCYSSTHSTRYVTVKDQWHSRG |
| | pack5_Disulf1 | SINLTRETSNLNCMVTITTARSKYPYHFFATSTGDVVDISPFYNGTNRN |
| | Bg-KOs: I156H, H157R, | ASYFGENADKFFIFPNYTIVSDEGRPNSALETHRLVAFLERADSVISWD |
| | W240N, Y242T, C246S, | IQDEKNVTCQLTEWEASERTIRSEAEDSYHFISAKMTAFFLSKKQEVNM |
| | R457S, R460S | SDSALDCVRDEAINKLQQIFNTSYNQTYEKYGNVSVFETTGGLVVFWQG |
| | Cpack1-long: E686L, | IKQKSLVELERLANRSSLNLTANSTKSSTDGNNATHLSNMESVHNLVYA |
| | Y690F, R693V, V694L, | QLQFTYDTERGYINRALAQIAEAWCVDQRRTLEVFKELSKINPSAILSA |
| | K700A, V702Q | IYNKPIAARFMGDVLGLASCLTINQTSVKVLRDMNVKESPGRCYSRPVV |
| | dII-dIII-pack1: S367I, | IFNFANCSYVYYGQLGEDNEILLGNHRTEECQLPSLKIFIAGNSAYEYV |
| | T374F | DYLFKRMIDLSSISTVDSMIALDIDPLENTDERVLELYSQKELRSSNVF |
| | dI-dIV-dV-pack5: | DLEEIMRLENSFKQVLKYVEDAVQ |
| | N220S, V552L, Q591Y | |
| | Disulf1: D217C, S587C | |
| SEQ ID | gB-Towne_Bg- | YRVCSMAQGTDLIRFERNIVCTSMKPINEDLDEGIMVVYKRNIVAHTFK |
| NO: 225 | KOs_Cpack1-short_dII- | VRVYQKVLIFRRSYAYHRTTYLLGSNTEYVAPPMWEIHHINSHSQCYSS |
| | dIII-pack1_dI-dIV-dV- | YSRVIAGTVFVAYHRDSYENKTMQLMPDCYSSTHSTRYVTVKDQWHSRG |
| | pack6_Disulf1 | STNLTRETSNLNCMVTITTARSKYPYHFFATSTGDVVDISPFYNGTNRN |
| | Bg-KOs: I156H, H157R, | ASYFGENADKFFIFPNYTIVSDFGRPNSALETHRLVAFLERADSVISWD |
| | W240N, Y242T, C246S, | IQDEKNVTCQLTEWEASERTIRSEAEDSYHFISAKMTAFFLSKKQEVNM |
| | R457S, R460S | SDSALDCVRDEAINKLQQIFNTSYNQTYEKYGNVSVFETTGGLVVEWQG |
| | Cpack1-short: E686L, | IKQKSLVELERLANRSSLNLTHNSTKSSTDGNNATHLSNMESVHNLVYA |
| | Y690F, R693V, V694L | QLQFTYDTERGYINRALAQIAEAWCVDQRRTLEVFKELSKINPSAILSA |
| | dII-dIII-pack1: S367I, | IYNKPIAARFMGDVLGLASCLTINQTSVKVLRDMNVKESPGRCYSRPVV |
| | T374F | IFNFANCSYVFYGQLGEDNEILLGNHRTEECQLPSLKIFIAGNSAYEYV |
| | dI-dIV-dV-pack6: | DYLFKRMIDLSSISTVDSMIALDIDPLENTDERVLELYSQKELRSSNVE |
| | N220S, V552L, Q591F | DLEEIMRLENSFKQVLKYV |
| | Disulf1: D217C, S587C | |
| SEQ ID | gB-Towne_Bg- | YRVCSMAQGTDLIRFERNIVCTSMKPINEDLDEGIMVVYKRNIVAHTEK |
| NO: 226 | KOs_Cpack1-long_dII- | VRVYQKVLIFRRSYAYHRTTYLLGSNTEYVAPPMWEIHHINSHSQCYSS |
| | dIII-pack1_dI-dIV-dV- | YSRVIAGTVFVAYHRDSYENKTMQLMPDCYSSTHSTRYVTVKDQWHSRG |
| | pack6_Disulf1 | SINLTRETSNLNCMVTITTARSKYPYHFFATSTGDVVDISPFYNGTNRN |
| | Bg-KOs: I156H, H157R, | ASYFGENADKFFIFPNYTIVSDEGRENSALETHRLVAFLERADSVISWD |
| | W240N, Y242T, C246S, | IQDEKNVTCQLTEWEASERTIRSEAEDSYHFISAKMTAFFLSKKQEVNM |
| | R457S, R460S | SDSALDCVRDEAINKLQQIFNTSYNQTYEKYGNVSVFETTGGLVVEWQG |
| | Cpack1-long: E686L, | IKQKSLVELERLANRSSLNLTANSTKSSTDGNNATHLSNMESVHNLVYA |
| | Y690F, R693V, V694L, | QLQFTYDTLRGYINRALAQIAEAWCVDQRRTLEVFKELSKINPSAILSA |
| | K700A, V702Q | IYNKPIAARFMGDVLGLASCLTINQTSVKVLRDMNVKESPGRCYSRPVV |
| | dII-dIII-pack1: S367I, | IFNFANCSYVFYGQLGEDNEILLGNHRTEECQLPSLKIFIAGNSAYEYV |
| | T374F | DYLFKRMIDLSSISTVDSMIALDIDPLENTDERVLELYSQKELRSSNVF |
| | dI-dIV-dV-pack6: | DLEEIMRLENSFKQVLKYVEDAVQ |
| | N220S, V552L, Q591F | |
| | Disulf1: D217C, S587C | |
| SEQ ID | gB-Towne_Bg- | YRVCSMAQGTDLIRFERNIVCTSMKPINEDLDEGIMVVYKRNIVAHTEK |
| NO: 227 | KOs_Cpack1-short_dI- | VRVYQKVLTFRRSYAYHRTTYLLGSNTEYVAPPMWEIHHINSHSQCYSS |
| | dIV-dV-pack4_Disulf1 | YSRVIAGTVFVAYHRDSYENKIMQLMPDCFSSTHSTRYVTVKDQWHSRG |
| | Bg-KOs: I156H, H157R, | STNLTRETSNLNCMVTITTARSKYPYHFFATSTGDVVDISPFYNGTNRN |
| | W240N, Y242T, C246S, | ASYFGENADKFFIFPNYTIVSDEGRPNSALETHRLVAFLERADSVISWD |
| | R457S, R460S | IQDEKNVTCQLTEWEASERTIRSEAEDSYHFSSAKMTATFLSKKQEVNM |
| | Cpack1-short: E686L, | SDSALDCVRDEAINKLQQIFNTSYNQTYEKYGNVSVFETTGGLVVFWQG |
| | Y690F, R693V, V694L | IKQKSLVELERLANRSSLNLTHNSTKSSTDGNNATHLSNMESVHNLVYA |
| | dI-dIV-dV-pack4: | QLQFTYDTLRGYINRALAQIAEAWCVDQRRTLEVFKELSKINPSAILSA |
| | Y218F, N220S, V552L, | IYNKPIAARFMGDVIGLASCLTINQTSVKVLRDMNVKESPGRCYSRPVV |
| | Q591Y, S668A | IFNFANCSYVYYGQLGEDNEILLGNHRTEECQLPSLKIFIAGNSAYEYV |
| | Disulf1: D217C, S587C | DYLFKRMIDLSSISTVDSMIALDIDPLENTDERVLELYAQKELRSSNVF |
| | | DLEEIMRLENSFKQVLKYV |
| SEQ ID | gB-Towne_Bg- | YRVCSMAQGTDLIRFERNIVCTSMKPINEDLDEGIMVVYKRNIVAHTEK |
| NO: 228 | KOs_Cpack1-long_dI- | VRVYQKVLTFRRSYAYHRTTYLLGSNTEYVAPPMWEIHHINSHSQCYSS |
| | dIV-dV-pack4_Disulf1 | YSRVIAGTVFVAYHRDSYENKTMQLMPDCFSSTHSTRYVTVKDQWHSRG |
| | Bg-KOs: I156H, H157R, | SINLTRETSNLNCMVTITTARSKYPYHFFATSTGDVVDISPFYNGTNRN |
| | W240N, Y242T, C246S, | ASYFGENADKFFIFPNYTIVSDEGRPNSALETHRLVAFLERADSVISWD |
| | R457S, R460S | IQDEKNVTCQLTFWEASERTIRSEAEDSYHFSSAKMTATFLSKKQEVNM |
| | Cpack1-long: E686L, | SDSALDCVRDEAINKLQQIFNTSYNQTYEKYGNVSVFETTGGLVVFWQG |
| | Y690F, R693V, V694L, | IKQKSLVELERLANRSSLNLTHNSTKSSTDGNNATHLSNMESVHNLVYA |
| | K700A, V702Q | QLQFTYDTLRGYINRALAQIAEAWCVDQRRTLEVEKELSKINPSAILSA |
| | dI-dIV-dV-pack4: | IYNKPIAARFMGDVLGLASCLTINQTSVKVLRDMNVKESPGRCYSRPVV |
| | Y218F, N220S, V552L, | IFNFANCSYVYYGQLGEDNEILLGNHRTEECQLPSLKIFIAGNSAYEYV |
| | Q591Y, S668A | DYLFKRMIDLSSISTVDSMIALDIDPLENTDFRVLELYAQKELRSSNVF |
| | Disulf1: D217C, S587C | DLEEIMRLENSFKQVLKYVEDAVQ |
| SEQ ID | gB-Towne_Bg- | YRVCSMAQGTDLIRFERNIVCTSMKPINEDLDEGIMVVYKRNIVAHTEK |
| NO: 229 | KOs_Cpack1-short_dI- | VRVYQKVLTFRRSYAYHRITYLLGSNTEYVAPPMWEIHHINSHSQCYSS |
| | dIV-dV-pack5_Disulf1 | YSRVIAGTVFVAYHRDSYENKTMQLMPDCYSSTHSTRYVTVKDQWHSRG |
| | Bg-KOs: I156H, H157R, | STNLTRETSNLNCMVTITTARSKYPYHFFATSTGDVVDISPFYNGINRN |
| | W240N, Y242T, C246S, | ASYFGENADKFFIFPNYTIVSDEGRPNSALETHRLVAFLERADSVISWD |
| | R457S, R460S | IQDEKNVICQLTFWEASERTIRSEAEDSYHFSSAKMTATFLSKKQEVNM |
| | Cpack1-short: E686L, | SDSALDCVRDEAINKLQQIFNTSYNQTYEKYGNVSVFETTGGLVVFWQG |
| | Y690F, R693V, V694L | IKQKSLVELERLANRSSLNLTHNSTKSSTDGNNATHLSNMESVHNLVYA |
| | dI-dIV-dV-pack5: | QLQFTYDTLRGYINRALAQIAEAWCVDQRRTLEVFKELSKINPSAILSA |
| | N220S, V552L, Q591Y | IYNKPIAARFMGDVLGLASCLTINQTSVKVLRDMNVKESPGRCYSRPVV |
| | Disulf1: D217C, S587C | IFNFANCSYVYYGQLGEDNEILLGNHRTEECQLPSLKIFIAGNSAYEYV |
| | | DYLFKRMIDLSSISTVDSMIALDIDPLENTDERVLELYSQKELRSSNVF |
| | | DLEEIMRLENSFKQVLKYV |
| SEQ ID | gB-Towne_Bg- | YRVCSMAQGTDLIRFERNIVCISMKPINEDLDEGIMVVYKRNIVAHTEK |
| NO: 230 | Kos Cpack1-long_dI- | VRVYQKVLTFRRSYAYHRTTYLLGSNTEYVAPPMWEIHHINSHSQCYSS |
| | dIV-dV-pack5_Disulf1 | YSRVIAGTVFVAYHRDSYENKTMQLMPDCYSSTHSTRYVTVKDQWHSRG |
| | Bg-KOs: I156H, H157R, | STNLTRETSNLNCMVTITTARSKYPYHFFATSTGDVVDISPFYNGINRN |
| | W240N, Y242T, C246S, | ASYFGENADKFFIFPNYTIVSDFGRPNSALETHRLVAFLERADSVISWD |
| | R457S, R460S | IQDEKNVTCQLTFWEASERTIRSEAEDSYHFSSAKMTATFLSKKQEVNM |
| | Cpack1-long: E686L, | SDSALDCVRDEAINKLQQIFNTSYNQTYEKYGNVSVFETTGGLVVFWQG |
| | Y690F, R693V, V694L, | IKQKSIVELERLANRSSLNLTHNSTKSSTDGNNATHLSNMESVHNLVYA |
| | K700A, V702Q | QLQFTYDTLRGYINRALAQIAEAWCVDQRRTLEVFKELSKINPSAILSA |
| | dI-dIV-dV-pack5: | IYNKPIAARFMGDVIGLASCLTINQTSVKVLRDMNVKESPGRCYSRPVV |
| | N220S, V552L, Q591Y | IFNFANCSYVYYGQLGEDNEILLGNHRTEECQLPSLKIFIAGNSAYEYV |
| | Disulf1: D217C, S587C | DYLFKRMIDLSSISTVDSMIALDIDPLENTDERVLELYSQKELRSSNVF |
| | | DLEEIMRLENSFKQVLKYVEDAVQ |
| SEQ ID | gB-Towne_Bg- | YRVCSMAQGTDLIRFERNIVCTSMKPINEDLDEGIMVVYKRNIVAHTEK |
| NO: 231 | KOs_Cpack1-short_dI- | VRVYQKVLTFRRSYAYHRTTYLLGSNTEYVAPPMWEIHHINSHSQCYSS |
| | dIV-dV-pack6_Disulf1 | YSRVIAGTVFVAYHRDSYENKTMQLMPDCYSSTHSTRYVTVKDQWHSRG |
| | Bg-KOs: I156H, H157R, | SINLTRETSNLNCMVTITTARSKYPYHFFATSTGDVVDISPFYNGTNRN |
| | W240N, Y242T, C246S, | ASYFGENADKFFIFPNYTIVSDFGRPNSALETHRLVAFLERADSVISWD |
| | R457S, R460S | IQDEKNVTCQLTEWEASERTIRSEAEDSYHFSSAKMTATFLSKKQEVNM |
| | Cpack1-short: E686L, | SDSALDCVRDEAINKLQQIFNTSYNQTYEKYGNVSVFETTGGLVVFWQG |
| | Y690F, R693V, V694L | IKQKSLVELERLANRSSLNLTANSTKSSTDGNNATHLSNMESVHNLVYA |
| | dI-dIV-dV-pack6: | QLQFTYDTLRGYINRALAQIAEAWCVDQRRTLEVFKELSKINPSAILSA |
| | N220S, V552L, Q591F | IYNKPIAARFMGDVLGLASCLTINQTSVKVLRDMNVKESPGRCYSRPVV |
| | Disulf1: D217C, S587C | IFNFANCSYVFYGQLGEDNEILLGNHRTEECQLPSLKIFIAGNSAYEYV |
| | | DYLFKRMIDLSSISTVDSMIALDIDPLENTDERVLELYSQKELRSSNVE |
| | | DLEEIMRLENSFKQVLKYV |
| SEQ ID | gB-Towne_Bg- | YRVCSMAQGTDLIRFERNIVCTSMKPINEDLDEGIMVVYKRNIVAHTEK |
| NO: 232 | KOs_Cpack1-long_dI- | VRVYQKVLTFRRSYAYHRTTYLLGSNTEYVAPPMWEIHHINSHSQCYSS |
| | dIV-dV-pack6_Disulf1 | YSRVIAGTVFVAYHRDSYENKTMQLMPDCYSSTHSTRYVTVKDQWHSRG |
| | Bg-KOs: I156H, H157R, | STNLTRETSNLNCMVTITTARSKYPYHFFATSTGDVVDISPFYNGINRN |
| | W240N, Y242T, C246S, | ASYFGENADKFFIFPNYTIVSDEGRPNSALETHRLVAFLERADSVISWD |
| | R457S, R460S | IQDEKNVICQLTFWEASERTIRSEAEDSYHFSSAKMTATFLSKKQEVNM |
| | Cpack1-long: E686L, | SDSALDCVRDEAINKLQQIFNTSYNQTYEKYGNVSVFETTGGLVVFWQG |
| | Y690F, R693V, V694L, | IKQKSLVELERLANRSSLNLTHNSTKSSTDGNNATHLSNMESVHNLVYA |
| | K700A, V702Q | QLQFTYDTLRGYINRALAQIAEAWCVDQRRTLEVFKELSKINPSAILSA |
| | dI-dIV-dV-pack6: | IYNKPIAARFMGDVLGLASCLTINQTSVKVLRDMNVKESPGRCYSRPVV |
| | N220S, V552L, Q591F | IFNFANCSYVFYGQLGEDNEILLGNHRTEECQLPSLKIFIAGNSAYEYV |
| | Disulf1: D217C, S587C | DYLFKRMIDLSSISTVDSMIALDIDPLENTDERVLELYSQKELRSSNVF |
| | | DLEEIMRLENSFKQVLKYVEDAVQ |
| SEQ ID | gB-Towne_dII-dIII- | YRVCSMAQGTDLIRFERNIVCTSMKPINEDLDEGIMVVYKRNIVAHTFK |
| NO: 233 | pack1 | VRVYQKVLTFRRSYAYIHTTYLLGSNTEYVAPPMWEIHHINSHSQCYSS |
| | dII-dIII-pack1: S367I, | YSRVIAGTVFVAYHRDSYENKTMQLMPDDYSNTHSTRYVTVKDQWHSRG |
| | T374F | STWLYRETCNLNCMVTITTARSKYPYHFFATSTGDVVDISPFYNGTNRN |
| | | ASYFGENADKFFIFPNYTIVSDFGRPNSALETHRLVAFLERADSVISWD |
| | | IQDEKNVTCQLTFWEASERTIRSEAEDSYHFISAKMTAFFLSKKQEVNM |
| | | SDSALDCVRDEAINKLQQIFNTSYNQTYEKYGNVSVFETTGGLVVFWQG |
| | | IKQKSIVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNVF |
| | | DLEEIMREENSYKQRVKYV |
| SEQ ID | gB-Towne_Cpack1-short | YRVCSMAQGTDLIRFERNIVCTSMKPINEDLDEGIMVVYKRNIVAHTFK |
| NO: 234 | Cpack1-short: E686L, | VRVYQKVLTFRRSYAYIHTTYLLGSNTEYVAPPMWEIHHINSHSQCYSS |
| | Y690F, R693V, V694L | YSRVIAGTVFVAYHRDSYENKTMQLMPDDYSNTHSTRYVTVKDQWHSRG |
| | | STWLYRETCNLNCMVTITTARSKYPYHFFATSTGDVVDISPFYNGTNRN |
| | | ASYFGENADKFFIFPNYTIVSDEGRPNSALETHRLVAFLERADSVISWD |
| | | IQDEKNVICQLTFWEASERTIRSEAEDSYHFSSAKMTATFLSKKQEVNM |
| | | SDSALDCVRDEAINKLQQIFNTSYNQTYEKYGNVSVFETTGGLVVFWQG |
| | | IKQKSLVELERLANRSSLNLTHNRTKRSTDGNNATHLSNMESVHNLVYA |
| | | QLQFTYDTLRGYINRALAQIAEAWCVDQRRTLEVEKELSKINPSAILSA |
| | | IYNKPIAARFMGDVLGLASCVTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDFRVLELYSQKELRSSNVF |
| | | DLEEIMRLENSFKQVLKYV |
| SEQ ID | gB-Towne_Cpack1-long | YRVCSMAQGTDLIRFERNIVCTSMKPINEDLDEGIMVVYKRNIVAHTEK |
| NO: 235 | Cpack1-long: E686L, | VRVYQKVLTERRSYAYIHTTYLLGSNTEYVAPPMWEIHHINSHSQCYSS |
| | Y690F, R693V, V694L, | YSRVIAGTVFVAYHRDSYENKTMQLMPDDYSNTHSTRYVTVKDQWHSRG |
| | K700A, V702Q | STWLYRETCNLNCMVTITTARSKYPYHFFATSTGDVVDISPFYNGTNRN |
| | | ASYFGENADKFFIFPNYTIVSDEGRPNSALETHRLVAFLERADSVISWD |
| | | IQDEKNVTCQLTFWEASERTIRSEAEDSYHFSSAKMTATFLSKKQEVNM |
| | | SDSALDCVRDEAINKLQQIFNTSYNQTYEKYGNVSVFETTGGLVVFWQG |
| | | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVERDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNVE |
| | | DLEEIMRLENSFKQVLKYVEDAVQ |
| SEQ ID | gB-Towne_Cpack2-short | YRVCSMAQGTDLIRFERNIVCTSMKPINEDLDEGIMVVYKRNIVAHTEK |
| NO: 236 | Cpack2-short: D679N, | VRVYQKVLTFRRSYAYIHTTYLLGSNTEYVAPPMWEIHHINSHSQCYSS |
| | L680E, E681N, R685Q, | YSRVIAGIVEVAYHRDSYENKTMQLMPDDYSNTHSTRYVTVKDQWHSRG |
| | E686L, Y690F, R693V, | STWLYRETCNLNCMVTITTARSKYPYHFFATSTGDVVDISPFYNGTNRN |
| | V694L, K695D | ASYFGENADKFFIFPNYTIVSDFGRPNSALETHRLVAFLERADSVISWD |
| | | IQDEKNVTCQLIFWEASERTIRSEAEDSYHFSSAKMTATFLSKKQEVNM |
| | | SDSALDCVRDEAINKLQQIFNTSYNQTYEKYGNVSVFETTGGLVVFWQG |
| | | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNVF |
| | | NENEIMQLENSFKQVLDYV |
| SEQ ID | gB-Towne_Cpack2-long | YRVCSMAQGTDLIRFERNIVCTSMKPINEDLDEGIMVVYKRNIVAHTFK |
| NO: 237 | Cpack2-long: D679N, | VRVYQKVLTFRRSYAYIHTTYLLGSNTEYVAPPMWEIHHINSHSQCYSS |
| | L680E, E681N, R685Q, | YSRVIAGTVFVAYHRDSYENKTMQLMPDDYSNTHSTRYVTVKDQWHSRG |
| | E686L, Y690F, R693V, | STWLYRETCNLNCMVTITTARSKYPYHFFATSTGDVVDISPFYNGTNRN |
| | V694L, K695D, K700A, | ASYFGENADKFFIFPNYTIVSDFGRPNSALETHRLVAFLERADSVISWD |
| | V701L, V702Q | IQDEKNVICQLTFWEASERTIRSEAEDSYHFSSAKMTATFLSKKQEVNM |
| | | SDSALDCVRDEAINKLQQIFNTSYNQTYEKYGNVSVFETTGGLVVFWQG |
| | | IKQKSLVELERLANRSSLNLTANRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNVE |
| | | NENEIMQLENSFKQVLDYVEDALQ |
| SEQ ID | gB-Towne_Cpack3-short | YRVCSMAQGTDLIRFERNIVCTSMKPINEDLDEGIMVVYKRNIVAHTEK |
| NO: 238 | Cpack3-short: V677T, | VRVYQKVLTFRRSYAYIHTTYLLGSNTEYVAPPMWEIHHINSHSQCYSS |
| | F678N, D679N, L680E, | YSRVIAGTVFVAYHRDSYENKTMQLMPDDYSNTHSTRYVTVKDQWHSRG |
| | E681N, E682S, R685Q, | STWLYRETCNLNCMVTITTARSKYPYHFFATSTGDVVDISPFYNGTNRN |
| | E686L, N688E, Y690F, | ASYFGENADKFFIFPNYTIVSDEGRPNSALETHRLVAFLERADSVISWD |
| | R693V, V694L, K695D | IQDEKNVICQLTFWEASERTIRSEAEDSYHFSSAKMTATFLSKKQEVNM |
| | | SDSALDCVRDEAINKLQQIFNTSYNQTYEKYGNVSVFETTGGLVVFWQG |
| | | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNTN |
| | | NENSIMQLFESFKQVLDYV |
| SEQ ID | gB-Towne_Cpack3-long | YRVCSMAQGTDLIRFERNIVCTSMKPINEDLDEGIMVVYKRNIVAHTEK |
| NO: 239 | Cpack3-long: V677T, | VRVYQKVLTFRRSYAYIHTTYLLGSNTEYVAPPMWEIHHINSHSQCYSS |
| | F678N, D679N, L680E, | YSRVIAGTVFVAYHRDSYENKTMQLMPDDYSNTHSTRYVTVKDQWHSRG |
| | E681N, E682S, R685Q, | STWLYRETCNLNCMVTITTARSKYPYHFFATSTGDVVDISPFYNGTNRN |
| | E686L, N688E, Y690F, | ASYFGENADKFFIFPNYTIVSDFGRPNSALETHRLVAFLERADSVISWD |
| | R693V, V694L, K695D, | IQDEKNVTCQLTFWEASERTIRSEAEDSYHFSSAKMTATFLSKKQEVNM |
| | D699K, K700A, V701L, | SDSALDCVRDEAINKLQQIFNTSYNQTYEKYGNVSVFETTGGLVVFWQG |
| | V702Q | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNIN |
| | | NENSIMQLFESFKQVLDYVEKALQ |
| SEQ ID | gB-Towne_Cpack4-short | YRVCSMAQGTDLIRFERNIVCTSMKPINEDLDEGIMVVYKRNIVAHTEK |
| NO: 240 | Cpack4-short: V677T, | VRVYQKVLIFRRSYAYIHTTYLLGSNTEYVAPPMWEIHHINSHSQCYSS |
| | F678N, D679H, L680E, | YSRVIAGTVFVAYHRDSYENKTMQLMPDDYSNTHSTRYVTVKDQWHSRG |
| | E681N, E682S, M684S, | STWLYRETCNLNCMVTITTARSKYPYHFFATSTGDVVDISPFYNGTNRN |
| | R685Q, E686L, F687A, | ASYFGENADKFFIFPNYTIVSDFGRPNSALETHRLVAFLERADSVISWD |
| | N688E, Y690F, Q692S, | IQDEKNVTCQLTEWEASERTIRSEAEDSYHFSSAKMTATFLSKKQEVNM |
| | R693V, V694L, K695D, | SDSALDCVRDEAINKLQQIFNTSYNQTYEKYGNVSVFETTGGLVVEWQG |
| | Y696R | IKQKSLVELERLANRSSLNLTANRIKRSTDGNNATHLSNMESVHNLVYA |
| | | QLQFTYDTERGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNTN |
| | | HENSISQLAESFKSVLDRV |
| SEQ ID | gB-Towne_Cpack4-long | YRVCSMAQGTDLIRFERNIVCTSMKPINEDLDEGIMVVYKRNIVAHTEK |
| NO: 241 | Cpack4-long: V677T, | VRVYQKVLTFRRSYAYIHTTYLLGSNTEYVAPPMWEIHHINSHSQCYSS |
| | F678N, D679H, L680E, | YSRVIAGTVFVAYHRDSYENKTMQLMPDDYSNTHSTRYVIVKDQWHSRG |
| | E681N, E682S, M684S, | STWLYRETCNLNCMVTITTARSKYPYHFFATSTGDVVDISPFYNGTNRN |
| | R685Q, E686L, F687A, | ASYFGENADKFFIFPNYTIVSDFGRPNSALETHRLVAFLERADSVISWD |
| | N688E, Y690F, Q692S, | IQDEKNVTCQLTFWEASERTIRSEAEDSYHFSSAKMTATFLSKKQEVNM |
| | R693V, V694L, K695D, | SDSALDCVRDEAINKLQQIFNTSYNQTYEKYGNVSVFETTGGLVVFWQG |
| | Y696R, D699K, K700A, | IKQKSLVELERLANRSSLNLTHNRTKRSTDGNNATHLSNMESVHNLVYA |
| | V701L, V702E | QLQFTYDTLRGYINRALAQIAEAWCVDQRRTLEVFKELSKINPSAILSA |
| | | IYNKPIAARFMGDVLGLASCVTINQTSVKVERDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNTN |
| | | HENSISQLAESFKSVLDRVEKALE |
| SEQ ID | gB-Towne_Cpack1- | YRVCSMAQGTDLIRFERNIVCTSMKPINEDLDEGIMVVYKRNIVAHTEK |
| NO: 242 | short_dII-dIII-pack1 | VRVYQKVLTFRRSYAYIHTTYLLGSNTEYVAPPMWEIHHINSHSQCYSS |
| | dII-dIII-pack1: S367I, | YSRVIAGTVFVAYHRDSYENKTMQLMPDDYSNTHSTRYVTVKDQWHSRG |
| | T374F | STWLYRETCNLNCMVTITTARSKYPYHFFATSTGDVVDISPFYNGTNRN |
| | Cpack1-short: E686L, | ASYFGENADKFFIFPNYTIVSDEGRPNSALETHRLVAFLERADSVISWD |
| | Y690F, R693V, V694L | IQDEKNVICQLTFWEASERTIRSEAEDSYHFISAKMTAFFLSKKQEVNM |
| | | SDSALDCVRDEAINKLQQIFNTSYNQTYEKYGNVSVFETTGGLVVFWQG |
| | | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTEK |
| NO: 243 | long_dII-dIII-pack1 | VRVYQKVLIFRRSYAYIHTTYLLGSNTEYVAPPMWEIHHINSHSQCYSS |
| | dII-dIII-pack1: S367I, | YSRVIAGTVFVAYHRDSYENKTMQLMPDDYSNTHSTRYVTVKDQWHSRG |
| | T374F | STWLYRETCNLNCMVTITTARSKYPYHFFATSTGDVVDISPFYNGTNRN |
| | Cpack1-long: S686L, | ASYFGENADKFFIFPNYTIVSDEGRPNSALETHRLVAFLERADSVISWD |
| | Y690F, R693V, V694L, | IQDEKNVICQLTFWEASERTIRSEAEDSYHFISAKMTAFFLSKKQEVNM |
| | K700A, V702Q | SDSALDCVRDEAINKLQQIFNTSYNQTYEKYGNVSVFETTGGLVVFWQG |
| | | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVERDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNVE |
| | | DLEEIMRLENSFKQVLKYVEDAVQ |
| SEQ ID | gB-Towne_Cpack2- | YRVCSMAQGTDLIRFERNIVCTSMKPINEDLDEGIMVVYKRNIVAHTEK |
| NO: 244 | short_dII-dIII-pack1 | VRVYQKVLTFRRSYAYIHTTYLLGSNTEYVAPPMWEIHHINSHSQCYSS |
| | dII-dIII-pack1: S367I, | YSRVIAGTVFVAYHRDSYENKTMQLMPDDYSNTHSTRYVTVKDQWHSRG |
| | T374F | STWLYRETCNLNCMVTITTARSKYPYHFFATSTGDVVDISPFYNGINRN |
| | Cpack2-short: D679N, | ASYFGENADKFFIFPNYTIVSDEGRPNSALETHRLVAFLERADSVISWD |
| | L680E, E681N, R685Q, | IQDEKNVICQLTFWEASERTIRSEAEDSYHFISAKMTAFFLSKKQEVNM |
| | E686L, Y690F, R693V, | SDSALDCVRDEAINKLQQIFNTSYNQTYEKYGNVSVFETTGGLVVFWQG |
| | V694L, K695D | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVERDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDFRVLELYSQKELRSSNVF |
| | | NENEIMQLENSFKQVLDYV |
| SEQ ID | gB-Towne_Cpack2- | YRVCSMAQGTDLIRFERNIVCTSMKPINEDLDEGIMVVYKRNIVAHTFK |
| NO: 245 | long_dII-dIII-pack1 | VRVYQKVLTFRRSYAYIHTTYLLGSNTEYVAPPMWEIHHINSHSQCYSS |
| | dII-dIII-pack1: S367I, | YSRVIAGTVFVAYHRDSYENKTMQLMPDDYSNTHSTRYVTVKDQWHSRG |
| | T374F | STWLYRETCNLNCMVTITTARSKYPYHFFATSTGDVVDISPFYNGTNRN |
| | Cpack2-long: D679N, | ASYFGENADKFFIFPNYTIVSDEGRPNSALETHRLVAFLERADSVISWD |
| | L680E, E681N, R685Q, | IQDEKNVTCQLTFWEASERTIRSEAEDSYHFISAKMTAFFLSKKQEVNM |
| | E686L, Y690F, R693V, | SDSALDCVRDEAINKLQQIFNTSYNQTYEKYGNVSVFETTGGLVVFWQG |
| | V694L, K695D, K700A, | IKQKSLVELERLANRSSLNLTHNRTKRSTDGNNATHLSNMESVHNLVYA |
| | V701L, V702Q | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNVF |
| | | NENEIMQLENSFKQVLDYVEDALQ |
| SEQ ID | gB-Towne_Cpack3- | YRVCSMAQGTDLIRFERNIVCTSMKPINEDLDEGIMVVYKRNIVAHTEK |
| NO: 246 | short_dII-dIII-pack1 | VRVYQKVLTFRRSYAYIHTTYLLGSNTEYVAPPMWEIHHINSHSQCYSS |
| | dII-dIII-pack1: S367I, | YSRVIAGTVFVAYHRDSYENKIMQLMPDDYSNTHSTRYVTVKDQWHSRG |
| | T374F | STWLYRETCNLNCMVTITTARSKYPYHFFATSTGDVVDISPFYNGTNRN |
| | Cpack3-short: V677T, | ASYFGENADKFFIFPNYTIVSDEGRPNSALETHRLVAFLERADSVISWD |
| | F678N, D679N, L680E, | IQDEKNVICQLTFWEASERTIRSEAEDSYHFISAKMTAFFLSKKQEVNM |
| | E681N, E682S, R685Q, | SDSALDCVRDEAINKLQQIFNTSYNQTYEKYGNVSVFETTGGLVVFWQG |
| | E686L, N688E, Y690F, | IKQKSLVELERLANRSSLNLTHNRTKRSTDGNNATHLSNMESVHNLVYA |
| | R693V, V694L, K695D | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDFRVLELYSQKELRSSNTN |
| | | NENSIMQLFESFKQVLDYV |
| SEQ ID | gB-Towne_Cpack3- | YRVCSMAQGTDLIRFERNIVCTSMKPINEDLDEGIMVVYKRNIVAHTFK |
| NO: 247 | long_dII-dIII-pack1 | VRVYQKVLTFRRSYAYIHTTYLLGSNTEYVAPPMWEIHHINSHSQCYSS |
| | dII-dIII-pack1: S367I, | YSRVIAGTVFVAYHRDSYENKTMQLMPDDYSNTHSTRYVTVKDQWHSRG |
| | T374F | STWLYRETCNLNCMVTITTARSKYPYHFFATSTGDVVDISPFYNGTNRN |
| | Cpack3-long: V677T, | ASYFGENADKFFIFPNYTIVSDEGRENSALETHRIVAFLERADSVISWD |
| | F678N, D679N, L680E, | IQDEKNVICQLTFWEASERTIRSEAEDSYHFISAKMTAFFLSKKQEVNM |
| | E681N, E682S, R685Q, | SDSALDCVRDEAINKLQQIFNTSYNQTYEKYGNVSVFETTGGLVVFWQG |
| | E686L, N688E, Y690F | IKQKSIVELERLANRSSLNLTHNRTKRSTDGNNATHLSNMESVHNLVYA |
| | R693V, V694L, K695D, | QLQFTYDTLRGYINRALAQIAEAWCVDQRRTLEVFKELSKINPSAILSA |
| | D699K, K700A, V701L, | IYNKPIAARFMGDVLGLASCVTINQTSVKVERDMNVKESPGRCYSRPVV |
| | V702Q | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNTN |
| | | NENSIMQLFESFKQVLDYVEKALQ |
| SEQ ID | gB-Towne_Cpack4- | YRVCSMAQGTDLIRFERNIVCTSMKPINEDLDEGIMVVYKRNIVAHTEK |
| NO: 248 | short_dII-dIII-pack1 | VRVYQKVLTFRRSYAYIHTTYLLGSNTEYVAPPMWEIHHINSHSQCYSS |
| | dII-dIII-pack1: S367I, | YSRVIAGTVFVAYHRDSYENKTMQLMPDDYSNTHSTRYVTVKDQWHSRG |
| | T374F | STWLYRETCNLNCMVTITTARSKYPYHFFATSTGDVVDISPFYNGTNRN |
| | Cpack4-short: V677T, | ASYFGENADKFFIFPNYTIVSDEGRPNSALETHRLVAFLERADSVISWD |
| | F678N, D679H, L680E, | IQDEKNVTCQLTFWEASERTIRSEAEDSYHFISAKMTAFFLSKKQEVNM |
| | E681N, E682S, M684S, | SDSALDCVRDEAINKLQQIFNTSYNQTYEKYGNVSVFETTGGLVVFWQG |
| | R685Q, E686L, F687A, | IKQKSLVELERLANRSSLNLTHNRTKRSTDGNNATHLSNMESVHNLVYA |
| | N688E, Y690F, Q692S, | QLQFTYDTLRGYINRALAQIAEAWCVDQRRTLEVEKELSKINPSAILSA |
| | R693V, V694L, K695D, | IYNKPIAARFMGDVLGLASCVTINQTSVKVLRDMNVKESPGRCYSRPVV |
| | Y696R | IFNFANSSYVQYGQLGEDNEILLGNHRTEECQLPSLKIFIAGNSAYEYV |
| | | DYLFKRMIDLSSISTVDSMIALDIDPLENTDFRVLELYSQKELRSSNIN |
| | | HENSISQLAESFKSVLDRV |
| SEQ ID | gB-Towne_Cpack4- | YRVCSMAQGTDLIRFERNIVCTSMKPINEDLDEGIMVVYKRNIVAHTEK |
| NO: 249 | long_dII-dIII-pack1 | VRVYQKVLTERRSYAYIHTTYLLGSNTEYVAPPMWEIHHINSHSQCYSS |
| | dII-dIII-pack1: S367I, | YSRVIAGIVEVAYHRDSYENKTMQLMPDDYSNTHSTRYVTVKDQWHSRG |
| | T374F | STWLYRETCNLNCMVTITTARSKYPYHFFATSTGDVVDISPFYNGTNRN |
| | Cpack4-long: V677T, | ASYFGENADKFFIFPNYTIVSDEGRPNSALETHRLVAFLERADSVISWD |
| | F678N, D679H, L680E, | IQDEKNVTCQLTFWEASERTIRSEAEDSYHFISAKMTAFFLSKKQEVNM |
| | E681N, E682S, M684S, | SDSALDCVRDEAINKLQQIFNTSYNQTYEKYGNVSVFETTGGLVVFWQG |
| | R685Q, E686L, F687A, | IKQKSIVELERLANRSSLNLTHNRTKRSTDGNNATHLSNMESVHNLVYA |
| | N688E, Y690F, Q692S, | QLQFTYDTERGYINRALAQIAEAWCVDQRRTLEVFKELSKINPSAILSA |
| | R693V, V694L, K695D, | IYNKPIAARFMGDVLGLASCVTINQTSVKVLRDMNVKESPGRCYSRPVV |
| | Y696R, D699K, K700A, | IFNFANSSYVQYGQLGEDNEILLGNHRTEECQLPSLKIFIAGNSAYEYV |
| | V701L, V702E | DYLFKRMIDLSSISTVDSMIALDIDPLENTDERVLELYSQKELRSSNTN |
| | | HENSISQLAESFKSVLDRVEKALE |
| SEQ ID | gB-Towne_Cpack1- | YRVCSMAQGTDLIRFERNIVCTSMKPINEDLDEGIMVVYKRNIVAHTEK |
| NO: 250 | short_dI-dIV-dV-pack1 | VRVYQKVLTFRRSYAYINTTYLLGSNTEYVAPPMWEIHHINSHSQCYSS |
| | Cpack1-short: E686L, | YSRVIAGTVFVAYHRDSYENKTMQLMPDDESNTHSTRYVTVKDQWHSRG |
| | Y690F, R693V, V694L | STWLYRETCNLNCMVTITTARSKYPYHFFATSIGDVVDISPFYNGTNRN |
| | dI-dIV-dV-pack1: | ASYFGENADKFFIFPNYTIVSDFGRPNSALETHRLVAFLERADSVISWD |
| | Y218F, Q591F, S668A | IQDEKNVTCQLTFWEASERTIRSEAEDSYHFSSAKMTATFLSKKQEVNM |
| | | SDSALDCVRDEAINKLQQIFNTSYNQTYEKYGNVSVFETTGGLVVFWQG |
| | | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSYVFYGQLGEDNEILLGNHRTEECQLPSLKIFIAGNSAYEYV |
| | | DYLFKRMIDLSSISTVDSMIALDIDPLENTDERVLELYAQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTFK |
| NO: 251 | long_dI-dIV-dV-pack1 | VRVYQKVLIFRRSYAYIHTTYLLGSNTEYVAPPMWEIHHINSHSQCYSS |
| | Cpack1-long: E686L, | YSRVIAGTVFVAYHRDSYENKTMQLMPDDESNTHSTRYVTVKDQWHSRG |
| | Y690F, R693V, V694L, | STWLYRETCNLNCMVTITTARSKYPYHFFATSTGDVVDISPFYNGTNRN |
| | K700A, V702Q | ASYFGENADKFFIFPNYTIVSDFGRPNSALETHRLVAFLERADSVISWD |
| | dI-dIV-dV-pack1: | IQDEKNVTCQLTEWEASERTIRSEAEDSYHFSSAKMTATFLSKKQEVNM |
| | Y218F, Q591F, S668A | SDSALDCVRDEAINKLQQIFNTSYNQTYEKYGNVSVFETTGGLVVFWQG |
| | | IKQKSLVELERLANRSSLNLTANRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSYVFYGQLGEDNEILLGNHRTEECQLPSLKIFIAGNSAYEYV |
| | | DYLFKRMIDLSSISTVDSMIALDIDPLENTDERVLELYAQKELRSSNVE |
| | | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTFK |
| NO: 252 | short_Disulf1 | VRVYQKVLTFRRSYAYIHTTYLLGSNTEYVAPPMWEIHHINSHSQCYSS |
| | Cpack1-short: E686L, | YSRVIAGIVEVAYHRDSYENKTMQLMPDCYSNTHSTRYVTVKDQWHSRG |
| | Y690F, R693V, V694L | STWLYRETCNLNCMVTITTARSKYPYHFFATSTGDVVDISPFYNGTNRN |
| | Disulf1: D217C, S587C | ASYFGENADKFFIFPNYTIVSDFGRPNSALETHRLVAFLERADSVISWD |
| | | IQDEKNVTCQLTFWEASERTIRSEAEDSYHFSSAKMTATFLSKKQEVNM |
| | | SDSALDCVRDEAINKLQQIFNTSYNQTYEKYGNVSVFETTGGLVVFWQG |
| | | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANCSYVQYGQLGEDNEILLGNHRTEECQLPSLKIFIAGNSAYEYV |
| | | DYLFKRMIDLSSISTVDSMIALDIDPLENTDERVLELYSQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTFK |
| NO: 253 | long_Disulf1 | VRVYQKVLTFRRSYAYIHTTYLLGSNTEYVAPPMWEIHHINSHSQCYSS |
| | Cpack1-long: E686L, | YSRVIAGTVFVAYHRDSYENKTMQLMPDCYSNTHSTRYVTVKDQWHSRG |
| | Y690F, R693V, V694L, | STWLYRETCNLNCMVTITTARSKYPYHFFATSTGDVVDISPFYNGTNRN |
| | K700A, V702Q | ASYFGENADKFFIFPNYTIVSDEGRPNSALETHRLVAFLERADSVISWD |
| | Disulf1: D217C, S587C | IQDEKNVICQLTFWEASERTIRSEAEDSYHFSSAKMTATFLSKKQEVNM |
| | | SDSALDCVRDEAINKLQQIFNTSYNQTYEKYGNVSVFETTGGLVVFWQG |
| | | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANCSYVQYGQLGEDNEILLGNHRTEECQLPSLKIFIAGNSAYEYV |
| | | DYLFKRMIDLSSISTVDSMIALDIDPLENTDERVLELYSQKELRSSNVF |
| | | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTEK |
| NO: 254 | short Disulf2 | VRVYQKVLIFRRSYAYIHTTYLLGSNTEYVAPPMWEIHHINSHSQCYSS |
| | Cpack1-short: E686L, | YSRVIAGTVFVAYHRDSYENKIMQLMPDCYSNTHSTRYVTVKDQWHSRG |
| | Y690F, R693V, V694L | STWLYRETCNLNCMVTITTARSKYPYHFFATSTGDVVDISPFYNGTNRN |
| | Disulf2: D217C, Y589C | ASYFGENADKFFIFPNYTIVSDEGRPNSALETHRLVAFLERADSVISWD |
| | | IQDEKNVTCQLTFWEASERTIRSEAEDSYHFSSAKMTATFLSKKQEVNM |
| | | SDSALDCVRDEAINKLQQIFNTSYNQTYEKYGNVSVFETTGGLVVFWQG |
| | | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSCVQYGQLGEDNEILLGNHRTEECQLPSLKIFIAGNSAYEYV |
| | | DYLFKRMIDLSSISTVDSMIALDIDPLENTDERVLELYSQKELRSSNVE |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTEK |
| NO: 255 | long_Disulf2 | VRVYQKVLTFRRSYAYIHTTYLLGSNTEYVAPPMWEIHHINSHSQCYSS |
| | Cpack1-long: E686L, | YSRVIAGTVFVAYHRDSYENKTMQLMPDCYSNTHSTRYVTVKDQWHSRG |
| | Y690F, R693V, V694L, | STWLYRETCNLNCMVTITTARSKYPYHFFATSTGDVVDISPFYNGINRN |
| | K700A, V702Q | ASYFGENADKFFIFPNYTIVSDEGRPNSALETHRLVAFLERADSVISWD |
| | Disulf2: D217C, Y589C | IQDEKNVICQLTFWEASERTIRSEAEDSYHFSSAKMTATFLSKKQEVNM |
| | | SDSALDCVRDEAINKLQQIFNTSYNQTYEKYGNVSVFETTGGLVVFWQG |
| | | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSCVQYGQLGEDNEILLGNHRTEECQLPSLKIFIAGNSAYEYV |
| | | DYLFKRMIDLSSISTVDSMIALDIDPLENTDERVLELYSQKELRSSNVE |
| | | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTEK |
| NO: 256 | short_dI-dIV-dV- | VRVYQKVLTFRRSYAYIHTTYLLGSNTEYVAPPMWEIHHINSHSQCYSS |
| | pack1_Disulf1 | YSRVIAGTVFVAYHRDSYENKTMQLMPDCFSNTHSTRYVTVKDQWHSRG |
| | Cpack1-short: E686L, | STWLYRETCNLNCMVTITTARSKYPYHFFATSTGDVVDISPFYNGTNRN |
| | Y690F, R693V, V694L | ASYFGENADKFFIFPNYTIVSDEGRENSALETHRLVAFLERADSVISWD |
| | dI-dIV-dV-pack1: | IQDEKNVICQLTFWEASERTIRSEAEDSYHFSSAKMTATFLSKKQEVNM |
| | Y218F, Q591F, S668A | SDSALDCVRDEAINKLQQIFNTSYNQTYEKYGNVSVFETTGGLVVFWQG |
| | Disulf1: D217C, S587C | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANCSYVFYGQLGEDNEILLGNHRTEECQLPSLKIFIAGNSAYEYV |
| | | DYLFKRMIDLSSISTVDSMIALDIDPLENTDERVLELYAQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTEK |
| NO: 257 | long_dI-dIV-dV- | VRVYQKVLTFRRSYAYIHTTYLLGSNTEYVAPPMWEIHHINSHSQCYSS |
| | pack1_Disulf1 | YSRVIAGTVFVAYHRDSYENKIMQLMPDCFSNTHSTRYVTVKDQWHSRG |
| | Cpack1-long: S686L, | STWLYRETCNLNCMVTITTARSKYPYHFFATSTGDVVDISPFYNGTNRN |
| | Y690F, R693V, V694L, | ASYFGENADKFFIFPNYTIVSDEGRPNSALETHRLVAFLERADSVISWD |
| | K700A, V702Q | IQDEKNVTCQLTEWEASERTIRSEAEDSYHFSSAKMTATFLSKKQEVNM |
| | dI-dIV-dV-pack1: | SDSALDCVRDEAINKLQQIFNTSYNQTYEKYGNVSVFETTGGLVVFWQG |
| | Y218F, Q591F, S668A | IKQKSLVELERLANRSSLNLTHNRTKRSTDGNNATHLSNMESVHNLVYA |
| | Disulf1: D217C, S587C | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANCSYVFYGQLGEDNEILLGNHRTEECQLPSLKIFIAGNSAYEYV |
| | | DYLFKRMIDLSSISTVDSMIALDIDPLENTDFRVLELYAQKELRSSNVF |
| | | DLEEIMRLFNSFKQVLKYVEDAVQ |
| SEQ ID | gB-Towne_Cpack1- | YRVCSMAQGTDLIRFERNIVCTSMKPINEDLDEGIMVVYKRNIVAHTEK |
| NO: 258 | short_dI-dIV-dV- | VRVYQKVLTFRRSYAYIHTTYLLGSNTEYVAPPMWEIHHINSHSQCYSS |
| | pack1_Disulf2 | YSRVIAGTVFVAYHRDSYENKTMQLMPDCFSNTHSTRYVTVKDQWHSRG |
| | Cpack1-short: E686L, | STWLYRETCNLNCMVTITTARSKYPYHFFATSTGDVVDISPFYNGTNRN |
| | Y690F, R693V, V694L | ASYFGENADKFFIFPNYTIVSDEGRPNSALETHRLVAFLERADSVISWD |
| | dI-dIV-dV-pack1: | IQDEKNVICQLTFWEASERTIRSEAEDSYHFSSAKMTATFLSKKQEVNM |
| | Y218F, Q591F, S668A | SDSALDCVRDEAINKLQQIFNTSYNQTYEKYGNVSVFETTGGLVVFWQG |
| | Disulf2: D217C, Y589C | IKQKSLVELERLANRSSLNLTHNRTKRSTDGNNATHLSNMESVHNLVYA |
| | | QLQFTYDTLRGYINPALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSCVFYGQLGEDNEILLGNHRTEECQLPSLKIFIAGNSAYEYV |
| | | DYLFKRMIDLSSISTVDSMIALDIDPLENTDERVLELYAQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTEK |
| NO: 259 | long_dI-dIV-dV- | VRVYQKVLTFRRSYAYIHTTYLLGSNTEYVAPPMWEIHHINSHSQCYSS |
| | pack1_Disulf2 | YSRVIAGTVFVAYHRDSYENKTMQLMPDCFSNTHSTRYVTVKDQWHSRG |
| | Cpack1-long: E686L, | STWLYRETCNLNCMVTITTARSKYPYHFFATSTGDVVDISPFYNGTNRN |
| | Y690F, R693V, V694L, | ASYFGENADKFFIFPNYTIVSDEGRPNSALETARLVAFLERADSVISWD |
| | K700A, V702Q | IQDEKNVTCQLTFWEASERTIRSEAEDSYHFSSAKMTATFLSKKQEVNM |
| | dI-dIV-dV-pack1: | SDSALDCVRDEAINKLQQIFNTSYNQTYEKYGNVSVFETTGGLVVFWQG |
| | Y218F, Q591F, S668A | IKQKSLVELERLANRSSLNLTHNRTKRSTDGNNATHLSNMESVHNLVYA |
| | Disulf2: D217C, Y589C | QLQFTYDTERGYINRALAQIAEAWCVDQRRTLEVEKELSKINPSAILSA |
| | | IYNKPIAARFMGDVLGLASCVTINQTSVKVLRDMNVKESPGRCYSRPVV |
| | | IFNFANSSCVFYGQLGEDNEILLGNHRTEECQLPSLKIFIAGNSAYEYV |
| | | DYLFKRMIDLSSISTVDSMIALDIDPLENTDFRVLELYAQKELRSSNVF |
| | | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTFK |
| NO: 260 | short_dI-dIV-dV- | VRVYQKVLTFRRSYAYIHTTYLLGSNTTYVAPPMWEIHHINSHSQCYSS |
| | pack1_N165glyc | YSRVIAGTVFVAYHRDSYENKTMQLMPDDESNTHSTRYVTVKDQWHSRG |
| | Cpack1-short: E686L, | STWLYRETCNLNCMVTITTARSKYPYHFFATSTGDVVDISPFYNGTNRN |
| | Y690F, R693V, V694L | ASYFGENADKFFIFPNYTIVSDEGRPNSALETHRLVAFLERADSVISWD |
| | dI-dIV-dV-pack1: | IQDEKNVTCQLTFWEASERTIRSEAEDSYHFSSAKMTATFLSKKQEVNM |
| | Y218F, Q591F, S668A | SDSALDCVRDEAINKLQQIFNTSYNQTYEKYGNVSVFETTGGLVVFWQG |
| | N165glyc: E167T | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSYVFYGQLGEDNEILLGNHRTEECQLPSLKIFIAGNSAYEYV |
| | | DYLFKRMIDLSSISTVDSMIALDIDPLENTDERVLELYAQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTEK |
| NO: 261 | long_dI-dIV-dV- | VRVYQKVLTFRRSYAYIHTTYLLGSNTTYVAPPMWEIHHINSHSQCYSS |
| | pack1_N165glyc | YSRVIAGTVFVAYHRDSYENKTMQLMPDDESNTHSTRYVTVKDQWHSRG |
| | Cpack1-long: E686L, | STWLYRETCNLNCMVTITTARSKYPYHFFATSTGDVVDISPFYNGTNRN |
| | Y690F, R693V, V694L, | ASYFGENADKFFIFPNYTIVSDEGRPNSALETHRLVAFLERADSVISWD |
| | K700A, V702Q | IQDEKNVICQLTFWEASERTIRSEAEDSYHFSSAKMTATFLSKKQEVNM |
| | dI-dIV-dV-pack1: | SDSALDCVRDEAINKLQQIFNTSYNQTYEKYGNVSVFETTGGLVVFWQG |
| | Y218F, Q591F, S668A | IKQKSLVELERLANRSSLNLTHNRTKRSTDGNNATHLSNMESVHNLVYA |
| | N165glyc: E167T | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSYVFYGQLGEDNEILLGNHRTEECQLPSLKIFIAGNSAYEYV |
| | | DYLFKRMIDLSSISTVDSMIALDIDPLENTDFRVLELYAQKELRSSNVF |
| | | DLEEIMRLENSFKQVLKYVEDAVQ |
| SEQ ID | GB-Towne_Cpack1- | YRVCSMAQGTDLIRFERNIVCISMKPINEDLDEGIMVVYKRNIVAHTFK |
| NO: 262 | short_dII-dIII- | VRVYQKVLTFRRSYAYIHTTYLLGSNTEYVAPPMWEIHHINSHSQCYSS |
| | pack1_dI-dIV-dV-pack1 | YSRVIAGTVFVAYHRDSYENKTMQLMPDDESNTHSTRYVTVKDQWHSRG |
| | dII-dIII-pack1: S367I, | STWLYRETCNLNCMVTITTARSKYPYHFFATSTGDVVDISPFYNGTNRN |
| | T374F | ASYFGENADKFFIFPNYTIVSDFGRPNSALETHRLVAFLERADSVISWD |
| | Cpack1-short: E686L, | IQDEKNVTCQLTFWEASERTIRSEAEDSYHFISAKMTAFFLSKKQEVNM |
| | Y690F, R693V, V694L | SDSALDCVRDEAINKLQQIFNTSYNQTYEKYGNVSVFETTGGLVVFWQG |
| | dI-dIV-dV-pack1: | IKQKSLVELERLANRSSLNLTANRTKRSTDGNNATHLSNMESVHNLVYA |
| | Y218F, Q591F, S668A | QLQFTYDTLRGYINRALAQIAEAWCVDQRRTLEVEKELSKINPSAILSA |
| | | IYNKPIAARFMGDVLGLASCVTINQTSVKVLRDMNVKESPGRCYSRPVV |
| | | IFNFANSSYVFYGQLGEDNEILLGNHRTEECQLPSLKIFIAGNSAYEYV |
| | | DYLFKRMIDLSSISTVDSMIALDIDPLENTDERVLELYAQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTEK |
| NO: 263 | long_dII-dIII- | VRVYQKVLTFRRSYAYIHTTYLLGSNTEYVAPPMWEIHHINSHSQCYSS |
| | pack1_dI-dIV-dV-pack1 | YSRVIAGTVFVAYHRDSYENKTMQLMPDDESNTHSTRYVTVKDQWHSRG |
| | dII-dIII-pack1: S367I, | STWLYRETCNLNCMVTITTARSKYPYHFFATSTGDVVDISPFYNGINRN |
| | T374F | ASYFGENADKFFIFPNYTIVSDFGRPNSALETHRLVAFLERADSVISWD |
| | Cpack1-long: E686L, | IQDEKNVICQLTFWEASERTIRSEAEDSYHFISAKMTAFELSKKQEVNM |
| | Y690F, R693V, V694L, | SDSALDCVRDEAINKLQQIFNTSYNQTYEKYGNVSVFETTGGLVVFWQG |
| | K700A, V702Q | IKQKSLVELERLANRSSLNLTHNRTKRSTDGNNATHLSNMESVHNLVYA |
| | dI-dIV-dV-pack1: | QLQFTYDTLRGYINRALAQIAEAWCVDQRRTLEVFKELSKINPSAILSA |
| | Y218F, Q591F, S668A | IYNKPIAARFMGDVLGLASCVTINQTSVKVLRDMNVKESPGRCYSRPVV |
| | | IFNFANSSYVFYGQLGEDNEILLGNHRTEECQLPSLKIFIAGNSAYEYV |
| | | DYLFKRMIDLSSISTVDSMIALDIDPLENTDERVLELYAQKELRSSNVE |
| | | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTFK |
| NO: 264 | short_dII-dIII- | VRVYQKVLTFRRSYAYIHTTYLLGSNTEYVAPPMWEIHHINSHSQCYSS |
| | pack1_Disulf1 | YSRVIAGTVFVAYHRDSYENKTMQLMPDCYSNTHSTRYVTVKDQWHSRG |
| | dII-dIII-pack1: S367I, | STWLYRETCNLNCMVTITTARSKYPYHFFATSTGDVVDISPFYNGTNRN |
| | T374F | ASYFGENADKFFIFPNYTIVSDFGRENSALETHRLVAFLERADSVISWD |
| | Cpack1-short: E686L, | IQDEKNVICQLTFWEASERTIRSEAEDSYHFISAKMTAFFLSKKQEVNM |
| | Y690F, R693V, V694L | SDSALDCVRDEAINKLQQIFNTSYNQTYEKYGNVSVFETTGGLVVFWQG |
| | Disulf1: D217C, S587C | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANCSYVQYGQLGEDNEILLGNHRTEECQLPSLKIFIAGNSAYEYV |
| | | DYLFKRMIDLSSISTVDSMIALDIDPLENTDFRVLELYSQKELRSSNVE |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTEK |
| NO: 265 | long_dII-dIII- | VRVYQKVLTFRRSYAYIHTTYLLGSNTEYVAPPMWEIHHINSHSQCYSS |
| | pack1_Disulf1 | YSRVIAGTVFVAYHRDSYENKTMQLMPDCYSNTHSTRYVTVKDQWHSRG |
| | dII-dIII-pack1: S367I, | STWLYRETCNLNCMVTITTARSKYPYHFFATSTGDVVDISPFYNGTNRN |
| | T374F | ASYFGENADKFFIFPNYTIVSDFGRPNSALETHRLVAFLERADSVISWD |
| | Cpack1-long: E686L, | IQDEKNVICQLTEWEASERTIRSEAEDSYHFISAKMTAFFLSKKQEVNM |
| | Y690F, R693V, V694L, | SDSALDCVRDEAINKLQQIFNTSYNQTYEKYGNVSVFETTGGLVVEWQG |
| | K700A, V702Q | IKQKSLVELERLANRSSLNLTANRIKRSTDGNNATHLSNMESVHNLVYA |
| | Disulf1: D217C, S587C | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANCSYVQYGQLGEDNEILLGNHRTEECQLPSLKIFIAGNSAYEYV |
| | | DYLFKRMIDLSSISTVDSMIALDIDPLENTDERVLELYSQKELRSSNVE |
| | | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTEK |
| NO: 266 | short_dII-dIII- | VRVYQKVLTFRRSYAYIHTTYLLGSNTEYVAPPMWEIHHINSHSQCYSS |
| | pack1_Disulf2 | YSRVIAGTVFVAYHRDSYENKTMQLMPDCYSNTHSTRYVTVKDQWHSRG |
| | dII-dIII-pack1: S367I, | STWLYRETCNLNCMVTITTARSKYPYHFFATSTGDVVDISPFYNGTNRN |
| | T374F | ASYFGENADKFFIFPNYTIVSDEGRPNSALETHRLVAFLERADSVISWD |
| | Cpack1-short: E686L, | IQDEKNVTCQLTFWEASERTIRSEAEDSYHFISAKMTAFELSKKQEVNM |
| | Y690F, R693V, V694L | SDSALDCVRDEAINKLQQIFNTSYNQTYEKYGNVSVFETTGGLVVFWQG |
| | Disulf2: D217C, Y589C | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSCVQYGQLGEDNEILLGNHRTEECQLPSLKIFIAGNSAYEYV |
| | | DYLFKRMIDLSSISTVDSMIALDIDPLENTDERVLELYSQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTFK |
| NO: 267 | long_dII-dIII- | VRVYQKVLTFRRSYAYIHTTYLLGSNTEYVAPPMWEIHHINSHSQCYSS |
| | pack1_Disulf2 | YSRVIAGTVFVAYHRDSYENKTMQLMPDCYSNTHSTRYVTVKDQWHSRG |
| | dII-dIII-pack1: S367I, | STWLYRETCNLNCMVTITTARSKYPYHFFATSTGDVVDISPFYNGTNRN |
| | T374F | ASYFGENADKFFIFPNYTIVSDFGRPNSALETHRLVAFLERADSVISWD |
| | Cpack1-long: E686L, | IQDEKNVICQLTEWEASERTIRSEAEDSYHFISAKMTAFFLSKKQEVNM |
| | Y690F, R693V, V694L, | SDSALDCVRDEAINKLQQIFNTSYNQTYEKYGNVSVFETTGGLVVFWQG |
| | K700A, V702Q | IKQKSLVELERLANRSSLNLTHNRTKRSTDGNNATHLSNMESVHNLVYA |
| | Disulf2: D217C, Y589C | QLQFTYDTERGYINRALAQIAEAWCVDQRRTLEVFKELSKINPSAILSA |
| | | IYNKPIAARFMGDVLGLASCVTINQTSVKVLRDMNVKESPGRCYSRPVV |
| | | IFNFANSSCVQYGQLGEDNEILLGNHRTEECQLPSLKIFIAGNSAYEYV |
| | | DYLFKRMIDLSSISTVDSMIALDIDPLENTDERVLELYSQKELRSSNVE |
| | | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTFK |
| NO: 268 | short_dII-dIII- | VRVYQKVLTFRRSYAYIHTTYLLGSNTEYVAPPMWEIHHINSHSQCYSS |
| | pack1_dI-dIV-dV- | YSRVIAGTVFVAYHRDSYENKIMQLMPDCFSNTHSTRYVTVKDQWHSRG |
| | pack1_Disulf1 | STWLYRETCNLNCMVTITTARSKYPYHFFATSTGDVVDISPFYNGTNRN |
| | dII-dIII-pack1: S367I, | ASYFGENADKFFIFPNYTIVSDFGRPNSALETHRLVAFLERADSVISWD |
| | T374F | IQDEKNVTCQLTFWEASERTIRSEAEDSYHFISAKMTAFFLSKKQEVNM |
| | Cpack1-short: E686L, | SDSALDCVRDEAINKLQQIFNTSYNQTYEKYGNVSVFETTGGLVVFWQG |
| | Y690F, R693V, V694L | IKQKSIVELERLANRSSLNLTHNRTKRSTDGNNATHLSNMESVHNLVYA |
| | dI-dIV-dV-pack1: | QLQFTYDTLRGYINRALAQIAEAWCVDQRRTLEVFKELSKINPSAILSA |
| | Y218F, Q591F, S668A | IYNKPIAARFMGDVLGLASCVTINQTSVKVERDMNVKESPGRCYSRPVV |
| | Disulf1: D217C, S587C | IFNFANCSYVFYGQLGEDNEILLGNHRTEECQLPSLKIFIAGNSAYEYV |
| | | DYLFKRMIDLSSISTVDSMIALDIDPLENTDERVLELYAQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTEK |
| NO: 269 | long_dII-dIII- | VRVYQKVLIFRRSYAYIHTTYLLGSNTEYVAPPMWEIHHINSHSQCYSS |
| | pack1_dI-dIV-dV- | YSRVIAGTVFVAYHRDSYENKIMQLMPDCFSNTHSTRYVTVKDQWHSRG |
| | pack1_Disulf1 | STWLYRETCNLNCMVTITTARSKYPYHFFATSTGDVVDISPFYNGTNRN |
| | dII-dIII-pack1: S367I, | ASYFGENADKFFIFPNYTIVSDFGRPNSALETHRLVAFLERADSVISWD |
| | T374F | IQDEKNVTCQLTFWEASERTIRSEAEDSYHFISAKMTAFFLSKKQEVNM |
| | Cpack1-long: E686L, | SDSALDCVRDEAINKLQQIFNTSYNQTYEKYGNVSVFETTGGLVVEWQG |
| | Y690F, R693V, V694L, | IKQKSLVELERLANRSSLNLTHNRTKRSTDGNNATHLSNMESVHNLVYA |
| | K700A, V702Q | QLQFTYDTLRGYINRALAQIAEAWCVDQRRTLEVFKELSKINPSAILSA |
| | dI-dIV-dV-pack1: | IYNKPIAARFMGDVLGLASCVTINQTSVKVLRDMNVKESPGRCYSRPVV |
| | Y218F, Q591F, S668A | IFNFANCSYVFYGQLGEDNEILLGNHRTEECQLPSLKIFIAGNSAYEYV |
| | Disulf1: D217C, S587C | DYLFKRMIDLSSISTVDSMIALDIDPLENTDERVLELYAQKELRSSNVE |
| | | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTEK |
| NO: 270 | short_dII-dIII- | VRVYQKVLTFRRSYAYINTTYLLGSNTEYVAPPMWEIHHINSHSQCYSS |
| | pack1_dI-dIV-dV- | YSRVIAGTVFVAYHRDSYENKTMQLMPDCFSNTHSTRYVTVKDQWHSRG |
| | pack1_Disulf2 | STWLYRETCNLNCMVTITTARSKYPYHFFATSTGDVVDISPFYNGTNRN |
| | dII-dIII-pack1: S367I, | ASYFGENADKFFIFPNYTIVSDEGRENSALETHRLVAFLERADSVISWD |
| | T374F | IQDEKNVTCQLTFWEASERTIRSEAEDSYHFISAKMTAFFLSKKQEVNM |
| | Cpack1-short: E686L, | SDSALDCVRDEAINKLQQIFNTSYNQTYEKYGNVSVFETTGGLVVFWQG |
| | Y690F, R693V, V694L | IKQKSLVELERLANRSSLNLTHNRTKRSTDGNNATHLSNMESVHNLVYA |
| | dI-dIV-dV-pack1: | QLQFTYDTLRGYINRALAQIAEAWCVDQRRTLEVFKELSKINPSAILSA |
| | Y218F, Q591F, S668A | IYNKPIAARFMGDVLGLASCVTINQTSVKVLRDMNVKESPGRCYSRPVV |
| | Disulf2: D217C, Y589C | IFNFANSSCVFYGQLGEDNEILLGNHRTEECQLPSLKIFIAGNSAYEYV |
| | | DYLFKRMIDLSSISTVDSMIALDIDPLENTDERVLELYAQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTEK |
| NO: 271 | long_dII-dIII- | VRVYQKVLTFRRSYAYIHTTYLLGSNTEYVAPPMWEIHHINSHSQCYSS |
| | pack1_dI-dIV-dV- | YSRVIAGTVFVAYHRDSYENKIMQLMPDCFSNTHSTRYVTVKDQWHSRG |
| | pack1_Disulf2 | STWLYRETCNLNCMVTITTARSKYPYHFFATSTGDVVDISPFYNGINRN |
| | dII-dIII-pack1: S367I, | ASYFGENADKFFIFPNYTIVSDEGRPNSALETHRLVAFLERADSVISWD |
| | T374F | IQDEKNVTCQLTFWEASERTIRSEAEDSYHFISAKMTAFFLSKKQEVNM |
| | Cpack1-long: E686L, | SDSALDCVRDEAINKLQQIFNTSYNQTYEKYGNVSVFETTGGLVVFWQG |
| | Y690F, R693V, V694L, | IKQKSLVELERLANRSSLNLTHNRTKRSTDGNNATHLSNMESVHNLVYA |
| | K700A, V702Q | QLQFTYDTLRGYINRALAQIAEAWCVDQRRTLEVFKELSKINPSAILSA |
| | dI-dIV-dV-pack1: | IYNKPIAARFMGDVLGLASCVTINQTSVKVLRDMNVKESPGRCYSRPVV |
| | Y218F, Q591F, S668A | IFNFANSSCVFYGQLGEDNEILLGNHRTEECQLPSLKIFIAGNSAYEYV |
| | Disulf2: D217C, Y589C | DYLFKRMIDLSSISTVDSMIALDIDPLENTDERVLELYAQKELRSSNVF |
| | | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTFK |
| NO: 272 | short_dII-dIII- | VRVYQKVLTFRRSYAYIHTTYLLGSNTTYVAPPMWEIHHINSHSQCYSS |
| | pack1_dI-dIV-dV- | YSRVIAGTVFVAYHRDSYENKTMQLMPDDFSNTHSTRYVTVKDQWHSRG |
| | pack1_N165glyc | STWLYRETCNLNCMVTITTARSKYPYHFFATSTGDVVDISPFYNGTNRN |
| | dII-dIII-pack1: S367I, | ASYFGENADKFFIFPNYTIVSDFGRPNSALETHRLVAFLERADSVISWD |
| | T374F | IQDEKNVTCQLTEWEASERTIRSEAEDSYHFISAKMTAFFLSKKQEVNM |
| | Cpack1-short: E686L, | SDSALDCVRDEAINKLQQIFNTSYNQTYEKYGNVSVFETTGGLVVFWQG |
| | Y690F, R693V, V694L | IKQKSLVELERLANRSSLNLTHNRTKRSTDGNNATHLSNMESVHNLVYA |
| | dI-dIV-dV-pack1: | QLQFTYDTLRGYINRALAQIAEAWCVDQRRTLEVFKELSKINPSAILSA |
| | Y218F, Q591F, S668A | IYNKPIAARFMGDVLGLASCVTINQTSVKVLRDMNVKESPGRCYSRPVV |
| | N165glyc: E167T | IFNFANSSYVFYGQLGEDNEILLGNHRTEECQLPSLKIFIAGNSAYEYV |
| | | DYLFKRMIDLSSISTVDSMIALDIDPLENTDERVLELYAQKELRSSNVE |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTFK |
| NO: 273 | long_dII-dIII- | VRVYQKVLTFRRSYAYIHTTYLLGSNITYVAPPMWEIHHINSHSQCYSS |
| | pack1_dI-dIV-dV- | YSRVIAGTVFVAYHRDSYENKTMQLMPDDFSNTHSTRYVTVKDQWHSRG |
| | pack1_N165glyc | STWLYRETCNLNCMVTITTARSKYPYHFFATSTGDVVDISPFYNGINRN |
| | dII-dIII-pack1: S367I, | ASYFGENADKFFIFPNYTIVSDFGRPNSALETHRLVAFLERADSVISWD |
| | T374F | IQDEKNVICQLTFWEASERTIRSEAEDSYHFISAKMTAFFLSKKQEVNM |
| | Cpack1-long: E686L, | SDSALDCVRDEAINKLQQIFNTSYNQTYEKYGNVSVFETTGGLVVFWQG |
| | Y690F, R693V, V694L, | IKQKSLVELERLANRSSLNLTHNRTKRSTDGNNATHLSNMESVHNLVYA |
| | K700A, V702Q | QLQFTYDTLRGYINRALAQIAEAWCVDQRRTLEVFKELSKINPSAILSA |
| | dI-dIV-dV-pack1: | IYNKPIAARFMGDVLGLASCVTINQTSVKVLRDMNVKESPGRCYSRPVV |
| | Y218F, Q591F, S668A | IFNFANSSYVFYGQLGEDNEILLGNHRTEECQLPSLKIFIAGNSAYEYV |
| | N165glyc: E167T | DYLFKRMIDLSSISTVDSMIALDIDPLENTDERVLELYAQKELRSSNVF |
| | gB-Towne_Cpack1- | DLEEIMRLENSFKQVLKYVEDAVQ |
| SEQ ID | short_dI-dIV-dV-pack2 | YRVCSMAQGTDLIRFERNIVCTSMKPINEDLDEGIMVVYKRNIVAHTEK |
| NO: 274 | Cpack1-short: E686L, | VRVYQKVLTFRRSYAYIHTTYLLGSNTEYVAPPMWEIHHINSHSQCYSS |
| | Y690F, R693V, V694L | YSRVIAGTVFVAYHRDSYENKTMQLMPDDYSSTHSTRYVTVKDQWHSRG |
| | dI-dIV-dV-pack2: | STWLYRETCNLNCMVTITTARSKYPYHFFATSTGDVVDISPFYNGTNRN |
| | N220S, V552L | ASYFGENADKFFIFPNYTIVSDEGRPNSALETHRLVAFLERADSVISWD |
| | | IQDEKNVICQLTFWEASERTIRSEAEDSYHFSSAKMTATFLSKKQEVNM |
| | | SDSALDCVRDEAINKLQQIFNTSYNQTYEKYGNVSVFETTGGLVVFWQG |
| | | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCLTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTEK |
| NO: 275 | long_dI-dIV-dV-pack2 | VRVYQKVLIFRRSYAYIHTTYLLGSNTEYVAPPMWEIHHINSHSQCYSS |
| | Cpack1-long: E686L, | YSRVIAGTVFVAYHRDSYENKTMQLMPDDYSSTHSTRYVTVKDQWHSRG |
| | Y690F, R693V, V694L, | STWLYRETCNLNCMVTITTARSKYPYHFFATSTGDVVDISPFYNGTNRN |
| | K700A, V702Q | ASYFGENADKFFIFPNYTIVSDFGRPNSALETHRLVAFLERADSVISWD |
| | dI-dIV-dV-pack2: | IQDEKNVICQLTFWEASERTIRSEAEDSYHFSSAKMTATFLSKKQEVNM |
| | N220S, V552L | SDSALDCVRDEAINKLQQIFNTSYNQTYEKYGNVSVFETTGGLVVFWQG |
| | | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCLTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNVF |
| | | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTEK |
| NO: 276 | short_dI-dIV-dV-pack3 | VRVYQKVLTFRRSYAYIHTTYLLGSNTEYVAPPMWEIHHINSHSQCYSS |
| | Cpack1-short: E686L, | YSRVIAGTVFVAYHRDSYENKTMQLMPDDESSTHSTRYVTVKDQWHSRG |
| | Y690F, R693V, V694L | STWLYRETCNLNCMVTITTARSKYPYHFFATSTGDVVDISPFYNGTNRN |
| | dI-dIV-dV-pack3: | ASYFGENADKFFIFPNYTIVSDEGRPNSALETHRLVAFLERADSVISWD |
| | Y218F, N220S, V552L, | IQDEKNVTCQLTFWEASERTIRSEAEDSYHFSSAKMTATFLSKKQEVNM |
| | Q591F, S668A | SDSALDCVRDEAINKLQQIFNTSYNQTYEKYGNVSVFETTGGLVVFWQG |
| | | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCLTINQTSVKVLRDMNVKESPGRCYSRPVV |
| | | IFNFANSSYVFYGQLGEDNEILLGNERTEECQLPSLKIFIAGNSAYEYV |
| | | DYLFKRMIDLSSISTVDSMIALDIDPLENTDERVLELYAQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTEK |
| NO: 277 | long_dI-dIV-dV-pack3 | VRVYQKVLTFRRSYAYIHTTYLLGSNTEYVAPPMWEIHHINSHSQCYSS |
| | Cpack1-long: E686L, | YSRVIAGTVFVAYHRDSYENKTMQLMPDDFSSTHSTRYVTVKDQWHSRG |
| | Y690F, R693V, V694L, | STWLYRETCNLNCMVTITTARSKYPYHFFATSTGDVVDISPFYNGTNRN |
| | K700A, V702Q | ASYFGENADKFFIFPNYTIVSDEGRPNSALETHRLVAFLERADSVISWD |
| | dI-dIV-dV-pack3: | IQDEKNVICQLTFWEASERTIRSEAEDSYHFSSAKMTATFLSKKQEVNM |
| | Y218F, N220S, V552L, | SDSALDCVRDEAINKLQQIFNTSYNQTYEKYGNVSVFETTGGLVVFWQG |
| | Q591F, S668A | IKQKSLVELERLANRSSLNLTHNRTKRSTDGNNATHLSNMESVHNLVYA |
| | | QLQFTYDTLRGYINPALAQIAEAWCVDQRRTLEVFKELSKINPSAILSA |
| | | IYNKPIAARFMGDVLGLASCLTINQTSVKVLRDMNVKESPGRCYSRPVV |
| | | IFNFANSSYVFYGQLGEDNEILLGNHRTEECQLPSLKIFIAGNSAYEYV |
| | | DYLFKRMIDLSSISTVDSMIALDIDPLENTDERVLELYAQKELRSSNVF |
| | | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTEK |
| NO: 278 | short_dII-dIII- | VRVYQKVLTFRRSYAYIHTTYLLGSNTEYVAPPMWEIHHINSHSQCYSS |
| | pack1_dI-dIV-dV-pack2 | YSRVIAGTVFVAYHRDSYENKTMQLMPDDYSSTHSTRYVTVKDQWHSRG |
| | dII-dIII-pack1: S367I, | STWLYRETCNLNCMVTITTARSKYPYHFFATSTGDVVDISPFYNGTNRN |
| | T374F | ASYFGENADKFFIFPNYTIVSDEGRPNSALETHRLVAFLERADSVISWD |
| | Cpack1-short: E686L, | IQDEKNVTCQLTFWEASERTIRSEAEDSYHFISAKMTAFFLSKKQEVNM |
| | Y690F, R693V, V694L | SDSALDCVRDEAINKLQQIFNTSYNQTYEKYGNVSVFETTGGLVVFWQG |
| | dI-dIV-dV-pack2: | IKQKSLVELERLANRSSLNLTHNRTKRSTDGNNATHLSNMESVHNLVYA |
| | N220S, V552L | QLQFTYDTERGYINRALAQIAEAWCVDQRRTLEVFKELSKINPSAILSA |
| | | IYNKPIAARFMGDVLGLASCLTINQTSVKVLRDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDFRVLELYSQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTEK |
| NO: 279 | long_dII-dIII- | VRVYQKVLTFRRSYAYIHTTYLLGSNTEYVAPPMWEIHHINSHSQCYSS |
| | pack1_dI-dIV-dV-pack2 | YSRVIAGTVFVAYHRDSYENKTMQLMPDDYSSTHSTRYVTVKDQWHSRG |
| | dII-dIII-pack1: S367I, | STWLYRETCNLNCMVTITTARSKYPYHFFATSTGDVVDISPFYNGTNRN |
| | T374F | ASYFGENADKFFIFPNYTIVSDEGRPNSALETHRLVAFLERADSVISWD |
| | Cpack1-long: E686L, | IQDEKNVTCQLIFWEASERTIRSEAEDSYHFISAKMTAFFLSKKQEVNM |
| | Y690F, R693V, V694L, | SDSALDCVRDEAINKLQQIFNTSYNQTYEKYGNVSVFETTGGLVVFWQG |
| | K700A, V702Q | IKQKSIVELERLANRSSLNLTHNRTKRSTDGNNATHLSNMESVHNLVYA |
| | dI-dIV-dV-pack2: | QLQFTYDTLRGYINRALAQIAEAWCVDQRRTLEVFKELSKINPSAILSA |
| | N220S, V552L | IYNKPIAARFMGDVIGLASCLTINQTSVKVERDMNVKESPGRCYSRPVV |
| | | IFNFANSSYVQYGQLGEDNEILLGNHRTEECQLPSLKIFIAGNSAYEYV |
| | | DYLFKRMIDLSSISTVDSMIALDIDPLENTDERVLELYSQKELRSSNVF |
| | | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTFK |
| NO: 280 | short_dII-dIII- | VRVYQKVLTFRRSYAYIHTTYLLGSNTEYVAPPMWEIHHINSHSQCYSS |
| | pack1_dI-dIV-dV-pack3 | YSRVIAGTVFVAYHRDSYENKTMQLMPDDESSTHSTRYVTVKDQWHSRG |
| | dII-dIII-pack1: S367I, | STWLYRETCNLNCMVTITTARSKYPYHFFATSTGDVVDISPFYNGINRN |
| | T374F | ASYFGENADKFFIFPNYTIVSDFGRPNSALETHRLVAFLERADSVISWD |
| | Cpack1-short: E686L, | IQDEKNVICQLTFWEASERTIRSEAEDSYHFISAKMTAFFLSKKQEVNM |
| | Y690F, R693V, V694L | SDSALDCVRDEAINKLQQIFNTSYNQTYEKYGNVSVFETTGGLVVFWQG |
| | dI-dIV-dV-pack3: | IKQKSLVELERLANRSSLNLTHNRTKRSTDGNNATHLSNMESVHNLVYA |
| | Y218F, N220S, V552L, | QLQFTYDTLRGYINRALAQIAEAWCVDQRRTLEVFKELSKINPSAILSA |
| | Q591F, S668A | IYNKPIAARFMGDVLGLASCLTINQTSVKVLRDMNVKESPGRCYSRPVV |
| | | IFNFANSSYVFYGQLGEDNEILLGNHRTEECQLPSLKIFIAGNSAYEYV |
| | | DYLFKRMIDLSSISTVDSMIALDIDPLENTDFRVLELYAQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTFK |
| NO: 281 | long_dII-dIII- | VRVYQKVLTFRRSYAYIHTTYLLGSNTEYVAPPMWEIHHINSHSQCYSS |
| | pack1_dI-dIV-dV-pack3 | YSRVIAGIVEVAYHRDSYENKTMQLMPDDESSTHSTRYVTVKDQWHSRG |
| | dII-dIII-pack1: S367I, | STWLYRETCNLNCMVTITTARSKYPYHFFATSTGDVVDISPFYNGTNRN |
| | T374F | ASYFGENADKFFIFPNYTIVSDEGRENSALETHRLVAFLERADSVISWD |
| | Cpack1-long: E686L, | IQDEKNVTCQLTFWEASERTIRSEAEDSYHFISAKMTAFELSKKQEVNM |
| | Y690F, R693V, V694L, | SDSALDCVRDEAINKLQQIFNTSYNQTYEKYGNVSVFETTGGLVVFWQG |
| | K700A, V702Q | IKQKSIVELERLANRSSLNLTHNRTKRSTDGNNATHLSNMESVHNLVYA. |
| | dI-dIV-dV-pack3: | QLQFTYDTERGYINRALAQIAEAWCVDQRRTLEVFKELSKINPSAILSA |
| | Y218F, N220S, V552L, | IYNKPIAARFMGDVLGLASCLTINQTSVKVLRDMNVKESPGRCYSRPVV |
| | Q591F, S668A | IFNFANSSYVFYGQLGEDNEILLGNHRTEECQLPSLKIFIAGNSAYEYV |
| | | DYLFKRMIDLSSISTVDSMIALDIDPLENTDERVLELYAQKELRSSNVE |
| | | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTEK |
| NO: 282 | short_dI-dIV-dV- | VRVYQKVLTFRRSYAYIHTTYLLGSNTEYVAPPMWEIHHINSHSQCYSS |
| | pack3_Disulf1 | YSRVIAGTVFVAYHRDSYENKTMQLMPDCFSSTHSTRYVTVKDQWHSRG |
| | Cpack1-short: E686L, | STWLYRETCNLNCMVTITTARSKYPYHFFATSTGDVVDISPFYNGTNRN |
| | Y690F, R693V, V694L | ASYFGENADKFFIFPNYTIVSDEGRPNSALETHRLVAFLERADSVISWD |
| | dI-dIV-dV-pack3: | IQDEKNVICQLTFWEASERTIRSEAEDSYHFSSAKMTATFLSKKQEVNM |
| | Y218F, N220S, V552L, | SDSALDCVRDEAINKLQQIFNTSYNQTYEKYGNVSVFETTGGLVVFWQG |
| | Q591F, S668A | IKQKSLVELERLANRSSLNLTHNRTKRSTDGNNATHLSNMESVHNLVYA |
| | Disulf1: D217C, 3587C | QLQFTYDTLRGYINRALAQIAEAWCVDQRRTLEVFKELSKINPSAILSA |
| | | IYNKPIAARFMGDVLGLASCLTINQTSVKVLRDMNVKESPGRCYSRPVV |
| | | IFNFANCSYVFYGQLGEDNEILLGNHRTEECQLPSLKIFIAGNSAYEYV |
| | | DYLFKRMIDLSSISTVDSMIALDIDPLENTDERVLELYAQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTFK |
| NO: 283 | long_dI-dIV-dV- | VRVYQKVLTFRRSYAYIHTTYLLGSNTEYVAPPMWEIHHINSHSQCYSS |
| | pack3_Disulf1 | YSRVIAGTVFVAYHRDSYENKTMQLMPDCFSSTHSTRYVTVKDQWHSRG |
| | Cpack1-long: E686L, | STWLYRETCNLNCMVTITTARSKYPYHFFATSTGDVVDISPFYNGTNRN |
| | Y690F, R693V, V694L, | ASYFGENADKFFIFPNYTIVSDEGRENSALETHRLVAFLERADSVISWD |
| | K700A, V702Q | IQDEKNVTCQLTEWEASERTIRSEAEDSYHFSSAKMTATFLSKKQEVNM |
| | dI-dIV-dV-pack3: | SDSALDCVRDEAINKLQQIFNTSYNQTYEKYGNVSVFETTGGLVVFWQG |
| | Y218F, N220S, V552L, | IKQKSLVELERLANRSSLNLTANRTKRSTDGNNATHLSNMESVHNLVYA |
| | Q591F, S668A | QLQFTYDTLRGYINRALAQIAEAWCVDQRRTLEVFKELSKINPSAILSA |
| | Disulf1: D217C, S587C | IYNKPIAARFMGDVLGLASCLTINQTSVKVLRDMNVKESPGRCYSRPVV |
| | | IFNFANCSYVFYGQLGEDNEILLGNHRTEECQLPSLKIFIAGNSAYEYV |
| | | DYLFKRMIDLSSISTVDSMIALDIDPLENTDERVLELYAQKELRSSNVE |
| | | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTEK |
| NO: 284 | short_dII-dIII- | VRVYQKVLTERRSYAYIHTTYLLGSNTEYVAPPMWEIHHINSHSQCYSS |
| | pack1_dI-dIV-dV- | YSRVIAGTVFVAYHRDSYENKTMQLMPDCFSSTHSTRYVTVKDQWHSRG |
| | pack3_Disulf1 | STWLYRETCNLNCMVTITTARSKYPYHFFATSTGDVVDISPFYNGTNRN |
| | Cpack1-short: E686L, | ASYFGENADKFFIFPNYTIVSDFGRPNSALETHRLVAFLERADSVISWD |
| | Y690F, R693V, V694L | IQDEKNVTCQLIFWEASERTIRSEAEDSYHFISAKMTAFFLSKKQEVNM |
| | dII-dIII-pack1: S367I, | SDSALDCVRDEAINKLQQIFNTSYNQTYEKYGNVSVFETTGGLVVFWQG |
| | T374F | IKQKSLVELERLANRSSLNLTHNRTKRSTDGNNATHLSNMESVHNLVYA |
| | dI-dIV-dV-pack3: | QLQFTYDTLRGYINRALAQIAEAWCVDQRRTLEVFKELSKINPSAILSA |
| | Y218F, N220S, V552L, | IYNKPIAARFMGDVIGLASCLTINQTSVKVLRDMNVKESPGRCYSRPVV |
| | Q591F, S668A | IFNFANCSYVFYGQLGEDNEILLGNHRTEECQLPSLKIFIAGNSAYEYV |
| | Disulf1: D217C, S587C | DYLFKRMIDLSSISTVDSMIALDIDPLENTDERVLELYAQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTEK |
| NO: 285 | long_dII-dIII- | VRVYQKVLTFRRSYAYIHTTYLLGSNTEYVAPPMWEIHHINSHSQCYSS |
| | pack1_dI-dIV-dV- | YSRVIAGTVFVAYHRDSYENKTMQLMPDCFSSTHSTRYVTVKDQWHSRG |
| | pack3_Disulf1 | STWLYRETCNLNCMVTITTARSKYPYHFFATSTGDVVDISPFYNGTNRN |
| | Cpack1-long: E686L, | ASYFGENADKFFIFPNYTIVSDFGRPNSALETHRLVAFLERADSVISWD |
| | Y690F, R693V, V694L, | IQDEKNVTCQLTFWEASERTIRSEAEDSYHFISAKMTAFFLSKKQEVNM |
| | K700A, V702Q | SDSALDCVRDEAINKLQQIFNTSYNQTYEKYGNVSVFETTGGLVVFWQG |
| | dII-dIII-pack1: S367I, | IKQKSLVELERLANRSSLNLTHNRTKRSTDGNNATHLSNMESVHNLVYA |
| | T374F | QLQFTYDTERGYINRALAQIAEAWCVDQRRTLEVFKELSKINPSAILSA |
| | dI-dIV-dV-pack3: | IYNKPIAARFMGDVLGLASCLTINQTSVKVLRDMNVKESPGRCYSRPVV |
| | Y218F, N220S, V552L, | IFNFANCSYVFYGQLGEDNEILLGNHRTEECQLPSLKIFIAGNSAYEYV |
| | Q591F, S668A | DYLFKRMIDLSSISTVDSMIALDIDPLENTDERVLELYAQKELRSSNVE |
| | Disulf1: D217C, S587C | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTEK |
| NO: 286 | short_dII-dIII- | VRVYQKVLTFRRSYAYIHTTYLLGSNTEYVAPPMWEIHHINSHSQCYSS |
| | pack1_dI-dIV-dV- | YSRVIAGTVFVAYHRDSYENKTMQLMPDCFSSTHSTRYVTVKDQWHSRG |
| | pack4_Disulf1 | STWLYRETCNLNCMVTITTARSKYPYHFFATSTGDVVDISPFYNGTNRN |
| | Cpack1-short: E686L, | ASYFGENADKFFIFPNYTIVSDFGRPNSALETHRLVAFLERADSVISWD |
| | Y690F, R693V, V694L | IQDEKNVICQLTFWEASERTIRSEAEDSYHFISAKMTAFELSKKQEVNM |
| | dII-dIII-pack1: S367I, | SDSALDCVRDEAINKLQQIFNTSYNQTYEKYGNVSVFETTGGLVVFWQG |
| | T374F | IKQKSLVELERLANRSSLNLTHNRTKRSTDGNNATHLSNMESVHNLVYA |
| | dI-dIV-dV-pack4: | QLQFTYDTLRGYINRALAQIAEAWCVDQRRTLEVFKELSKINPSAILSA |
| | Y218F, N220S, V552L, | IYNKPIAARFMGDVLGLASCLTINQTSVKVLRDMNVKESPGRCYSRPVV |
| | Q591Y, S668A | IFNFANCSYVYYGQLGEDNEILLGNHRTEECQLPSLKIFIAGNSAYEYV |
| | Disulf1: D217C, S587C | DYLFKRMIDLSSISTVDSMIALDIDPLENTDERVLELYAQKELRSSNVE |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTEK |
| NO: 287 | long_dII-dIII- | VRVYQKVLTFRRSYAYIHTTYLLGSNTEYVAPPMWEIHHINSHSQCYSS |
| | pack1_dI-dIV-dV- | YSRVIAGTVFVAYHRDSYENKTMQLMPDCFSSTHSTRYVTVKDQWHSRG |
| | pack4_Disulf1 | STWLYRETCNLNCMVTITTARSKYPYHFFATSTGDVVDISPFYNGTNRN |
| | Cpack1-long: E686L, | ASYFGENADKFFIFPNYTIVSDFGRPNSALETHRLVAFLERADSVISWD |
| | Y690F, R693V, V694L, | IQDEKNVTCQLTFWEASERTIRSEAEDSYHFISAKMTAFFLSKKQEVNM |
| | K700A, V702Q | SDSALDCVRDEAINKLQQIFNTSYNQTYEKYGNVSVFETTGGLVVFWQG |
| | dII-dIII-pack1: S367I, | IKQKSLVELERLANRSSLNLTHNRTKRSTDGNNATHLSNMESVHNLVYA |
| | T374F | QLQFTYDTLRGYINRALAQIAEAWCVDQRRTLEVFKELSKINPSAILSA |
| | dI-dIV-dV-pack4: | IYNKPIAARFMGDVLGLASCLTINQTSVKVLRDMNVKESPGRCYSRPVV |
| | Y218F, N220S, V552L, | IFNFANCSYVYYGQLGEDNEILLGNHRTEECQLPSLKIFIAGNSAYEYV |
| | Q591Y, S668A | DYLFKRMIDLSSISTVDSMIALDIDPLENTDERVLELYAQKELRSSNVF |
| | Disulf1: D217C, S587C | DLEEIMRLENSFKQVLKYVEDAVQ |
| SEQ ID | gB-Towne_Cpack1- | YRVCSMAQGTDLIRFERNIVCISMKPINEDLDEGIMVVYKRNIVAHTFK |
| NO: 288 | short_dII-dIII- | VRVYQKVLTFRRSYAYIHTTYLLGSNTEYVAPPMWEIHHINSHSQCYSS |
| | pack1_dI-dIV-dV- | YSRVIAGTVFVAYHRDSYENKTMQLMPDCYSSTHSTRYVTVKDQWHSRG |
| | pack5_Disulf1 | STWLYRETCNLNCMVTITTARSKYPYHFFATSTGDVVDISPFYNGTNRN |
| | Cpack1-short: E686L, | ASYFGENADKFFIFPNYTIVSDEGRPNSALETHRLVAFLERADSVISWD |
| | Y690F, R693V, V694L | IQDEKNVTCQLTFWEASERTIRSEAEDSYHFISAKMTAFFLSKKQEVNM |
| | dII-dIII-pack1: S367I, | SDSALDCVRDEAINKLQQIFNTSYNQTYEKYGNVSVFETTGGLVVFWQG |
| | T374F | IKQKSLVELERLANRSSLNLTHNRTKRSTDGNNATHLSNMESVHNLVYA |
| | dI-dIV-dV-pack5: | QLQFTYDTLRGYINRALAQIAEAWCVDQRRTLEVFKELSKINPSAILSA |
| | N220S, V552L, Q591Y | IYNKPIAARFMGDVLGLASCLTINQTSVKVLRDMNVKESPGRCYSRPVV |
| | Disulf1: D217C, S587C | IFNFANCSYVYYGQLGEDNEILLGNHRTEECQLPSLKIFIAGNSAYEYV |
| | | DYLFKRMIDLSSISTVDSMIALDIDPLENTDERVLELYSQKELRSSNVE |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTEK |
| NO: 289 | long_dII-dIII- | VRVYQKVLTFRRSYAYIHTTYLLGSNTEYVAPPMWEIHHINSHSQCYSS |
| | pack1_dI-dIV-dV- | YSRVIAGTVFVAYHRDSYENKTMQLMPDCYSSTHSTRYVTVKDQWHSRG |
| | pack5_Disulf1 | STWLYRETCNLNCMVTITTARSKYPYHFFATSTGDVVDISPFYNGTNRN |
| | Cpack1-long: E686L, | ASYFGENADKFFIFPNYTIVSDEGRPNSALETHRLVAFLERADSVISWD |
| | Y690F, R693V, V694L, | IQDEKNVTCQLTFWEASERTIRSEAEDSYHFISAKMTAFFLSKKQEVNM |
| | K700A, V702Q | SDSALDCVRDEAINKLQQIFNTSYNQTYEKYGNVSVFETTGGLVVFWQG |
| | dII-dIII-pack1: S367I, | IKQKSLVELERLANRSSLNLTHNRTKRSTDGNNATHLSNMESVHNLVYA |
| | T374F | QLQFTYDTERGYINRALAQIAEAWCVDQRRTLEVFKELSKINPSAILSA |
| | dI-dIV-dV-pack5: | IYNKPIAARFMGDVLGLASCLTINQTSVKVLRDMNVKESPGRCYSRPVV |
| | N220S, V552L, Q591Y | IFNFANCSYVYYGQLGEDNEILLGNHRTEECQLPSLKIFIAGNSAYEYV |
| | Disulf1: D217C, S587C | DYLFKRMIDLSSISTVDSMIALDIDPLENTDERVLELYSQKELRSSNVF |
| | | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTEK |
| NO: 290 | short_dII-dIII- | VRVYQKVLTFRRSYAYIHTTYLLGSNTEYVAPPMWEIHHINSHSQCYSS |
| | pack1_dI-dIV-dV- | YSRVIAGTVFVAYHRDSYENKTMQLMPDCYSSTHSTRYVTVKDQWHSRG |
| | pack6_Disulf1 | STWLYRETCNLNCMVTITTARSKYPYHFFATSTGDVVDISPFYNGINRN |
| | Cpack1-short: E686L, | ASYFGENADKFFIFPNYTIVSDFGRPNSALETHRLVAFLERADSVISWD |
| | Y690F, R693V, V694L | IQDEKNVTCQLTFWEASERTIRSEAEDSYHFISAKMTAFFLSKKQEVNM |
| | dII-dIII-pack1: S3671, | SDSALDCVRDEAINKLQQIFNTSYNQTYEKYGNVSVFETTGGLVVFWQG |
| | T374F | IKQKSLVELERLANRSSLNLTHNRTKRSTDGNNATHLSNMESVHNLVYA |
| | dI-dIV-dV-pack6: | QLQFTYDTLRGYINRALAQIAEAWCVDQRRTLEVFKELSKINPSAILSA |
| | N220S, V552L, 0591F | IYNKPIAARFMGDVLGLASCLTINQTSVKVLRDMNVKESPGRCYSRPVV |
| | Disulf1: D217C, S587C | IFNFANCSYVFYGQLGEDNEILLGNHRTEECQLPSLKIFIAGNSAYEYV |
| | | DYLFKRMIDLSSISTVDSMIALDIDPLENTDERVLELYSQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTFK |
| NO: 291 | long_dII-dIII- | VRVYQKVLTFRRSYAYIHTTYLLGSNTEYVAPPMWEIHHINSHSQCYSS |
| | pack1_dI-dIV-dV- | YSRVIAGTVFVAYHRDSYENKTMQLMPDCYSSTHSTRYVTVKDQWHSRG |
| | pack6_Disulf1 | STWLYRETCNLNCMVTITTARSKYPYHFFATSTGDVVDISPFYNGINRN |
| | Cpack1-long: E686L, | ASYFGENADKFFIFPNYTIVSDEGRPNSALETHRLVAFLERADSVISWD |
| | Y690F, R693V, V694L, | IQDEKNVTCQLTEWEASERTIRSEAEDSYHFISAKMTAFFLSKKQEVNM |
| | K700A, V702Q | SDSALDCVRDEAINKLQQIFNTSYNQTYEKYGNVSVFETTGGLVVFWQG |
| | dII-dIII-pack1: S367I, | IKQKSIVELERLANRSSINLTANRTKRSTDGNNATHLSNMESVHNLVYA |
| | T374F | QLQFTYDTERGYINRALAQIAEAWCVDQRRTLEVFKELSKINPSAILSA |
| | dI-dIV-dV-pack6: | IYNKPIAARFMGDVLGLASCLTINQTSVKVLRDMNVKESPGRCYSRPVV |
| | N220S, V552L, Q591F | IFNFANCSYVFYGQLGEDNEILLGNHRTEECQLPSLKIFIAGNSAYEYV |
| | Disulf1: D217C, S587C | DYLFKRMIDLSSISTVDSMIALDIDPLENTDERVLELYSQKELRSSNVE |
| | | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTEK |
| NO: 292 | short_dI-dIV-dV- | VRVYQKVLTFRRSYAYIHTTYLLGSNTEYVAPPMWEIHHINSHSQCYSS |
| | pack4_Disulf1 | YSRVIAGTVFVAYHRDSYENKTMQLMPDCFSSTHSTRYVTVKDQWHSRG |
| | Cpack1-short: E686L, | STWLYRETCNLNCMVTITTARSKYPYHFFATSTGDVVDISPFYNGINRN |
| | Y690F, R693V, V694L | ASYFGENADKFFIFPNYTIVSDEGRPNSALETHRLVAFLERADSVISWD |
| | dI-dIV-dV-pack4: | IQDEKNVICQLTFWEASERTIRSEAEDSYHFSSAKMTATFLSKKQEVNM |
| | Y218F, N220S, V552L, | SDSALDCVRDEAINKLQQIFNTSYNQTYEKYGNVSVFETTGGLVVFWQG |
| | Q591Y, S668A | IKQKSLVELERLANRSSLNLTHNRTKRSTDGNNATHLSNMESVHNLVYA |
| | Disulf1: D217C, S587C | QLQFTYDTLRGYINRALAQIAEAWCVDQRRTLEVFKELSKINPSAILSA |
| | | IYNKPIAARFMGDVLGLASCLTINQTSVKVLRDMNVKESPGRCYSRPVV |
| | | IFNFANCSYVYYGQLGEDNEILLGNHRTEECQLPSLKIFIAGNSAYEYV |
| | | DYLFKRMIDLSSISTVDSMIALDIDPLENTDFRVLELYAQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTEK |
| NO: 293 | long_dI-dIV-dV- | VRVYQKVLTFRRSYAYIHTTYLLGSNTEYVAPPMWEIHHINSHSQCYSS |
| | pack4_Disulf1 | YSRVIAGTVFVAYHRDSYENKIMQLMPDCFSSTHSTRYVTVKDQWHSRG |
| | Cpack1-long: E686L, | STWLYRETCNLNCMVTITTARSKYPYHFFATSTGDVVDISPFYNGTNRN |
| | Y690F, R693V, V694L, | ASYFGENADKFFIFPNYTIVSDEGRPNSALETHRLVAFLERADSVISWD |
| | K700A, V702Q | IQDEKNVICQLTFWEASERTIRSEAEDSYHFSSAKMTATFLSKKQEVNM |
| | dI-dIV-dV-pack4: | SDSALDCVRDEAINKLQQIFNTSYNQTYEKYGNVSVFETTGGLVVFWQG |
| | Y218F, N220S, V552L, | IKQKSLVELERLANRSSLNLTHNRTKRSTDGNNATHLSNMESVHNLVYA |
| | Q591Y, S668A | QLQFTYDTLRGYINRALAQIAEAWCVDQRRTLEVFKELSKINPSAILSA |
| | Disulf1: D217C, S587C | IYNKPIAARFMGDVLGLASCLTINQTSVKVLRDMNVKESPGRCYSRPVV |
| | | IFNFANCSYVYYGQLGEDNEILLGNHRTEECQLPSLKIFIAGNSAYEYV |
| | | DYLFKRMIDLSSISTVDSMIALDIDPLENTDERVLELYAQKELRSSNVE |
| | | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTEK |
| NO: 294 | short_dI-dIV-dV- | VRVYQKVLTFRRSYAYIHTTYLLGSNTEYVAPPMWEIHHINSHSQCYSS |
| | pack5_Disulf1 | YSRVIAGTVFVAYHRDSYENKTMQLMPDCYSSTHSTRYVTVKDQWHSRG |
| | Cpack1-short: E686L, | STWLYRETCNLNCMVTITTARSKYPYHFFATSTGDVVDISPFYNGTNRN |
| | Y690F, R693V, V694L | ASYFGENADKFFIFPNYTIVSDEGRPNSALETHRLVAFLERADSVISWD |
| | dI-dIV-dV-pack5: | IQDEKNVTCQLTFWEASERTIRSEAEDSYHFSSAKMTATFLSKKQEVNM |
| | N220S, V552L, Q591Y | SDSALDCVRDEAINKLQQIFNTSYNQTYEKYGNVSVFETTGGLVVFWQG |
| | Disulf1: D217C, S587C | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCLTINQTSVKVLRDMNVKESPGRCYSRPVV |
| | | IFNFANCSYVYYGQLGEDNEILLGNHRTEECQLPSLKIFIAGNSAYEYV |
| | | DYLFKRMIDLSSISTVDSMIALDIDPLENTDERVLELYSQKELRSSNVE |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTEK |
| NO: 295 | long_dI-dIV-dV- | VRVYQKVLIFRRSYAYIHTTYLLGSNTEYVAPPMWEIHHINSHSQCYSS |
| | pack5_Disulf1 | YSRVIAGTVFVAYHRDSYENKTMQLMPDCYSSTHSTRYVTVKDQWHSRG |
| | Cpack1-long: E686L, | STWLYRETCNLNCMVTITTARSKYPYHFFATSTGDVVDISPFYNGTNRN |
| | Y690F, R693V, V694L, | ASYFGENADKFFIFPNYTIVSDFGRPNSALETHRLVAFLERADSVISWD |
| | K700A, V702Q | IQDEKNVTCQLTEWEASERTIRSEAEDSYHFSSAKMTATFLSKKQEVNM |
| | dI-dIV-dV-pack5: | SDSALDCVRDEAINKLQQIFNTSYNQTYEKYGNVSVFETTGGLVVFWQG |
| | N220S, V552L, Q591Y | IKQKSLVELERLANRSSLNLTANRTKRSTDGNNATHLSNMESVHNLVYA |
| | Disulf1: D217C, S587C | QLQFTYDTLRGYINRALAQIAEAWCVDQRRTLEVFKELSKINPSAILSA |
| | | IYNKPIAARFMGDVLGLASCLTINQTSVKVLRDMNVKESPGRCYSRPVV |
| | | IFNFANCSYVYYGQLGEDNEILLGNHRTEECQLPSLKIFIAGNSAYEYV |
| | | DYLFKRMIDLSSISTVDSMIALDIDPLENTDERVLELYSQKELRSSNVE |
| | | DLEEIMRLENSFKQVLKYVEDAVQ |
| SEQ ID | gB-Towne_Cpack1- | YRVCSMAQGTDLIRFERNIVCTSMKPINEDLDEGIMVVYKRNIVAHTEK |
| NO: 296 | short_dI-dIV-dV- | VRVYQKVLTFRRSYAYIHTTYLLGSNTEYVAPPMWEIHHINSHSQCYSS |
| | pack6_Disulf1 | YSRVIAGTVFVAYHRDSYENKIMQLMPDCYSSTHSTRYVTVKDQWHSRG |
| | Cpack1-short: E686L, | STWLYRETCNLNCMVTITTARSKYPYHFFATSTGDVVDISPFYNGTNRN |
| | Y690F, R693V, V694L | ASYFGENADKFFIFPNYTIVSDFGRPNSALETHRLVAFLERADSVISWD |
| | dI-dIV-dV-pack6: | IQDEKNVTCQLTFWEASERTIRSEAEDSYHFSSAKMTATFLSKKQEVNM |
| | N220S, V552L, Q591F | SDSALDCVRDEAINKLQQIFNTSYNQTYEKYGNVSVFETTGGLVVFWQG |
| | Disulf1: D217C, S587C | IKQKSLVELERLANRSSLNLTHNRTKRSTDGNNATHLSNMESVHNLVYA |
| | | QLQFTYDTLRGYINRALAQIAEAWCVDQRRTLEVFKELSKINPSAILSA |
| | | IYNKPIAARFMGDVLGLASCLTINQTSVKVERDMNVKESPGRCYSRPVV |
| | | IFNFANCSYVFYGQLGEDNEILLGNERTEECQLPSLKIFIAGNSAYEYV |
| | | DYLFKRMIDLSSISTVDSMIALDIDPLENTDERVLELYSQKELRSSNVF |
| | | DLEEIMRLENSFKQVLKYV |
| SEQ ID | gB-Towne_Cpack1- | YRVCSMAQGTDLIRFERNIVCTSMKPINEDLDEGIMVVYKRNIVAHTEK |
| NO: 297 | long_dI-dIV-dV- | VRVYQKVLTFRRSYAYIHTTYLLGSNTEYVAPPMWEIHHINSHSQCYSS |
| | pack6_Disulf1 | YSRVIAGTVFVAYHRDSYENKTMQLMPDCYSSTHSTRYVTVKDQWHSRG |
| | Cpackl-long: E686L, | STWLYRETCNLNCMVTITTARSKYPYHFFATSTGDVVDISPFYNGTNRN |
| | Y690F, R693V, V694L, | ASYFGENADKFFIFPNYTIVSDEGRPNSALETHRLVAFLERADSVISWD |
| | K700A, V702Q | IQDEKNVICQLTFWEASERTIRSEAEDSYHFSSAKMTATFLSKKQEVNM |
| | dI-dIV-dV-pack6: | SDSALDCVRDEAINKLQQIFNTSYNQTYEKYGNVSVFETTGGLVVFWQG |
| | N220S, V552L, Q591F | IKQKSIVELERLANRSSLNLTHNRTKRSTDGNNATHLSNMESVHNLVYA |
| | Disulf1: D217C, S587C | QLQFTYDTLRGYINPALAQIAEAWCVDQRRTLEVFKELSKINPSAILSA |
| | | IYNKPIAARFMGDVLGLASCLTINQTSVKVLRDMNVKESPGRCYSRPVV |
| | | IFNFANCSYVFYGQLGEDNEILLGNHRTEECQLPSLKIFIAGNSAYEYV |
| | | DYLFKRMIDLSSISTVDSMIALDIDPLENTDERVLELYSQKELRSSNVF |
| | | DLEEIMRLENSFKQVLKYVEDAVQ |

In some embodiments, the polypeptide comprises, as a C-terminal fusion to the ectodomain, a trimerization domain. In some embodiments, the polypeptide comprises, as a C-terminal fusion to the ectodomain, a nanostructure assembly domain.

In another aspect, the disclosure provides a fusion protein comprising the polypeptide or antigenic fragment thereof of the disclosure and a multimerization domain.

In some embodiments, the multimerization domain of the fusion protein of the disclosure comprises a polypeptide with sequence identity of at least 50%, 55%, 60%, 65%, 70%, 75%, 80%, 85%, 90%, 95%, 96%, 97%, 98%, 99%, or 100% to the sequence of a protein listed in Table 12, wherein residues in parentheses are optional

### Protein Nanostructures and Assembly Domains

The gB protein sequences disclosed herein may be displayed on a nanostructure, either as a fusion protein with a component of the nanostructure or by conjugation to the nanostructure. It is contemplated that any of the polypeptides with amino acid substitutions disclosed herein may be used on any of the nanostructures described herein, known in the art, or later developed.

Various protein nanostructures are known in the art and described, for example in U.S. Pat. Pub. Nos. US2015/0356240A1: US2016/0122392A1. US20180030429A1, US20190341124A1, and US2022/0072120A1, the contents of which are incorporated by reference herein. In some embodiment, the protein nanostructure comprises, as an assembly domain, a variant of KDPG aldolase (Protein Data Bank code 1WA3) engineered to self-assemble into a protein nanostructure.

### One-Component Nanostructures

In its native form. 1WA3 non-covalently assembles to form a trimer via a first interface (the trimer interface). When 20 copies of the trimer (60 monomers) are computationally docked to form a one-component icosahedral protein nanostructure, sets of five monomers of 1WA3 contact one another via a second interface (the pentamer interface). By introducing amino acid substitutions, the pentamer interface may be stabilized such that the protein nanostructure will spontaneously self-assemble, e.g., within the expressing cell or when isolated trimers (or monomers) are mixed under suitable conditions.

In some embodiments, the pentamer interface comprises 1, 2, 3, 4 or more interface residues, such as amino acid residues in positions 33, 61, 187, and 190 numbered according to SEQ ID NO: 8. In some embodiments, the assembly domain comprises a polypeptide sequence at least 70%, at least 80%, at least 90%, at least 95%, at least 98% identical to SEQ ID NO: 8. In some embodiments, the assembly domain comprises amino acid substitutions at 1, 2, 3, 4 of positions 33, 61, 187, and 190 compared to SEQ ID NO: 8. In some embodiments, a plurality of the amino acid substitutions are substitutions of a polar residue for a non-polar residue (e.g., A, L, I, M, V, F, or W). In some embodiment, some or all of the amino acid substitutions are substitutions of a polar residue for a small, non-polar residue (e.g., A, L, I, M, or V). In some embodiment, the protein nanostructure comprises amino acid substitutions E33L or E33V; K61L or K61M; D187A or D187V; and/or R190A. In some embodiments, the protein nanostructure comprises amino acid substitutions E33L, K61M. D187V, and R190A. In some embodiments, the protein nanostructure comprises amino acid substitutions E33V, K61L, D187A, and R190A. In some embodiments, the assembly domain comprises an amino acid substitution to negate the enzymatic activity of the assembly domain (e.g., K129A). In some embodiments, the assembly domain may comprise further amino acid substitutions (e.g., MI3; E56M or E56K; P186I; E191A; and/or K194A).

In some embodiments, the assembly domain comprises a polypeptide sequence at least 75%, 80%, 85%, 90%, 95%, 96%, 97%, 98%, 99%, or 100% identical to 13-01 (SEQ ID NO: 8).

SEQ ID NO: 8 is shown below with illustrative pentamer interface positions in bold:

(SEQ ID NO: 8)

| | |
|---|---|
| 1 | 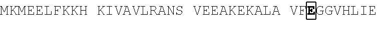 |
| 41 | 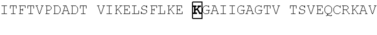 |
| 81 | ESGAEFIVSP HLDEEISQFC KEKGVFYMPG VMTPTELVKA |
| 121 | MKLGHTILKL FPGEVVGPQF VKAMKGPFPN VKFVPTGGVN |
| 161 | 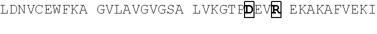 |
| 201 | RGCTE |

In some embodiments, the assembly domain comprises amino acid substitutions that remove cysteine residues. In some embodiments, the assembly domain comprises C76A and/or C100A substitutions. In some embodiments, the assembly domain comprises C76A, C100A, C165A, and/or C203A substitutions.

Illustrative assembly domain sequences are provided in Table 11. In each case, the N-terminal MK is optional and not included when calculating sequence identity, but is shown only for numbering purposes, i.e., MK is included in the reference sequence but not necessarily in the assembly domain of the nanostructure.

TABLE 11

| SEQ | | |
|---|---|---|
| ID | | Pentamer |
| NO: | Sequence | Interface |
| 9 | (MK)MEELFKKHKIVAVLRANSVEEAKKKALAVFLGGVHLIE | none |
| | ITFTVPDADTVIKELSFLKEMGAIIGAGTVTSVEQCRKAVES | |
| | GAEFIVSPHLDEEISQFCKEKGVFYMPGVMTPTELVKAMKLG | |
| | HTILKLFPGEVVGPQFVKAMKGPFPNVKFVPTGGVNLDNVCE | |
| | WFKAGVLAVGVGSALVKGTPVEVAEKAKAFVEKIRGCTE | |
| 10 | (MK)IEELFKKHKIVAVLRANSVEEAKKKALAVFLGGVHLIE | E33L, K61M, |
| | ITFTVPDADTVIKELSFLKEMGAIIGAGTVTSVEQCRKAVES | D187V, R190A |
| | GAEFIVSPHLDEEISQFCKEKGVFYMPGVMTPTELVKAMKLG | |
| | HTILKLFPGEVVGPQFVKAMKGPFPNVKFVPTGGVNLDNVCE | |
| | WFKAGVLAVGVGSALVKGTPVEVAEKAKAFVEKIRGCTE | |
| 11 | (MK)MEELFKKHKIVAVLRANSVEEAKKKALAVFLGGVHLIE | E33L, K61M, |
| | ITFTVPDADTVIKELSFLKEMGAIIGAGTVTSVEQARKAVES | D187V, R190A |
| | GAEFIVSPHLDEEISQFAKEKGVFYMPGVMTPTELVKAMKLG | |
| | HTILKLFPGEVVGPQFVKAMKGPFPNVKFVPTGGVNLDNVCE | |
| | WFKAGVLAVGVGSALVKGTPVEVAEKAKAFVEKIRGCTE | |
| 12 | (MK)MEELFKKHKIVAVLRANSVEEAKMKALAVFVGGVHLIE | E33V, K61L, |
| | ITFTVPDADTVIKELSFLKELGAIIGAGTVTSVEQCRKAVES | D187A, R190A |
| | GAEFIVSPHLDEEISQFCKEKGVFYMPGVMTPTELVKAMKLG | |
| | HTILKLFPGEVVGPQFVKAMKGPFPNVKFVPTGGVNLDNVCE | |
| | WFKAGVLAVGVGSALVKGTIAEVAAKAAAFVEKIRGCTE | |
| 13 | (MK)MEELFKKHKIVAVLRANSVEEAKKKALAVFLGGVDLIE | E33L, K61M, |
| | ITFTVPDADTVIKELSFLKEMGAIIGAGTVTSVEQCRKAVES | D187V, R190A |
| | GAEFIVSPHLDEEISQFCKEKGVFYMPGVMTPTELVKAMKLG | |
| | HTILKLFPGEVVGPQFVKAMKGPFPNVKFVPTGGVNLDNVCE | |
| | WEKAGVQAVGVGEALNKGTPVEVAEKAKAFVEKIRGCTE | |
| 14 | (MK)MEELFKKHKIVAVLRANSVEEAKKKALAVFMGGVDLIE | E33M, K61L, |
| | ITFTVPDADTVIKELSFLKELGAIIGAGTVTSVEQCRKAVES | D187A, R190A |
| | GAEFIVSPHLDEEISQFCKEKGVFYMPGVMTPTELVKAMKLG | |
| | HTILKLFPGEVVGPQFVKAMKGPFPNVKFVPTGGVNLDNVCE | |
| | WFKAGVQAVGVGEALNKGTPAEVAEKAKAFVEKIRGCTE | |

### Two-Component Nanostructures

In some embodiments, the assembly domain comprises amino acid substitutions that remove hydrophobic residues and/or add polar residues. Illustrative assembly domains having such substitutions are provided in Int'l Pat. Appl. No. WO2021163481A1, the entire contents of which are incorporated by reference herein. In some embodiments, such amino acid substitutions increase secretion of the assembly domain when the polynucleotides encoding it are expressed in cell.

SEQ ID NO: 9 is shown below with illustrative positions in bold:

(SEQ ID NO: 9)

| | |
|---|---|
| 1 | 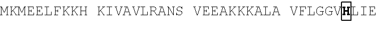 |
| 41 | ITFTVPDADT VIKELSFLKE MGAIIGAGTV TSVEQCRKAV |
| 81 | ESGAEFIVSP HLDEEISQFC KEKGVFYMPG VMTPTELVKA |
| 121 | MKLGHTILKL FPGEVVGPQF VKAMKGPFPN VKFVPTGGVN |
| 161 | 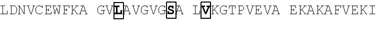 |
| 201 | RGCTE |

In some embodiments, the assembly domain comprises F32Y; H37D/E/K/N/Q/R; F43Q; F168D/E/K/N/Q/R/S/T/Y; K169D/E/N/Q; L173D/E/N/Q/S; A174S; S179D/E; K183D/E, and/or T185D/E/K/N/Q/S amino acid substitutions.

In some embodiments, the assembly domain comprises 1, 2, 3, or 4 of H37D, L173Q, S179E, and V182N substitutions. In some embodiments, the assembly domain comprises H37D, L173Q, S179E, and V182N amino acid substitutions—for example as in SEQ ID NO: 13:

(SEQ ID NO: 13)

| | |
|---|---|
| 1 | 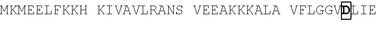 |
| 41 | ITFTVPDADT VIKELSFLKE MGAIIGAGTV TSVEQCRKAV |
| 81 | ESGAEFIVSP HLDEEISQFC KEKGVFYMPG VMTPTELVKA |
| 121 | MKLGHTILKL FPGEVVGPQF VKAMKGPFPN VKFVPTGGVN |
| 161 | 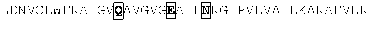 |
| 201 | RGCTE |

In some embodiments, the mulitimerization domain comprises polypeptides at least 50%, 55%, 60%, 65%, 70%, 75%, 80%, 85%, 90%, 95%, 96%, 97%, 98%, 99%, or 100% sequence identity to the amino acid sequence of a protein below (Table 12), wherein residues in parentheses are optional. In some embodiments, the mulitimerization domain is covalently linked to a polypeptide or antigenic fragment thereof of CMV gB protein.

TABLE 12

| SEQ | | |
|---|---|---|
| ID | | |
| NO: | Sequence | |
| 15 | (MG)KYDGSKLRIGILHARWNAEIILALVLGALKRLQEFGVK | I53_dn5A* |
| | RENIIIETVPGSFELPYGSKLFVEKQKRLGKPLDAIIPIGVL | |
| | IKGSTMHFEYICDSTTHQLMKLNFELGIPVIFGVLTCLTDEQ | |
| | AEARAGLIEGKMHNHGEDWGAAAVEMATKFN | |
| 16 | (MG)KYDGSKLRIGILHARGNAEIILALVLGALKRLQEFGVK | I53_dn5A.1 |
| | RENIIIETVPGSFELPYGSKLFVEKQKRLGKPLDAIIPIGVL | |
| | IRGSTPHFDYIADSTTHQLMKLNFELGIPVIFGVITADTDEQ | |
| | AEARAGLIEGKMHNHGEDWGAAAVEMATKFN | |
| 17 | (MG)KYDGSKLRIGILHARGNAEIILELVLGALKRLQEFGVK | I53_dn5A.2 |
| | RENIIIETVPGSFELPYGSKLFVEKQKRIGKPLDAIIPIGVL | |
| | IRGSTAHFDYIADSTTHQLMKLNFELGIPVIFGVLTTESDEQ | |
| | AEERAGTKAGNHGEDWGAAAVEMATKFN | |
| 18 | (M)EEAELAYLLGELAYKLGEYRIAIRAYRIALKRDPNNAEA | I53_dn5B |
| | WYNLGNAYYKQGRYREAIEYYQKALELDPNNAEAWYNLGNAY | |
| | YERGEYEEAIEYYRKALRLDPNNADAMQNLLNAKMREE | |
| 19 | (M)KMEELFKKHKIVAVLRANSVEEAIEKAVAVFAGGVHLIE | I53-50A |
| | ITFTVPDADTVIKALSVLKEKGAIIGAGTVTSVEQCRKAVES | |
| | GAEFIVSPHLDEEISQFCKEKGVFYMPGVMTPTELVKAMKLG | |
| | HTILKLFPGEVVGPQFVKAMKGPFPNVKFVPTGGVNLDNVCE | |
| | WFKAGVLAVGVGSALVKGTPDEVREKAKAFVEKIRGCTE | |
| 20 | (M)NQHSHKDYETVRIAVVRARWHAEIVDACVSAFEAAMADI | I53-50B |
| | GGDRFAVDVFDVPGAYEIPLHARTLAETGRYGAVLGTAFVVN | |
| | GGIYRHEFVASAVIDGMMNVQLSTGVPVLSAVLTPHRYRDSD | |
| | AHTLLFLALFAVKGMEAARACVEILAAREKIAA | |
| 21 | (M)KMEELFKKHKIVAVLRANSVEEAIEKAVAVFAGGVHLIE | I53-50A.1 |
| | ITFTVPDADTVIKALSVLKEKGAIIGAGTVTSVEQCRKAVES | |
| | GAEFIVSPHLDEEISQFCKEKGVFYMPGVMTPTELVKAMKLG | |
| | HDILKLFPGEVVGPQFVKAMKGPFPNVKFVPTGGVNLDNVCE | |
| | WFKAGVLAVGVGDALVKGDPDEVREKAKKFVEKIRGCTE | |
| 22 | (M)KMEELFKKHKIVAVIRANSVEEAIEKAVAVFAGGVHLIE | I53- |
| | ITFTVPDADTVIKALSVLKEKGAIIGAGTVTSVEQCRKAVES | 50A.1NegT2 |
| | GAEFIVSPHIDEEISQFCKEKGVFYMPGVMTPTELVKAMKLG | |
| | HDILKLFPGEVVGPEFVEAMKGPFPNVKFVPTGGVDLDDVCE | |
| | WFDAGVLAVGVGDALVEGDPDEVREDAKEFVEEIRGCTE | |
| 23 | (M)KMEELFKKHKIVAVLRANSVEEAIEKAVAVFAGGVHLIE | I53- |
| | ITFTVPDADTVIKALSVLKEKGAIIGAGTVTSVEQCRKAVES | 50A.1PosT1 |
| | GAEFIVSPHIDEEISQFCKEKGVFYMPGVMTPTELVKAMKLG | |
| | HDILKLFPGEVVGPQFVKAMKGPFPNVKFVPTGGVNLDNVCK | |
| | WEKAGVLAVGVGKALVKGKPDEVREKAKKFVKKIRGCTE | |
| 24 | (M)NQHSHKDHETVRIAVVRARWHAEIVDACVSAFEAAMRDI | I53-50B.1 |
| | GGDRFAVDVFDVPGAYEIPLHARTLAETGRYGAVLGTAFVVN | |
| | GGIYRHEFVASAVIDGMMNVQLDTGVPVLSAVLTPHRYRDSD | |
| | AHTLLFLALFAVKGMEAARACVEILAAREKIAA | |
| 25 | (M)NQHSHKDHETVRIAVVRARWHAEIVDACVSAFEAAMRDI | I53- |
| | GGDRFAVDVFDVPGAYEIPLHARTLAETGRYGAVLGTAFVVD | 50B.1NegT2 |
| | GGIYDHEFVASAVIDGMMNVQLDTGVPVLSAVLTPHEYEDSD | |
| | ADTLLFLALFAVKGMEAARACVEILAAREKIAA | |
| 26 | (M)NQHSHKDHETVRIAVVRARWHAEIVDACVSAFEAAMRDI | I53- |
| | GGDRFAVDVFDVPGAYEIPLHARTLAETGRYGAVLGTAFVVN | 50B.4PosT1 |
| | GGIYRHEFVASAVINGMMNVQLNTGVPVLSAVLTPHNYDKSK | |
| | AHTLLFLALFAVKGMEAARACVEILAAREKIAA | |

In another aspect, the disclosure provides a nanostructure comprising any polypeptide of the disclosure. In some embodiments, the assembly domain comprises a polypeptide sequence at least 75%, 80%, 85%, 90%, 95%, 96%, 97%, 98%, 99%, or 100% identical to 153-50A (SEQ ID NO: 19), 153-50A.1 (SEQ ID NO: 21), 153-50A.1NegT2 (SEQ ID NO: 22), or 153-50A.1PosT1 (SEQ ID NO: 23). In some embodiments, the assembly domain comprises a polypeptide sequence at least 75%, 80%, 85%, 90%, 95%, 96%, 97%, 98%, 99%, or 100% identical to 153_dn5B (SEQ ID NO: 18). In some embodiments, the nanostructure comprises a second polypeptide. In some embodiments, the second polypeptide comprises a polypeptide sequence at least 75%, 80%, 85%, 90%, 95%, 96%, 97%, 98%, 99%, or 100% identical to 153-50B (SEQ ID NO: 20), 153-50B.1 (SEQ ID NO: 24), 153-50B.1NegT2 (SEQ ID NO: 25), or 153-50B.4PosT1 (SEQ ID NO: 26). In some embodiments, the second polypeptide comprises a polypeptide sequence at least 75%, 80%, 85%, 90%, 95%, 96%, 97%, 98%, 99%, or 100% identical to 153_dn5A* (SEQ ID NO: 15), 153_dn5A.1 (SEQ ID NO: 16), or 153_dn5A.2 (SEQ ID NO: 17).

### Ferritin-Based Nanostructures

In some embodiments, the assembly domain is a ferritin polypeptide. In some embodiments, the assembly domain of a ferritin protein nanostructure comprises a polypeptide sequence at least 70%, at least 80%, at least 90%, at least 95%, at least 98% identical, at least 99% identical, at least 100% identical to any one of the following amino acid sequences:

(SEQ ID NO: 27)
MLSKDIIKLLNEQVNKEMNSSNLYMSMSSWCYTHSLDGAGLFLFDHAAEE
YEHAKKLIIFLNENNVPVQLTSISAPEHKFEGLTQIFQKAYEHEQHISES
INNIVDHAIKSKDHATFNFLQWYVAEQHEEEVLFKDILDKVELIGNENHG
LYLADQYVKGIAKSRKS
(SEQ ID NO: 28)
MLKPEMIEKLNEQMNLELYSSLLYQQMSAWCSYHTFEGAAAFLRRHAQEE
MTHMQRLFDYLTDTGNLPRINTVESPFAEYSSLDELFQETYKHEQLITQK
INELAHAAMTNQDYPTFNFLQWYVSEQHEEEKLFKSIIDKLSLAGKSGEG
LYFIDKELSTLDAQN
(SEQ ID NO: 29)
NFHQDCEAGLNRTVNLKFHSSYVYLSMASYFNRDDVALSNFAKFFRERSE
EEKEHAEKLIEYQNQRGGRVFLQSVEKPERDDWANGLEALQTALKLQKSV
NQALLDLHAVAADKSDPHMTDFLESPYLSESVETIKKLGDHITSLKKLWS
SHPGMAEYLFNKHTLG
(SEQ ID NO: 30)
QFSKDIEKLLNEQVNKEMQSSNLYMSMSSWCYTHSLDGAGLFLFDHAAEE
YEHAKKLIIFLNENNVPVQLTSISAPEHKFEGLTQIFQKAYEHEQHISES
INNIVDHAIKSKDHATFNFLQWYVAEQHEEEVLFKDILDKIELIGNENHG
LYLADQYVKGIAKSRKSGS
(SEQ ID NO: 31)
SGESQVRQNFKPEMEEKLNEQMNLELYSSLLYQQMSAWCSYHTFEGAAAF
LRRHAQEEMTHMQRLFDYLTDTGNLPRINTVESPFAEYSSLDELFQETYK
HEQLITQKINELAHAAMTNQDYPTFNFLQWYVSEQHEEEKLFKSIIDKLS
LAGKSGEGLYFIDKELSTLDGS

In some embodiments, the assembly domain comprises a polypeptide sequence at least 75%, 80%, 85%, 90%, 95%, 96%, 97%, 98%, 99%, or 100% identical to SEQ ID NO: 27, SEQ ID NO: 28, SEQ ID NO: 29, SEQ ID NO: 30, or SEQ ID NO: 31.

### Linker

In some embodiments, the encoded polypeptide includes a linker between regions. A wide variety of polypeptide sequences can be used and are well known in the art. In some embodiments, the linker comprises a Gly-Ser linker (i.e., a linker consisting of glycine and serine residues) of any suitable length. In some embodiments, the Gly-Ser linker is 3, 4, 5, 6, 7, 8, 9, 10, 11, 12, 13, 14, 15, 16, 17, 18, 19, 20 or more amino acids in length. Non-limiting examples of Glys-Ser linkers are presented below:

(SEQ ID NO: 298)
GS
(SEQ ID NO: 299)
GSS
(SEQ ID NO: 300)
GSGS
(SEQ ID NO: 301)
GSGGSGSGSGGS
(SEQ ID NO: 302)
GSGGSGSGSGGS
(SEQ ID NO: 303)
GSEKAAKAEEAARK

In another aspect, the disclosure provides a composition comprising one or more of the polypeptides or fusion proteins of the disclosure linked to a scaffold. The scaffold may be any surface to which the polypeptides or fusion proteins may be bound, including but not limited to a bead, glass, polystyrene, a nanoparticle, a protein scaffold, etc.

In some embodiments, the scaffold comprises a protein scaffold.

In some embodiments, the polypeptide is covalently linked to a protein subunit of the protein scaffold to form a fusion protein.

In some embodiments, the protein subunit of the protein scaffold comprises a polypeptide with sequence identity of at least 50%, 55%, 60%, 65%, 70%, 75%, 80%, 85%, 90%, 95%, 96%, 97%, 98%, 99%, or 100% to the sequence of a protein listed in Table 12, wherein residues in parentheses are optional.

### Polynucleotide

In another aspect, the disclosure provides a polynucleotide encoding any polypeptide of the disclosure or any nanostructure of the disclosure. The polynucleotide sequence may comprise RNA or DNA. Isolated polynucleotides refers to those that have been removed from their normal surrounding nucleic acid sequences in the genome or in cDNA sequences.

Such polynucleotide sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what polynucleotide sequences will encode the proteins of the disclosure.

In some embodiments, the polynucleotide is a messenger RNA (mRNA).

In another aspect, the disclosure provides a lipid nanoparticle (LNP) comprising an mRNA encoding any polypeptide of the disclosure or any nanostructure of the disclosure.

In another aspect, the disclosure provides an expression vector comprising any polynucleotide of the disclosure operatively linked to a suitable control sequence. “Recombinant expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid responsive). The construction of expression vectors for use in transfecting prokaryotic cells is also well known in the art, and thus can be accomplished via standard techniques. (See, for example, Sambrook, Fritsch, and Maniatis, in: Molecular Cloning, A Laboratory Manual, Cold Spring Harbor Laboratory Press, 1989: Gene Transfer and Expression Protocols. pp. 109-128, ed. E. J. Murray, The Humana Press Inc., Clifton, N.J.), and the Ambion 1998 Catalog (Ambion, Austin, TX). The expression vector can be replicable in the host organisms either as an episome or by integration into host chromosomal DNA. Suitable expression vectors and hosts are well known in the art. In some embodiment, the expression vector comprises a plasmid. However, the disclosure is intended to include other expression vectors that serve equivalent functions, such as viral vectors.

In another aspect, the disclosure provides a host cell comprising any polynucleotide of the disclosure, any expression vector of the disclosure, and/or any polypeptide of the disclosure. The cells can be transiently or stably transfected or transduced. Such transfection or transduction of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2nd Ed. (R. I. Freshney. 1987. Liss, Inc. New York, NY).

In some embodiments, the polynucleotide is a messenger RNA (mRNA). Methods of generating a polynucleotide either by chemical synthesis or by in vitro transcription (IVT) (for mRNA) are well known in the art.

The basic structure of an encoding mRNA can resemble “mature” eukaryotic mRNA and can include some or all of the following features including (i) a protein-encoding open reading frame (ORF), flanked by (ii) S′ and 3′ untranslated regions (UTRs), and at the end sides (iii) a 7-methyl guanosine 5′ cap structure and (iv) a 3′ poly(A) tail. The non-coding structural features can be individually optimized to modulate the mRNA stability, translation efficiency, and immunogenicity. By incorporating modified nucleosides, mRNA transcripts referred to as “nucleoside-modified mRNA” can be produced with reduced immunostimulatory activity, and therefore an improved safety profile can be obtained. In addition, modified nucleosides allow the design of mRNA vaccines with strongly enhanced stability and translation capacity, as they can avoid the direct antiviral pathways that are induced by type IFNs and are programmed to degrade and inhibit invading mRNA. For instance, the replacement of uridine with pseudouridine reduces the activity of 2′-5′-oligoadenylate synthetase, which regulates the mRNA cleavage by RNase L. In addition, lower activities are measured for protein kinase R, an enzyme that is associated with the inhibition of the mRNA translation process.

Polynucleotides of the disclosure may include one or more modified (e.g., altered or alternative) nucleobases, nucleosides, nucleotides, or combinations thereof. The polynucleotides can include any useful modification or alteration, such as to the nucleobase, the sugar, or the internucleoside linkage (e.g., to a linking phosphate/to a phosphodiester linkage/to the phosphodiester backbone). In certain some embodiment, alterations (e.g., one or more alterations) are present in each of the nucleobase, the sugar, and the internucleoside linkage. Alterations according to the present disclosure may be alterations of polynucleotides, e.g., the substitution of the 2′-OH of the ribofuranosyl of an RNA ring to 2′-H, threose nucleic acids (TNAs), glycol nucleic acids (GNAs), peptide nucleic acids (PNAs), locked nucleic acids (LNAs), or hybrids thereof.

The polynucleotide may include a 5′-cap structure. The 5′-cap structure of a polynucleotide is involved in nuclear export and increasing polynucleotide stability and binds the mRNA Cap Binding Protein (CBP). A S′-UTR may be provided as a flanking region to the mRNA. In some embodiment the polynucleotide sequence is codon-optimized. The polynucleotide may include a “polyA sequence” or “polyadenylation signal,” terms used interchangeably.

### Pharmaceutical Compositions

In another aspect, the present disclosure provides pharmaceutical compositions, comprising one or more polypeptides, fusion proteins, protein nanostructures, compositions, nucleic acids, LNPs, expression vectors, and/or host cells of the disclosure and a pharmaceutically acceptable carrier. The pharmaceutical compositions of the disclosure can be used, for example, in the methods of the disclosure described below. The pharmaceutical composition may comprise in addition to the polypeptide of the disclosure (a) a lyoprotectant; (b) a surfactant: (c) a bulking agent: (d) a tonicity adjusting agent: (e) a stabilizer; (f) a preservative and/or (g) a buffer, for example.

In some embodiments, the buffer is a Tris buffer, a histidine buffer, a phosphate buffer, a citrate buffer or an acetate buffer. The pharmaceutical composition may also include a lyoprotectant, e.g, sucrose, sorbitol or trehalose. In certain some embodiments, the pharmaceutical composition includes a preservative e.g, benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In vet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer. e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polypeptides, fusion proteins, protein nanostructures, compositions, nucleic acids, expression vectors, and/or host cells may be the sole active agent in the pharmaceutical composition, or the composition or vaccine may further comprise one or more other active agents suitable for an intended use.

The polypeptides, fusion proteins, protein nanostructures, compositions, pharmaceutical compositions, nucleic acids, expression vectors, and/or host cells of the disclosure may be used for any suitable purpose, including but not limited to treat or limit development of CMV infections. For example, the polypeptides, fusion proteins, protein nanostructures, compositions, pharmaceutical compositions, nucleic acids, expression vectors, and/or host cells may be used to elicit an immune response to CMV. One type of immune response is a B-cell response, which results in the production of antibodies against the antigen that elicited the immune response.

Protective antibodies elicited by methods of this disclosure can protect against viral infections by affecting any step in the life cycle of the virus. For example, protective antibodies may prevent CMV from attaching to a cell, entering a cell, releasing viral ribonucleoproteins into the cytoplasm, forming new viral particles in the infected cell, and/or budding new viral particles from the infected host cell membrane. Antibodies elicited by the methods of this disclosure preferably prevent CMV from attaching to or entering the host cell, prevent fusion of viral membranes with endosomal membranes, or prevent release of newly formed virus from the infected host cell.

One aspect of this disclosure is a vaccine composition (vaccine) comprising any polypeptide, fusion protein, or composition disclosed herein, to protect subjects against infection by CMV. Vaccines of this disclosure can also contain other components such as adjuvants, buffers and the like. Exemplary adjuvants include aluminum phosphate, benzyalkonium chloride, ubenimex, and QS21: genetic adjuvants such as the IL-2 gene or fragments thereof, the granulocyte macrophage colony-stimulating factor (GM-CSF) gene or fragments thereof, the IL-18 gene or fragments thereof, the chemokine (C-C motif) ligand 21 (CCL21) gene or fragments thereof, the IL-6 gene or fragments thereof, CpG. LPS. TLR agonists, and other immune stimulatory genes: protein adjuvants such IL-2 or fragments thereof, the granulocyte macrophage colony-stimulating factor (GM-CSF) or fragments thereof, IL-18 or fragments thereof, the chemokine (C-C motif) ligand 21 (CCL21) or fragments thereof, IL-6 or fragments thereof, CpG, LPS, TLR agonists and other immune stimulatory cytokines or fragments thereof; lipid adjuvants such as cationic liposomes, N3 (cationic lipid), monophosphoryl lipid A (MPL1); other adjuvants including cholera toxin, enterotoxin. Fms-like tyrosine kinase-3 ligand (Flt-3L), bupivacaine, marcaine, and levamisole.

### Methods of Use and Treatment

This disclosure provides methods of vaccinating a subject against CMV, the method comprising administering polypeptides, fusion proteins, protein nanostructurex, compositions, pharmaceutical compositions, nucleic acids, LNPs, expression vectors, and/or host cells to the subject such that an immune response against CMV is produced in the subject.

The subject may be any suitable subject, including but not limited to humans and other primates, including non-human primates such as chimpanzees and other apes and monkey species: farm animals such as cattle, sheep, pigs, seals, goats and horses: domestic mammals such as dogs and cats; laboratory animals including rodents such as mice, rats and guinea pigs; birds, including domestic, wild and game birds such as chickens, turkeys and other gallinaceous birds, ducks, geese, and the like. In some embodiments, the subject is a newborn. In some embodiments, the subject is an infant. In some embodiments, the subject is pregnant. In some embodiments, the subject is immunocompromised or is at risk of becoming immunocompromised such as a candidate for organ, stem cell or bone marrow transplant.

In the vaccination methods of this disclosure, the subject being vaccinated may have been exposed to CMV. As used herein, the term “exposed” indicate the subject has come in contact with a person or animal that is known to be infected with CMV. Vaccines of this disclosure may be administered by any suitable technique, by means including, but not limited to, traditional syringes, needleless injection devices, or microprojectile bombardment gene guns. Suitable routes of administration include, but are not limited to, parenteral delivery, such as intramuscular, intradermal, subcutaneous, or intramedullary injections, as well as intrathecal, direct intraventricular, intravenous, intraperitoneal, intranasal, or intraocular injection.

A composition provided herein may be co-administered with other treatments, such as other vaccines. In some embodiments, a subject treated in accordance with a method provided herein may also be administered one or more seasonal or pandemic vaccines such as an influenza vaccine or a SARS-Cov2 vaccine. In some embodiments, a subject treated in accordance with the methods provided herein may also be administered a pneumococcal. Recombinant Zoster (Shingles), or Tdap vaccine. One, two, or more vaccines may be co-administered with an immunogenic composition provided herein. “Co-administration” includes both concurrently as well as subsequent administration. For example, the one, two, or more vaccines and an immunogenic composition provided herein may be administered on the same day. In some embodiment, the one, two, or more vaccines and an immunogenic composition provided herein are administered within 1 hour, within 2 hours, within 3 hours, within 4 hours, within 5 hours, within 6 hours, within 8 hours, withing 10 hours, or within 12 hours.

In another aspect, provide herein is a method of treating a subject suffering from viral infection. As used herein, “treat” or “treating” includes, but is not limited to accomplishing one or more of the following: (a) reducing viral titer in the subject: (b) limiting any increase of viral titer in the subject: (c) reducing the severity of viral infection symptoms; (d) limiting or preventing development of symptoms after viral infection: (e) inhibiting worsening of symptoms of viral infection: (f) limiting or preventing recurrence of symptoms of viral infection in subjects that were previously symptomatic for viral infection; and/or (g) increasing survival. In some embodiments, a method of vaccinating decreases the subject's risk of becoming infected with a virus. In some embodiments, a method of vaccinating limits the development of a viral infection. In some embodiments, a method of vaccinating decreases the severity of the symptoms of a viral infection.

In some embodiment, the methods provided herein may be used to prevent a viral infection or illness (e.g., pneumonia or acute respiratory disease) in a subject. As used herein, “prevent” or “preventing” includes, but is not limited to accomplishing one or more of the following: (a) generating an immune response (antibody and/or cell-based, e.g., CD4 T cells, memory B cells, and/or CD8 T cells) to a virus in the subject expected to confer protection against infection caused by or associated with Cytomegalovirus in the subject: (b) generating neutralizing antibodies against Cytomegalovirus in the subject expected to reduce the severity of one or more Cytomegalovirus-caused symptoms in the subject; (c) preventing

Cytomegalovirus infection in a subject, detected as an increase in the titer of the virus of the subject or by an increase in one or more symptoms of viral infection: (d) reducing the risk of Cytomegalovirus infection within a population of subjects; or (e) causing a seroresponse (or seroconversion) of a subject, such as generating neutralizing antibodies against Cytomegalovirus at least 4-fold higher than a baseline antibody level in the subject. Prevention may be assessed by comparing immune responses, especially correlates of protection, in subjects administered a vaccine to the same subjects before administration (termed baseline), subjects administered a placebo, or subjects administered a comparator vaccine.

As used herein, “limiting” the development a viral infection, refers to accomplishing one or more of the following: (a) generating an immune response (antibody and/or cell-based, e.g., CD4 T cells, memory B cells, and/or CD8 T cells) to Cytomegalovirus in the subject expected to limit an increase in viral titer or symptoms in the subject: (b) generating neutralizing antibodies against Cytomegalovirus in the subject at a level expected to limit an increase in viral titer or symptoms in the subject: (c) causing reduced viral titers in the subject after exposure to Cytomegalovirus compared to subjects not administered the protein complex; and (d) caused reduced incidence or severity of symptoms after Cytomegalovirus infection. Illustrative symptoms of Cytomegalovirus infection include, but are not limited to, fever, fatigue, swollen glands, sore throat and muscle ache.

Further, the methods provided herein may be used to prevent or limit development of infection with an original strain of Cytomegalovirus and/or infection with a variant strain of Cytomegalovirus.

Clinical efficacy of a Cytomegalovirus vaccine can be assessed by various means known in the art, including but not limited to placebo-controlled clinical efficacy studies to measure viral load or symptoms of viral disease in vaccinated versus control subjects. Correlates of protection may also be defined, such as neutralizing antibody titers (typically expressed as geometric mean titers), fold increases above baselines (typically expressed as geometric fold rise), and seroresponse rate (a percentage of subjects that achieve a fold rise in neutralizing antibody titers above a predetermined threshold). Guidance on direct and surrogate measures of clinical efficacy for respiratory diseases are available, for example, in Guidance for Industry: Clinical Data Needed to Support the Licensure of Seasonal Inactivated Influenza Vaccines. U.S. Food & Drug Administration (May 2007) and Respiratory Syncytial Virus Infection: Developing Antiviral Drugs for Prophylaxis and Treatment Guidance for Industry. U.S. Food & Drug Administration (October 2017).

In some embodiments, the methods described herein generate an immune response in a subject in the subject not known to be infected with a virus, wherein the immune response serves to limit development of infection and symptoms of a viral infection. In some embodiments, the immune response comprises generation of neutralizing antibodies and/or cell-based responses against a virus. In some embodiments, the immune response comprises generation of gB protein-specific or other trimer-specific responses with a mean geometric titer of at least 1×10³, at least 1×10⁴, at least 1×10⁵, at least 1×10⁶, at least 1×10⁷, at least 1×10⁸, or at least 1×10⁹. In a further embodiment, the immune response comprises generation of antibodies against multiple antigenic epitopes on the gB protein-specific or other viral protein trimer.

In another aspect, the disclosure provides a method of generating an immune response to CMV gB protein in a subject in need thereof, comprising administering to the subject, an effective amount of the pharmaceutical composition of the disclosure.

In one aspect, the methods provided herein may result in an increase in antibody titers in a subject, e.g., in an increase in virus-specific neutralizing antibodies or virus-specific binding antibodies. Antibody titers may be determined using any suitable assays known in the art or described herein including, without limitation, binding enzyme-linked immunosorbent assays (ELISA), enzyme-linked immune absorbent spot (ELISpot), competition ELISAs, immunoprecipitation, immunoblotting, and agglutination assays.

In some embodiments, a neutralization or microneutralization (MN) assay may be used to measure increases in neutralizing antibodies in a subject after administration of a protein complex as described herein. Microneutralization refers to a neutralizing assay performed in a miniaturized format, such as a 96-well plate. A (micro) neutralization assay is used to test for the inhibition of a virus by antibodies (e.g., purified antibodies, serum, or plasma). The assay measures the level of antibodies present in a sample that are able to neutralize a virus in vitro. Generally, microneutralization assays for clinical samples are performed with a serial dilution of serum mixed with a fixed concentration of virus. Methods for performing (micro) neutralization assays are well known. Illustrative microneutralization assays are described, see, e.g., van Baleen et al. Vaccine 35 (2017) 46-52.

If neutralizing antibodies specific for Cytomegalovirus are present in a sample, the virus will be neutralized, and infection of cells (e.g., HEp-2 cells) is inhibited. Immunofluorescence levels indicating viral infection may be analyzed using, e.g., a CTL ImmunoSpot® UV analyzer, equipped with BioSpot® analysis software for automated counting of infected cells. Results are generally reported in international units per milliliter (IU/mL).

By “baseline” is meant a measurement of antibodies prior to administration of the first dose of an immunogenic composition provided herein.

In another aspect, the methods provided herein may result in an increase in immune cells in a subject (e.g., an increase in virus-specific memory B cells and/or virus-specific T cells). The number of immune cells in a subject may be determined using any suitable assay known in the art or described herein, including, without limitation, FACS and flow cytometry.

In some embodiments, the composition is administered via any suitable route, including intranasally, sublingually, orally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally.

In another aspect, the disclosure provides a method of treating or preventing CMV infection in a subject in need thereof, comprising administering to the subject, an effective amount of the pharmaceutical composition of the disclosure.

In another aspect, the disclosure provides a vaccine comprising one or more of the polypeptides, fusion protein, protein nanostrucure, composition, nucleic acid, LNP expression vector, and/or the host cell of the disclosure and a pharmaceutically acceptable carrier.

In some embodiments, the vaccine of the disclosure further comprises any other component as appropriate for an intended use, including but not limited to any other CMV antigens, including but not limited to one or more of CMV proteins gH, gL, UL128, UL130 and UL131, or antigenic portions thereof; or a pentamer complex of CMV proteins gH, gL, UL128, UL130 and UL131 or antigenic portions thereof.

In another aspect, the disclosure provides a method for treating or limiting development of an CMV infection, comprising administering to a subject in need thereof an amount effective to treat or limit development of the CMV infection of a polypeptide, fusion protein, composition, vaccine, nucleic acid, expression vector, host cell, pharmaceutical composition, and/or vaccine of the disclosure.

### Kits

The disclosure further provides kits, which may be used to prepare and administer compositions of the disclosure. In some embodiments, a kit provided herein comprises a composition as disclosed herein, and instructions for use in a method of the disclosure. In some embodiments, a kit comprises one or more unit doses as disclosed herein, and instructions for use in a method of the disclosure. In some embodiments, the kit comprises a vial comprising a single dose of a pharmaceutical composition provided herein. In some embodiments, a kit comprises a vial comprising multiple doses provided herein. In some embodiments, a kit further comprises instructions for use of the pharmaceutical composition. In some embodiments, a kit further comprises a diluent for preparing dilutions of the pharmaceutical composition prior to administration. In some embodiments, the pharmaceutical composition comprises an adjuvant. In some embodiments, a kit comprises a pharmaceutical composition and an adjuvant which may be mixed prior to administration. In another aspect, the disclosure provides a composition, method, or use as described herein.

### EXAMPLES

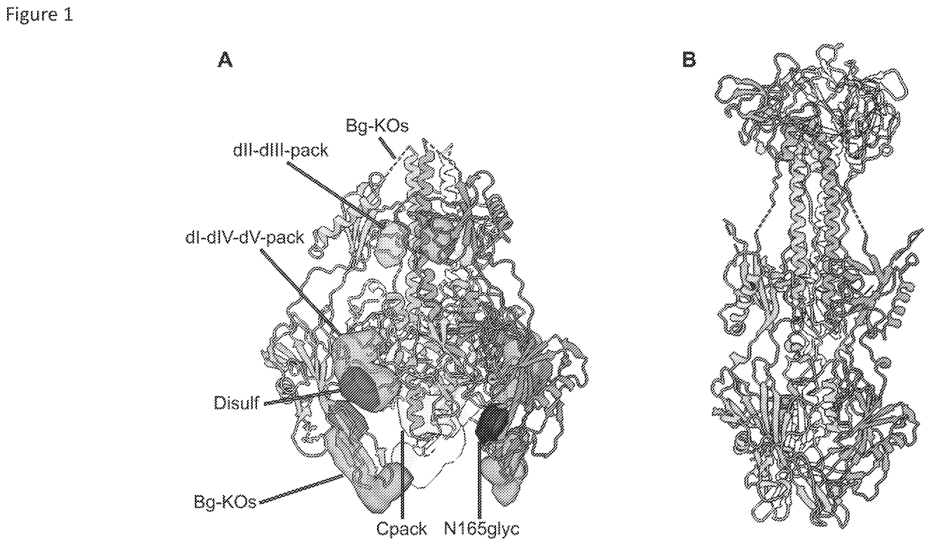

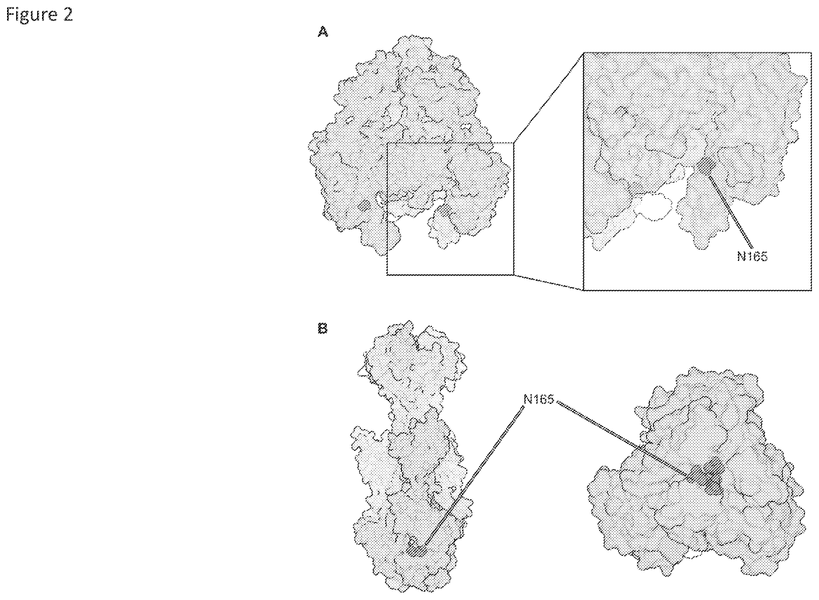

Provided are sequences of recombinant CMV gB proteins in which, while not wishing to be held by theory, mutations have been added to disrupt the postfusion state and/or stabilize the prefusion state of the gB protein. Regions of gB (FIG. 1A) were identified with sub-optimal local stability for the prefusion state, and specific mutations which are intended to both improve these structural features and/or destabilize the postfusion state are provided. For example, insertion of a glycosylation site at a position exposed in the postfusion conformation is shown in FIG. 2B (compared to prefusion. FIG. 2A). Designed gB antigens have been characterized by size exclusion chromatography (SEC) as trimeric but show distinctly different biophysical profiles as assessed by nano differential scanning fluorimetry (nanoDSF) and dynamic light scattering (DLS). Further negative stain electron microscopy (NS-EM) confirms that designed CMV antigens less often favor the postfusion state, while instead showing minor conformations that better resemble the prefusion state. Together, the mutations provided herein are potentially useful for diverse forms of vaccines containing recombinant gB antigens, including protein-based, nucleic acid-based or vectored vaccines.

### Example 1: Materials and Methods

Expression and protein purification: Genes encoding mutant CMV gB antigens (corresponding to residues 20-698, 20-702, or 20-704 of SEQ ID NO: 1) were cloned in the pCMV/R vector using the XbaI and AvrII restriction sites. In addition to various designed mutations, all sequences also contained the mutations I156H, H157R, W240N. Y242T, C246S, R457S, R460S to make fusion loops more polar, remove an unpaired cysteine, and remove a poly-basic cleavage site. All sequences were preceded by a “MGILPSPGMPALLSLVSLLSVLLMGCVAETGT” (SEQ ID NO: 304) secretion signal and C-terminally tagged with either a “GGSHHHHHHHH” (SEQ ID NO: 305) sequence to allow for purification, a “GSGYIPEAPRDGQAYVRKDGEWVLLSTFLGGHHHHHHHH” (SEQ ID NO: 306) histidine-tagged T4 fibritin foldon sequence to allow for both improved trimerization and purification, or a “GSRMKQIEDKIEEILSKIYHIENEIARIKKLIGERGGHHHHHHHH” (SEQ ID NO: 307) histidine-tagged GCN4 sequence to allow for both improved trimerization and purification.

All proteins were produced in Expi293F cells grown in suspension using Expi293F expression medium (Life Technologies) at 37° C., 70% humidity. 8% CO₂ rotating at 150 rpm. Cell cultures were transfected using PEI-MAX (Polyscience) with cells grown to a density of 3.0 million cells per mL and cultivated for 3 days. Supernatants were clarified by centrifugations at 4000 ref, addition of PDADMAC to a final concentration of 0.0375% (Sigma Aldrich) and a second spin at 4000 ref.

His-tagged proteins were purified from clarified supernatants via a batch bind method where each clarified supernatant was supplemented with 1 M Tris-HCl pH 8.0 to a final concentration of 45 mM and 5 M NaCl to a final concentration of ˜310 mM. Talon cobalt affinity resin (Takara) was added to the treated supernatants and allowed to incubate for 15 minutes with gentle shaking. Resin was collected using vacuum filtration with a 0.2 μm filter and transferred to a gravity column. The resin was washed with 20 mM Tris pH 8.0, 300 mM NaCl, and the protein was eluted with 3 column volumes of 20 mM Tris pH 8.0, 300 mM NaCl, 300 mM imidazole. Protein purity after affinity purification was assessed by SDS-PAGE (both reducing and non-reducing). Eluates were concentrated and applied to a Superose 6 Increase 10/300 column (Cytiva) pre-equilibrated with 25 mM tris pH 8.0, 150 mM NaCl, 5% glycerol for preparative size exclusion chromatography (SEC). Peaks corresponding to the trimeric species were identified based on elution volume. Purified protein was stored at 4° C.

Thermal melts: All proteins were diluted to 1 mg/mL in 25 mM tris pH 8.0, 150 mM NaCl, 5% glycerol prior to thermal measurements. Measurements of melting temperatures were determined from thermal denaturation melt curves using an UNcle™ (UNchained Labs) based on the barycentric mean (BCM) of intrinsic tryptophan fluorescence, with data collected from 20-95° C. using a thermal ramp of 1° C. per minute in a background of 25 mM Tris pH 8.0, 150 mM NaCl and 5% glycerol. Melting temperatures were calculated using UNcle™ analysis software.

Negative stain electron microscopy: Protein was adsorbed to glow-discharged carbon-coated copper grids for 1 min prior to a 3× wash with water and 2% uranyl formate staining Micrographs were recorded using the Leginon software on a 100 kV FEI Tecnai G2 Spirit with a Gatan Ultrascan 4000 4k×4k CCD camera at 67,000 nominal magnification. The defocus ranged from 1.0 to 2.0 mm and the pixel size was 1.6 Å. Particles were picked automatically in a reference free manner using DogPicker. Contrast transfer function (CTF) estimation was performed using GCTF. Class averages were generated using Relion2.1.

### Example 2: Computational Design of gB Protein

Rationale structure-based design using the crystal structure of CMV gB protein (PDB 7KDP) was used to identify amino-acid substitutions that would stabilize the prefusion conformation or destabilize the postfusion conformation of gB protein. Briefly, the prefusion (PDB 7KDP) and postfusion (PDB 7KDD) structures of CMV gB were analyzed using PyMol software to identify regions with different structural conformations in either state. For “C terminus” and “domain-domain” groups (Table 13), multiple residues in these regions were selected for design using computational protocols from Rosetta software (Leman et al. Nat. Methods 17 (7): 665-680 (2020)), in which selected residues were allowed to be simultaneously mutated by Rosetta to improve noncovalent interactions within the prefusion structure. Output sequences from Rosetta with different types and/or numbers of mutations were scored using Rosetta in both prefusion and postfusion structures to predict energetic effects towards both states (Table 13. “energetic difference”), and sets of mutations that were predicted to stabilize the prefusion state and/or destabilize the postfusion state were selected for experimental analysis. For the “disulfide” group (Table 13), pairs of residues were selected that are distant in the postfusion structure, but in close enough proximity in the prefusion structure to form a disulfide bond if both mutated to cysteine. For the “glycosylation” group (Table 13), the E167T mutation was made to glycosylate N165. N165 is strongly buried in the postfusion structure and not capable of accommodating an added glycan without introducing steric clashes, but solvent-exposed in the prefusion structure to allow ample space for an added glycan.

Table 13 shows predicted computationally predicted stabilization of CMV gB protein. Representative wild-type sequence (SEQ ID NO: 1-3) was modified to include amino acid substitutions at I156H, H157R. W240N. Y242T, C246S, R457S, R460S. This representative sequence was further modified as in Table 13.

TABLE 13
Summary of CMV mutation data

| | | | Energenic difference | | |
|---|---|---|---|---|---|
| | | | | (preTrimer − | (mutPre − |
| | | | | preMonomer) − | wtPre) − |
| | | | preTrimer − | (postTrimer − | (mutPost − |
| Group | Mutant | Mutations | preMonomer | postMonomer) | wtPost) |
| C | Cpack1- | E686L, Y690F, | −156 REU | postfusion | −32 REU |
| terminus | short | R693V, V694L | | 14 REU | |
| | Cpack1- | E686L, Y690F, | −212 REU | prefusion | −406 REU |
| | long | R693V, V694L, | | −41 REU | |
| | | K700A, V702Q | | | |
| | Cpack2- | D679N, L680E, | −166 REU | postfusion | −24 REU |
| | short | E681N, R685Q, | | 6 REU | |
| | | E686L, Y690F, | | | |
| | | R693V, V694L, | | | |
| | | K695D | | | |
| | Cpack2- | D679N, L680E, | −236 REU | prefusion | −396 REU |
| | long: | E681N, R685Q, | | −64 REU | |
| | | E686L, Y690F, | | | |
| | | R693V, V694L, | | | |
| | | K695D, K700A, | | | |
| | | V701L, V702Q | | | |
| | Cpack3- | V677T, F678N, | −166 REU | prefusion | −168 REU |
| | short | D679N, L680E, | | −32 REU | |
| | | E681N, E682S, | | | |
| | | R685Q, E686L, | | | |
| | | N688E, Y690F, | | | |
| | | R693V, V694L, | | | |
| | | K695D | | | |
| | Cpack3- | V677T, F678N, | −179 REU | prefusion | −573 REU |
| | long | D679N, L680E, | | −113 REU | |
| | | E681N, E682S, | | | |
| | | R685Q, E686L, | | | |
| | | N688E, Y690F, | | | |
| | | R693V, V694L, | | | |
| | | K695D, D699K, | | | |
| | | K700A, V701L, | | | |
| | | V702Q | | | |
| | Cpack4- | V677T, F678N, | −243 REU | prefusion | −325 REU |
| | short | D679H, L680E, | | −104 REU | |
| | | E681N, E682S, | | | |
| | | M684S, R685Q, | | | |
| | | E686L, F687A, | | | |
| | | N688E, Y690F, | | | |
| | | Q692S, R693V, | | | |
| | | V694L, K695D, | | | |
| | | Y696R | | | |
| | Cpack4- | V677T, F678N, | −261 REU | prefusion | −562 REU |
| | long | D679H, L680E, | | −122 REU | |
| | | E681N, E682S, | | | |
| | | M684S, R685Q, | | | |
| | | E686L, F687A, | | | |
| | | N688E, Y690F, | | | |
| | | Q692S, R693V, | | | |
| | | V694L, K695D, | | | |
| | | Y696R, D699K, | | | |
| | | K700A, V701L, | | | |
| | | V702E | | | |
| Domain- | dII-dIII- | S367I, T374F | No effect on trimer stability in either | | −67 REU |
| domain | pack1 | | prefusion or postfusion conformation. | | |
| | dI-dIV- | Y218F, Q591F, | Slightly | postfusion | −62 REU |
| | dV-pack1 | S668A | stabilizes the | 9 REU | |
| | | | trimer | | |
| | | | −14 REU | | |
| | dI-dIV- | N220S, V552L | −50 REU | prefusion | −158 REU |
| | dV-pack2 | | | -38 REU | |
| | dI-dIV- | Y218F, N220S, | −62 REU | prefusion | −177 REU |
| | dV-pack3 | V552L, Q591F, | | -33 REU | |
| | | S668A | | | |
| | dI-dIV- | Y218F, N220S, | −61 REU | prefusion | −189 REU |
| | dV-pack4 | V552L, Q591Y, | | -32 REU | |
| | | S668A | | | |
| | dI-dIV- | N220S, V552L, | −45 REU | prefusion | −252 REU |
| | dV-pack5 | Q591Y | | -33 REU | |
| | dI-dIV- | N220S, V552L, | −48 REU | prefusion | −151 REU |
| | dV-pack6 | Q591F | | -36 REU | |
| | | | C-beta distance | C-beta distance in | |
| | | | in prefusion | postfusion | |
| Disulfide | Disulf1 | D217C, S587C | 3.9 Angstrom | 134.6 Angstrom | |
| | Disulf2 | D217C, Y589C | 4.4 Angstrom | 138.4 Angstrom | |
| Glycosylation | N165glyc | E167T | N/A | | |

The first value of Table 13 is the energetic difference between the prefusion trimer and the prefusion monomer (pre Trimer-preMonomer). The second value of Table 13 is the difference between the above and the equivalent difference for the postfusion state ((pre Trimer-preMonomer)−(post Trimer-postMonomer)). The final value of the Table 13 is the difference is energy change between pre- and postfusion states by adding the mutations ((mutPre-wtPre)−(mutPost-wtPost)). “REU” means “Rosetta Energy Units”. Methods for calculating these parameters are provided in Park et al. J. Chem. Theory & Computation 12 (12) 6201-12 (2016). Monomer calculations come from a model where each chain in the trimer is translated 1000 Angstrom apart to simulate a dissociated state.

### Example 3: Results

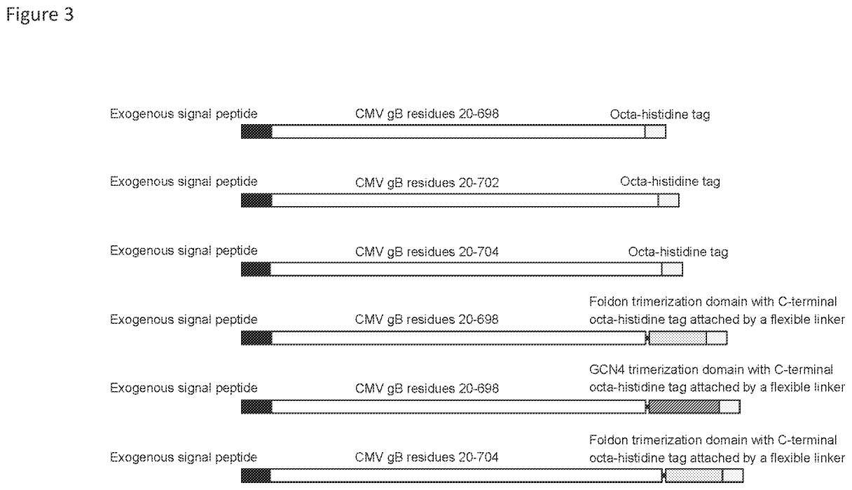

Referring to FIG. 3, recombinant gB designs tested all included at least the soluble ectodomain of gB from the Towne strain from residues 20-698, with some designs containing additional residues up to residue 704. All designs tested featured an exogenous N-terminal signal peptide and a C-terminal octa-histidine tag, with some designs also featuring an exogenous trimerization domain between the C-terminus of the gB ectodomain and the octa-histidine tag. All designs tested also featured polar mutations in the fusion loop to assist protein solubility (I156H. H157R, W240N, Y242T), a mutation to an unpaired cysteine (C246S) and mutations to a polybasic cleavage site (R457S, R460S). Collectively, these are termed “Bg-KO.” Without being bound by theory, these mutations should impact the expression level but not the prefusion:postfusion ratio. Therefore, it is expected that constructs lacking these amino acid substitutions would still have the desired prefusion preference induced by the designed mutations described in Example 2 and tested here. In other words, the Bg-KO substitutions are an optional feature of the constructs.

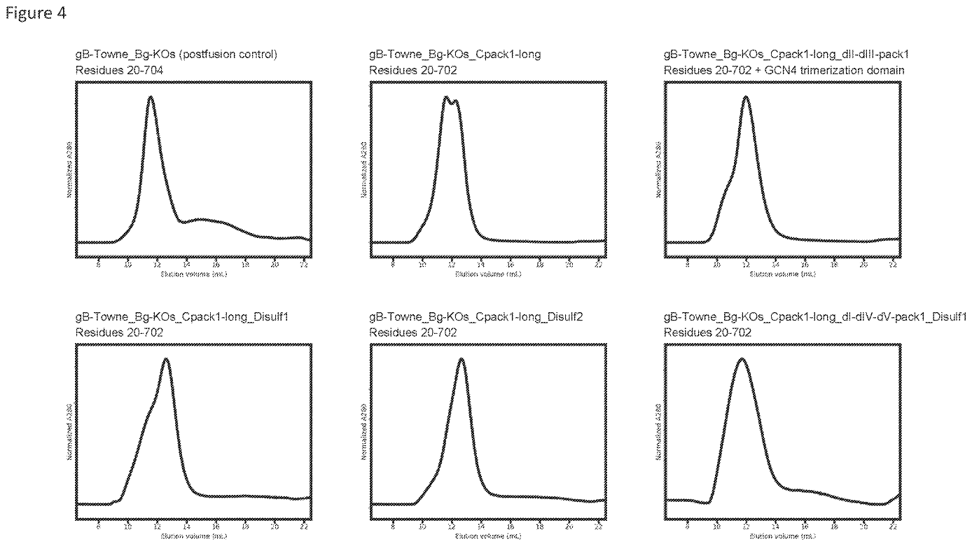

FIG. 4 shows size exclusion chromatography (SEC) (Superose 6 Increase 10/300 GL, Cytiva) of multiple gB antigens featuring designed mutations. The designed constructs had homogenous elution profiles consistent with trimeric gB antigens. A comparable postfusion gB antigen lacking designed mutations is shown as a reference. The dimensions of the postfusion structure of gB (PDB 7KDD) notably feature a great length (˜17 nm) and a relatively small width (˜7 nm). Because SEC elution volume is influenced by a molecule's radius of gyration and hydrodynamic diameter, prefusion gB should eluate later than postfusion gB. The five different designs with the Cpack1-long mutations alone or with additional mutations show later elution volumes, which is consistent with the samples containing an increase ratio of prefusion to postfusion gB.

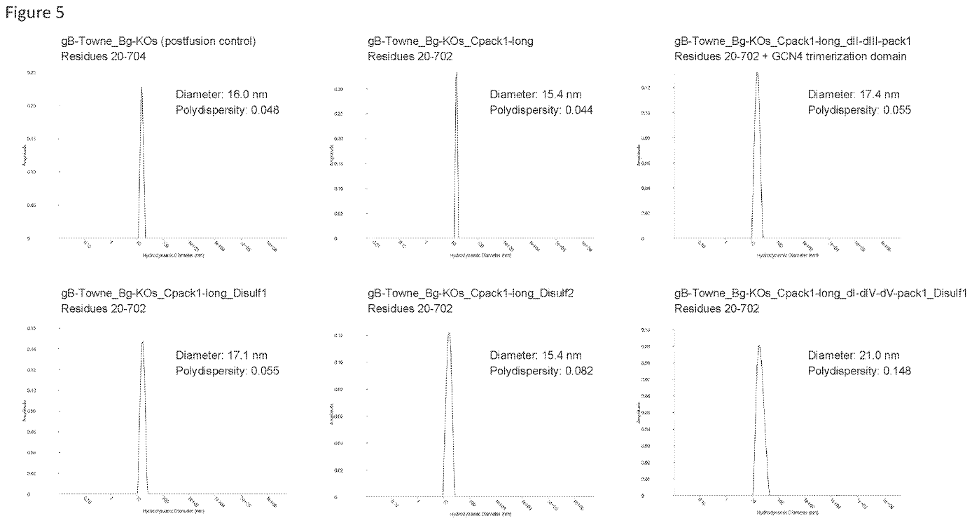

FIG. 5 shows dynamic light scattering (DLS) of multiple gB antigens featuring designed mutations, showing distinctly different hydrodynamic diameters and polydispersities, which is indicative of conformational changes. A comparable gB antigen lacking designed mutations is shown as a reference. Prefusion gB should have a larger hydrodynamic diameter than postfusion gB. Cpack1-long, in combination with Disulf1, dII-dIII pack 1 and GCN4, or Disulf 1 and di-dIV-dV-pack 1, show larger DLS diameter than the postfusion control.

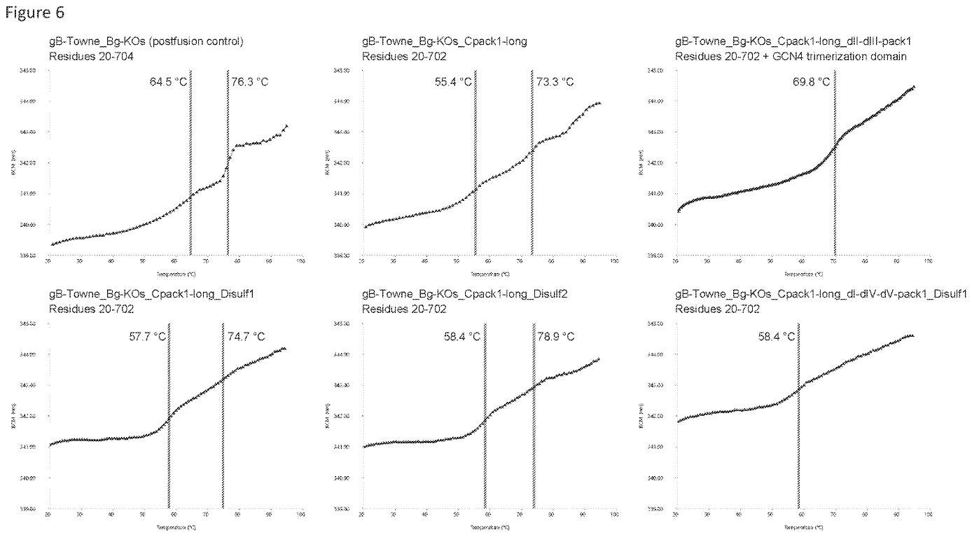

FIG. 6 shows nano differential scanning fluorimetry (nanoDSF) of multiple gB antigens featuring designed mutations, with nanoDSF measured using intrinsic tryptophan fluorescence. A comparable gB antigen lacking designed mutations is shown as a reference. Prefusion gB should be less thermally stable than postfusion gB, due to the low energy of the postfusion state needed for membrane fusion. Compared to the postfusion control, which showed two melting transitions, addition of designed mutations led to different thermal profiles in which one or more melting temperatures was lowered.

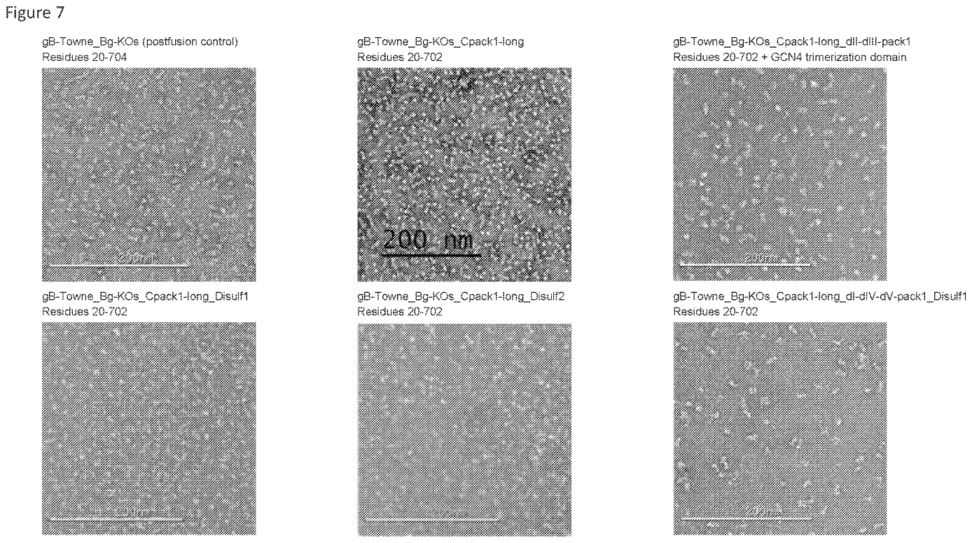

FIG. 7 shows negative stain electron micrographs (NS-EM) micrographs of multiple gB antigens featuring designed mutations. A comparable gB antigen lacking designed mutations is shown as a reference, which more monodispersely features an elongated profile consistent with the postfusion state. All constructs with designed mutations showed less postfusion character compared to the postfusion control. The combination of Cpack1-long and other design features led to greater prevalence of conformations that are not similar to the postfusion structure. Specifically, particles with more spherical character were observed with the combination of Cpack1-long, Disulf1 and dI-dIV-dV-pack 1 mutations, which is more similar to the prefusion structure.

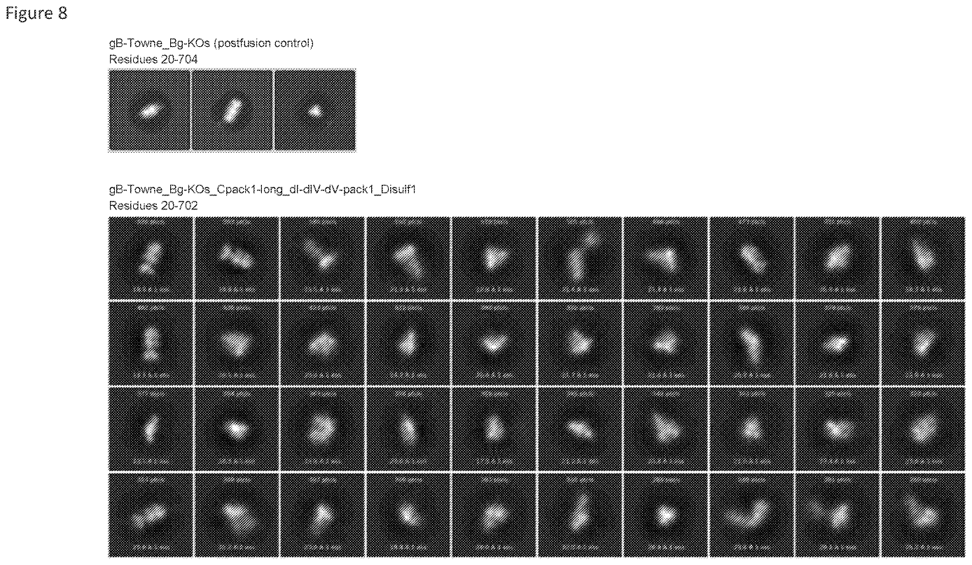

FIG. 8 shows NS-EM 2D class averages of one gB antigen featuring designed mutations and a comparable gB antigen lacking designed mutations.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure. Accordingly, the scope of the invention is limited only by the following claims.

What is claimed is:

1. A polypeptide, comprising an ectodomain of CMV gB in the prefusion conformation, wherein the ectodomain comprises, at positions relative to SEQ ID NO: 1:
a. 1, 2, 3, 4 or more amino acid substitutions selected from the group consisting of E686L, R693V, V694L, Y690F, K700A, V702E, D679H, E681N, K695D, L680E, R685Q, V701L, E682S, F678N, N688E, V677T, D699K, F687A, M684S, Q692S, and Y696R, or substitutions at the same amino acid positions;
b. the amino acid substitution S367I, the amino acid substitution T374F, or the amino acid substitutions S367I and T374F, or substitutions at positions 367 and/or 374;
c. 1, 2, 3, 4 or more amino acid substitutions selected from the group consisting of Q591F, Q591Y, S668A, Y218F, N220S, V552L, or substitutions at the same amino acid positions;
d, the amino acid substitution E167T; and/or
e. 1, 2, 3, 4 or more amino acid substitutions selected from the group consisting of C246S, H157R, I156H, R457S, R460S, W240N, and Y242T.

2. The polypeptide of claim 1, wherein the ectodomain comprises the amino acid subsitutions D217C and S587C.

3. The polypeptide of claim 1, wherein the ectodomain comprises the amino acid subsitutions D217C and Y589C.

4. The polypeptide of any one of claims 1-3, wherein the ectodomain comprises 1, 2, 3, 4 or more amino acid substitutions selected from the group consisting of E686L, R693V, V694L, Y690F, K700A, V702E, D679H, E681N, K695D, L680E, R685Q, V701L, E682S, F678N, N688E, V677T, D699K, F687A, M684S, Q692S, and Y696R.

5. The polypeptide of claim 4, wherein the ectodomain comprises the amino acid substitutions E686L, R693V, V694L, and Y690F.

6. The polypeptide of claim 4, wherein the ectodomain comprises the amino acid substitutions E686L, R693V, V694L, Y690F, K700A, and V702Q.

7. The polypeptide of claim 4, wherein the ectodomain comprises the amino acid substitutions E686L, R693V, V694L, Y690F, D679N, E681N, K695D, L680E, and R685Q.

8. The polypeptide of claim 4, wherein the ectodomain comprises the amino acid substitutions E686L, R693V, V694L, Y690F, K700A, V702Q, D679N, E681N, K695D, L680E, R685Q, and V701L.

9. The polypeptide of claim 4, wherein the ectodomain comprises the amino acid substitutions E686L, R693V, V694L, Y690F, D679N, E681N, K695D, L680E, R685Q, E682S, F678N, N688E, and V677T.

10. The polypeptide of claim 4, wherein the ectodomain comprises the amino acid substitutions E686L, R693V, V694L, Y690F, K700A, V702Q, D679N, E681N, K695D, L680E, R685Q, V701L, E682S, F678N, N688E, V677T, and D699K.

11. The polypeptide of claim 4, wherein the ectodomain comprises the amino acid substitutions E686L, R693V, V694L, Y690F, D679H, E681N, K695D, L680E, R685Q, E682S, F678N, N688E, V677T, F687A, M684S, Q692S, and Y696R.

12. The polypeptide of claim 4, wherein the ectodomain comprises the amino acid substitutions E686L, R693V, V694L, Y690F, K700A, V702E, D679H, E681N, K695D, L680E, R685Q, V701L, E682S, F678N, N688E, V677T, D699K, F687A, M684S, Q692S, and Y696R.

13. The polypeptide of any one of claims 1-12, wherein the ectodomain comprises the amino acid substitution S367I or the amino acid substitution T374F.

14. The polypeptide of any one of claims 1-12, wherein the ectodomain comprises the amino acid substitutions S367I and T374F.

15. The polypeptide of any one of claims 1-14, wherein the ectodomain comprises 1, 2, 3, 4 or more amino acid substitutions selected from the group consisting of Q591F, Q591Y, S668A, Y218F, N220S, and V552L.

16. The polypeptide of claim 15, wherein the ectodomain comprises the amino acid substitutions Q591F, S668A, and Y218F.

17. The polypeptide of claim 15, wherein the ectodomain comprises the amino acid substitutions N220S and V552L.

18. The polypeptide of claim 15, wherein the ectodomain comprises the amino acid substitutions Q591F, S668A, Y218F, N220S, and V552L.

19. The polypeptide of claim 15, wherein the ectodomain comprises the amino acid substitutions Q591Y, S668A, Y218F, N220S, and V552L.

20. The polypeptide of claim 15, wherein the ectodomain comprises the amino acid substitutions Q591Y, N220S, and V552L.

21. The polypeptide of claim 15, wherein the ectodomain comprises the amino acid substitutions Q591F, N220S, and V552L.

22. The polypeptide of any one of claims 1-21, wherein the ectodomain comprises amino acid substitution E167T or E167S.

23. The polypeptide of any one of claims 1-22, wherein the ectodomain comprises 1, 2, 3, 4 or more amino acid substitutions selected from the group consisting of C246S, H157R, I156H, R457S, R460S, W240N, and Y242T.

24. The polypeptide of claim any preceding claim, wherein the polypetide comprises any one combination of amino acid substitutions listed in Table 10.

25. The polypeptide of claim any preceding claim, wherein the polypetide comprises a polypeptide sequence at least 75%, 80%, 85%, 90%, 95%, 96%, 97%, 98%, 99%, or 100% identical to a polypeptide sequence in Table 10 without the signal peptide, or an antigenic fragment thereof.

26. The polypeptide of claim 1, wherein the CMV gB protein adopts a prefusion conformation in the absence of a fusion inhibitor, optionally N-(4-[({(1S)-1-[3,5-bis(trifluoromethyl)phenyl|ethyl}carbamothioyl) amino]phenyl}-1,3-thiazole-4-carboxamide.

27. The polypeptide of any one of claims 1-23, wherein the polypeptide comprises, as a C-terminal fusion to the ectodomain, a trimerization domain.

28. The polypeptide of any one of claims 1-23, wherein the polypeptide comprises, as a C-terminal fusion to the ectodomain, a nanostructure assembly domain.

29. The polypeptide of claim 28, wherein the assembly domain comprises a polypeptide sequence at least 75%, 80%, 85%, 90%, 95%, 96%, 97%, 98%, 99%, or 100% identical to 153-50A (SEQ ID NO: 19), 153-50A.1 (SEQ ID NO: 21), 153-50A.1NegT2 (SEQ ID NO: 22), or 153-50A.1PosT1 (SEQ ID NO: 23).

30. The polypeptide of claim 28, wherein the assembly domain comprises a polypeptide sequence at least 75%, 80%, 85%, 90%, 95%, 96%, 97%, 98%, 99%, or 100% identical to 13-01 (SEQ ID NO: 8).

31. The polypeptide of claim 28, wherein the assembly domain comprises a polypeptide sequence at least 75%, 80%, 85%, 90%, 95%, 96%, 97%, 98%, 99%, or 100% identical to SEQ ID NO: 27, SEQ ID NO: 28, SEQ ID NO: 29, SEQ ID NO: 30, or SEQ ID NO: 31.

32. The polypeptide of claim 28, wherein the assembly domain comprises a polypeptide sequence at least 75%, 80%, 85%, 90%, 95%, 96%, 97%, 98%, 99%, or 100% identical to 153_dn5B (SEQ ID NO: 18).

33. A nanostructure comprising the polypeptide of any one of claim 29-32.

34. The nanostructure of claim 33, wherein the nanostructure comprises a second polypeptide.

35. The nanostructure of claim 33, wherein the second polypetpide comprises a polypeptide sequence at least 75%, 80%, 85%, 90%, 95%, 96%, 97%, 98%, 99%, or 100% identical to 153-50B (SEQ ID NO: 20), 153-50B.1 (SEQ ID NO: 24), 153-50B.1NcgT2 (SEQ ID NO: 25), or 153-50B.4PosT1 (SEQ ID NO: 26).

36. The nanostructure of claim 33, wherein the second polypeptide comprises a polypeptide sequence at least 75%, 80%, 85%, 90%, 95%, 96%, 97%, 98%, 99%, or 100% identical to 153_dn5A* (SEQ ID NO: 15), 153_dn5A.1 (SEQ ID NO: 16), or 153_dn5A.2 (SEQ ID NO: 17).

37. A polynucleotide encoding the polypeptide of any one of claims 1-32 or the nanostructure of any one of claims 33-36.

38. The polynucleotide of claim 37 is a messenger RNA (mRNA).

39. A lipid nanoparticle (LNP) comprising an mRNA encoding the polypeptide of any one of claims 1-32 or the nanostructure of any one of claims 33-36.

40. A pharmaceutical composition comprising the polypetide of any one of claims 1-32, the nanostructure of any one of claims 33-36, the polynucleotide of any one of claims 37-38 or the LNP of claim 39.

41. A method of generating an immune response to CMV gB protein in a subject in need thereof, comprising administering to the subject, an effective amount of the pharmaceutical composition of claim 40.

42. A method of treating or preventing CMV infection in a subject in need thereof, comprising administering to the subject, an effective amount of the pharmaceutical composition of claim 40.

43. An expression vector comprising the polynucleotide of any one of claims 37-38 operatively linked to a suitable control sequence.

44. A host cell comprising the polynucleotide of any one of claims 37-38, the expression vector of claim 43, and/or the polypeptide of any proceeding claim.

45. A polypeptide comprising an amino acid sequence at least 90%, 91%, 92%, 93%, 94%, 95%, 96%, 97%, 98%, or 99% identical to the amino acid sequence of SEQ ID NO: 1 or SEQ ID NO:3 residues 91-702, or an antigenic fragment thereof, wherein
(a) residues 698-702 are optional if they do not include a mutation;
(b) SEQ ID NO:3 residue 456 is absent, and
(c) the polypeptide or antigenic fragment thereof comprises 1 or more combination of mutations relative to SEQ ID NO: 1 or SEQ ID NO:3 residues 91-702 listed in Table 2, Table 3, or Table 4.

46. The polypeptide of claim 45, wherein the polypeptide or antigenic fragment thereof comprises 1 or more combination of mutations relative to SEQ ID NO: 1 or SEQ ID NO: 3 residues 91-702 listed in Table 2 or Table 3.

47. The polypeptide of claim 45, wherein the polypeptide or antigenic fragment thereof comprises 1 or more combination of mutations relative to SEQ ID NO: 1 or SEQ ID NO: 3 residues 91-702 listed in Table 2.

48. The polypeptide of any one of claims 45-47, wherein the polypeptide or antigenic fragment thereof further comprises 1, 2, 3, 4, 5, 6, 7, 8, 9, 10, 11, 12, 13, 14, 15, 16, 17, 18, 19, 20, or more mutations relative to relative to SEQ ID NO: 1 or SEQ ID NO: 3 residues 91-702 listed in Table 5.

49. The polypeptide of any one of claims 45-48, wherein the polypeptide or antigenic fragment thereof further comprises 1, 2, 3, 4, 5, 6, 7, 8, 9, or all 10 mutations relative to relative to SEQ ID NO: 1 or SEQ ID NO:3 residues 91-702 listed in Table 6.

50. The polypeptide of any one of claims 45-48, wherein the polypeptide or antigenic fragment thereof further comprises 1, 2, 3, 4, 5, 6, 7, 8, 9, 10, 11, or all 12, relative to relative to SEQ ID NO: 1 or SEQ ID NO:3 residues 91-702 listed in Table 7.

51. The polypeptide of any one of claims 45-50, wherein the polypeptide or antigenic fragment thereof further comprises a combination of mutations relative to relative to SEQ ID NO: 1 or SEQ ID NO:3 residues 91-702 listed in Table 8.

52. The polypeptide of any one of claims 45-51, comprising an amino acid sequence at least 90%, 91%, 92%, 93%, 94%, 95%, 96%, 97%, 98%, or 99% identical to the amino acid sequence selected from the sequences listed in Table 9, or an antigenic fragment thereof.

53. The polypeptide of any one of claims 45-52, comprising an amino acid sequence at least 90%, 91%, 92%, 93%, 94%, 95%, 96%, 97%, 98%, or 99% identical to the amino acid sequence selected from the sequences listed in Table 10, or an antigenic fragment thereof.

54. The polypeptide of any one of claims 45-53, further comprising any other functional domain as appropriate for an intended use, including but not limited to a secretion signal located at the N-terminus of the polypeptide, wherein the signal sequence may be any suitable signal sequence as appropriate for an intended use.

55. A fusion protein comprising:
(a) the polypeptide or antigenic fragment thereof of any one of claims 45-54; and
(b) a multimerization domain.

56. The fusion protein of claim 55, wherein the multimerization domain comprises a polypeptide with sequence identity of at least 50%, 55%, 60%, 65%, 70%, 75%, 80%, 85%, 90%, 95%, 96%, 97%, 98%, 99%, or 100% to the sequence of a protein listed in Table 12, wherein residues in parentheses are optional.

57. A composition, comprising one or more of the polypeptides or fusion proteins of any preceding claim linked to a scaffold.

58. The composition of claim 57, wherein the scaffold comprises a protein scaffold.

59. The composition of claim 58, wherein the polypeptide is covalently linked to a protein subunit of the protein scaffold to form a fusion protein.

60. The composition of claim 59, wherein the protein subunit of the protein scaffold comprises a polypeptide with sequence identity of at least 50%, 55%, 60%, 65%, 70%, 75%, 80%, 85%, 90%, 95%, 96%, 97%, 98%, 99%, or 100% to the sequence of a protein listed in Table 12, wherein residues in parentheses are optional.

61. A nucleic acid encoding the polypeptide or fusion protein of any preceding claim.

62. An expression vector comprising the nucleic acid of claim 61 operatively linked to a suitable control sequence.

63. A host cell comprising the nucleic acid of claim 61, the expression vector of claim 62, and/or the polypeptide or fusion protein of any preceding claim.

64. A pharmaceutical composition, comprising
(a) one or more of the polypeptides, fusion protein, composition, nucleic acid, expression vector, and/or the host cell of any preceding claim; and
(b) a pharmaceutically acceptable carrier.

65. A vaccine comprising
(a) one or more of the polypeptides, fusion protein, composition, nucleic acid, expression vector, and/or the host cell of any preceding claim; and
(b) a pharmaceutically acceptable carrier.

66. The vaccine of claim 65 further comprising any other component as appropriate for an intended use, including but not limited to any other CMV antigens, including but not limited to one or more of CMV proteins gH, gL, UL128, UL130 and UL131, or antigenic portions thereof; or a pentamer complex of CMV proteins gH, gL, UL128, UL130 and UL131 or antigenic portions thereof.

67. A method for treating or limiting development of an CMV infection, comprising administering to a subject in need thereof an amount effective to treat or limit development of the CMV infection of a polypeptide, fusion protein, composition, vaccine, nucleic acid, expression vector, host cell, pharmaceutical composition, and/or vaccine of any preceding claim.

68. A composition, method, or use as described herein.
